(19) United States
(12) Patent Application Publication
(10) Pub. No.: US 2004/0082786 A1
(43) Pub. Date: Apr. 29, 2004

(54) Piperazine based inhibitors of factor xa

(76) Inventors: Bing-Yan Zhu, Palo Alto, CA (US); Zhaozhong Jon Jia, San Mateo, CA (US); Penglie Zhang, Foster City, CA (US); Wenrong Huang, Cupertino, CA (US); Yanhong Wu, Foster City, CA (US); Jingmei Fan Zuckett, Bellevue, MA (US); Erik A. Goldman, Berkeley, CA (US); Lingyan Wang, East Brunswick, NJ (US); Younghong Song, Foster City, CA (US); Robert M. Scarborough, Half Moon Bay, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/381,928

(22) PCT Filed: Oct. 1, 2001
(86) PCT No.: PCT/US01/30315

(51) Int. Cl. C 07D 4 3/02; C07D 241/04

(52) U.S. Cl. 544/360; 544/295; 544/370; 544/384; 544/386

(57) ABSTRACT

Novel compounds of the general formulae (I) or (II), including their pharmaceutically acceptable isomers, salts, hydrates, solvates and prodrug derivatives having activity against mammalian factor Xa are described. Compositions containing such compounds are also described. The compounds and the compositions are useful in vitro or in vivo for preventing or treating conditions in mammals characterized by undesired thrombosis.

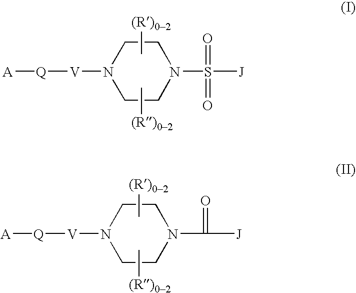

### FIELD OF THE INVENTION

The invention relates to novel piperazine-containing compounds including their pharmaceutically acceptable isomers, salts, hydrates, solvates and prodrug derivatives, and pharmaceutically acceptable compositions thereof which are potent and highly selective inhibitors of isolated factor Xa or when assembled in the prothrombinase complex. These compounds show selectivity for factor Xa versus other proteases of the coagulation (e.g. thrombin, fVIIa, fIXa) or the fibrinolytic cascades (e.g. plasminogen activators, plasmin). In another aspect, the present invention relates to novel piperazine-containing compounds including their pharmaceutically acceptable isomers, salts, hydrates, solvates and prodrug derivatives factor Xa-inhibiting compounds, and pharmaceutically acceptable compositions thereof which are useful as potent and specific inhibitors of blood coagulation in mammals. In yet another aspect, the invention relates to methods for using these inhibitors as therapeutic agents for disease states in mammals characterized by undesired thrombosis or coagulation disorders.

### BACKGROUND OF THE INVENTION

Hemostasis, the control of bleeding, occurs by surgical means, or by the physiological properties of vasoconstriction and coagulation. This invention is particularly concerned with blood coagulation and ways in which it assists in maintaining the integrity of mammalian circulation after injury, inflammation, disease, congenital defect, dysfunction or other disruption. Although platelets and blood coagulation are both involved in thrombus formation, certain components of the coagulation cascade are primarily responsible for the amplification or acceleration of the processes involved in platelet aggregation and fibrin deposition.

Thrombin is a key enzyme in the coagulation cascade as well as in hemostasis. Thrombin plays a central role in thrombosis through its ability to catalyze the conversion of fibrinogen into fibrin and through its potent platelet activation activity. Direct or indirect inhibition of thrombin activity has been the focus of a variety of recent anticoagulant strategies as reviewed by Claeson, G., “Synthetic Peptides and Peptidomimetics as Substrates and Inhibitors of Thrombin and Other Proteases in the Blood Coagulation System”, Blood Coag. Fibrinol. 5, 411-436 (1994). Several classes of anticoagulants currently used in the clinic directly or indirectly affect thrombin (i.e. heparins, low-molecular weight heparins, heparin-like compounds and coumarins).

A prothrombinase complex, including Factor Xa (a serine protease, the activated form of its Factor X precursor and a member of the calcium ion binding, gamma carboxyglutamyl (Gla)-containing, vitamin K dependent, blood coagulation glycoprotein family), converts the zymogen prothrombin into the active procoagulant thrombin. Unlike thrombin, which acts on a variety of protein substrates as well as at a specific receptor, factor Xa appears to have a single physiologic substrate, namely prothrombin. Since one molecule of factor Xa may be able to generate up to 138 molecules of thrombin (Elodi et al., Thromb. Res. 15, 617-619 (1979)), direct inhibition of factor Xa as a way of indirectly inhibiting the formation of thrombin may be an efficient anticoagulant strategy. Therefore, it has been suggested that compounds which selectively inhibit factor Xa may be useful as in vitro diagnostic agents, or for therapeutic administration in certain thrombotic disorders, see e.g., WO 94/13693.

Polypeptides derived from hematophagous organisms have been reported which are highly potent and specific inhibitors of factor Xa. U.S. Pat. No. 4,588,587 describes anticoagulant activity in the saliva of the Mexican leech, Haementeria officinalis. A principal component of this saliva was shown to be the polypeptide factor Xa inhibitor, antistasin (ATS), by Nutt, E. et al., “The Amino Acid Sequence of Antistasin, a Potent Inhibitor of Factor Xa Reveals a Repeated Internal Structure”, J. Biol. Chem., 263, 10162-10167 (1988). Another potent and highly specific inhibitor of Factor Xa, called tick anticoagulant peptide (TAP), has been isolated from the whole body extract of the soft tick Ornithidoros moubata, as reported by Waxman, L., et al., “Tick Anticoagulant Peptide (TAP) is a Novel Inhibitor of Blood Coagulation Factor Xa” Science, 248, 593-596 (1990).

Factor Xa inhibitory compounds which are not large polypeptide-type inhibitors have also been reported including: Tidwell, R. R. et al., “Strategies for Anticoagulation With Synthetic Protease Inhibitors. Xa Inhibitors Versus Thrombin Inhibitors”, Thromb. Res., 19, 339-349 (1980); Turner, A. D. et al., “p-Amidino Esters as Irreversible Inhibitors of Factor IXa and Xa and Thrombin”, Biochemistry, 25, 4929-4935 (1986); Hitomi, Y. et al., “Inhibitory Effect of New Synthetic Protease Inhibitor (FUT-175) on the Coagulation System”, Haemostasis, 15, 164-168 (1985); Sturzebecher, J. et al., “Synthetic Inhibitors of Bovine Factor Xa and Thrombin. Comparison of Their Anticoagulant Efficiency”, Thromb. Res., 54, 245-252 (1989); Kam, C. M. et al., “Mechanism Based Isocoumarin Inhibitors for Trypsin and Blood Coagulation Serine Proteases: New Anticoagulants”, Biochemistry, 27, 2547-2557 (1988); Hauptmann, J. et al., “Comparison of the Anticoagulant and Antithrombotic Effects of Synthetic Thrombin and Factor Xa Inhibitors”, Thromb. Haemost., 63, 220-223 (1990); and the like.

Others have reported Factor Xa inhibitors which are small molecule organic compounds, such as nitrogen containing heterocyclic compounds which have amidino substituent groups, wherein two functional groups of the compounds can bind to Factor Xa at two of its active sites. For example, WO 98/28269 describes pyrazole compounds having a terminal C(═NH)—NH₂ group; WO 97/21437 describes benzimidazole compounds substituted by a basic radical which are connected to a naphthyl group via a straight or branched chain alkylene, —C(═O) or —S(═O)₂ bridging group; WO 99/10316 describes compounds having a 4-phenyl-N-alkylamidino-piperidine and 4-phenoxy-N-alkylamidino-piperidine group connected to a 3-amidinophenyl group via a carboxamidealkyleneamino bridge; and EP 798295 describes compounds having a 4-phenoxy-N-alkylamidino-piperidine group connected to an amidinonaphthyl group via a substituted or unsubstituted sulfonamide or carboxamide bridging group.

There exists a need for effective therapeutic agents for the regulation of hemostasis, and for the prevention and treatment of thrombus formation and other pathological processes in the vasculature induced by thrombin such as restenosis and inflammation. In particular, there continues to be a need for compounds which selectively inhibit factor Xa or its precursors. Compounds are needed which selectively or preferentially bind to Factor Xa. Compounds with a higher affinity for binding to Factor Xa than to thrombin are desired, especially those compounds having good bioavailability or other pharmacologically desirable properties.

### SUMMARY OF THE INVENTION

The present invention relates to novel piperazine-containing compounds including their pharmaceutically acceptable isomers, salts, hydrates, solvate and prodrug derivatives, which have particular biological properties and are useful as potent and specific inhibitors of blood coagulation in mammals. According to the invention, the compounds can act as potent and highly selective inhibitors of isolated Factor Xa or when assembled in the prothrombinase complex. The invention also provides compositions containing such compounds. The compounds of the invention may be used as diagnostic reagents or as therapeutic reagents for disease states in mammals which have coagulation disorders. Thus, the invention further provides methods for preventing or treating a condition in a mammal characterized by undesired thrombosis by administration of a therapeutically effective amount of a compound of the invention and a pharmaceutically acceptable carrier. Optionally, the methods of the invention comprise administering a pharmaceutical composition of the invention in combination with an additional therapeutic agent such as an antithrombotic and/or a thrombolytic agent and/or an anticoagulant. According to the invention, such conditions include, for example, any thrombotically mediated acute coronary or cerebrovascular syndrome, any thrombotic syndrome occurring in the venous system, any coagulopathy, and any thrombotic complications associated with extracorporeal circulation or instrumentation, and for the inhibition of coagulation in biological samples (e.g. stored blood products and samples).

The invention provides a compound of the general formulae (I) or (II):

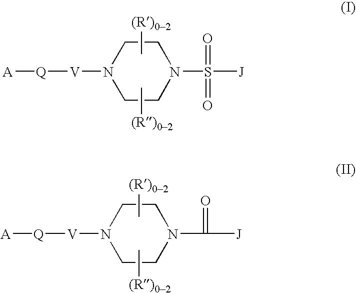

wherein:

A is a member selected from the group consisting of:

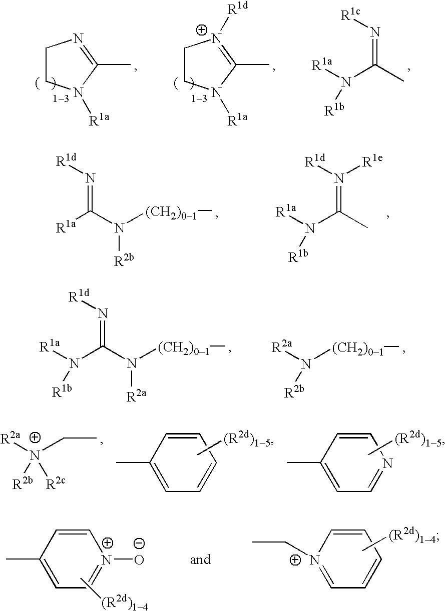

R^{1a}, R^{1b}, R^{1d}, and R^{1e} are each independently a H, C₁₋₆ alkyl, C₂₋₆ alkenyl, C₂₋₆ alkynyl, C₃₋₈cycloalkyl, aryl, —C₁₋₆alkylaryl, —C₁₋₆alkyl-OC₁₋₆alkyl, —C₁₋₆alkyl-NRₐR_{b}, —(CH₂)₁₋₆NRₐC(═O)C₁₋₆ alkyl, —(CH₂)₁₋₆C(═O)OH, —(CH₂)₁₋₆C(═O)OC₁₋₆alkyl, or —(CH₂)₁₋₆C(═O)NRₐR_{b}; or R^{1a} and R^{1b} or R^{1a} and R^{1c} or R^{1a} and R^{1d} or R^{1d} and R^{1e} taken together with the nitrogen atom to which they are each attached can form a substituted or unsubstituted 3 to 8 membered heterocyclic or heteroaromatic amine group which, optionally, contains at least one other heteroatom of N, O or S; wherein R^{1a}, R^{1b}, R^{1d}, or R^{1e} is optionally substituted with at least one of halo, alkyl, alkylideneamine, arylidenamine, cyano, hydroxy, alkoxy, amino, amidino, guanidino, imino, amido, acid, ester, keto, aldehyde, dioxolane, furanyl, piperidinyl, piperazinyl, pyrrolidinyl, aryl, morpholinyl, and thiomorpholinyldioxide;

R^{1c} is H, C₁₋₆alkyl or C₃₋₈cycloalkyl;

R^{2a}, R^{2b} and R^{2c} are each independently a H, —C₁₋₆alkyl, —C₂₋₆alkenyl, —C₂₋₆alkynyl, —C₃₋₈cycloalkyl, aryl, —C₁₋₆alkylaryl, —C₁₋₆alkyl-OC₁₋₆alkyl, —C₁₋₆alkyl-NRₐR_{b}, —(CH₂)₁₋₆NRₐC(═O)C₁₋₆ alkyl, —(CH₂)₁₋₆C(═O)OH, —(CH₂)₁₋₆C(═O)OC₁₋₆alkyl, or —(CH₂)₁₋₆C(═O)NRₐR_{b}; or R^{2a} and R^{2b} or R^{1a}, as set forth above, and R^{2a} or R^{1a}, as set forth above, and R^{2b} taken together with the nitrogen atom to which they are each attached can form a substituted or unsubstituted 3 to 8 membered heterocyclic or heteroaromatic amine group which, optionally, contains at least one other heteroatom of N, O or S; wherein R^{2a}, R^{2b} or R^{2c} is optionally substituted with at least one of halo, alkyl, alkylideneamine, arylidenamine, cyano, hydroxy, alkoxy, amino, amidino, guanidino, imino, amido, acid, ester, keto, aldehyde, dioxolane, furanyl, piperidinyl, piperazinyl, pyrrolidinyl, aryl, morpholinyl, and thiomorpholinyldioxide;

R^{2d} is —SO₂NRₐR_{b}, —SO₂C₁₋₆alkyl, —CN, —C₀₋₆alkylNRₐR_{b}, —C(═NH)—NRₐR_{b}, or —C(═O)—NRₐR_{b}, where Rₐ and R_{b} are each as set forth below;

Rₐ and R_{b} are independently H, —C₁₋₆alkyl, —C₂₋₆alkenyl, —C₂₋₆alkynyl, aryl; or Rₐ and R_{b} taken together with the nitrogen to which they are attached form azetidinyl, pyrrolidinyl, piperidinyl, morpholinyl, thiomorpholinyl and its oxidized forms, piperazinyl, 4-methyl-1-piperazinyl, morpholinylcarbaldehyde, piperazinylcarbaldehyde or thiomorpholinylcarbaldehyde and its oxidized forms;

V is —CH₂—, or —C(═O)—;

Q is a member selected from the group consisting of:

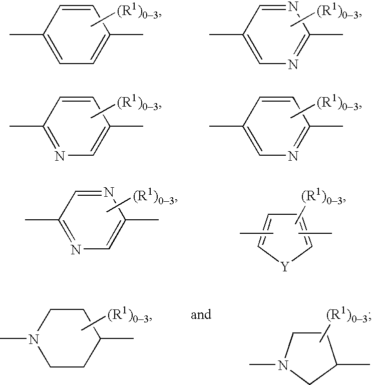

Y is NH, NMe, O, or S;

R¹ is H, —Cl, —Br, —I, —F, —C₁₋₆alkyl, —C₂₋₆alkenyl, —C₀₋₆alkylNRₐR_{b}, —C₀₋₆alkylOH, —C₀₋₆alkylOC₁₋₆alkyl, —SH, —SC₁₋₆alkyl, —SOC₁₋₆alkyl, —SO₂—C₁₋₆alkyl, —CN, —COOH, —COOC₁₋₆alkyl, —CONRₐR_{b}, where Rₐ and R_{b} are each as set forth above;

J is a member selected from the group consisting of:

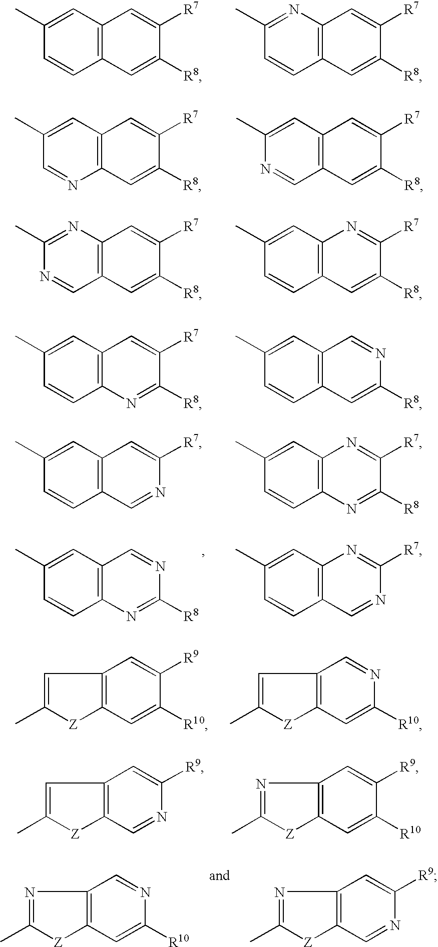

Z is —NR⁶—, —O— or —S—;

R⁶ is H, C₁₋₆alkyl or C₃₋₈cycloalkyl;

R⁷ and R⁸ are independently H, —Cl, —Br, —I or —F, where at least one of R⁷ and R⁸ is not hydrogen; and

R⁹ and R¹⁰ are independently H, —Cl, —Br, —I or —F, where at least one of R⁹ and R¹⁰ is not hydrogen;

R′ and R″ are independently selected from —H, —C₁₋₆alkyl, —C₁₋₆alkyl-OH, —C₁₋₆alkyl-NRₐR_{b}, —C₁₋₆alkylCN, —C₁₋₆alkylCO₂H, —C₁₋₆alkylCO₂C₁₋₆alkyl, and —C₁₋₆alkylCONRₐR_{b}, wherein Rₐ and R_{b} are the same as defined above;

and all pharmaceutically acceptable isomers, salts, hydrates, solvates and prodrug derivatives thereof.

### DETAILED DESCRIPTION OF THE INVENTION

Definitions

In accordance with the present invention and as used herein, the following terms are defined with the following meanings, unless explicitly stated otherwise.

The term “alkenyl” refers to a trivalent straight chain or branched chain unsaturated aliphatic radical. The term “alkynyl” (or “alkinyl”) refers to a straight or branched chain aliphatic radical that includes at least two carbons joined by a triple bond. If no number of carbons is specified alkenyl and alkinyl each refer to radicals having from 2-12 carbon atoms.

The term “alkyl ” refers to saturated aliphatic groups including straight-chain, branched-chain and cyclic groups having the number of carbon atoms specified, or if no number is specified, having up to 12 carbon atoms. The term “cycloalkyl” as used herein refers to a mono-, bi-, or tricyclic aliphatic ring having 3 to 14 carbon atoms and preferably 3 to 7 carbon atoms.

As used herein, the terms “carbocyclic ring structure” and “C₃₋₁₆ carbocyclic mono, bicyclic or tricyclic ring structure” or the like are each intended to mean stable ring structures having only carbon atoms as ring atoms wherein the ring structure is a substituted or unsubstituted member selected from the group consisting of: a stable monocyclic ring which is aromatic ring (“aryl”) having six ring atoms; a stable monocyclic non-aromatic ring having from 3 to 7 ring atoms in the ring; a stable bicyclic ring structure having a total of from 7 to 12 ring atoms in the two rings wherein the bicyclic ring structure is selected from the group consisting of ring structures in which both of the rings are aromatic, ring structures in which one of the rings is aromatic and ring structures in which both of the rings are non-aromatic; and a stable tricyclic ring structure having a total of from 10 to 16 atoms in the three rings wherein the tricyclic ring structure is selected from the group consisting of: ring structures in which three of the rings are aromatic, ring structures in which two of the rings are aromatic and ring structures in which three of the rings are non-aromatic. In each case, the non-aromatic rings when present in the monocyclic, bicyclic or tricyclic ring structure may independently be saturated, partially saturated or fully saturated. Examples of such carbocyclic ring structures include, but are not limited to, cyclopropyl, cyclobutyl, cyclopentyl, cyclohexyl, adamantyl, cyclooctyl, [3.3.0]bicyclooctane, [4.3.0]bicyclononane, [4.4.0]bicyclodecane (decalin), 2.2.2]bicyclooctane, fluorenyl, phenyl, naphthyl, indanyl, adamantyl, or tetrahydronaphthyl (tetralin). Moreover, the ring structures described herein may be attached to one or more indicated pendant groups via any carbon atom which results in a stable structure. The term “substituted” as used in conjunction with carbocyclic ring structures means that hydrogen atoms attached to the ring carbon atoms of ring structures described herein may be substituted by one or more of the substituents indicated for that structure if such substitution(s) would result in a stable compound.

The term “aryl” which is included with the term “carbocyclic ring structure” refers to an unsubstituted or substituted aromatic ring, substituted with one, two or three substituents selected from loweralkoxy, loweralkyl, loweralkylamino, hydroxy, halogen, cyano, hydroxyl, mercapto, nitro, thioalkoxy, carboxaldehyde, carboxyl, carboalkoxy and carboxamide, including but not limited to carbocyclic aryl, heterocyclic aryl, and biaryl groups and the like, all of which may be optionally substituted. Examples of suitable aryl groups include, but are not limited to, phenyl, halophenyl, loweralkylphenyl, naphthyl, biphenyl, phenanthrenyl and naphthacenyl.

The term “arylalkyl” which is included with the term “carbocyclic aryl” refers to one, two, or three aryl groups having the number of carbon atoms designated, appended to an alkyl group having the number of carbon atoms designated. Suitable arylalkyl groups include, but are not limited to, benzyl, picolyl, naphthylmethyl, phenethyl, benzylhydryl, trityl, and the like, all of which may be optionally substituted.

As used herein, the term “heterocyclic ring” or “heterocyclic ring system” is intended to mean a substituted or unsubstituted member selected from the group consisting of stable monocyclic ring having from 5-7 members in the ring itself and having from 1 to 4 hetero ring atoms selected from the group consisting of N, O and S; a stable bicyclic ring structure having a total of from 7 to 12 atoms in the two rings wherein at least one of the two rings has from 1 to 4 hetero atoms selected from N, O and S, including bicyclic ring structures wherein any of the described stable monocyclic heterocyclic rings is fused to a hexane or benzene ring; and a stable tricyclic heterocyclic ring structure having a total of from 10 to 16 atoms in the three rings wherein at least one of the three rings has from 1 to 4 hetero atoms selected from the group consisting of N, O and S. Any nitrogen and sulfur atoms present in a heterocyclic ring of such a heterocyclic ring structure may be oxidized. Unless indicated otherwise the terms “heterocyclic ring” or “heterocyclic ring system” include aromatic rings, as well as non-aromatic rings which can be saturated, partially saturated or fully saturated non-aromatic rings. Also, unless indicated otherwise the term “heterocyclic ring system” includes ring structures wherein all of the rings contain at least one hetero atom as well as structures having less than all of the rings in the ring structure containing at least one hetero atom, for example bicyclic ring structures wherein one ring is a benzene ring and one of the rings has one or more hetero atoms are included within the term “heterocyclic ring systems” as well as bicyclic ring structures wherein each of the two rings has at least one hetero atom. Moreover, the ring structures described herein may be attached to one or more indicated pendant groups via any hetero atom or carbon atom which results in a stable structure. Further, the term “substituted” means that one or more of the hydrogen atoms on the ring carbon atom(s) or nitrogen atom(s) of the each of the rings in the ring structures described herein may be replaced by one or more of the indicated substituents if such replacement(s) would result in a stable compound. Nitrogen atoms in a ring structure may be quaternized, but such compounds are specifically indicated or are included within the term “a pharmaceutically acceptable salt” for a particular compound. When the total number of O and S atoms in a single heterocyclic ring is greater than 1, it is preferred that such atoms not be adjacent to one another. Preferably, there are no more that 1 O or S ring atoms in the same ring of a given heterocyclic ring structure.

Examples of monocyclic and bicyclic heterocyclic ring systems, in alphabetical order, are acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolinyl, indolizinyl, indolyl, 3H-indolyl, isobenzofuranyl, isochromanyl, isoindazolyl, isoindolinyl, isoindolyl, isoquinolinyl (benzimidazolyl), isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyroazolidinyl, pyrazolinyl, pyrazolyl, pyridazinyl, pryidooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofliranyl, tetrahydroisoquinolinyl, tetrahydroquinolinyl, 6H-1,2,5-thiadazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl. Preferred heterocyclic ring structures include, but are not limited to, pyridinyl, furanyl, thienyl, pyrrolyl, pyrazolyl, pyrrolidinyl, imidazolyl, indolyl, benzimidazolyl, 1H-indazolyl, oxazolinyl, or isatinoyl. Also included are fused ring and spiro compounds containing, for example, the above heterocyclic ring structures.

As used herein the term “aromatic heterocyclic ring system” has essentially the same definition as for the monocyclic and bicyclic ring systems except that at least one ring of the ring system is an aromatic heterocyclic ring or the bicyclic ring has an aromatic or non-aromatic heterocyclic ring fused to an aromatic carbocyclic ring structure.

The terms “halo” or “halogen” as used herein refer to Cl, Br, F or I substituents. The term “haloalkyl”, and the like, refer to an aliphatic carbon radicals having at least one hydrogen atom replaced by a Cl, Br, F or I atom, including mixtures of different halo atoms. Trihaloalkyl includes trifluoromethyl and the like as preferred radicals, for example.

The term “methylene” refers to —CH₂—.

The term “pharmaceutically acceptable salts” includes salts of compounds derived from the combination of a compound and an organic or inorganic acid. These compounds are useful in both free base and salt form. In practice, the use of the salt form amounts to use of the base form; both acid and base addition salts are within the scope of the present invention.

“Pharmaceutically acceptable acid addition salt” refers to salts retaining the biological effectiveness and properties of the free bases and which are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium and magnesium salts. Salts derived from pharmaceutically acceptable organic nontoxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, polyamine resins and the like. Particularly preferred organic nontoxic bases are isopropylamine, diethylamine, ethanolamine, trimethamine, dicyclohexylamine, choline, and caffeine.

“Biological property” for the purposes herein means an in vivo effector or antigenic function or activity that is directly or indirectly performed by a compound of this invention that are often shown by in vitro assays. Effector functions include receptor or ligand binding, any enzyme activity or enzyme modulatory activity, any carrier binding activity, any hormonal activity, any activity in promoting or inhibiting adhesion of cells to an extracellular matrix or cell surface molecules, or any structural role. Antigenic functions include possession of an epitope or antigenic site that is capable of reacting with antibodies raised against it.

In the compounds of this invention, carbon atoms bonded to four non-identical substituents are asymmetric. Accordingly, the compounds may exist as diastereoisomers, enantiomers or mixtures thereof The syntheses described herein may employ racemates, enantiomers or diastereomers as starting materials or intermediates. Diastereomeric products resulting from such syntheses may be separated by chromatographic or crystallization methods, or by other methods known in the art. Likewise, enantiomeric product mixtures may be separated using the same techniques or by other methods known in the art. Each of the asymmetric carbon atoms, when present in the compounds of this invention, may be in one of two configurations (R or S) and both are within the scope of the present invention.

Compounds

The invention provides a compound of the general formulae (I) or (II):

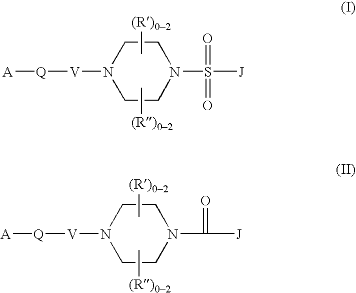

wherein:

A is a member selected from the group consisting of:

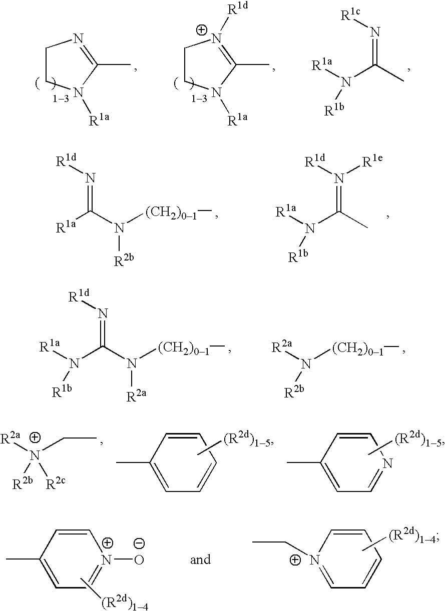

R^{1a}, R^{1b}, R^{1d}, and R^{1e} are each independently a H, C₁₋₆ alkyl, C₂₋₆ alkenyl, C₂₋₆ alkynyl, C₃₋₈cycloalkyl, aryl, —C₁₋₆alkylaryl, —C₁₋₆alkyl-OC₁₋₆alkyl, —C₁₋₆alkyl-NRₐR_{b}, —(CH₂)₁₋₆NRₐC(═O)C₁₋₆ alkyl, —(CH₂)₁₋₆C(═O)OH, —(CH₂)₁₋₆C(═O)OC₁₋₆alkyl, or —(CH₂)₁₋₆C(═O)NRₐR_{b}; or R^{1a} and R^{1b} or R^{1a} and R^{1c} or R^{1a} and R^{1d} or R^{1d} and R^{1e} taken together with the nitrogen atom to which they are each attached can form a substituted or unsubstituted 3 to 8 membered heterocyclic or heteroaromatic amine group which, optionally, contains at least one other heteroatom of N, O or S; wherein R^{1a}, R^{1b}, R^{1d}, or R^{1e} is optionally substituted with at least one of halo, alkyl, alkylideneamine, arylidenamine, cyano, hydroxy, alkoxy, amino, amidino, guanidino, imino, amido, acid, ester, keto, aldehyde, dioxolane, furanyl, piperidinyl, piperazinyl, pyrrolidinyl, aryl, morpholinyl, and thiomorpholinyldioxide;

R^{1c} is H, C₁₋₆alkyl or C₃₋₈cycloalkyl;

R^{2a}, R^{2b} and R^{2c} are each independently a H, —C₁₋₆alkyl, —C₂₋₆alkenyl, —C₂₋₆alkynyl, —C₃₋₈cycloalkyl, aryl, —C₁₋₆alkylaryl, —C₁₋₆alkyl-OC₁₋₆alkyl, —C₁₋₆alkyl-NRₐR_{b}, —(CH₂)₁₋₆NRₐC(═O)C₁₋₆alkyl, —(CH₂)₁₋₆C(═O)OH, —(CH₂)₁₋₆C(═O)OC₁₋₆alkyl, or —(CH₂)₁₋₆C(═O)NRₐR_{b}; or R^{2a} and R^{2b} or R^{1a}, as set forth above, and R^{2a} or R^{1a}, as set forth above, and R^{2b} taken together with the nitrogen atom to which they are each attached can form a substituted or unsubstituted 3 to 8 membered heterocyclic or heteroaromatic amine group which, optionally, contains at least one other heteroatom of N, O or S; wherein R^{2a}, R^{2b} or R^{2c} is optionally substituted with at least one of halo, alkyl, alkylideneamine, arylidenamine, cyano, hydroxy, alkoxy, amino, amidino, guanidino, imino, amido, acid, ester, keto, aldehyde, dioxolane, furanyl, piperidinyl, piperazinyl, pyrrolidinyl, aryl, morpholinyl, and thiomorpholinyldioxide;

R^{2d} is —SO₂NRₐR_{b}, —SO₂C₁₋₆alkyl, —CN, —C₀₋₆alkylNRₐR_{b}, —C(═NH)—NRₐR_{b}, or —C(═O)—NRₐR_{b}, where Rₐ and R_{b} are each as set forth below;

Rₐ and R_{b} are independently H, —C₁₋₆alkyl, —C₂₋₆alkenyl, —C₂₋₆alkynyl; or Rₐ and R_{b} taken together with the nitrogen to which they are attached form azetidinyl, pyrrolidinyl, piperidinyl, morpholinyl, thiomorpholinyl and its oxidized forms, piperazinyl, 4-methyl-1-piperazinyl, morpholinylcarbaldehyde, piperazinylcarbaldehyde or thiomorpholinylcarbaldehyde and its oxidized forms;

V is —CH₂—, or —C(═O)—;

Q is a member selected from the group consisting of:

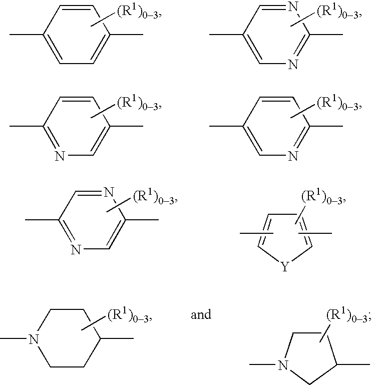

Y is NH, NMe, O, or S;

R¹ is H, —Cl, —Br, —I, —F, —C₁₋₆alkyl, —C₂₋₆alkenyl, —C₀₋₆alkylNRₐR_{b}, —C₀₋₆alkylOH, —C₀₋₆alkylOC₁₋₆alkyl, —SH, —SC₁₋₆alkyl, —SOC₁₋₆alkyl, —SO₂—C₁₋₆alkyl, —CN, —COOH, —COOC₁₋₆alkyl, —CONRₐR_{b}, where Rₐ and R_{b} are each as set forth above;

J is a member selected from the group consisting of:

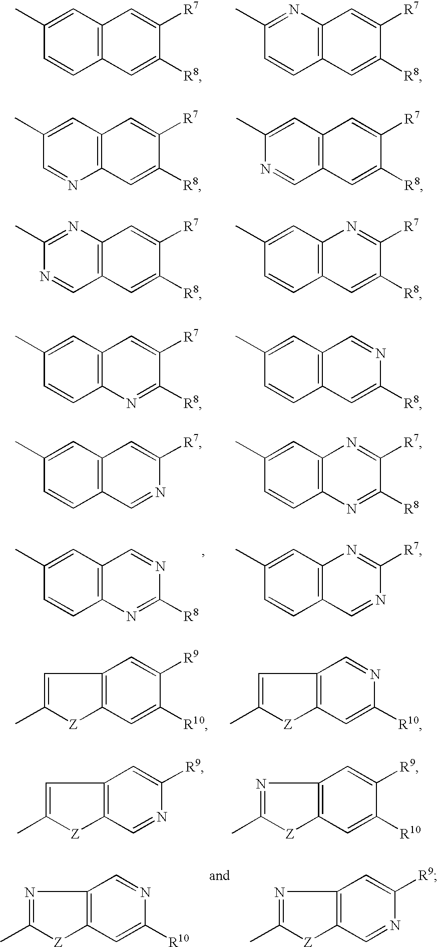

Z is —NR⁶—, —O— or —S—;

R⁶ is H, C₁₋₆alkyl or C₃₋₈cycloalkyl;

R⁷ and R⁸ are independently H, —Cl, —Br, —I or —F, where at least one of R⁷ and R⁸ is not hydrogen; and

R⁹ and R¹⁰ are independently H, —Cl, —Br, —I or —F, where at least one of R⁹ and R¹⁰ is not hydrogen;

R′ and R″ are independently selected from —H, —C₁₋₆alkyl, —C₁₋₆alkyl-OH, —C₁₋₆alkyl-NRₐR_{b}, —C₁₋₆alkylCN, —C₁₋₆alkylCO₂H, —C₁₋₆alkylCO₂C₁₋₆alkyl, and —C₁₋₆alkylCONRₐR_{b}, wherein Rₐ and R_{b} are the same as defined above;

and all pharmaceutically acceptable isomers, salts, hydrates, solvates and prodrug derivatives thereof.

The invention further provides compounds of the formulae (I) or (II) having the following structures:

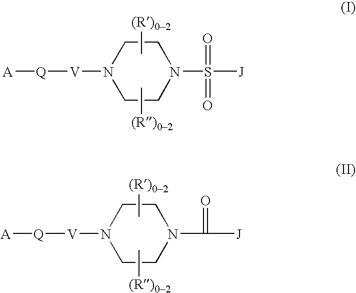

wherein:

A is the a member selected from the group consisting of:

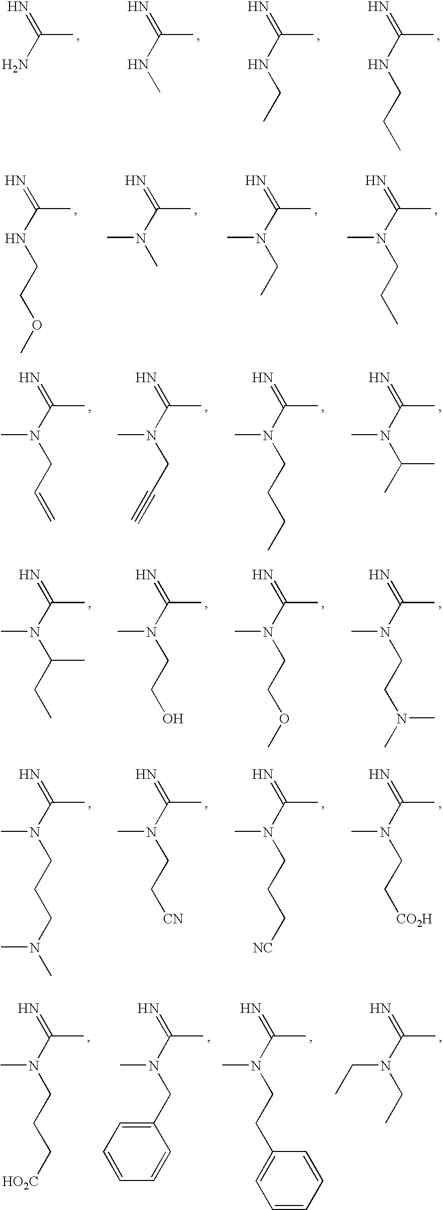

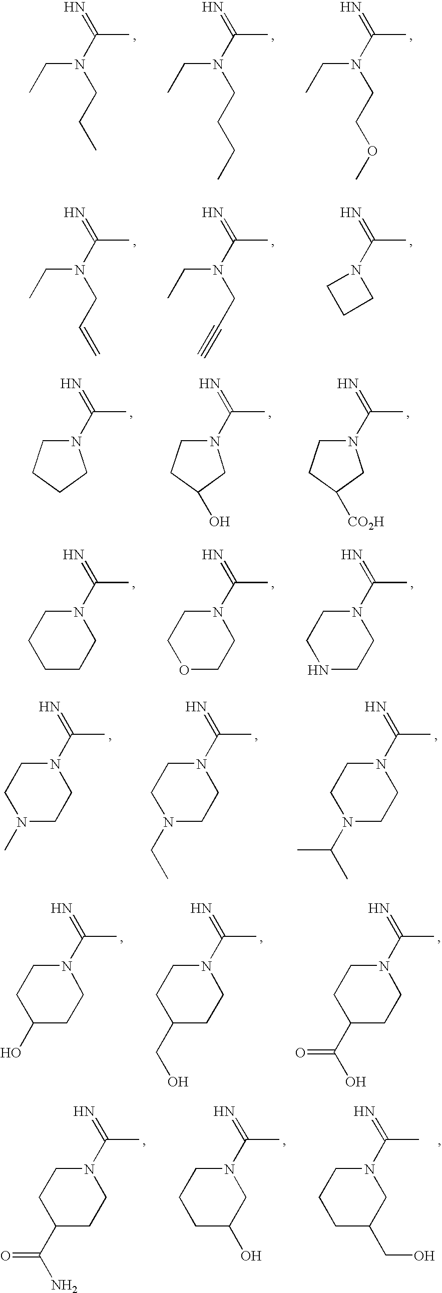

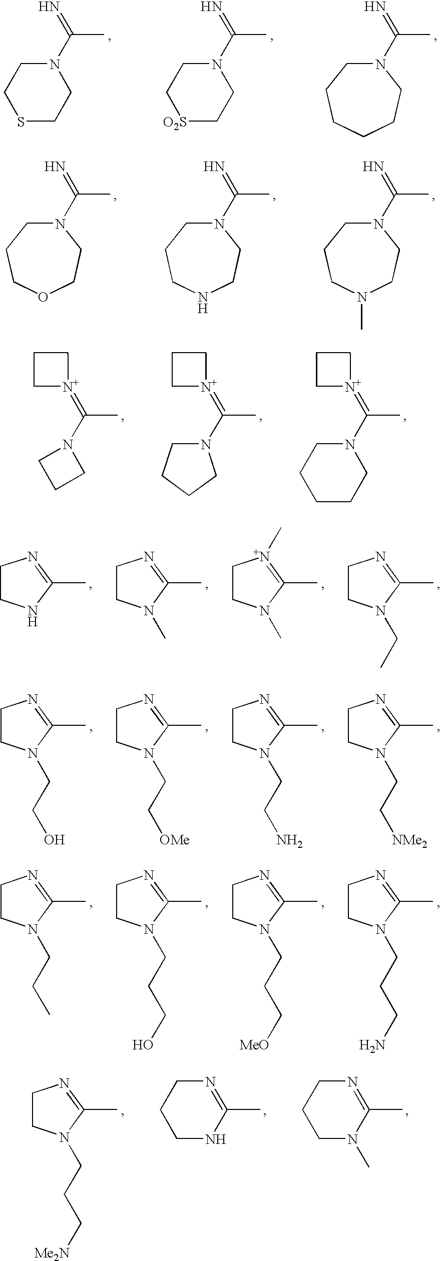

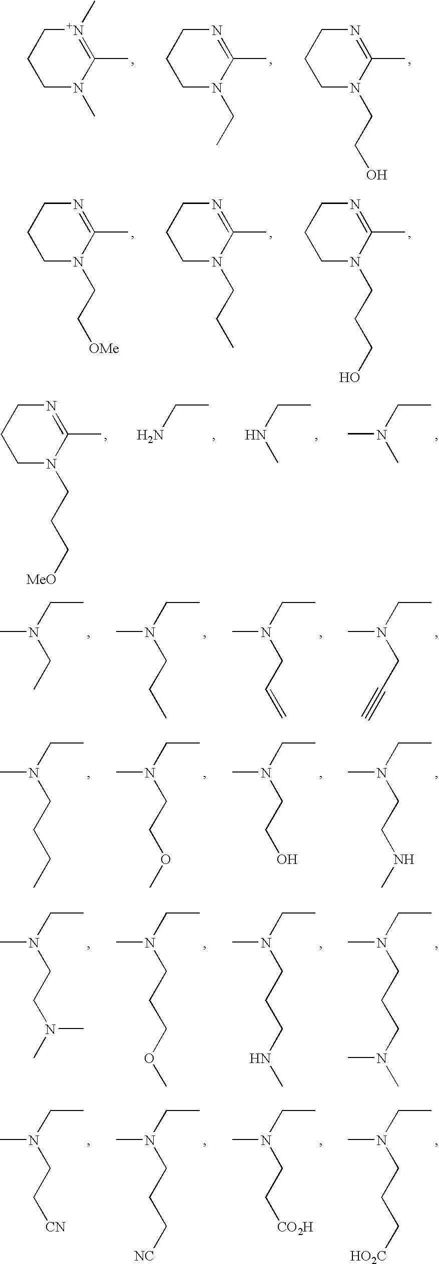

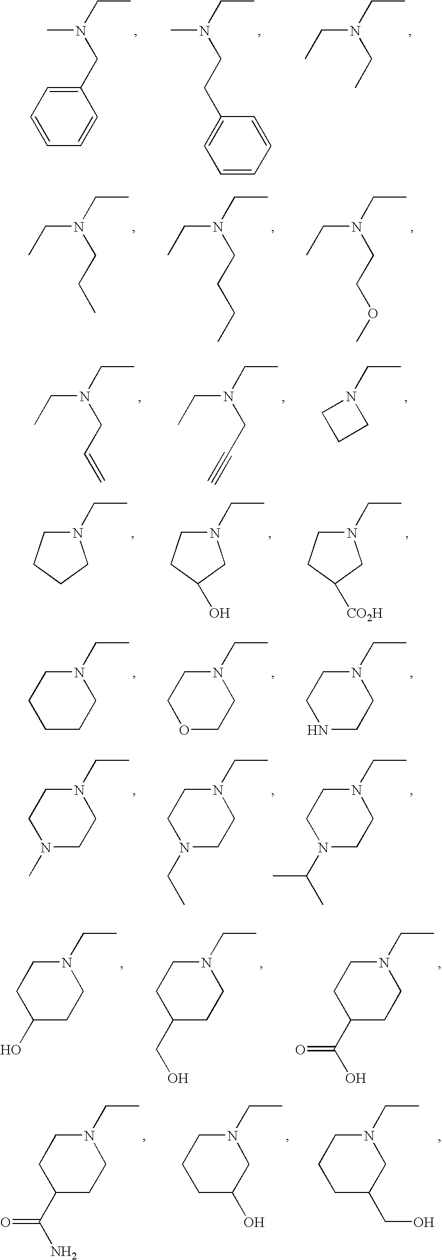

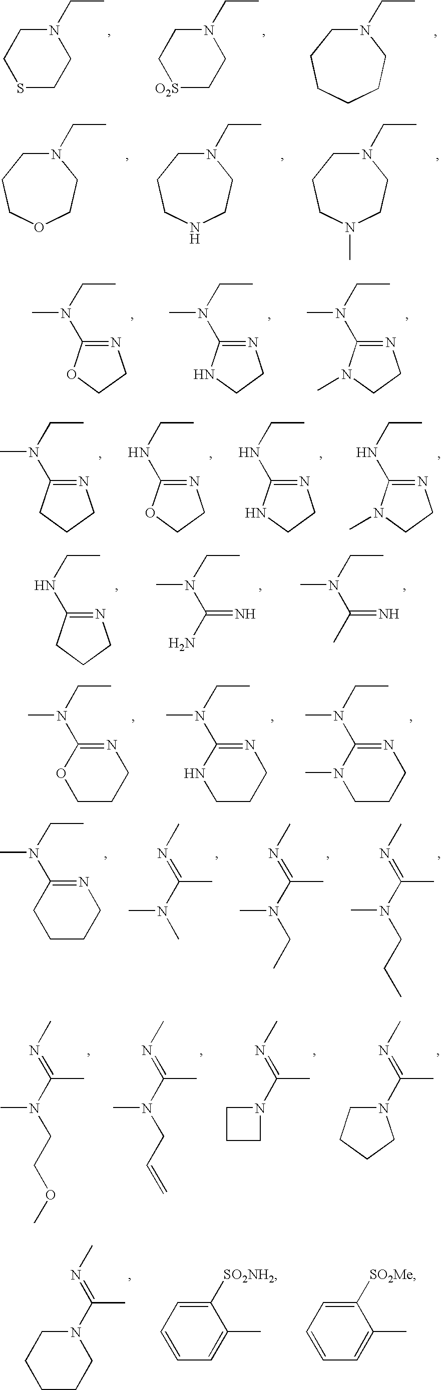

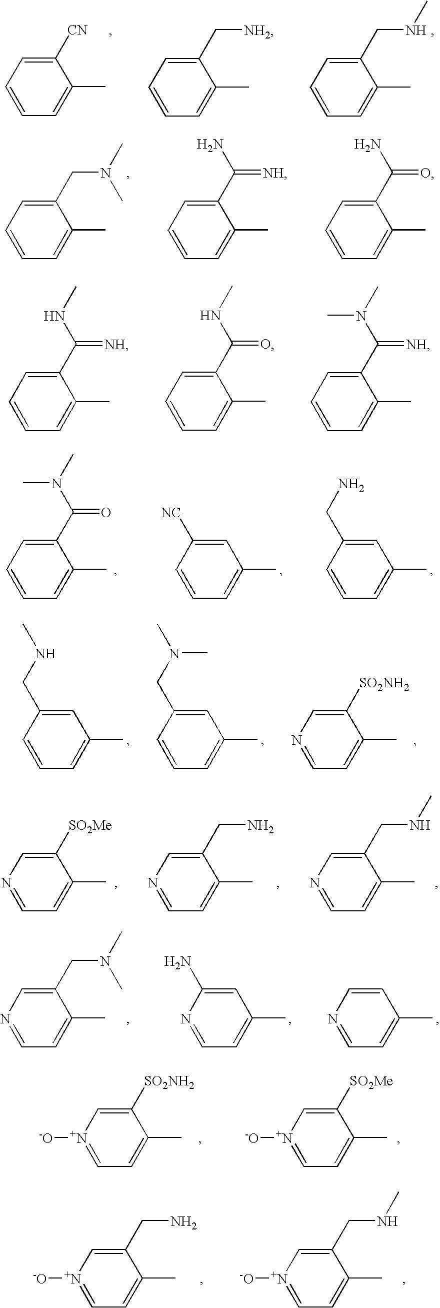

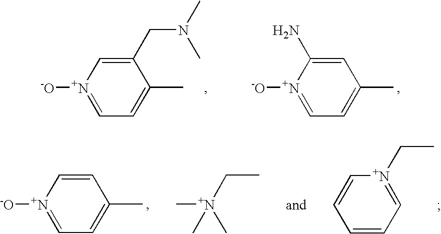

R′ and R″ are independently —H, —C₁₋₆alkyl, —C₁₋₆alkyl-OH, —C₁₋₆alkyl-NRₐR_{b}, —C₁₋₆alkylCN, —C₁₋₆alkylCO₂H, —C₁₋₆alkylCO₂C₁₋₆alkyl, and —C₁₋₆alkylCONRₐR_{b}, wherein Rₐ and R_{b} are the same as defined above;

Q is a member selected from the group consisting of:

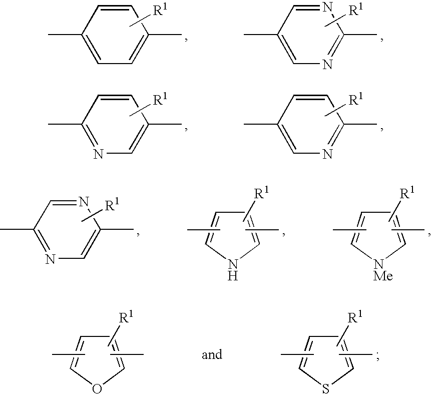

R¹ is a H, —F, —Cl, —Br, —I, —Me, —Et, —OH, —OMe, —OEt, —Opr, —OiPr, —NH₂, —NHMe, —NMe₂, —SH, —SMe, —Set, —SPh, —SO₂Me, —SO₂Et, —CH₂OH, —CH₂NH₂, —CO₂H, —CN, —CONH₂, pyrrolidinyl, piperidinyl, piperazinyl, morpholinyl and 4-methylpiperazinyl;

J is a member selected from the group consisting of:

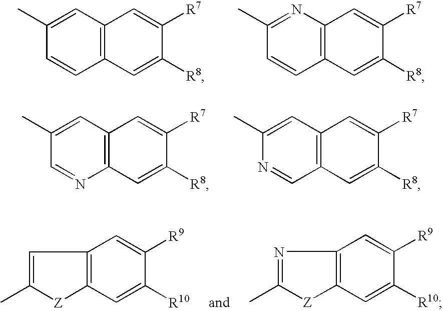

Z is a —NH, —NMe, —O— or —S—;

R⁷ and R⁸ are independently a H, —Cl, —Br, —I or —F, where at least one of R⁷ and R⁸ is not a hydrogen; and

R⁹ and R¹⁰ are independently a H, —Cl, —Br, —I or —F, where at least one of R⁹ and R¹⁰ is not a hydrogen;

and all pharmaceutically acceptable isomers, salts, hydrates, solvates and prodrug derivatives thereof.

The invention further provides compounds of the formulae (I) or (II) having the following structures:

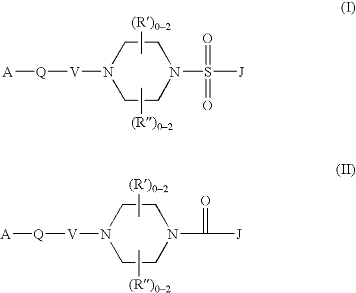

wherein:

A is a member selected from the group consisting of:

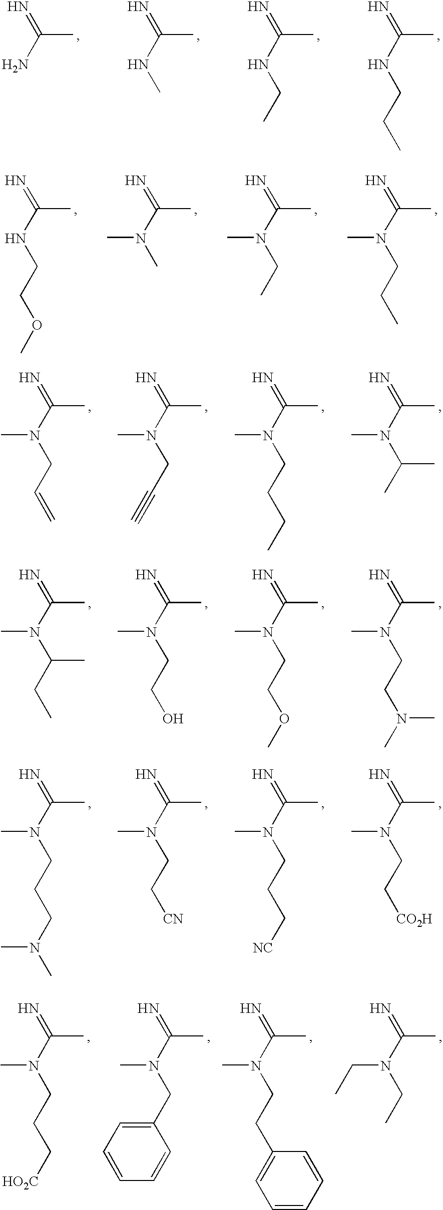

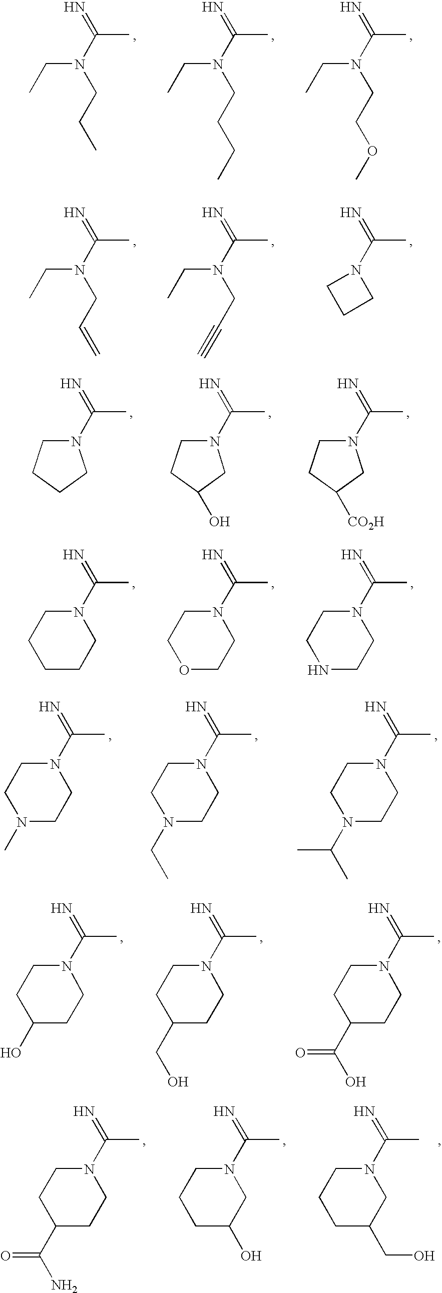

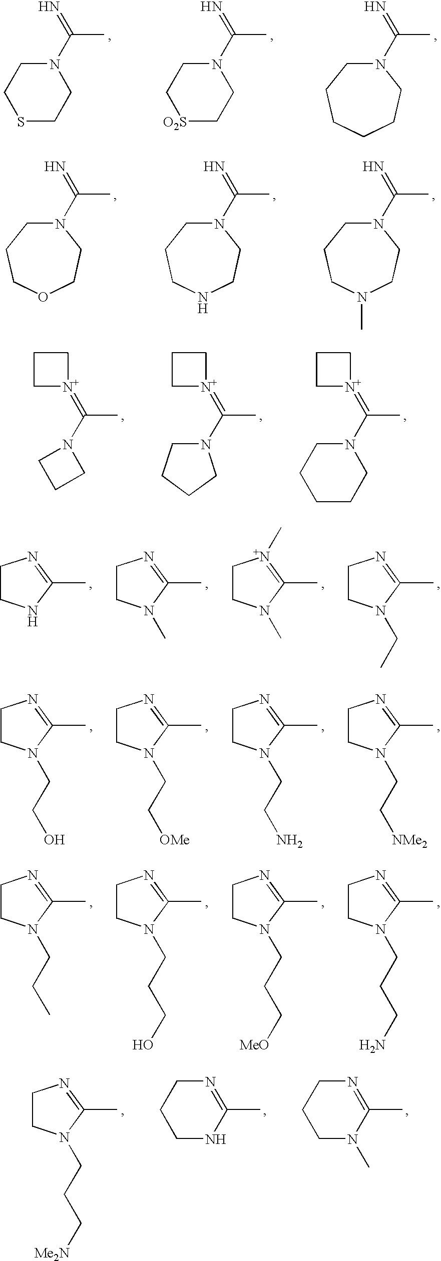

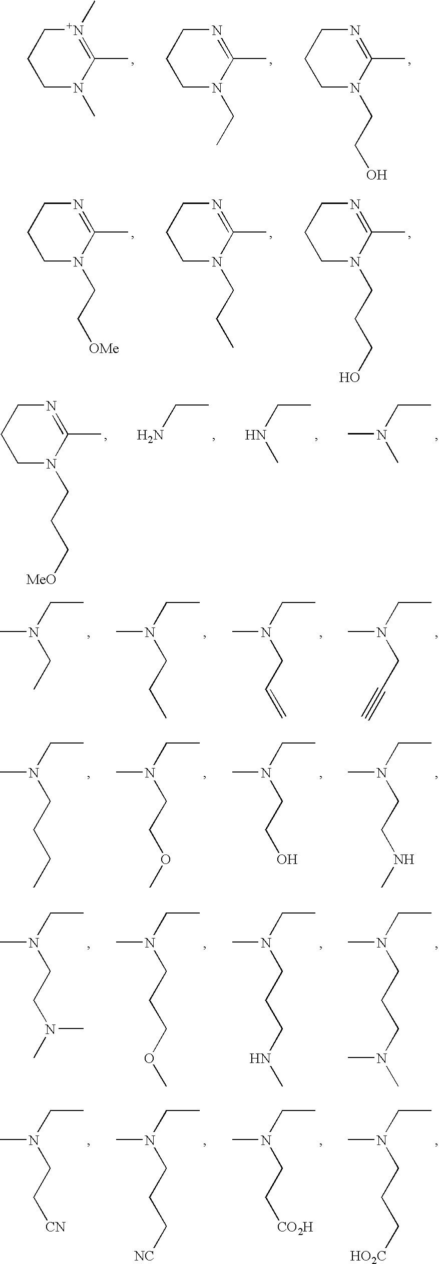

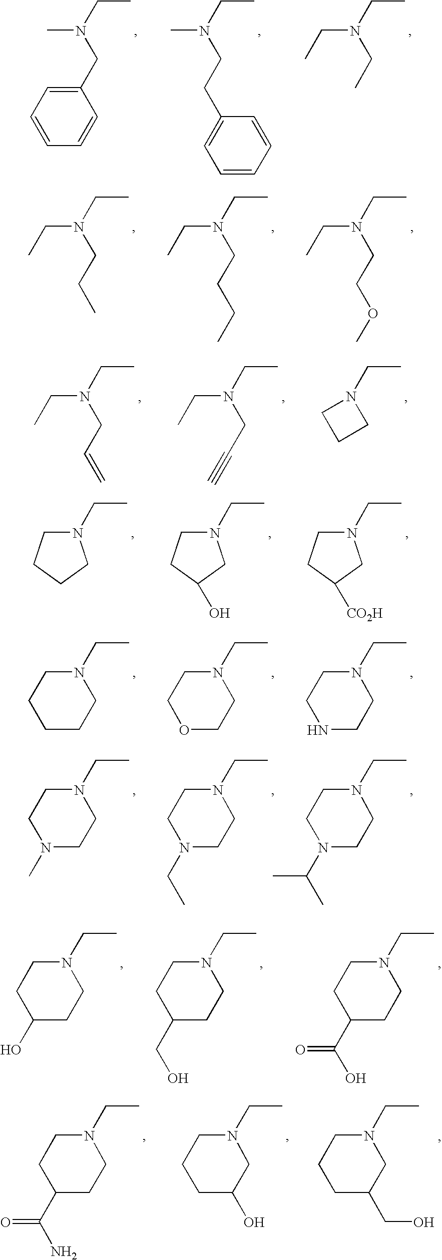

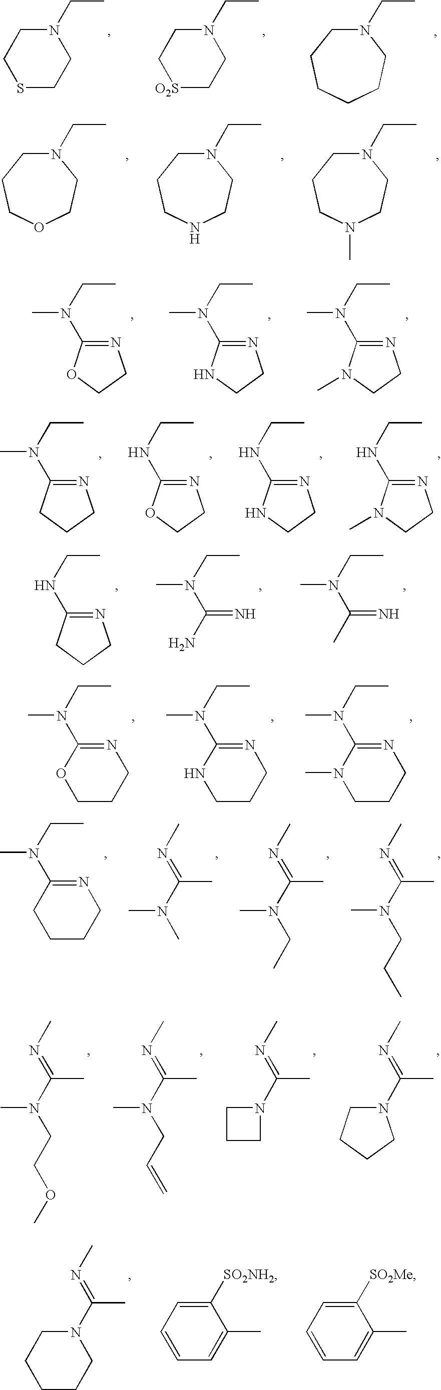

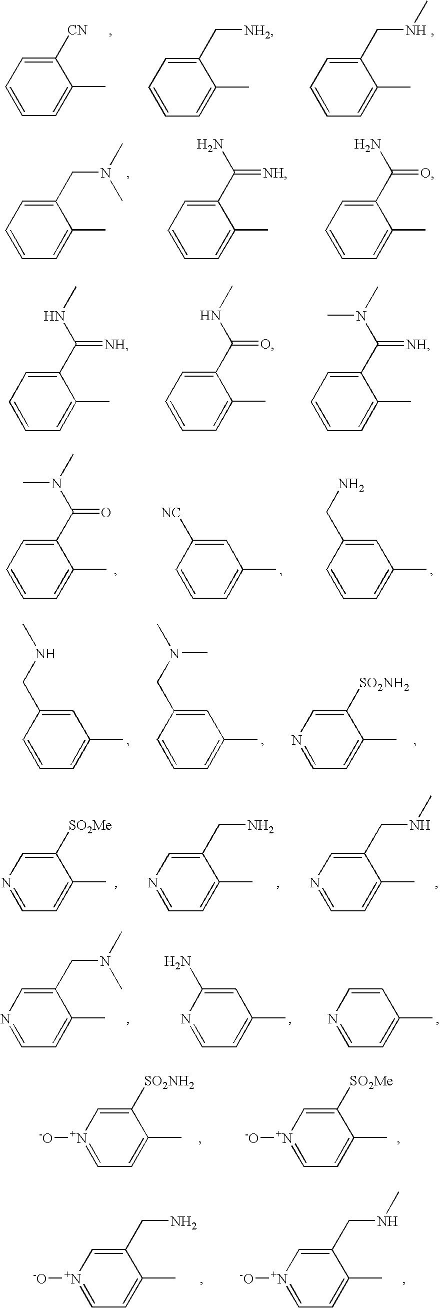

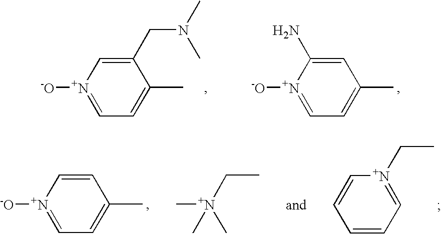

R′ and R″ are independently —H, —C₁₋₆alkyl, —C₁₋₆alkyl-OH, —C₁₋₆alkyl-NRₐR_{b}, —C₁₋₆alkylCN, —C₁₋₆alkylCO₂H, —C₁₋₆alkylCO₂C₁₋₆alkyl, and —C₁₋₆alkylCONRₐR_{b}, wherein Rₐ and R_{b} are the same as defined above;

Q is a member selected from the group consisting of:

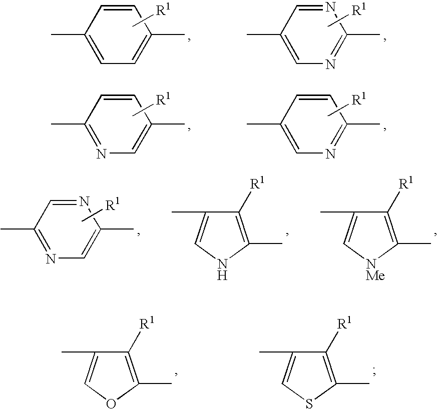

R¹ is a H, —F, —Cl, —Br, —I, —Me, —Et, —OH, —OMe, —OEt, —Opr, —OiPr, —NH₂, —NHMe, —NMe₂, —SH, —SMe, —Set, —SPh, —SO₂Me, —SO₂Et, —CH₂OH, —CH₂NH₂, —CO₂H, —CN, —CONH₂, pyrrolidinyl, piperidinyl, piperazinyl, morpholinyl and 4-methylpiperazinyl;

J is a member selected from the group consisting of:

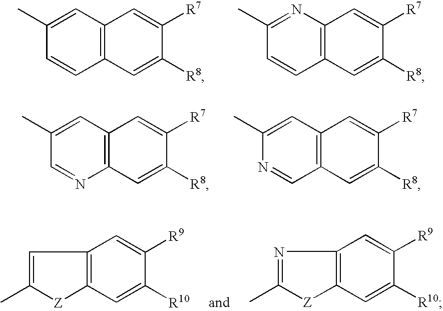

Z is a —NH, —NMe—, —O— or —S—;

R⁷ and R⁸ are independently a —H, —Cl, —Br, —I or —F;

R⁹ and R¹⁰ are independently a —H, —Cl, —Br, —I or —F;

and all pharmaceutically acceptable isomers, salts, hydrates, solvates and prodrug derivatives thereof.

The invention further provides the following compounds of formulae (I) or (II) which may be prepared using the synthetic schemes illustrated in Schemes 5-18, as set forth below:

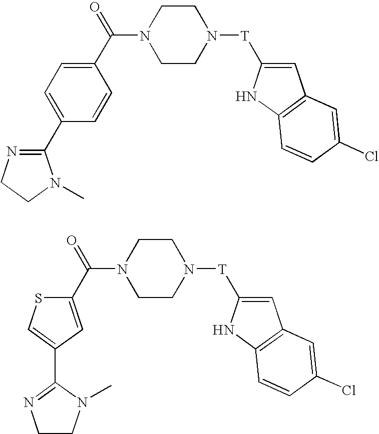

wherein:

T is —SO₂— or —C(═O)—,

and all pharmaceutically acceptable isomers, salts, hydrates, solvates and prodrug derivatives thereof.

The invention further provides the following compound of formula (I) which may be prepared using the synthetic schemes illustrated in Schemes 5, 6, and 7, as set forth below:

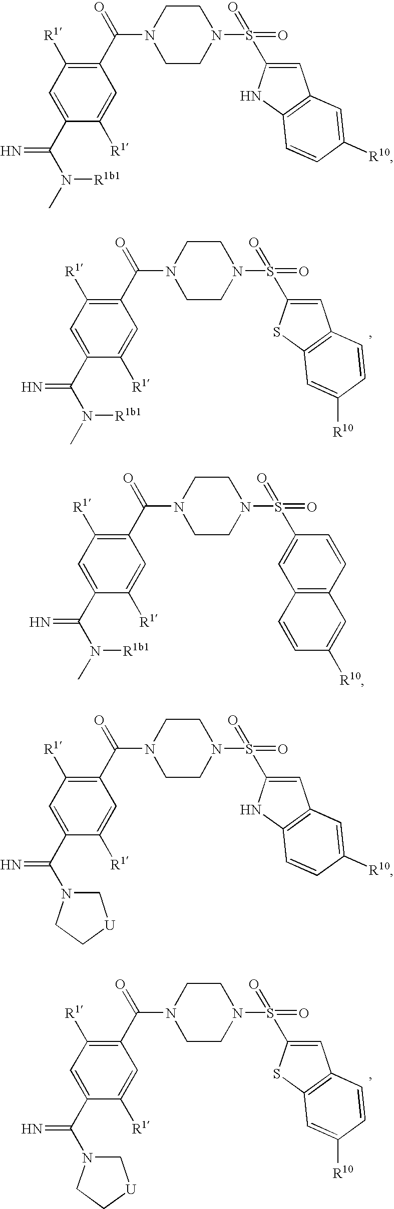

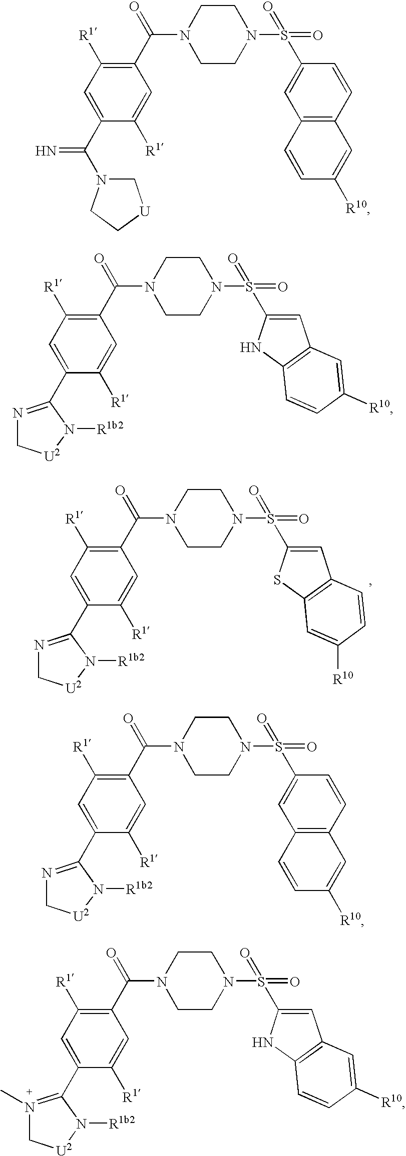

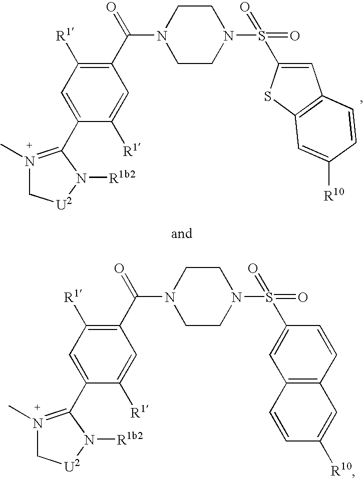

wherein:

R¹⁰ is —Cl or —Br;

R^{1b1} is H, —CH₃, —C₂H₅, —C₃H₇, —C₃H₅, —C₃H₃, —CH₂CH₂OCH₃, —CH₂CH₂N(CH₃)₂, —CH₂CH₂CH₂N(CH₃)₂, —CH₂CH₂CN, —CH₂CH₂CO₂H, —CH₂C₆H₅, or —CH₂CH₂C₆H₅;

R^{1b2} is H, —CH₃, —C₂H₅, —C₃H₇, —CH₂CH₂OH, —CH₂CH₂NH₂, —CH₂CH₂CH₂OH, —CH₂CH₂OCH₃, or —CH₂CH₂CH₂OCH₃;

R^{1′} and R^{1″} are independently H, —CH₃, —C₂H₅, —CH₂OH, —CH₂NH₂, —OH, —OCH₃, —OC₂H₅, —OC₃H₇, —SH, —SCH₃, —SC₂H₅, —SC₃H₇, —SC₆H₅, —SOCH₃, —SO₂CH₃, —SOC₂H₅, —SO₂C₂H₅, —SOC₃H₇, —SO₂C₃H₇, —SOC₆H₅, —SO₂C₆H₅, —CN, —CO₂H, —CONH₂, —NH₂, —NHCH₃, —N(CH₃)₂, pyrrolidinyl, piperidinyl, morpholinyl, piperazinyl, 4-methylpiperazinyl, —F or —Cl;

U is a direct link, —CH₂—, —CH₂CH₂—, —CH₂O—, —CH₂NH—, —CH₂N(CH₃)—, —CH(CO₂H)—CH₂—, —CH(CONH₂)—CH₂—, —CH(OH)CH₂ or —CH(CH₂OH)CH₂; and

U² is —CH₂— or —CH₂CH₂—,

and all pharmaceutically acceptable isomers, salts, hydrates, solvates and prodrug derivatives thereof.

The invention further provides the following compound of formula (I) which may be prepared using the synthetic schemes illustrated in Schemes 8 and 9, as set forth below:

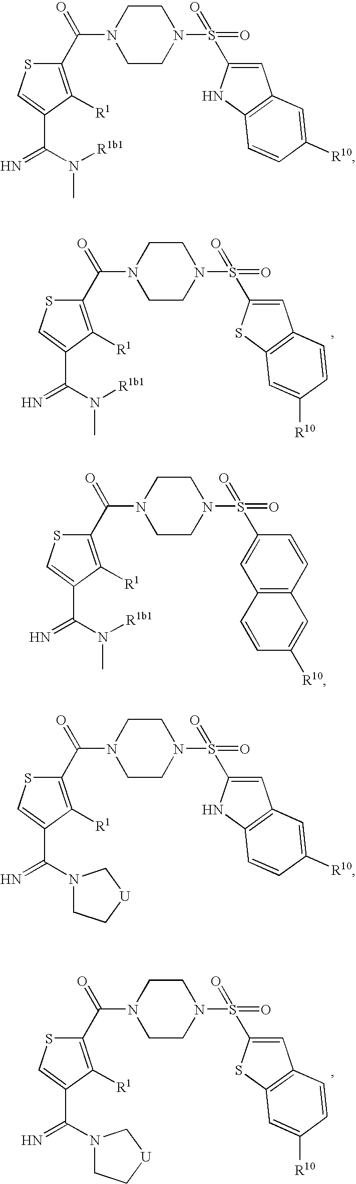

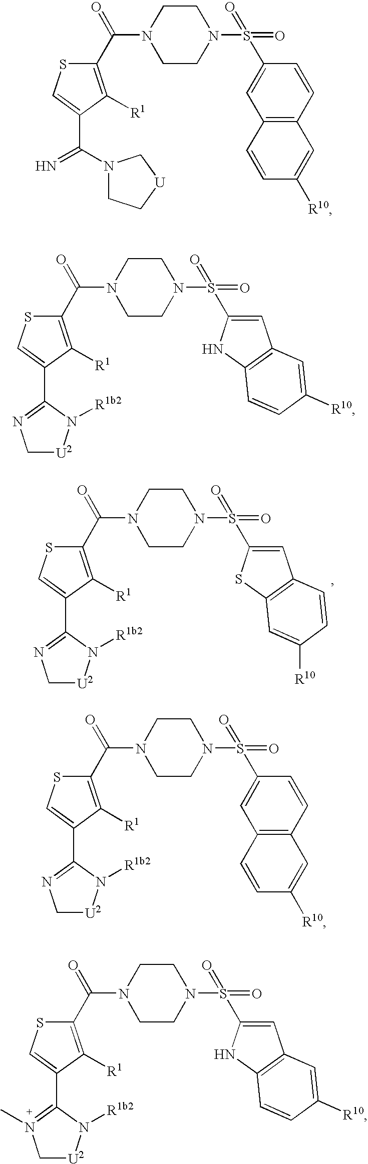

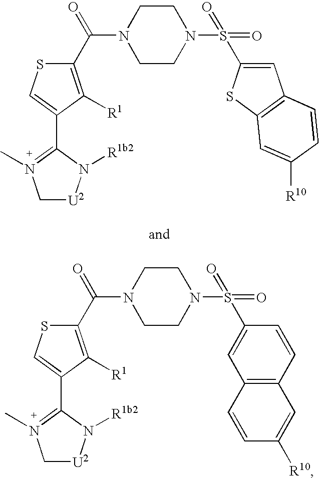

wherein:

R¹⁰ is —Cl or —Br;

R^{1b1} is H, —CH₃, —C₂H₅, —C₃H₇, —C₃H₅, —C₃H₃, —CH₂CH₂OCH₃, —CH₂CH₂N(CH₃)₂, —CH₂CH₂CH₂N(CH₃)₂, —CH₂CH₂CN, —CH₂CH₂CO₂H, —CH₂C₆H₅, or —CH₂CH₂C₆H₅;

R^{1b2} is H, —CH₃, —C₂H₅, —C₃H₇, —CH₂CH₂OH, —CH₂CH₂NH₂, —CH₂CH₂CH₂OH, —CH₂CH₂OCH₃, or —CH₂CH₂CH₂OCH₃;

R¹ is H, —CH₃, —C₂H₅, —CH₂OH, —CH₂NH₂, —OH, —OCH₃, —OC₂H₅, —OC₃H₇, —SH, —SCH₃, —SC₂H₅, —SC₃H₇, —SC₆H₅, —SOCH₃, —SO₂CH₃, —SOC₂H₅, —SO₂C₂H₅, —SOC₃H₇, —SO₂C₃H₇, —SOC₆H₅, —SO₂C₆H₅, —CN, —CO₂H, —CONH₂, —NH₂, —NHCH₃, —N(CH₃)₂, pyrrolidinyl, piperidinyl, morpholinyl, piperazinyl, 4-methylpiperazinyl, —F or —Cl;

U is a direct link, —CH₂—, —CH₂CH₂—, —CH₂O—, —CH₂NH—, —CH₂N(CH₃)—, —CH(CO₂H)—CH₂—, —CH(CONH₂)—CH₂—, —CH(OH)CH₂ or —CH(CH₂OH)CH₂; and

U² is —CH₂— or —CH₂CH₂—,

and all pharmaceutically acceptable isomers, salts, hydrates, solvates and prodrug derivatives thereof.

The invention further provides the following compound of formula (I) which may be prepared using the synthetic schemes illustrated in Schemes 5-12, as set forth below:

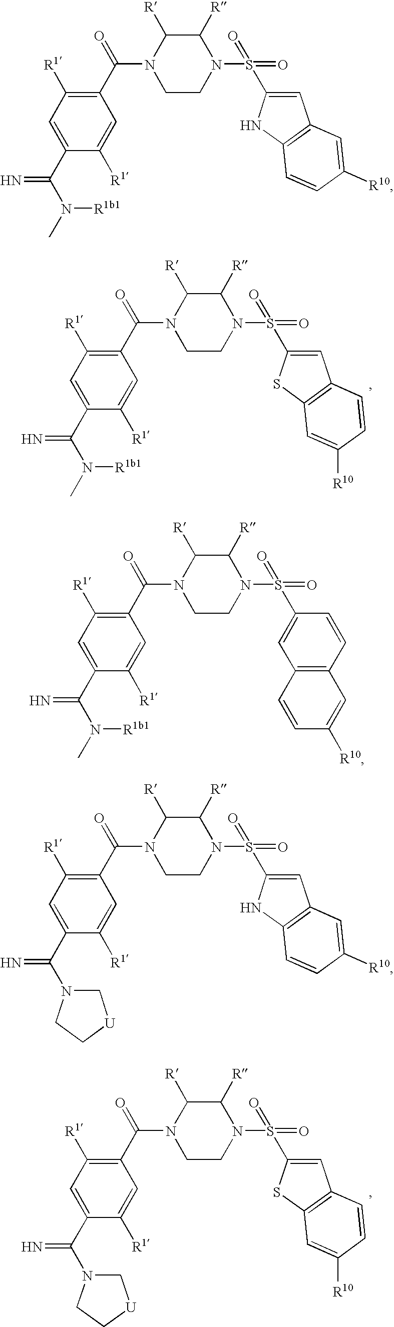

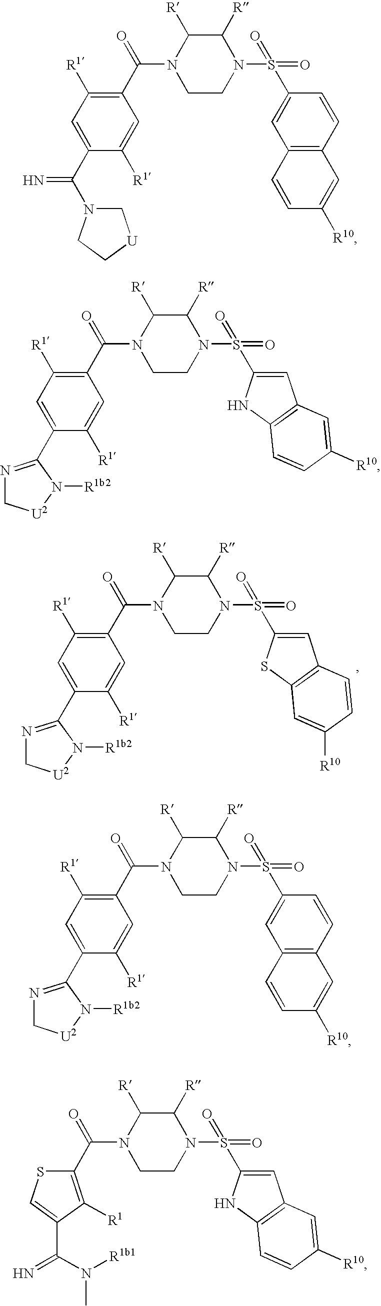

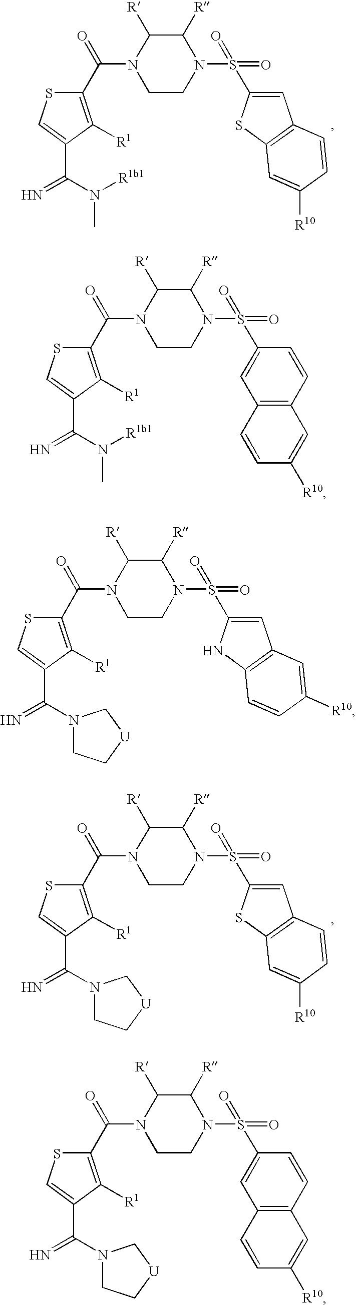

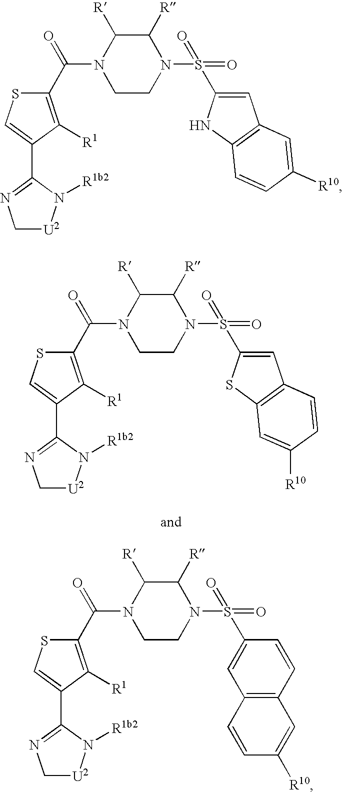

wherein:

R¹⁰ is —Cl or —Br;

R^{1b1} is H, —CH₃, —C₂H₅, —C₃H₇, —C₃H₅, —C₃H₃, —CH₂CH₂OCH₃, —CH₂CH₂N(CH₃)₂, —CH₂CH₂CH₂N(CH₃)₂, —CH₂CH₂CN, —CH₂CH₂CO₂H, —CH₂C₆H₅, or —CH₂CH₂C₆H₅; R^{1b2} is H, —CH₃, —C₂H₅, —C₃H₇, —CH₂CH₂OH, —CH₂CH₂NH₂, —CH₂CH₂CH₂OH, —CH₂CH₂OCH₃, or —CH₂CH₂CH₂OCH₃;

R′ and R″ are independently H, —CH₃, —C₂H₅, —CO₂CH₃, —CH₂CO₂CH₃, —CO₂H, —CH₂CO₂H, —CONRₐR_{b} or —CH₂CONRₐR_{b}, where Rₐ and R_{b} are independently H, —CH₃, —C₂H₅, —C₃H₇, —C₃H₅ or C₃H₃; or Rₐ and R_{b} taken together with the nitrogen to which they are attached form an azetidinyl, pyrrolidinyl, piperidinyl, morpholinyl, thiomorpholinyl and its oxidized forms, piperazinyl or 4-methyl-1-piperazinyl;

R¹, R^{1′} and R^{1″} are independently H, —CH₃, —C₂H₅, —CH₂OH, —CH₂NH₂, —OH, —OCH₃, —OC₂H₅, —OC₃H₇, —SH, —SCH₃, —SC₂H₅, —SC₃H₇, —SOCH₃, —SO₂CH₃, —SOC₂H₅, —SO₂C₂H₅, —SOC₃H₇, —SO₂C₃H₇, —CN, —CO₂H, —CONH₂, —F or —Cl;

U is a direct link, —CH₂—, —CH₂CH₂—, —CH₂O—, —CH₂NH—, —CH₂N(CH₃)—, —CH(CO₂H)—CH₂—, —CH(CONH₂)—CH₂—, —CH(OH)CH₂ or —CH(CH₂OH)CH₂; and

U² is —CH₂— or —CH₂CH₂—,

and all pharmaceutically acceptable isomers, salts, hydrates, solvates and prodrug derivatives thereof.

The invention further provides the following compound of formula (I) which may be prepared using the synthetic schemes illustrated in Scheme 7, as set forth below:

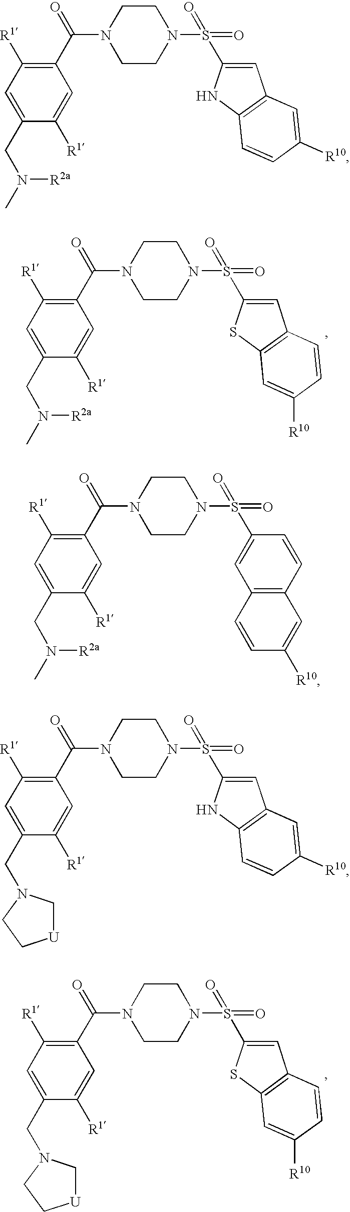

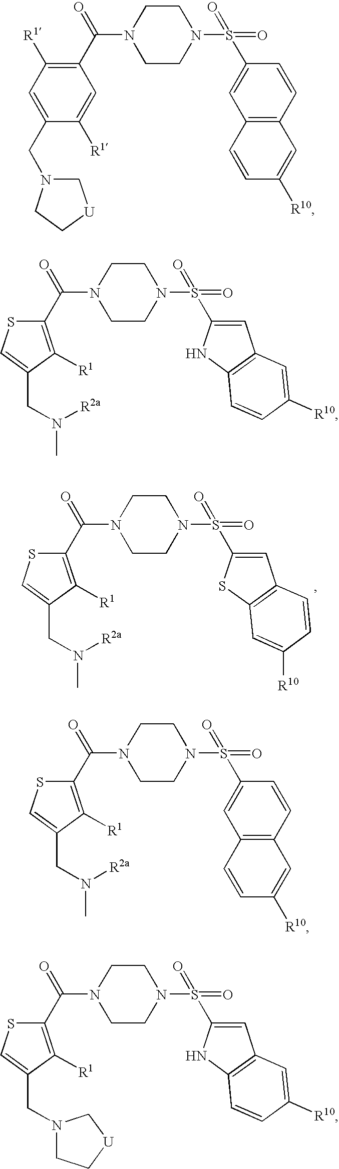

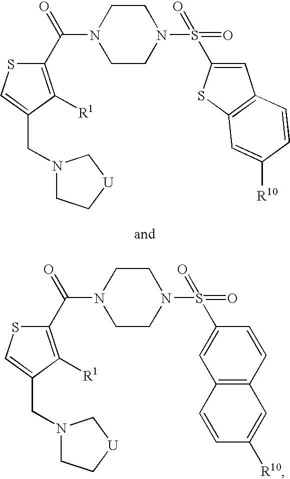

wherein

R¹⁰ is —Cl or —Br;

R^{2a} is H, —CH₃, —C₂H₅, —C₃H₇, —C₃H₅, —C₃H₃, —CH₂CH₂OCH₃, —CH₂CH₂N(CH₃)₂, —CH₂CH₂CH₂N(CH₃)₂, —CH₂CH₂CN, —CH₂CH₂CO₂H, —CH₂C₆H₅, —CH₂CH₂C₆H₅;

R¹, R^{1′}, and R^{1″} are independently H, —CH₃, —C₂H₅, —CH₂OH, —CH₂NH₂, —OH, —OCH₃, —OC₂H₅, —OC₃H₇, —SH, —SCH₃, —SC₂H₅, —SC₃H₇, —SOCH₃, —SO₂CH₃, —SOC₂H₅, —SO₂C₂H₅, —SOC₃H₇, —SO₂C₃H₇, —CN, —CO₂H, —CONH₂, —F or —Cl;

U is a direct link, —CH₂—, —CH₂CH₂—, —CH₂O—, —CH₂NH—, —CH₂N(CH₃)—, —CH(CO₂H)—CH₂—, —CH(CONH₂)—CH₂—, —CH(OH)CH₂ or —CH(CH₂OH)CH₂,

and all pharmaceutically acceptable isomers, salts, hydrates, solvates and prodrug derivatives thereof.

The invention further provides the following compound of formula (I) which may be prepared using the synthetic schemes illustrated in Schemes 5-18, as set forth below:

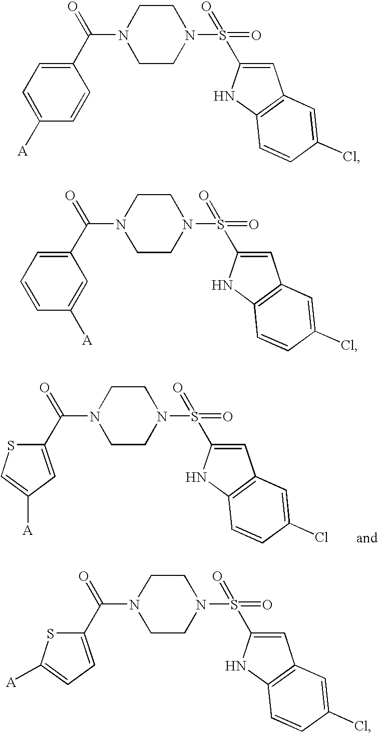

wherein:

A is selected from the group consisting of:

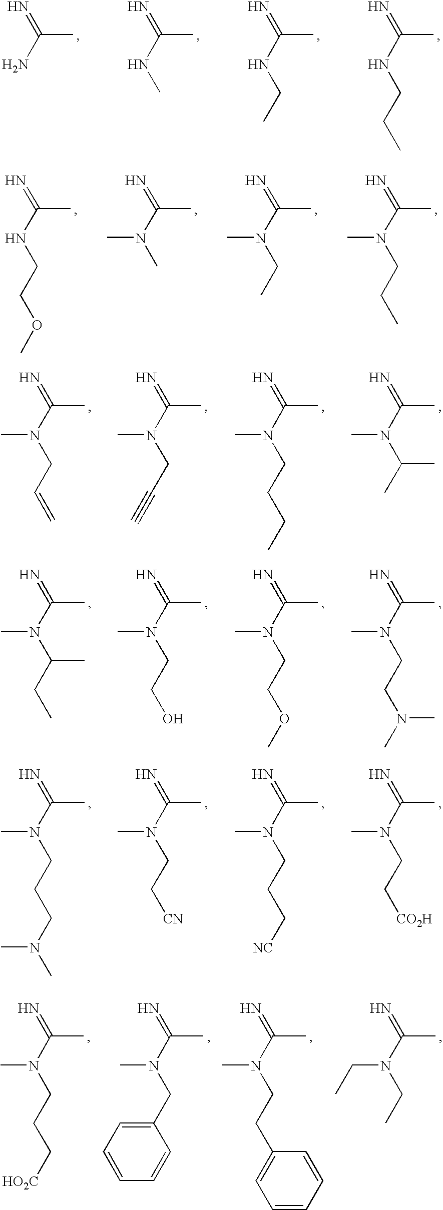

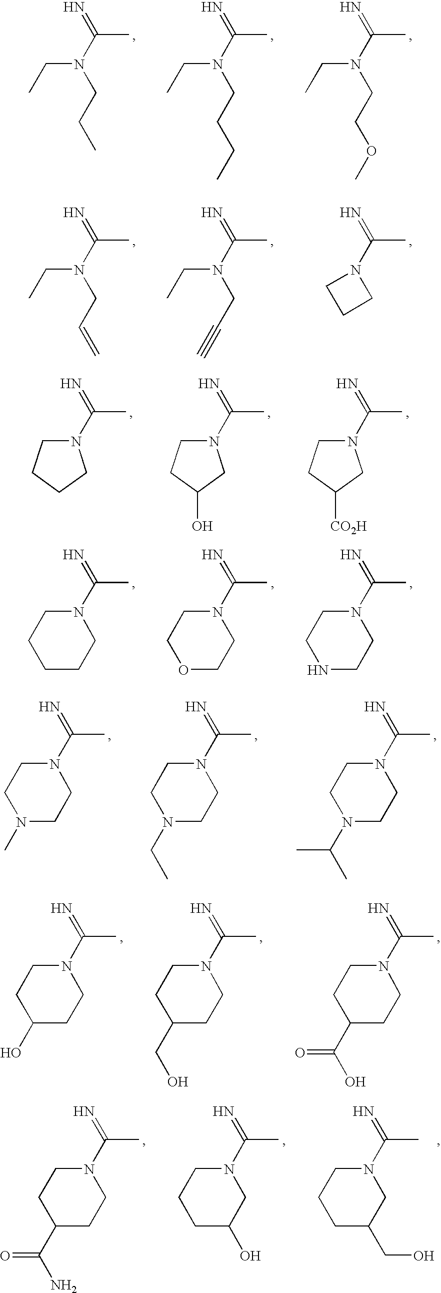

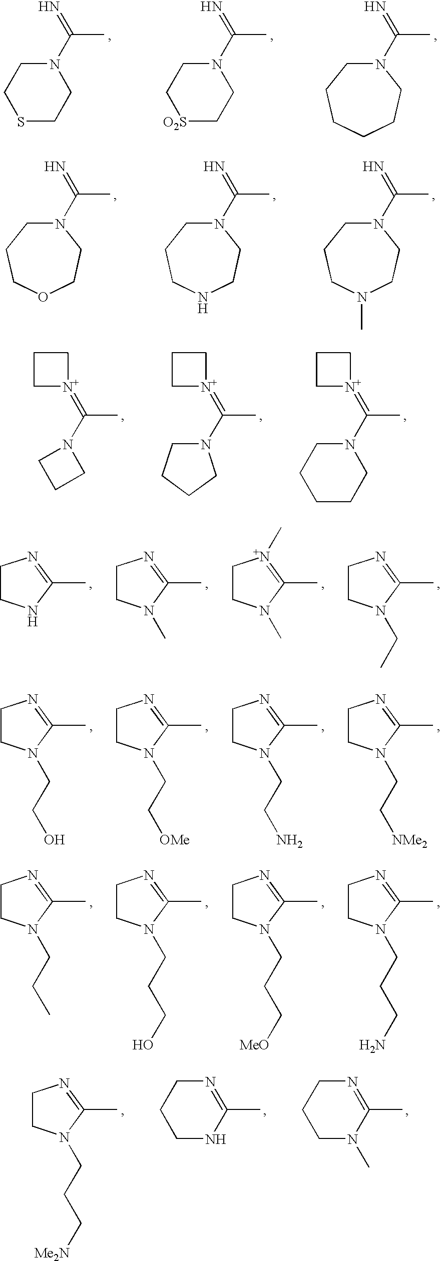

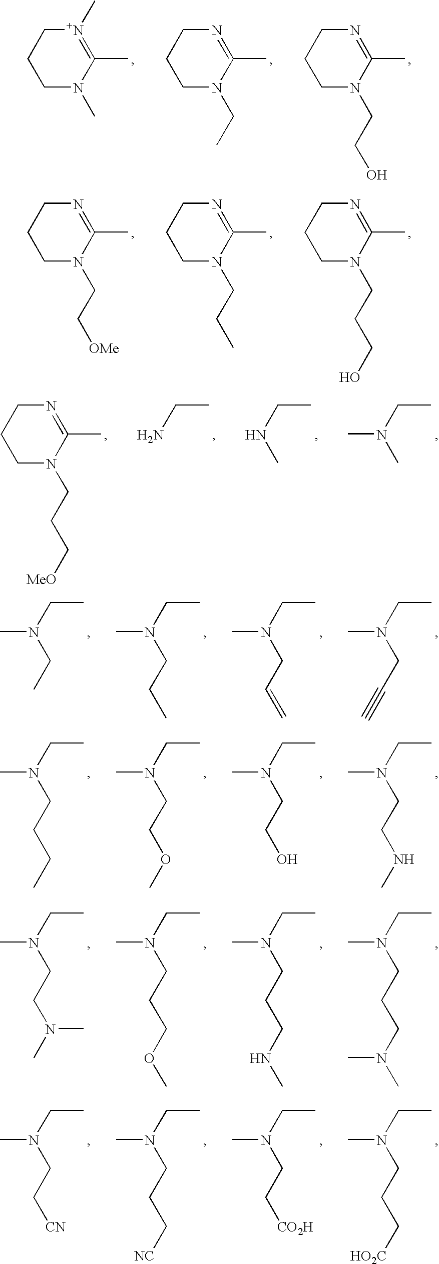

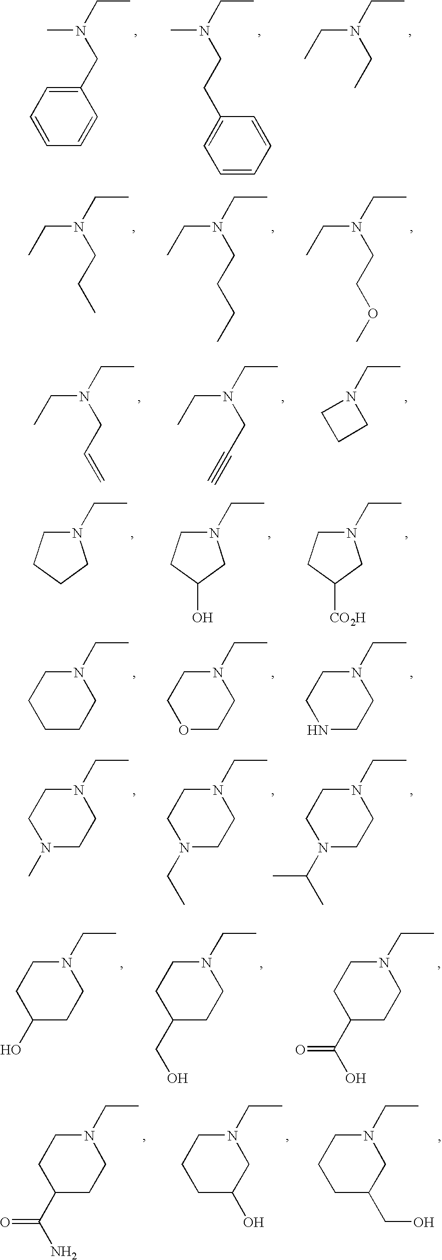

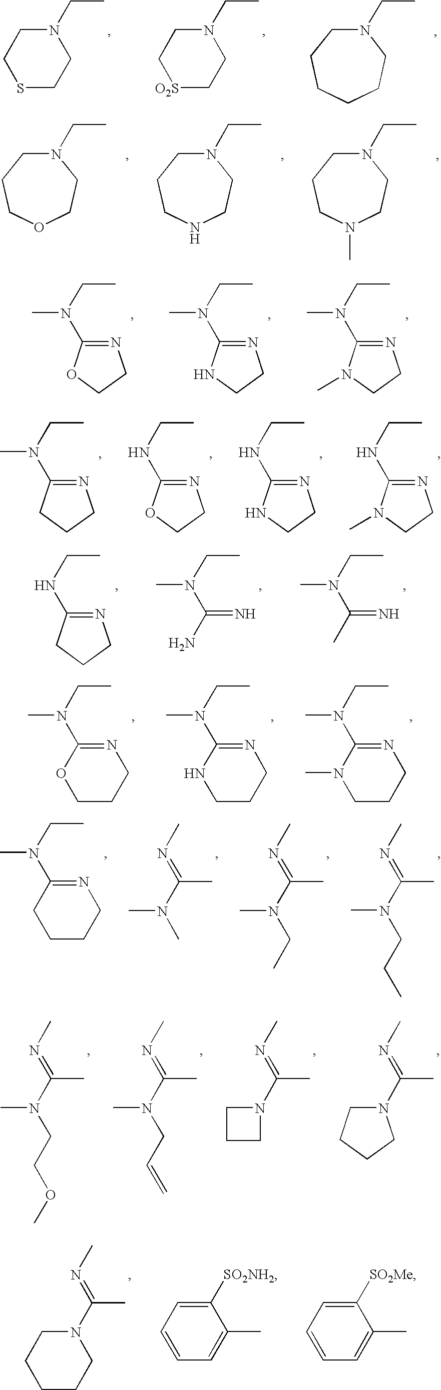

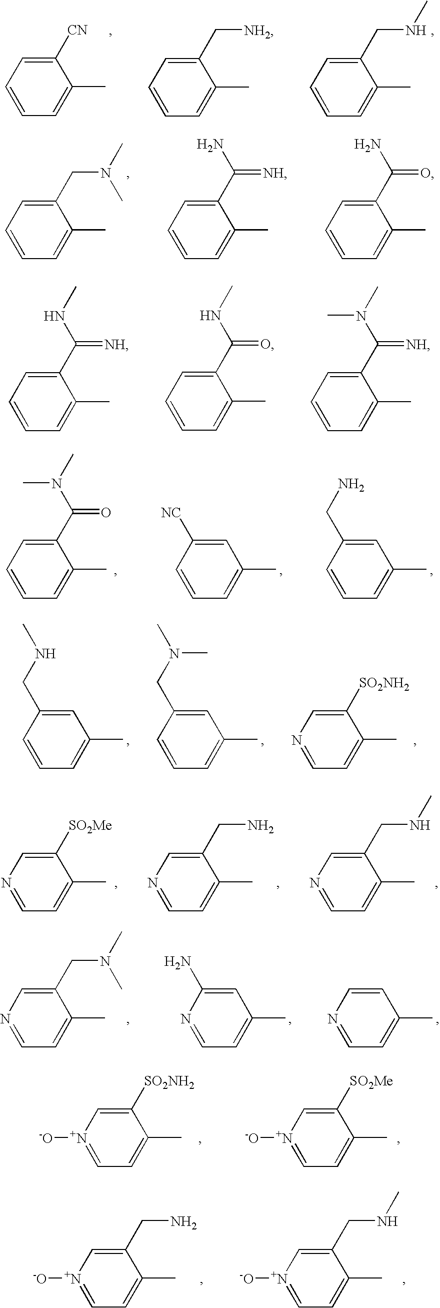

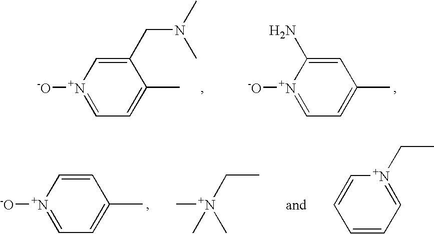

and all pharmaceutically acceptable isomers, salts, hydrates, solvates and prodrug derivatives thereof.

The invention further provides the following compound of formula (I) which may be prepared using the synthetic schemes illustrated in Schemes 5-18, as set forth below:

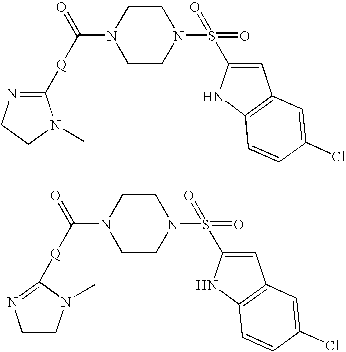

wherein:

Q is selected from the group consisting of:

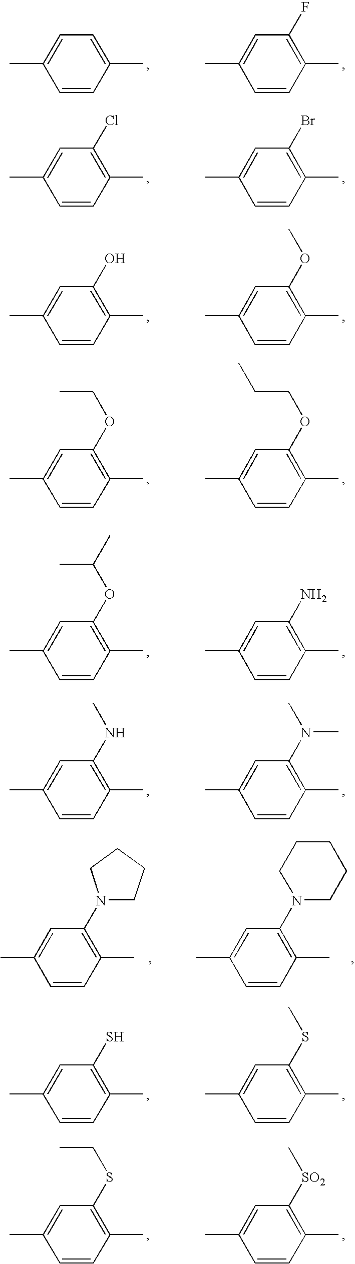

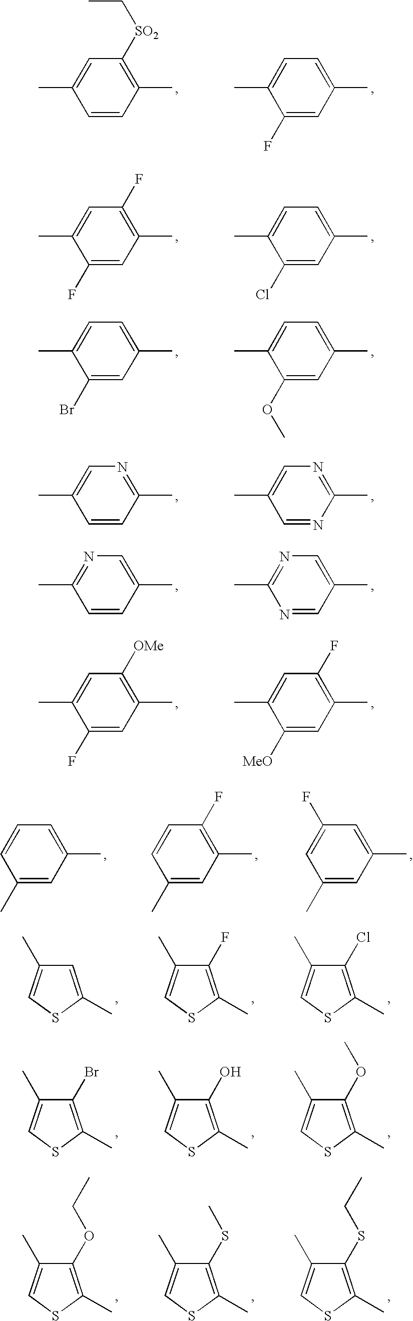

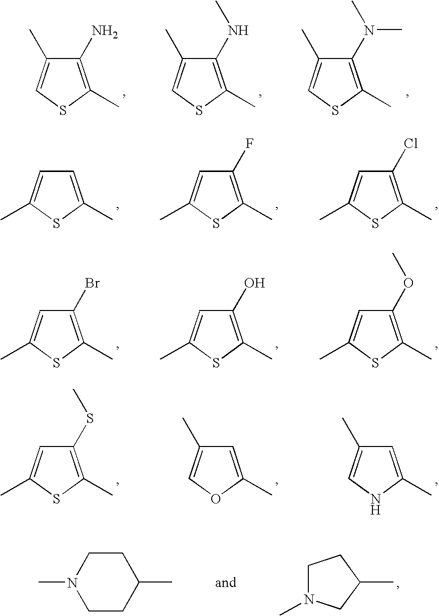

and all pharmaceutically acceptable isomers, salts, hydrates, solvates and prodrug derivatives thereof.

The invention further provides the following compound of formula (I) which may be prepared using the synthetic schemes illustrated in Schemes 5-18, as set forth below:

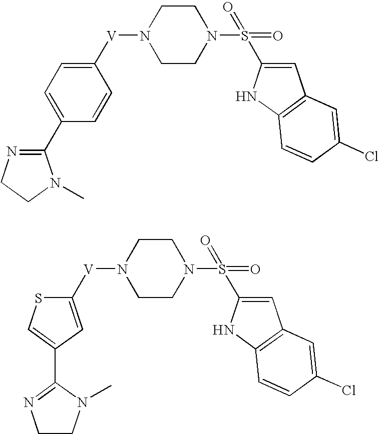

wherein:

V is —CH₂— or —C(═O)—,

and all pharmaceutically acceptable isomers, salts, hydrates, solvates and prodrug derivatives thereof.

The invention further provides the following compound of formula (I) which may be prepared using the synthetic schemes illustrated in Schemes 5-18

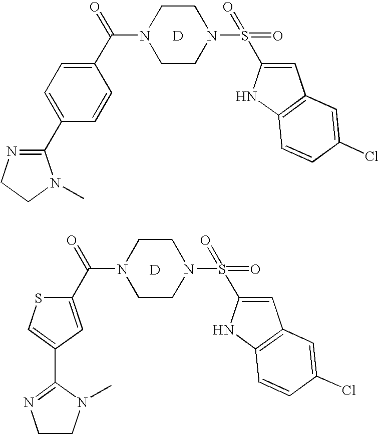

wherein:

D is selected from the group consisting of:

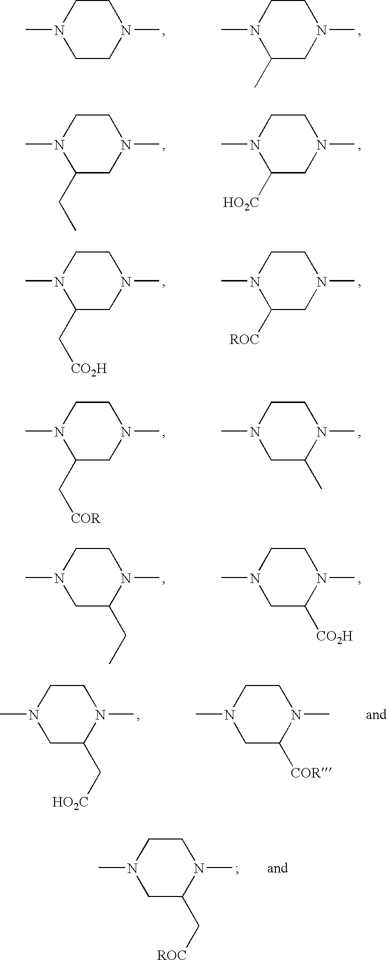

R′″ is selected from the group consisting of:

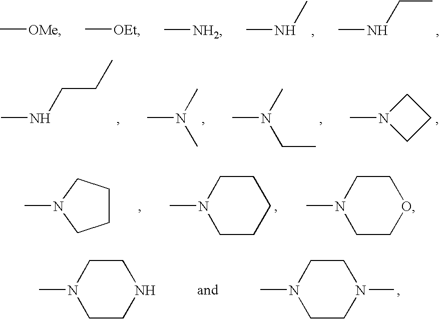

and all pharmaceutically acceptable isomers, salts, hydrates, solvates and prodrug derivatives thereof.

The invention further provides the following compound of formula (I) which may be prepared using the synthetic schemes illustrated in Schemes 5-18, as set forth below:

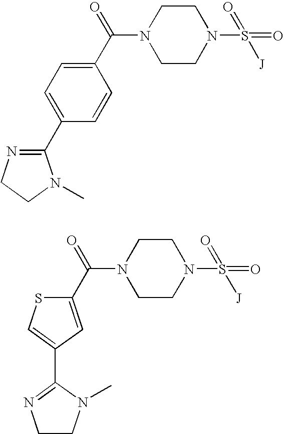

wherein:

J is selected from the group consisting of:

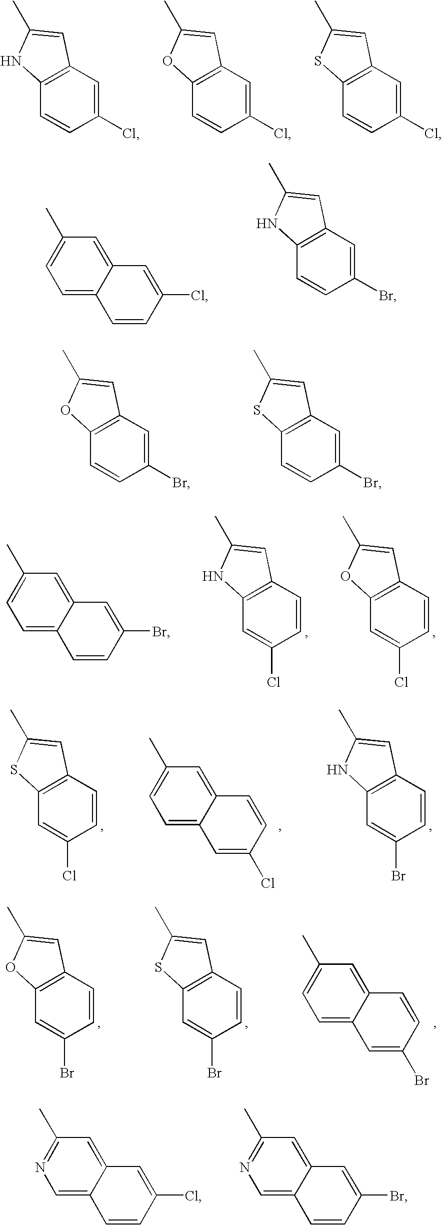

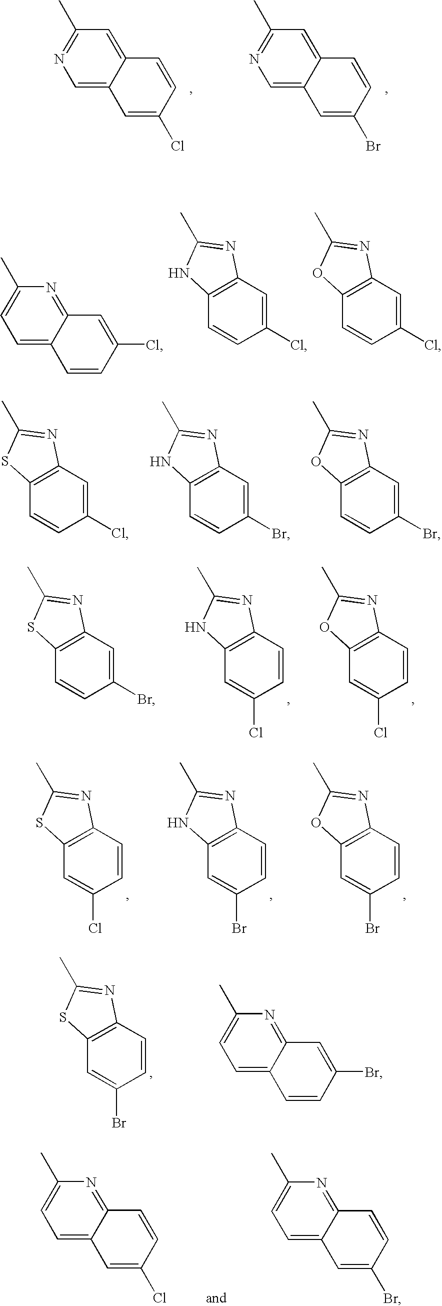

and all pharmaceutically acceptable isomers, salts, hydrates, solvates and prodrug derivatives thereof.

The invention also encompasses all pharmaceutically acceptable isomers, salts, hydrates, solvates and prodrug derivatives of the compounds of the invention as set forth herein. The compounds of the invention can exist in various isomeric and tautomeric forms, and all such forms are meant to be included in the invention, along with pharmaceutically acceptable salts, hydrates, solvates and prodrug derivatives of such isomers and tautomers.

The compounds of the invention may be isolated as the free acid or base or converted to salts of various inorganic and organic acids and bases. Such salts are within the scope of the invention. Non-toxic and physiologically compatible salts are particularly useful although other less desirable salts may have use in the processes of isolation and purification.

A number of methods are useful for the preparation of the salts of the compounds as described above and are known to those skilled in the art. For example, the free acid or free base form of a compound of one of the formulae above can be reacted with one or more molar equivalents of the desired acid or base in a solvent or solvent mixture in which the salt is insoluble, or in a solvent like water after which the solvent is removed by evaporation, distillation or freeze drying. Alternatively, the free acid or base form of the product may be passed over an ion exchange resin to form the desired salt or one salt form of the product may be converted to another using the same general process.

The invention also encompasses prodrug derivatives of the compounds contained herein. The term “prodrug” refers to a pharmacologically inactive derivative of a parent drug molecule that requires biotransformation, either spontaneous or enzymatic, within the organism to release the active drug. Prodrugs are variations or derivatives of the compounds of the invention which have groups cleavable under metabolic conditions. Prodrugs become the compounds of the invention which are pharmaceutically active in vivo, when they undergo solvolysis under physiological conditions or undergo enzymatic degradation. Prodrug compounds of the invention may be called single, double, triple etc., depending on the number of biotransformation steps required to release the active drug within the organism, and indicating the number of functionalities present in a precursor-type form. Prodrug forms often offer advantages of solubility, tissue compatibility, or delayed release in the mammalian organism (see, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif. 1992). Prodrugs commonly known in the art include acid derivatives well known to practitioners of the art, such as, for example, esters prepared by reaction of the parent acids with a suitable alcohol, or amides prepared by reaction of the parent acid compound with an amine, or basic groups reacted to form an acylated base derivative. Moreover, the prodrug derivatives of the invention may be combined with other features herein taught to enhance bioavailability.

The compounds of the present invention may also be used alone or in combination or in combination with other therapeutic or diagnostic agents. In certain preferred embodiments, the compounds of the invention may be coadministered along with other compounds typically prescribed for these conditions according to generally accepted medical practice such as anticoagulant agents, thrombolytic agents, or other antithrombotics, including platelet aggregation inhibitors, tissue plasminogen activators, urokinase, prourokinase, streptokinase, heparin, aspirin, or warfarin. The compounds of the present invention may act in a synergistic fashion to prevent reocclusion following a successful thrombolytic therapy and/or reduce the time to reperfusion. These compounds may also allow for reduced doses of the thrombolytic agents to be used and therefore minimize potential hemorrhagic side-effects. The compounds of the invention can be utilized in vivo, ordinarily in mammals such as primates (e.g. humans), sheep, horses, cattle, pigs, dogs, cats, rats and mice, or in vitro.

The biological properties of the compounds of the present invention can be readily characterized by methods that are well known in the art, for example by the in vitro protease activity assays and in vivo studies to evaluate antithrombotic efficacy, and effects on hemostasis and hematological parameters, such as are illustrated in the examples.

Diagnostic applications of the compounds of the invention will typically utilize formulations in the form of solutions or suspensions. In the management of thrombotic disorders the compounds of the invention may be utilized in compositions such as tablets, capsules or elixirs for oral administration, suppositories, sterile solutions or suspensions or injectable administration, and the like, or incorporated into shaped articles. Subjects in need of treatment (typically mammalian) using the compounds of the invention can be administered dosages that will provide optimal efficacy. The dose and method of administration will vary from subject to subject and be dependent upon such factors as the type of mammal being treated, its sex, weight, diet, concurrent medication, overall clinical condition, the particular compounds employed, the specific use for which these compounds are employed, and other factors which those skilled in the medical arts will recognize.

Preparation of Compounds

The compounds of the present invention may be synthesized by standard organic chemical synthetic methods as described and referenced in standard textbooks. These methods are well known in the art. See, e.g., Morrison and Boyd, “Organic Chemistry”, Allyn and Bacon, Inc., Boston, 1959, et seq.

Starting materials used in any of these methods are commercially available from chemical vendors such as Aldrich, Sigma, Nova Biochemicals, Bachem Biosciences, and the like, or may be readily synthesized by known procedures.

Reactions are carried out in standard laboratory glassware and reaction vessels under reaction conditions of standard temperature and pressure, except where otherwise indicated.

During the synthesis of these compounds, the functional groups of the substituents are optionally protected by blocking groups to prevent cross reaction during coupling procedures. Examples of suitable blocking groups and their use are described in “The Peptides: Analysis, Synthesis, Biology”, Academic Press, Vol. 3 (Gross, et al., Eds., 1981) and Vol. 9 (1987), the disclosures of which are incorporated herein by reference.

Non-limiting exemplary synthesis schemes are outlined directly below, and specific steps are described in the Examples. The reaction products are isolated and purified by conventional methods, typically by solvent extraction into a compatible solvent. The products may be further purified by column chromatography or other appropriate methods.

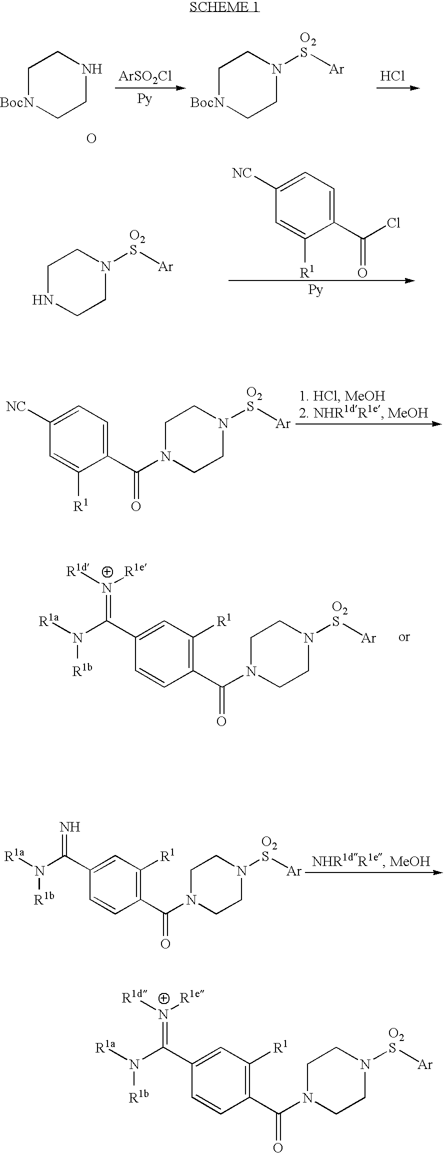

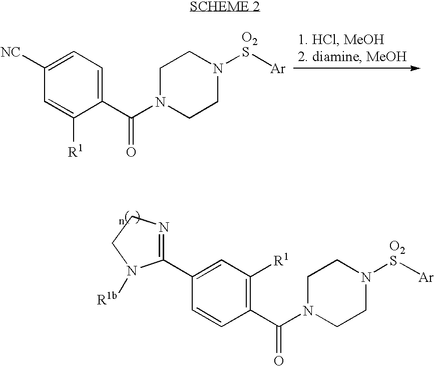

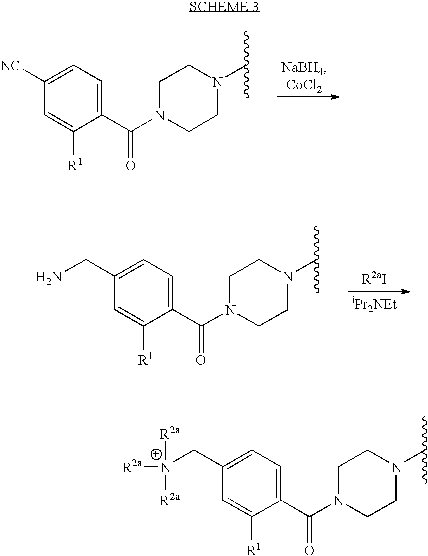

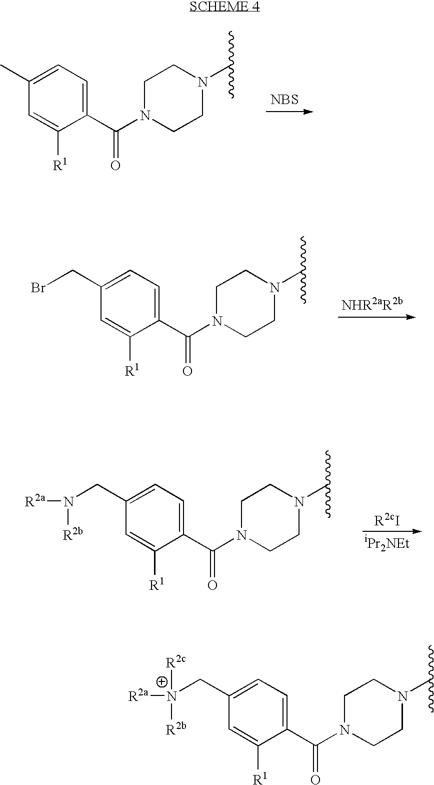

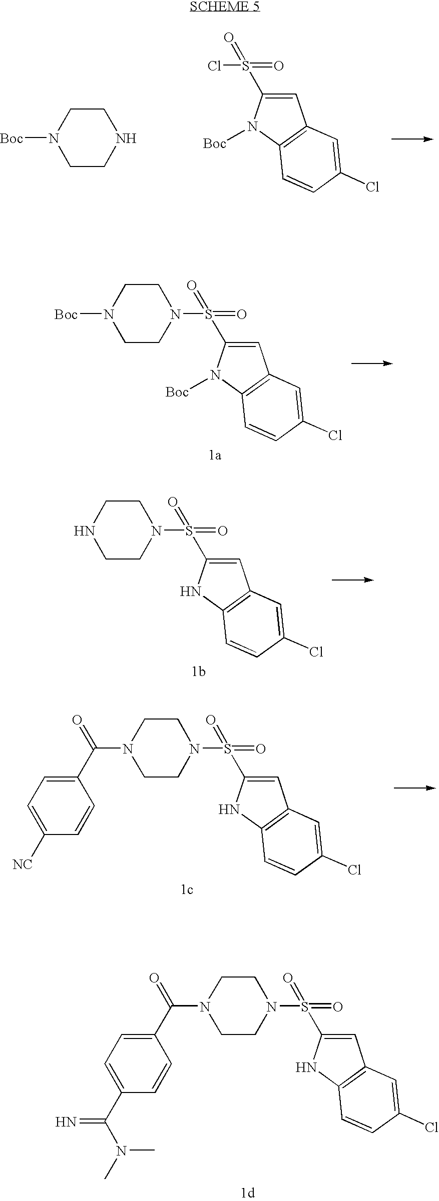

### EXAMPLE 1

### Preparation of 4-[(dimethylamino)iminomethyl]phenyl 4-[(5-chloroindol-2-yl)sulfonyl]piperazinyl ketone (1d).

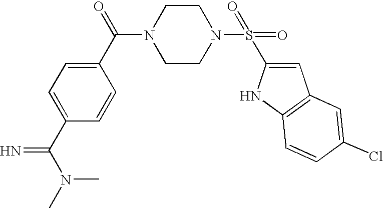

Step 1:

The mixture of 1-Boc-5-chloro-2-indolylsulfonylchloride (3.58 g, 10.2 mmol), 1-Boc-piperazine (2.86 g, 15.4) in 100 mL dry DCM was stirred at room temperature. To it was added 6.5 mL pyridine (80 mmol). The mixture was stirred for 3 hrs, diluted with 600 mL DCM, washed with 0.5N HCl×3. The organic phase was dried over MgSO₄, concentrated in vacuo to afford compound 1a as a powder (95%). MS found for C₂₂H₃₀ClN₃O₆S (M+Na)⁺ 522.

Step 2:

The above-prepared compound 1a was dissolved in 200 mL methanol. In ice bath it was charged with HCl gas till saturation. The solution was stirred for 2 days at room temperature and evaporated in vacuo to dryness to afford compound 1b (99%). MS found for C₁₂H₁₄ClN₃O₂S (M+H)⁺ 300.

Step 3:

Compound 1b (0.48 g, 1.6 mmol) was dissolved in 10 mL pyridine with catalytic amount of DMAP. At room temperature to it was added 4-cyanobenzoyl chloride (0.40 g, 2.4 mmol). The mixture was stirred for 4 hrs, concentrated in vacuo and purified with flash column to afford compound 1c (73%) as a solid. MS found for C₂₀H₁₇ClN₄O₃S (M+H)⁺ 429.

Step 4:

Compound 1c (40 mg, 0.1 mmol) was placed in 10 mL dry methanol. In ice bath it was stirred and charged with dry HCl gas directly from lecture bottle till saturation. The mixture was stirred for overnight. It was then evaporated in vacuo. The dry residue was dissolved in 5 mL dry methanol. To it was added dimethylamine (2M in methanol, 0.5 mL). The mixture was refluxed for 45 min and subjected to RP-HPLC separation to yield the title compound. MS found for 1d C₂₂H₂₄ClN₅O₃S (M+H)⁺ 474.

The following Examples 2-95 were similarly prepared by following the procedure of Example 1 as described above.

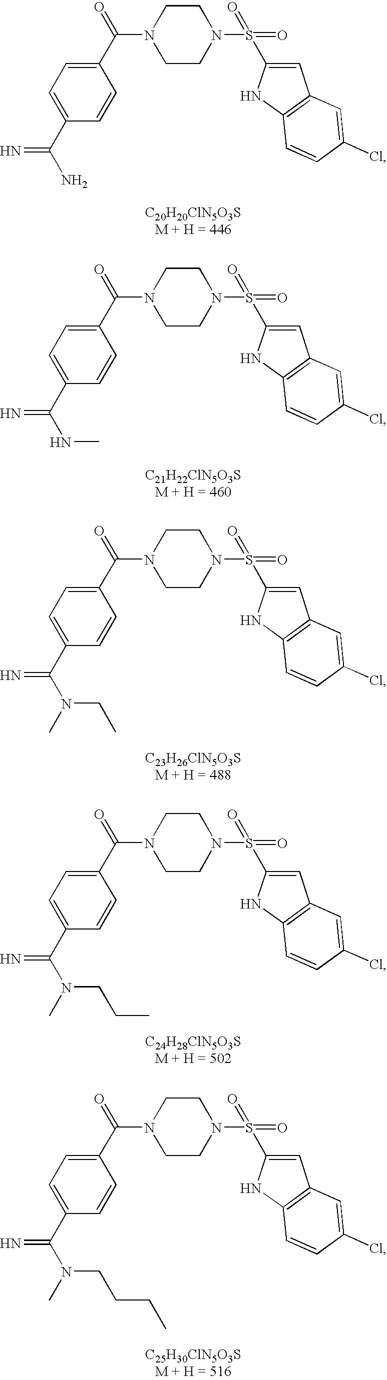

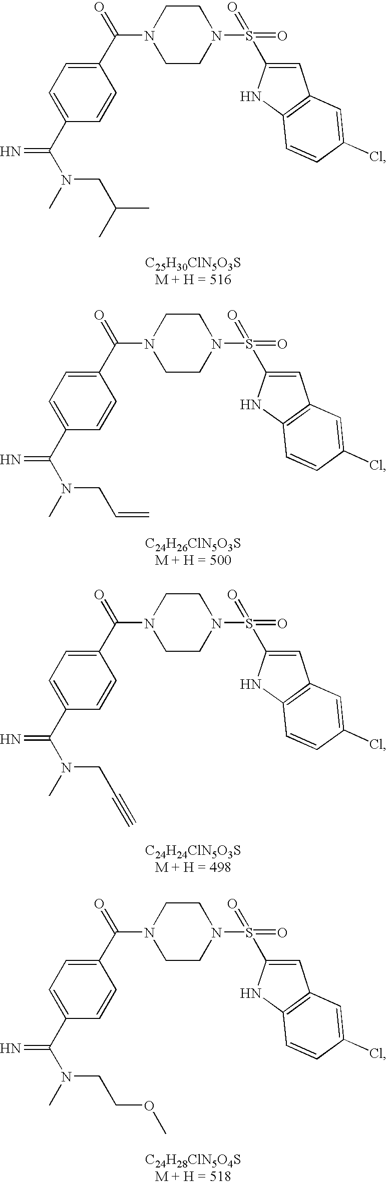

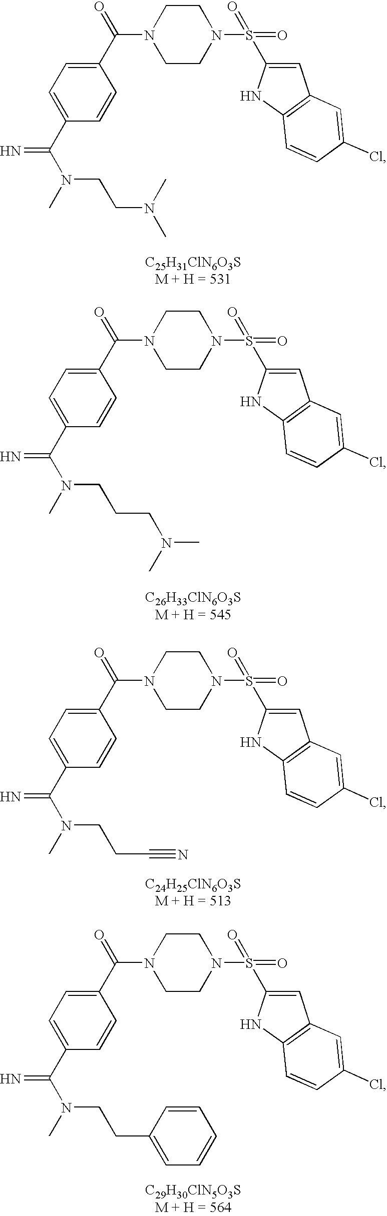

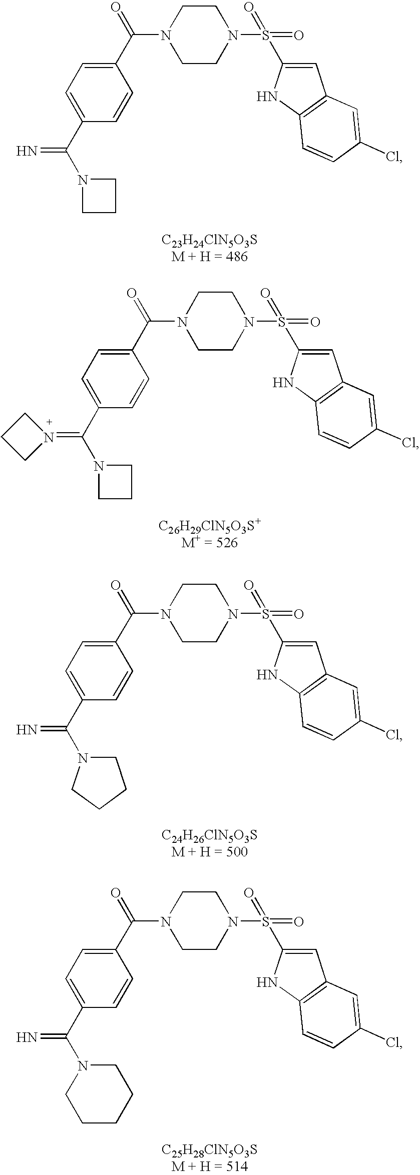

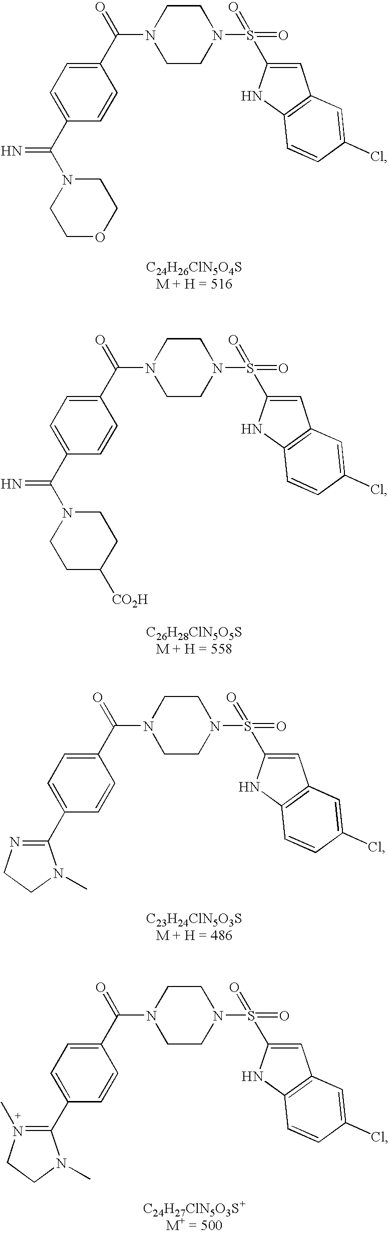

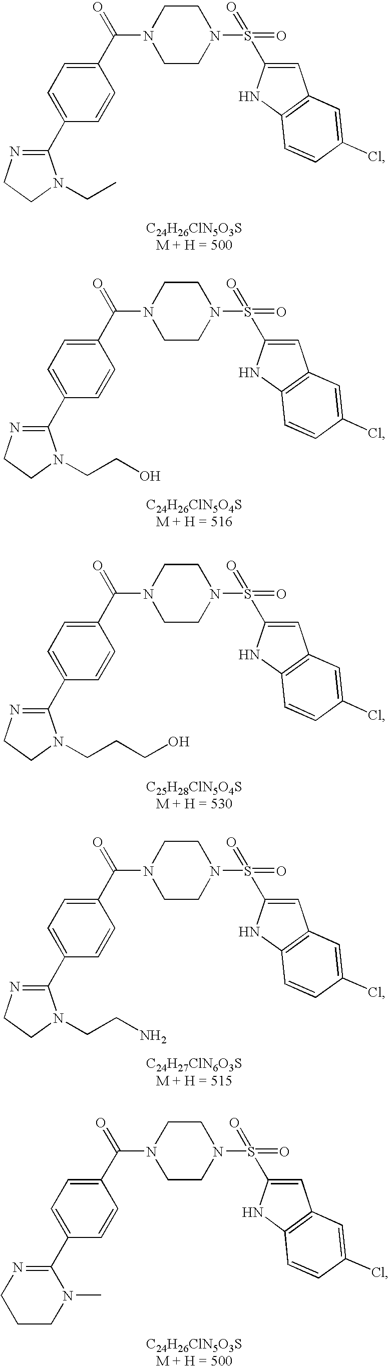

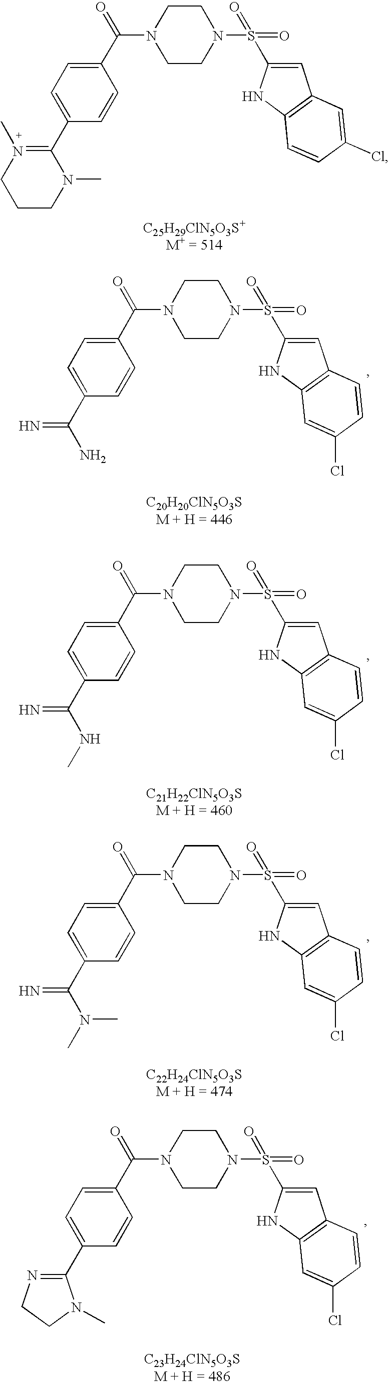

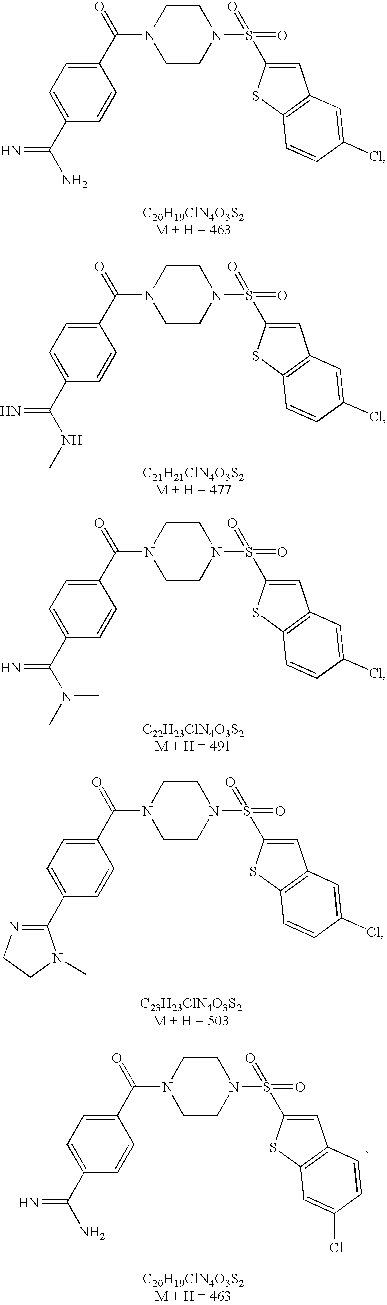

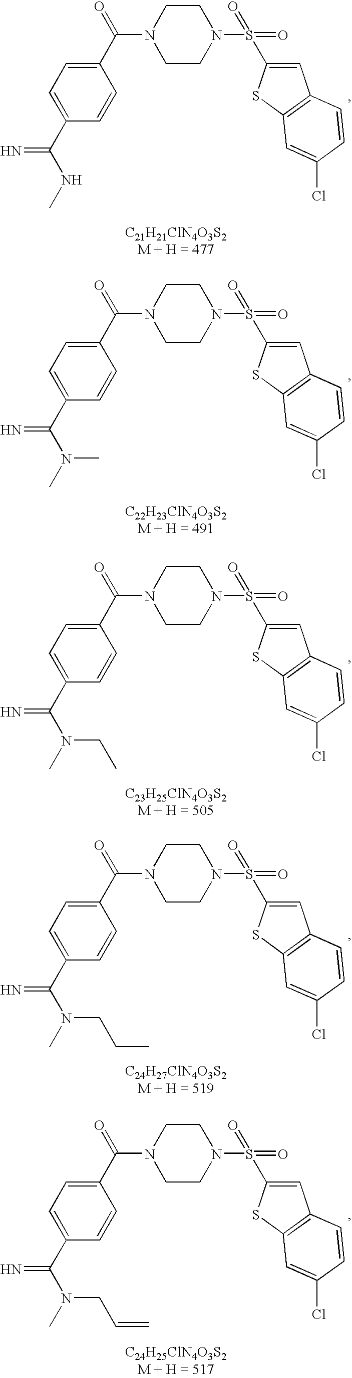

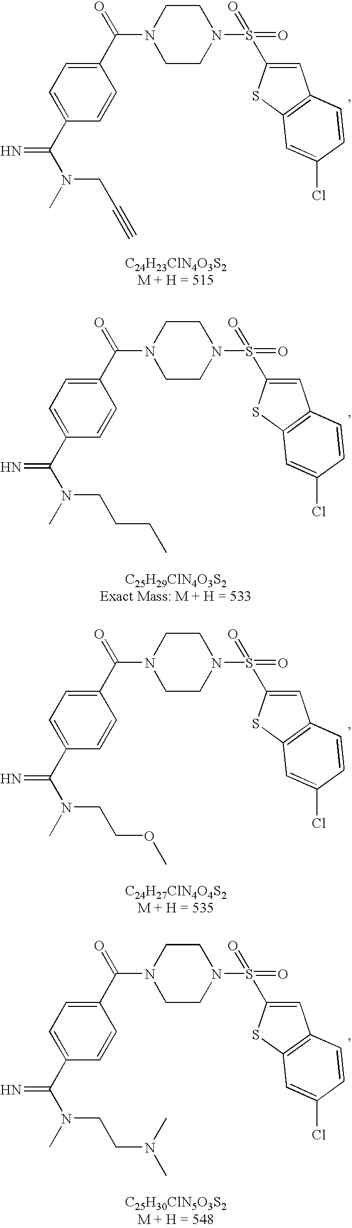

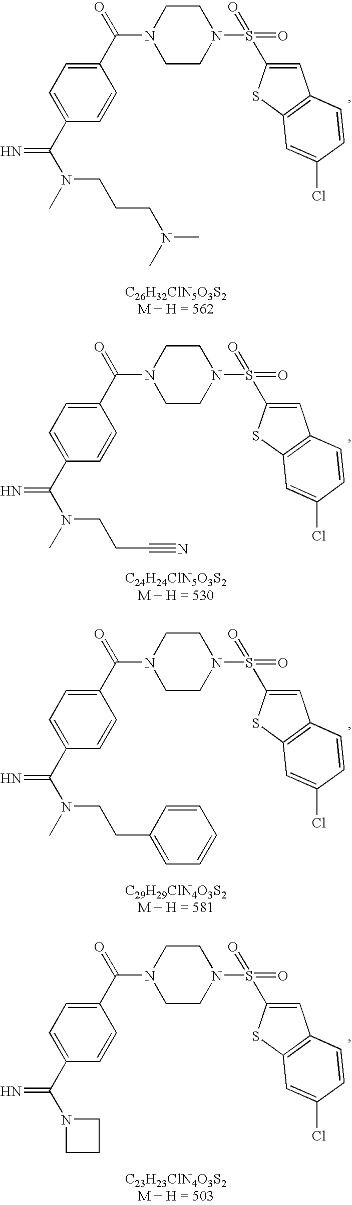

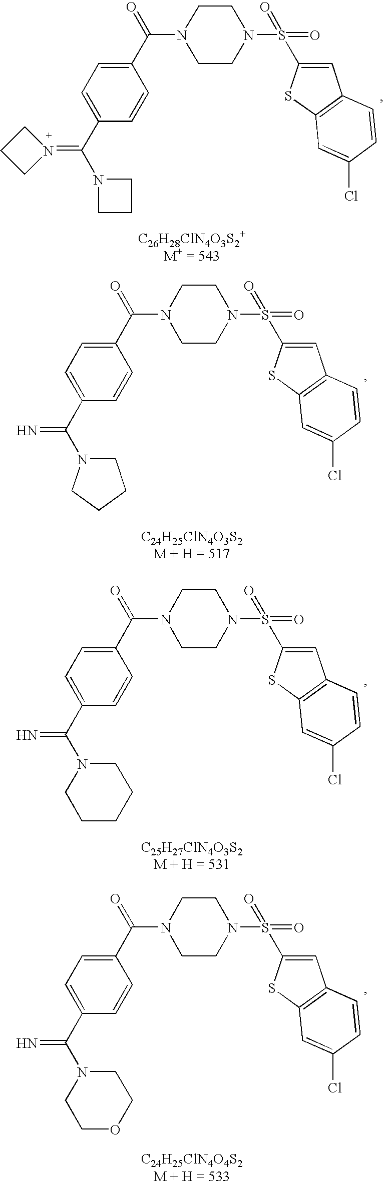

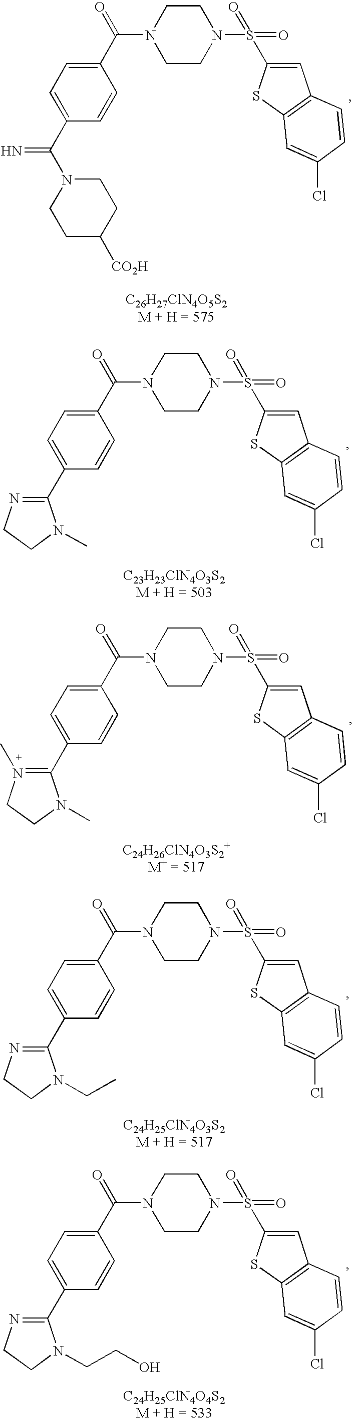

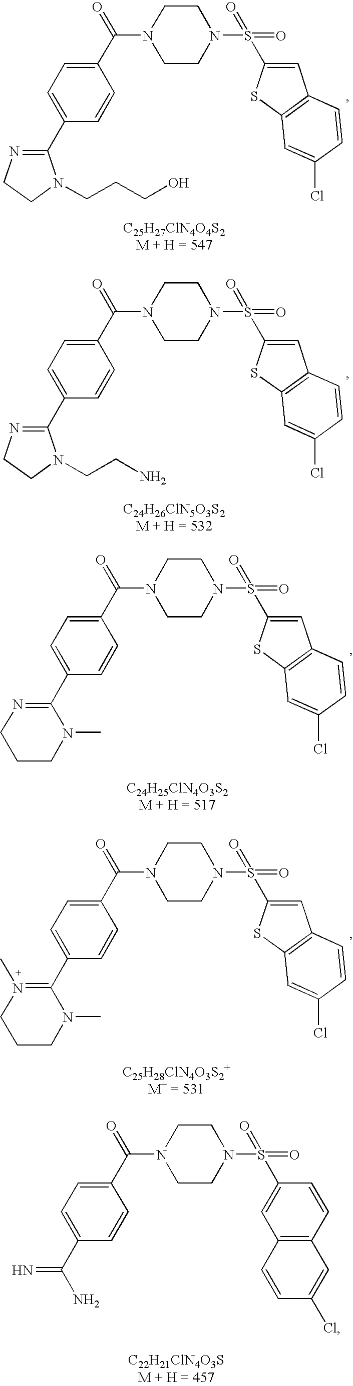

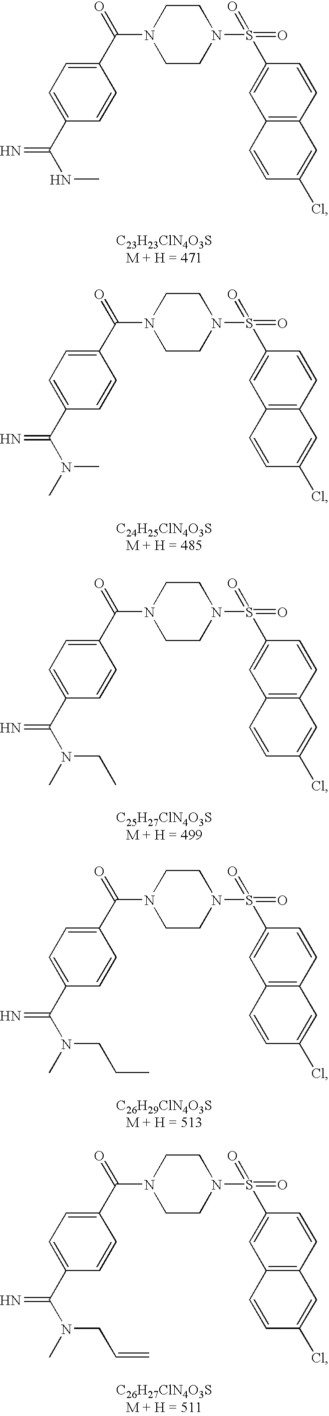

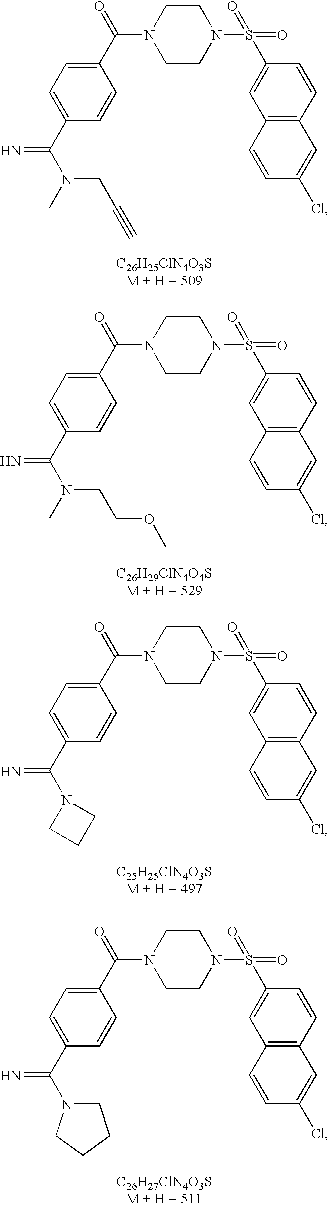

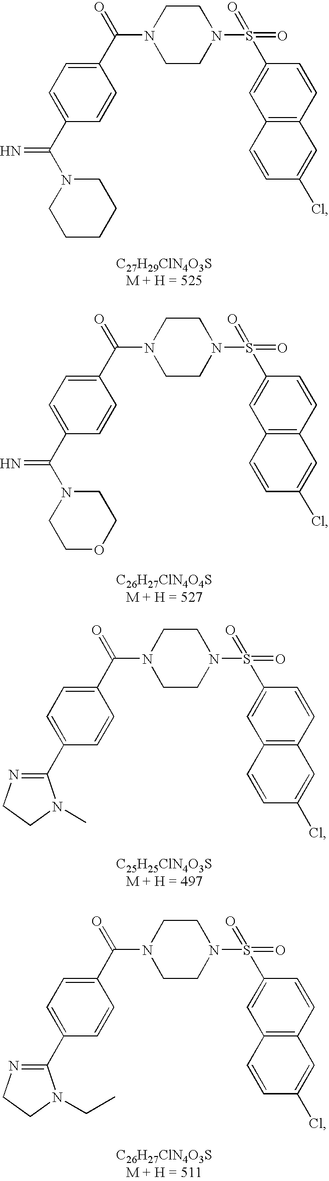

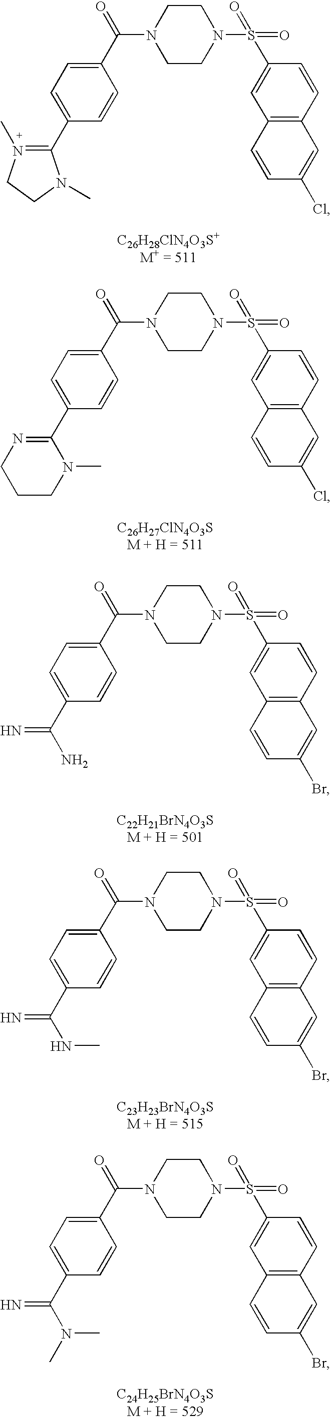

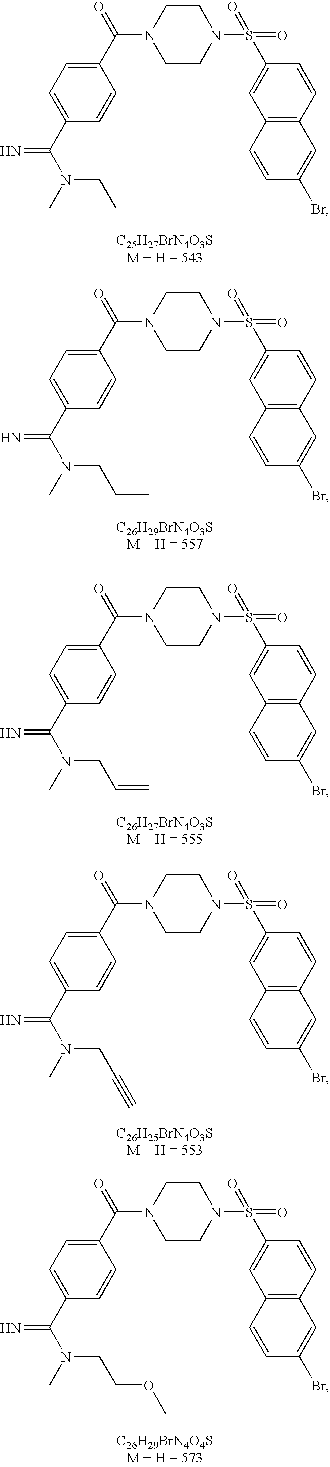

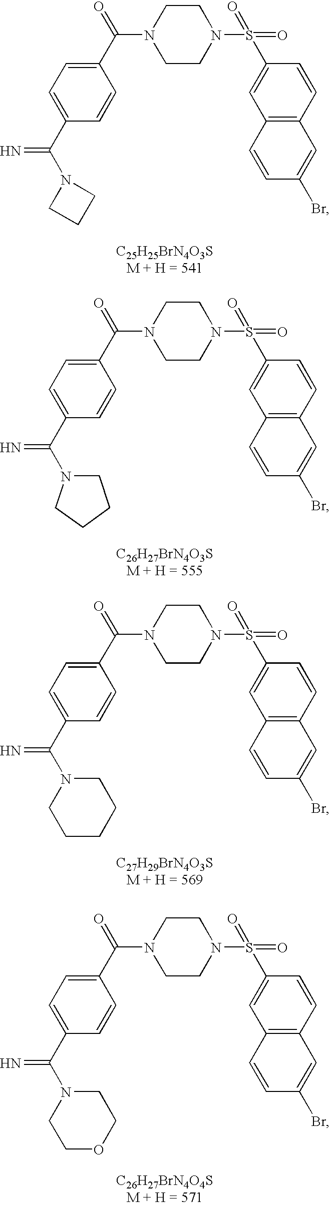

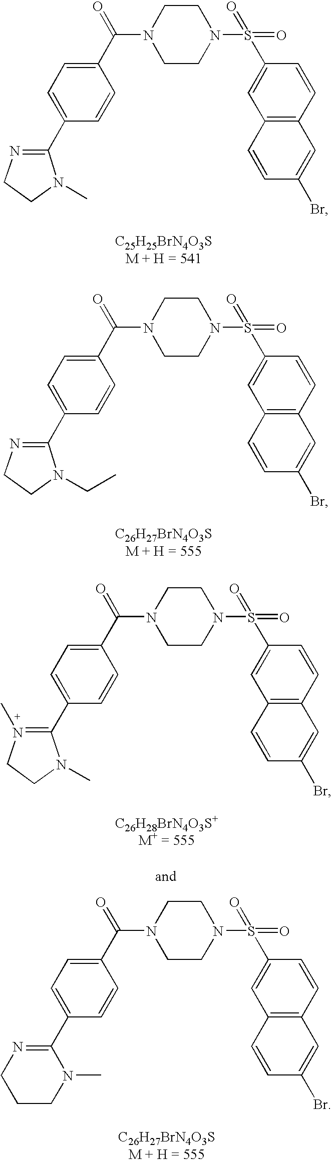

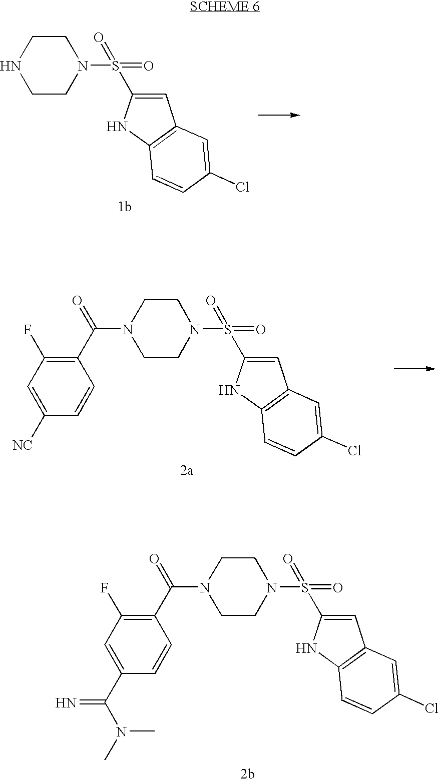

### EXAMPLE 96

### Preparation of 4-[(dimethylamino)iminomethyl]-2-fluorophenyl 4-[(5-chloroindol-2-yl)sulfonyl]piperazinyl ketone (2b)

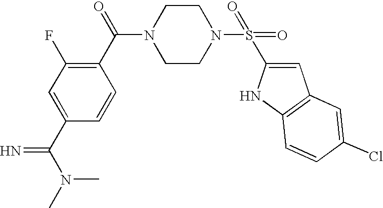

The synthetic procedure for these last 2 steps is the same as the Step 3 and Step 4 described in Scheme 5 and Example 1, using 4-cyano-2-fluorobenzoyl chloride to replace 4-cyanobenzoyl chloride. MS found for C₂₂H₂₃ClFN₅O₃S (M+H)⁺ 492.

The following Examples 97-134 were similarly prepared by following the procedures of Example 96.

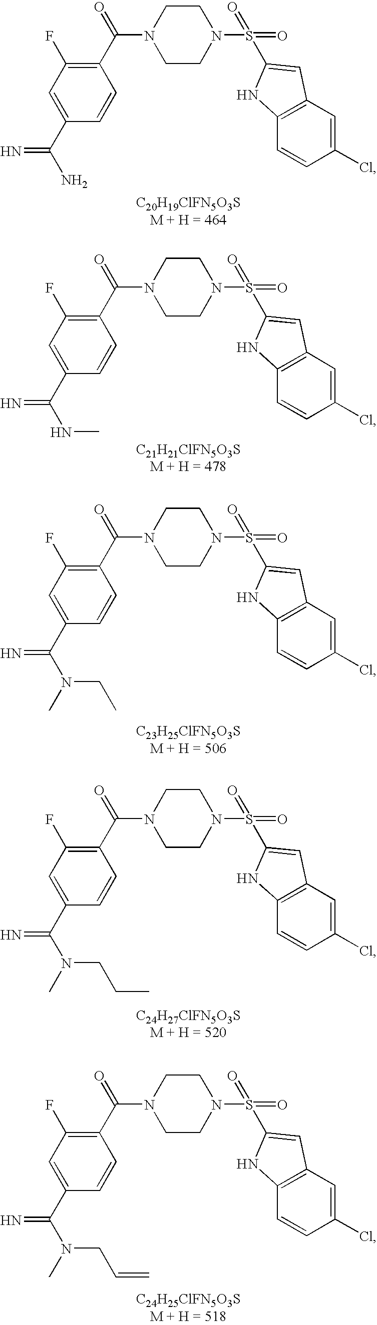

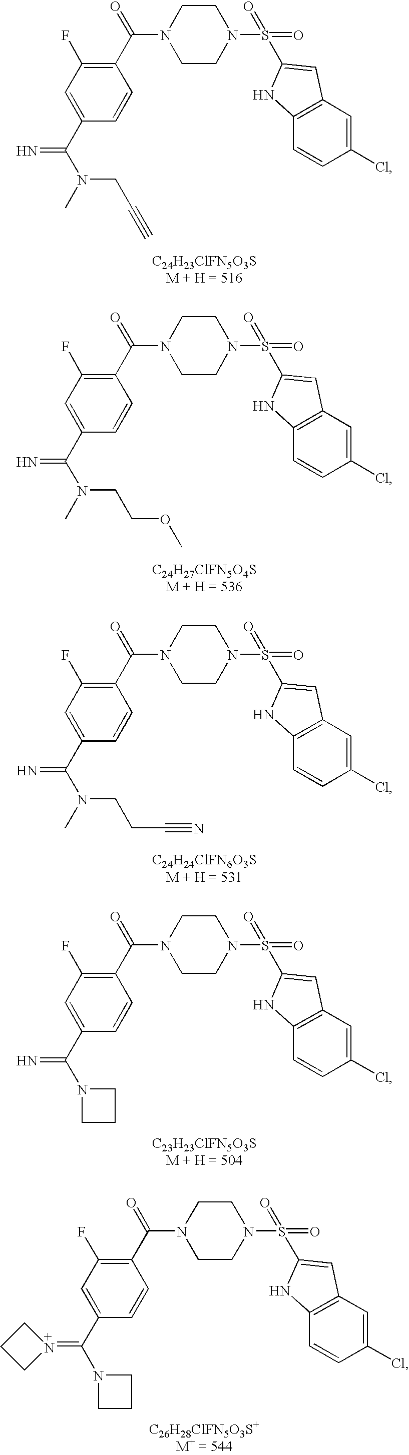

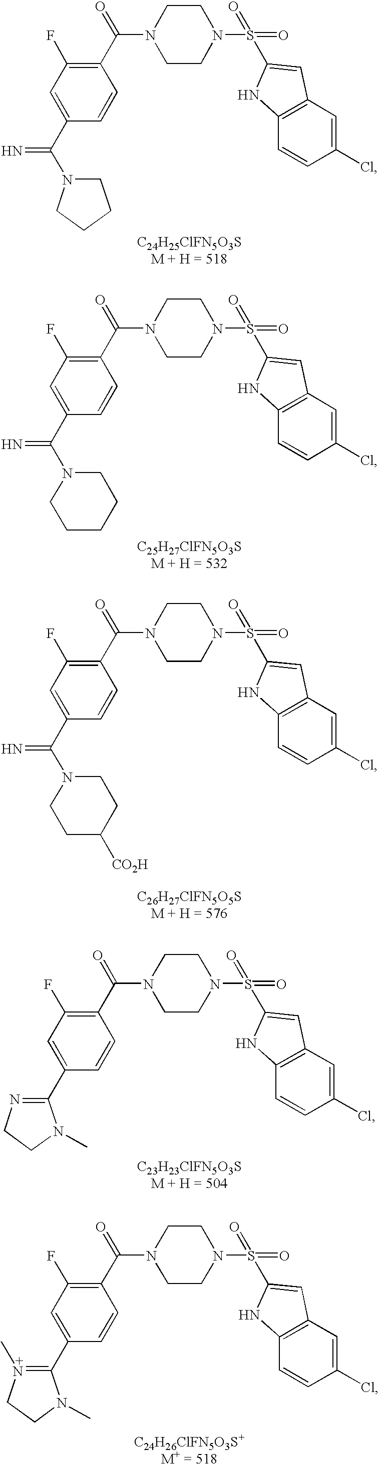

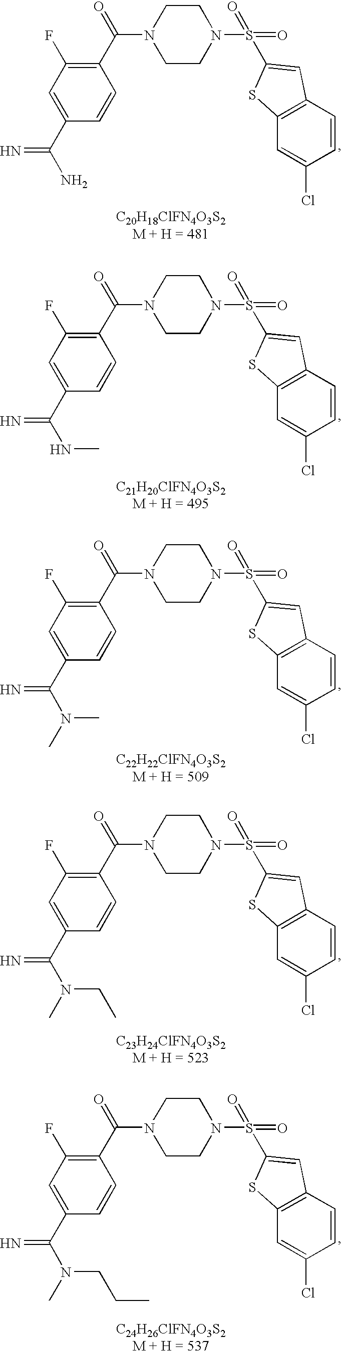

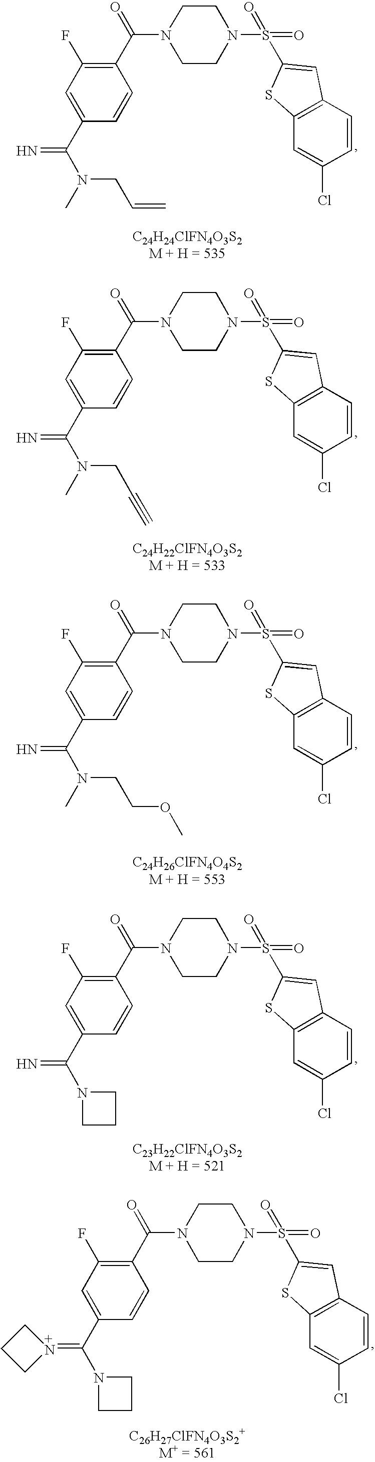

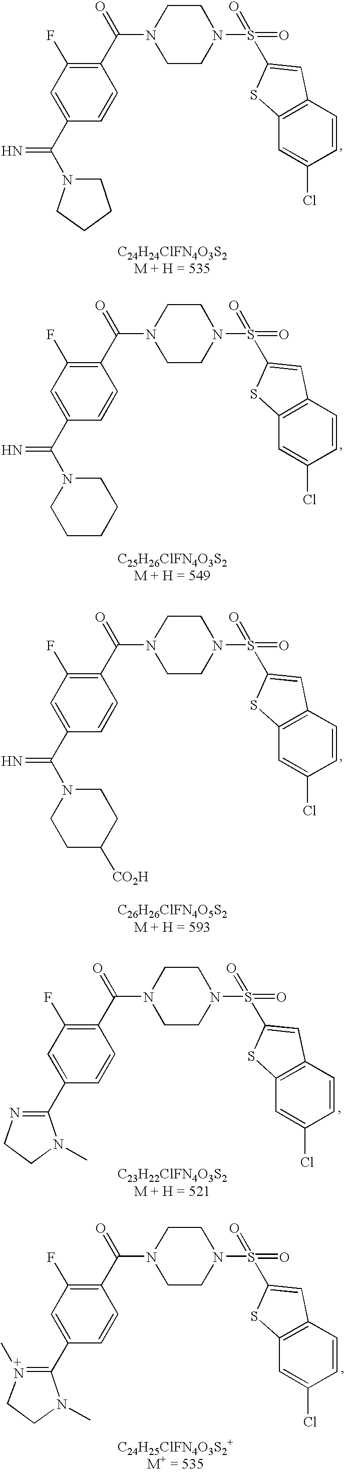

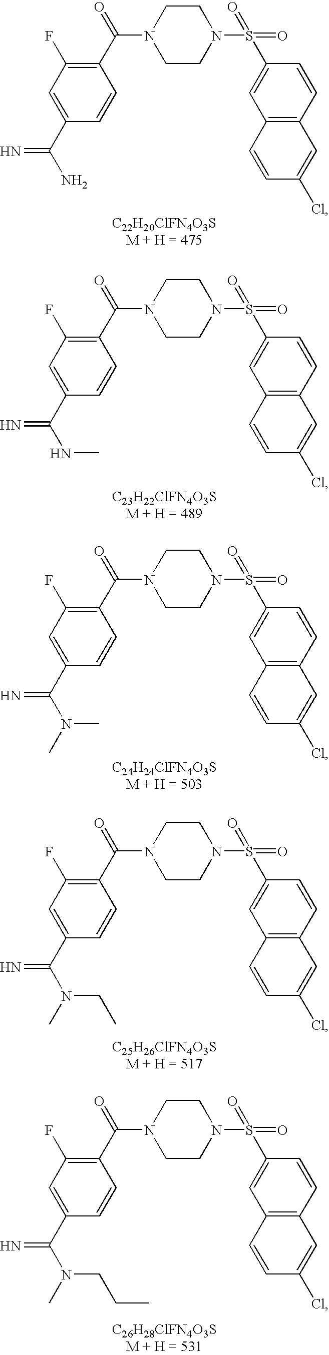

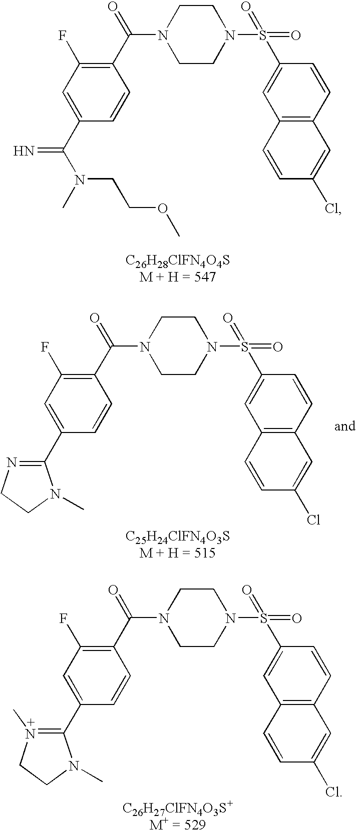

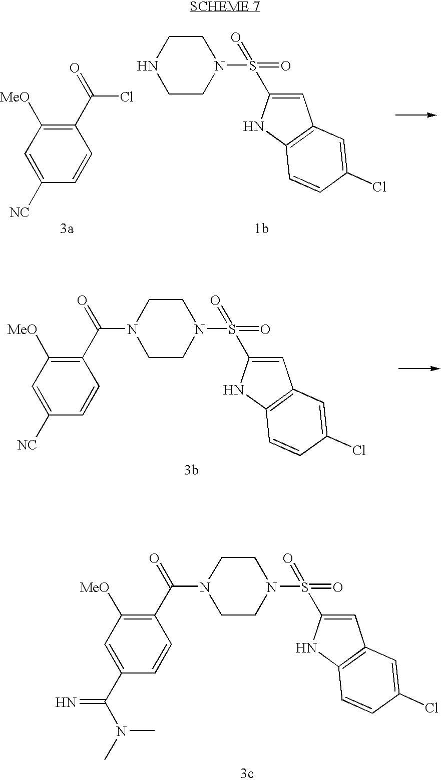

### EXAMPLE 135

### Preparation of 4-[(dimethylamino)iminomethyl]-2-methoxyphenyl 4-[(5-chloroindol-2-yl)sulfonyl]piperazinyl ketone (3c)

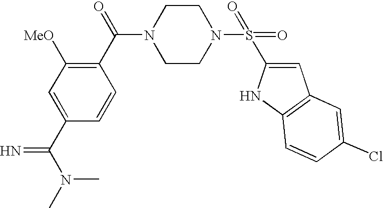

Preparation of 4-cyano-2-methoxybenzoyl chloride (3a). To the stirred mixture of CuCl (653 mg, 6.6 mmol) in 3 mL water at 0° C. was added NaCN (841 mg, 17 mmol, dissolved in 3 mL water). The mixture was stirred in ice bath. At the same time, commercial methyl 4-amino-2-inethoxybenzoate (1 g, 5.5 mmol) was stirred in 5 mL conc. HCl in ice bath. To it was added dropwise an ice-cold solution of NaNO₂ (457 mg, 6.6 mmol, in 3 mL water). The mixture was stirred in the ice bath for 1 hr. A dark orange solution was got. To it was added solid K₂CO₃ till no more CO₂ was released (pH=7). To the stirred cold suspension of freshly-prepared CuCN were added 5 mL toluene and then dropwise the neutralized diazonium slurry. The mixture was stirred in the ice bath for 20 min and then at room temperature for 1 hr. The mixture was diluted with 300 mL EtOAc, washed with water and brine, dried, purified by flash column to afford methyl 4-cyano-2-methoxybenzoate (71%). MS found for C₁₀H₉NO₃ (M+H)⁺ 192. This ester was then dissolved in 30 mL methanol. To it were added 5 mL water and LiOH.H₂O (50 mg). The mixture was stirred for 6 hrs at room temperature. It was then concentrated in vacuo, acidified with 2N HCl, extracted with EtOAc (100 mL×3). The organic extract was dried over MgSO₄ and concentrated in vacuo to dryness to afford 4-cyano-2-methoxybenzoic acid (95%). MS found for C₉H₇NO₃ (M+Na)⁺ 200. This acid was then stirred in 30 mL dry DCM. To it were added 0.1 mL DMF and dropwise 1 mL oxalyl chloride. The mixture was stirred for 3 hrs. A clear solution was got and evaporated in vacuo till dryness to produce 4-cyano-2-methoxybenzoyl chloride. It was ready for use without purification.

The synthetic procedure for the last 2 steps is the same as the Step 3 and Step 4 described in Scheme 5 and Example 1, using 4-cyano-2-methoxybenzoyl chloride to replace 4-cyanobenzoyl chloride. MS found for C₂₃H₂₆ClN₅O₄S (M+H)⁺ 504.

The following Examples 136-182 were similarly prepared by following the procedure of Example 135.

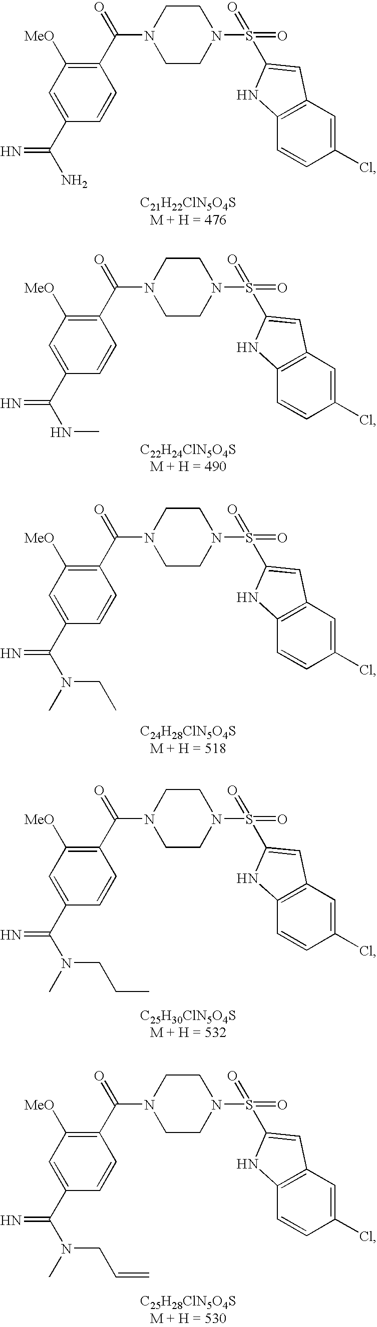

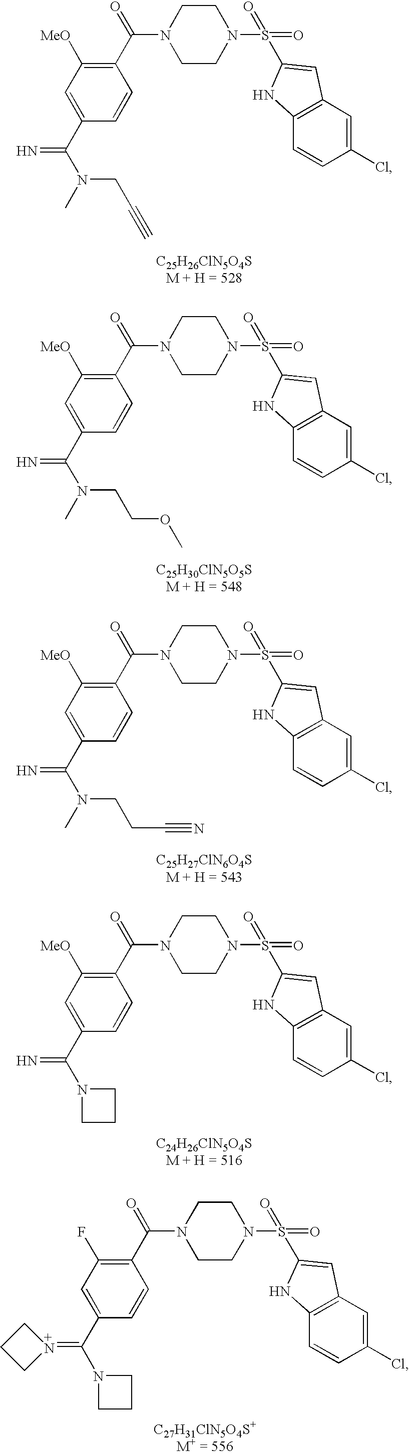

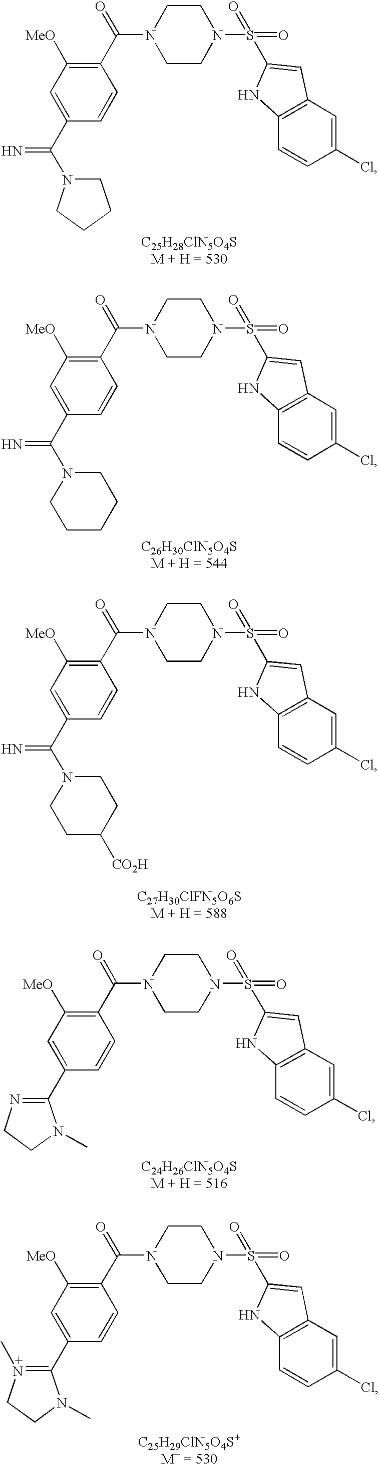

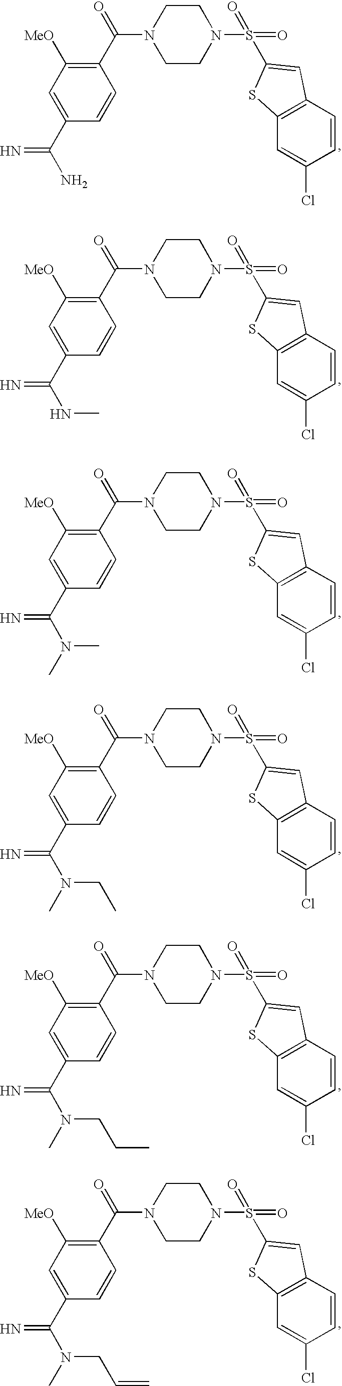

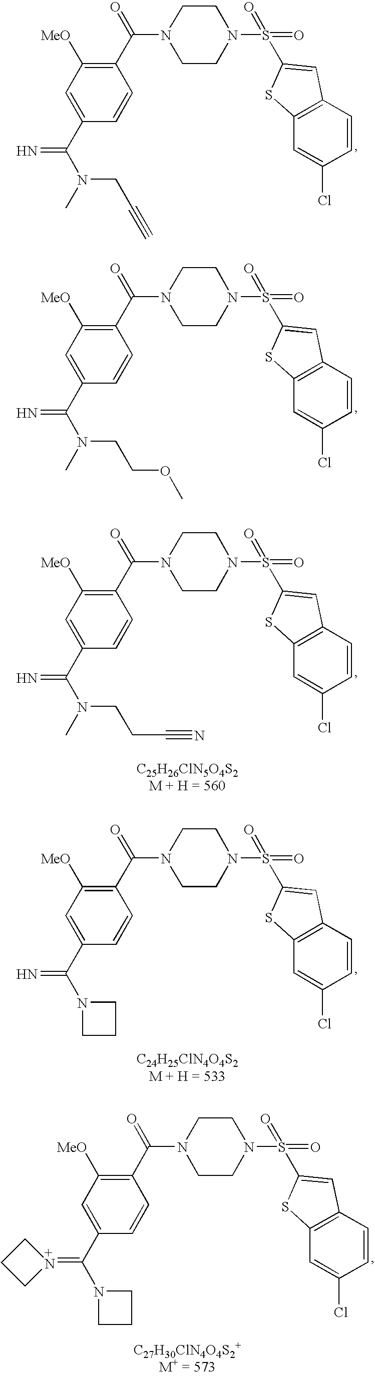

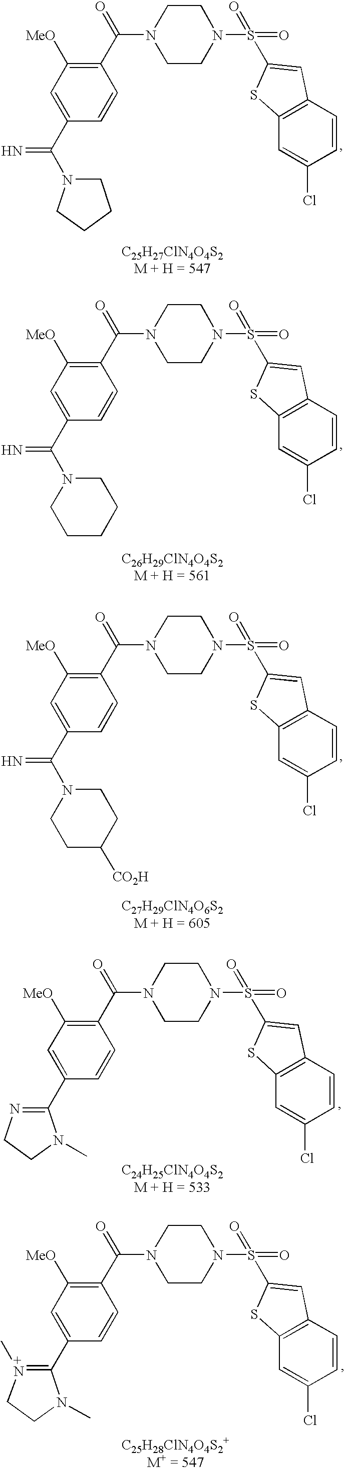

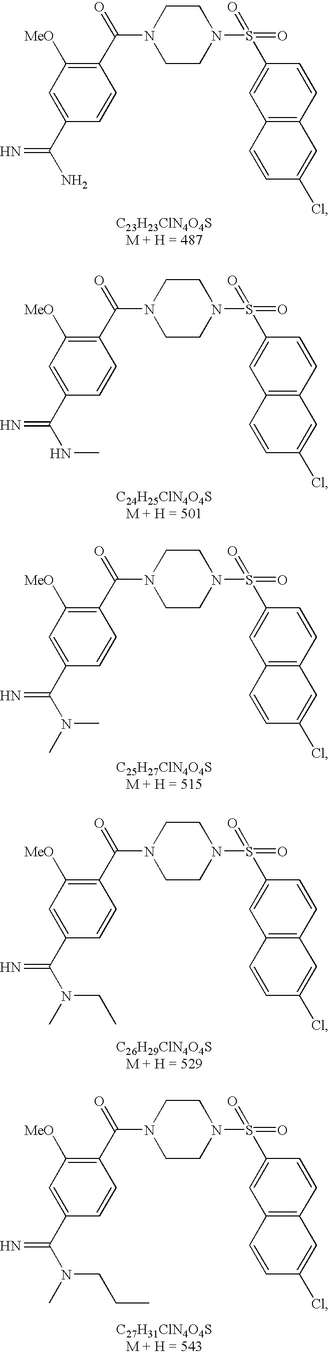

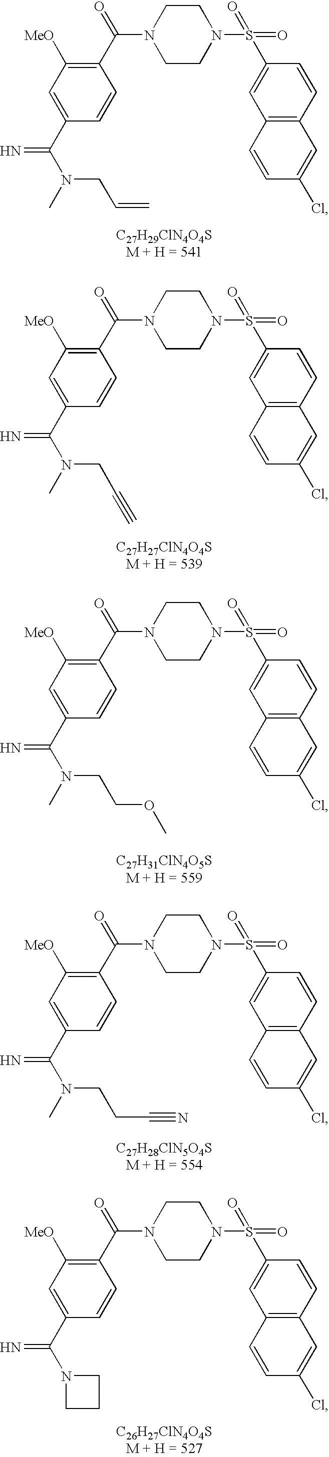

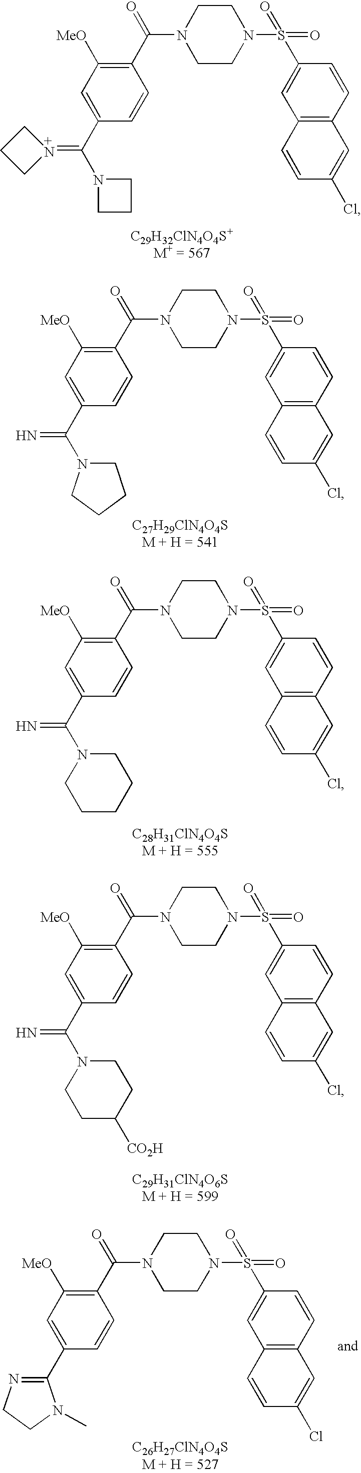

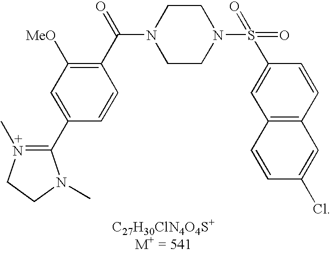

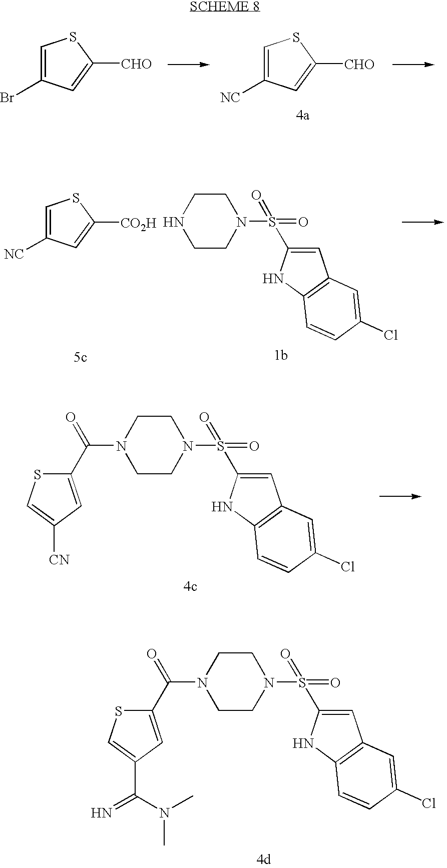

### EXAMPLE 183

### Preparation of 4-[(dimethylamino)iminomethyl](2-thienyl) 4-[(5-chloroindol-2-yl)sulfonyl]piperazinyl ketone (4d)

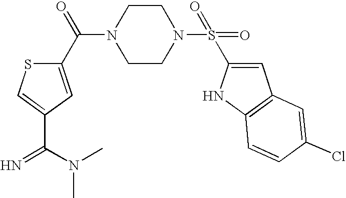

Step 1:

To a stirred solution of 4-bromo-2-thiophenecarboxaldehyde (4.5 g, 23 mmol) in anhydrous DMF (25 mL) were added CuCN (6.4 g, 71 mmol) and CuI (2.0 g, 10 mmol). The slurry was refluxed for 1 hr. It was diluted with 300 mL DMF and filtered through a celite-silica bed to remove the solid. The filtrate was concentrated in vacuo. The residue was washed with hot chloroform (200 mL×4). The chloroform extracts were decanted, combined and washed with water. The chloroform solution was dried over MgSO₄ and concentrated in vacuo to give 4a (2.2 g, 71%) as a solid. MS found for C₆H₃NOS (M+H)⁺ 138.

Step 2:

To a stirred solution of 4a (2.2 g, 16 mmol) in 20 mL acetone were added tBuOH (150 mL) and 2-methylbutene (30 mL). To the mixture was then added a solution of NaClO₂ (13 g, 150 mmol) and NaH₂PO₄ (12 g, 100 mmol) in 100 mL water at room temperature. The mixture was stirred for 1 hr. It was acidified with 1N HCl till pH=1. The mixture was rotovaped to remove organic solvents. The residue was extracted with EtOAc (250 mL×4). The organic extracts were dried over MgSO₄ and concentrated in vacuuo to afford 4b (2.8 g, 99%) as a white solid. MS found for C₆H₃NO₂S (M+Na)⁺ 176.

Step 3:

Acid 4b (123 mg, 0.83 mmol) was placed in 3 mL dry DCM. To it were added one drop of dry DMF and 0.3 mL oxalyl chloride. To mixture was stirred for 2 hrs and then evaporated in vacuuo. It was dissolved in 3 mL THF. To it were added 1b (165 mg, 0.55 mmol) and 0.3 mL pyridine. The mixture was stirred for 1 hr. It was diluted with 150 mL chloroform, washed with water×2, dried, evaporated and purified by flash column to afford 4c (180 mg, 75%). MS found for C₁₈H₁₅ClN₄O₃S₂ (M+H)⁺ 435.

Step 4:

Compound 4c (40 mg, 0.1 mmol) was placed in 10 mL dry methanol. In ice bath it was stirred and charged with dry HCl gas directly from lecture bottle till saturation. The mixture was stirred for overnight. It was then evaporated in vacuuo. The dry residue was dissolved in 5 mL dry methanol. To it was added dimethylamine (2M in methanol, 0.5 mL). The mixture was refluxed for 45 min and subjected to RP-HPLC separation to yield the title compound. MS found for 4d C₂₀H₂₂ClN₅O₃S₂ (M+H)⁺ 480.

The following Examples 184-231 were similarly prepared by following the procedure of Example 183.

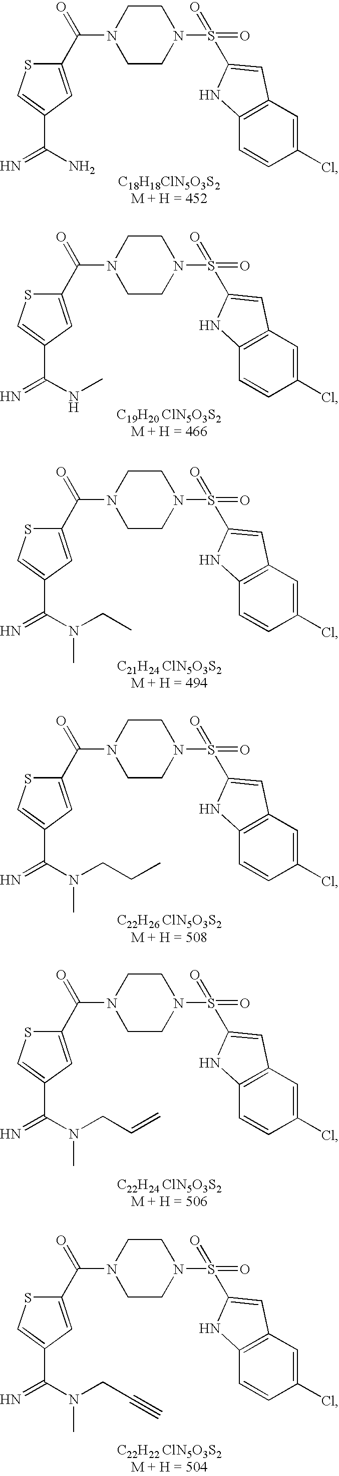

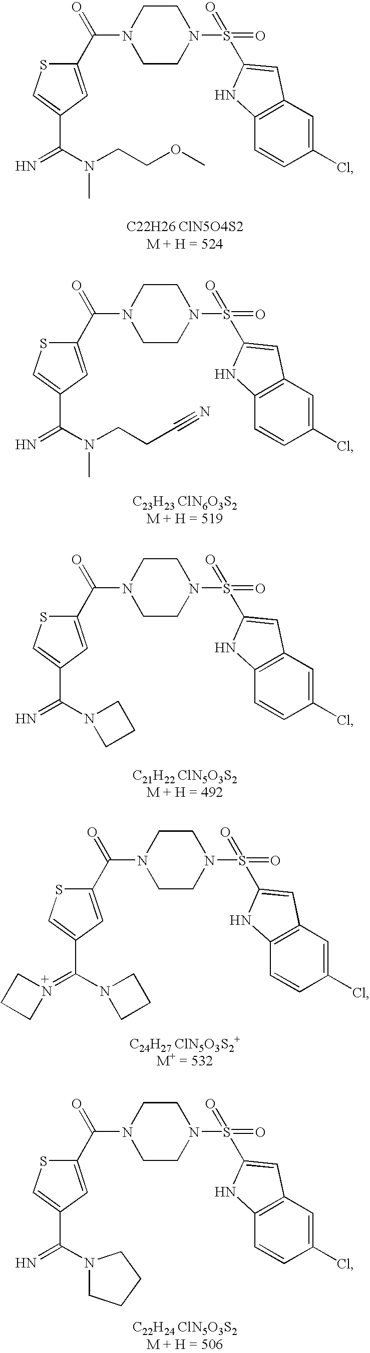

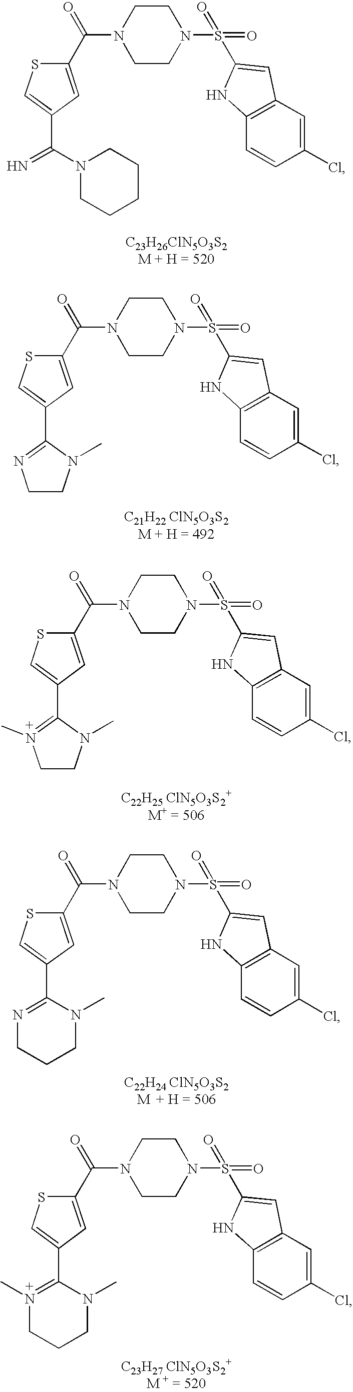

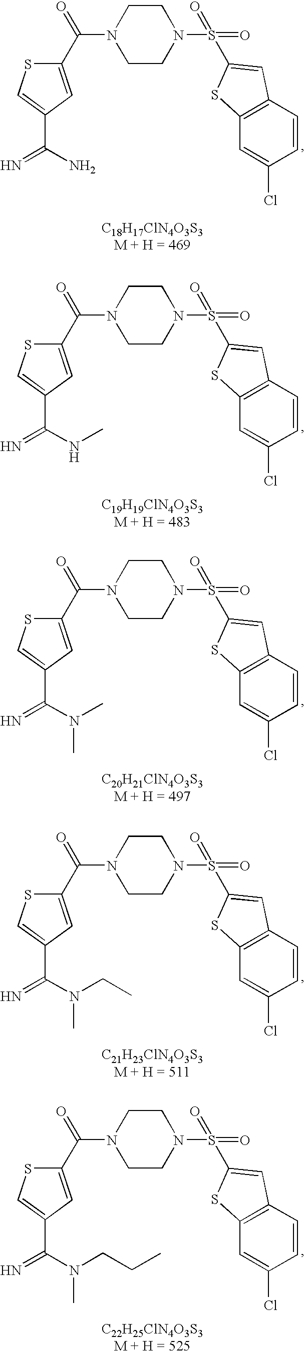

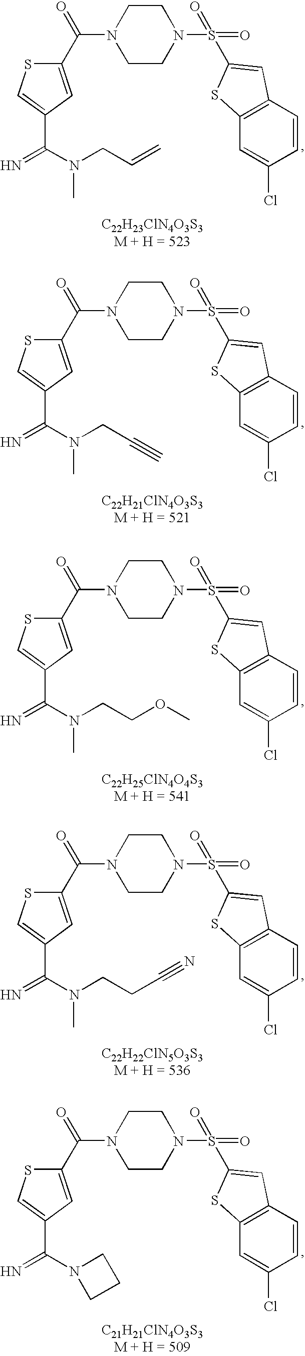

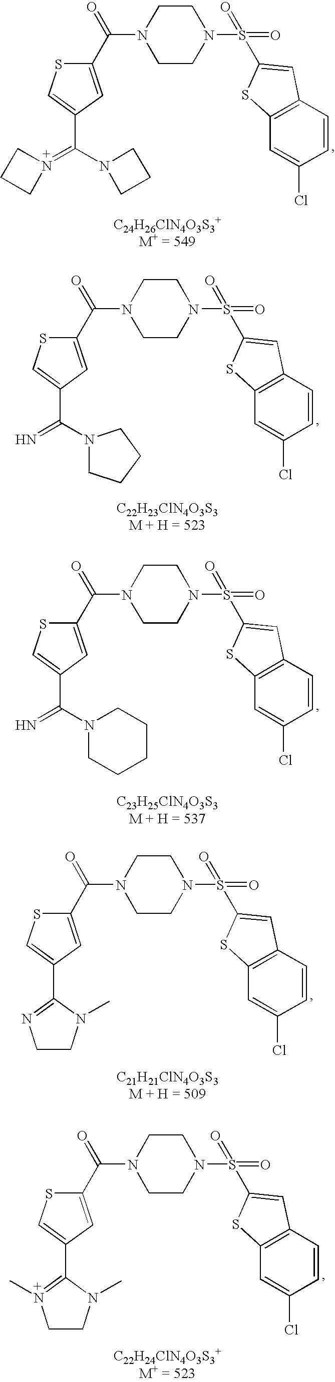

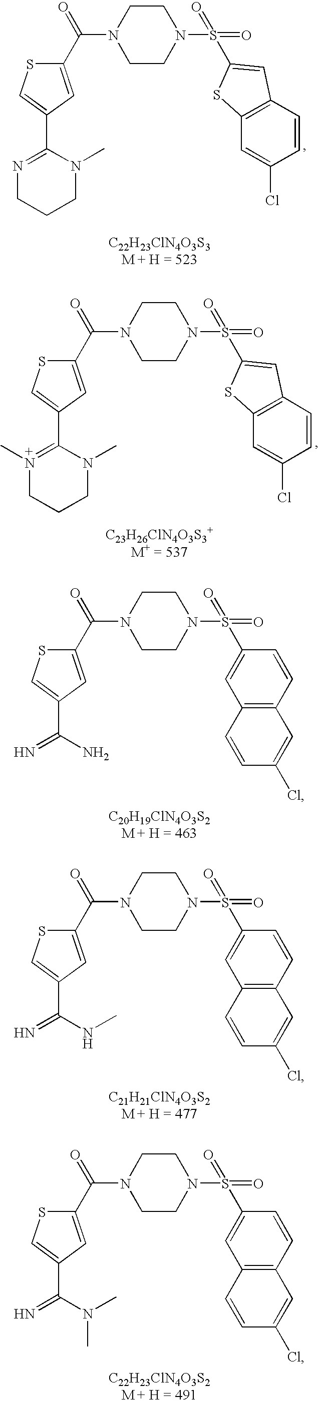

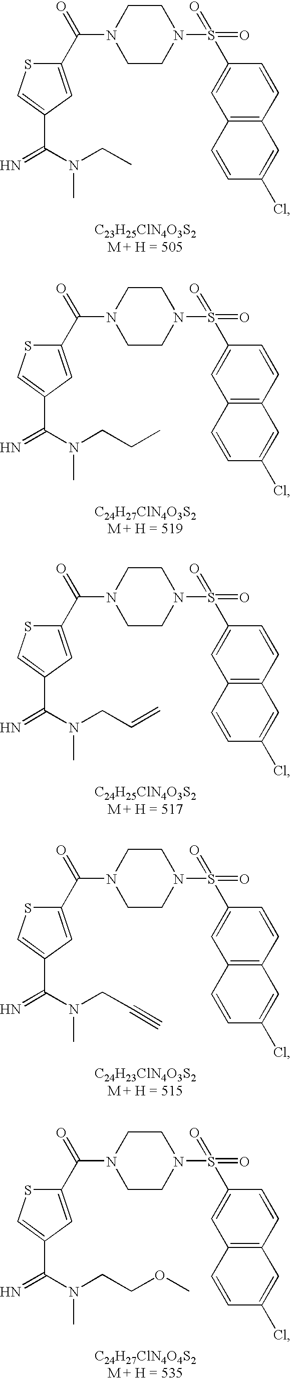

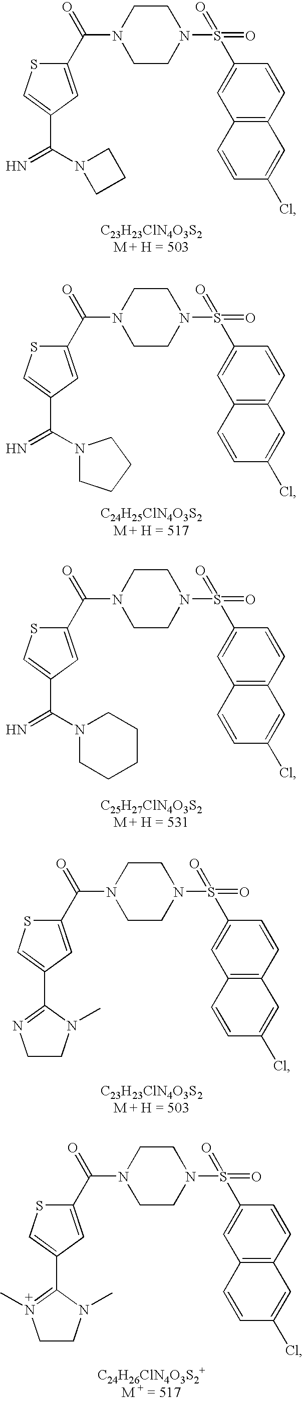

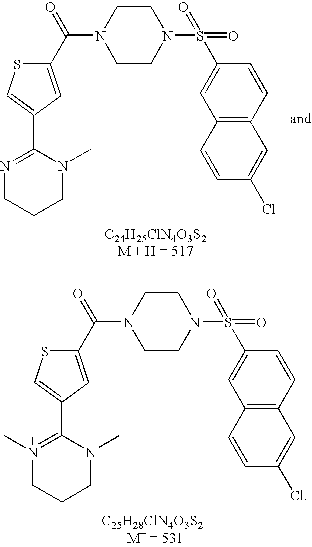

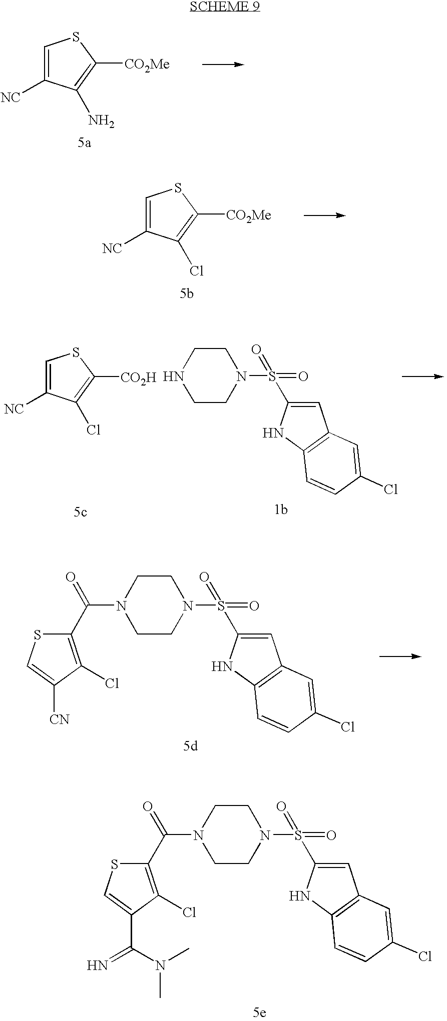

### EXAMPLE 232

### Preparation of 4-[(dimethylamino)iminomethyl]-3-chloro(2-thienyl) 4-[(5-chloroindol-2-yl)sulfonyl]piperazinyl ketone (5e)

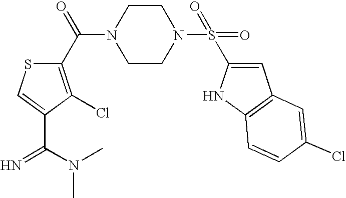

Step 1:

A mixture of CuCl (1.1 g, 11 mmol) and tBuONO (1.3 mL, 11 mmol) in 20 mL MeCN was refluxed for 30 min. To it was added commercial compound 5a (1.0 g, 5.5 mmol). The mixture was refluxed for 1 hr. It was diluted with 300 mL chloroform and filtered through celite. The filtrate was washed by water×3, dried over MgSO₄ and concentrated in vacuuo to afford compound 5b (0.68 g, 62%) as a solid. MS found for C₇H₄ClNO₂S (M+H)⁺ 202.

Step 2:

Compound 5b (0.43 g, 2.1 mmol) was dissolved in 30 mL methanol. To it were added 10 mL water and LiOH.H₂O (180 mg, 4.2 mmol). The mixture was stirred for 2 hrs. It was concentrated to remove methanol, acidified to pH=1 with 1N HCl and extracted with EtOAc×3. The organic extract was dried and concentrated in vacuuo to afford acid 5c (0.38 g, 95%) as a white solid. MS found for C₆H₂ClNO₂S (M+H)⁺ 188.

Step 3 and Step 4:

The synthetic procedure for these last 2 steps is the same as the Step 3 and Step 4 described in Example 183. MS found for C₂₀H₂₁Cl₂N₅O₃S₂ (M+H)⁺ 514.

The following Examples 233-276 were similarly prepared by following the procedure of Example 232.

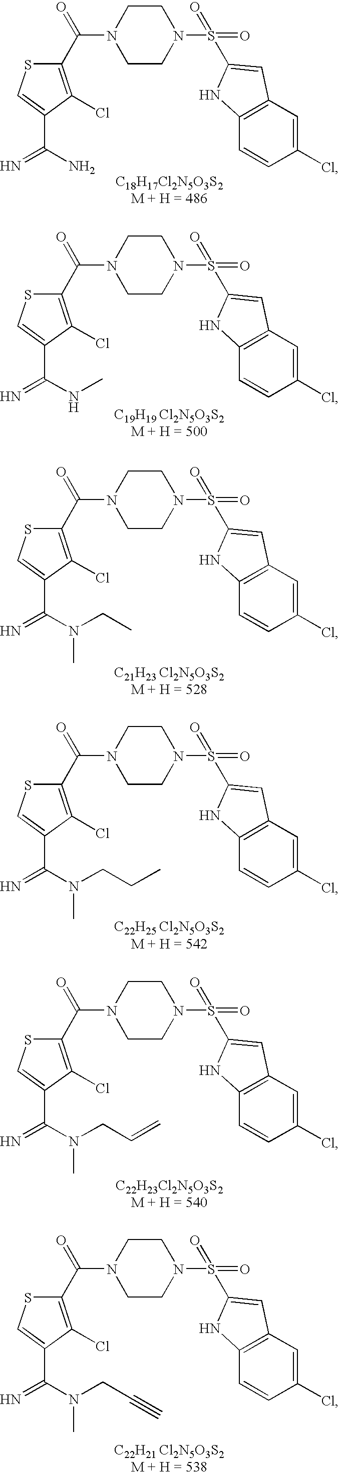

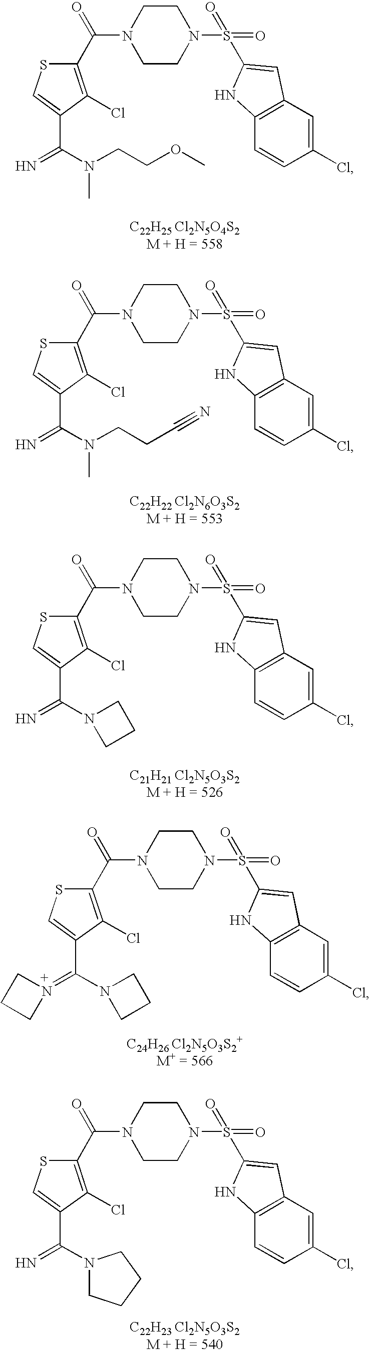

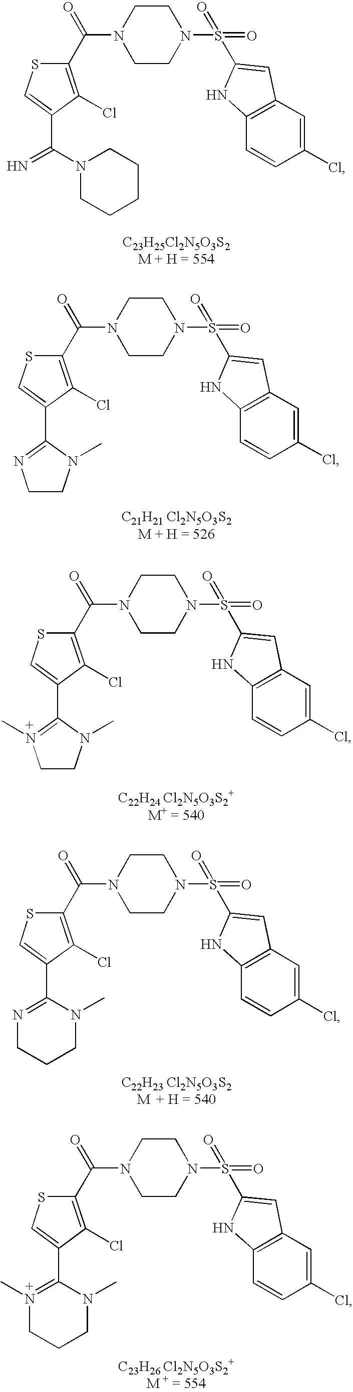

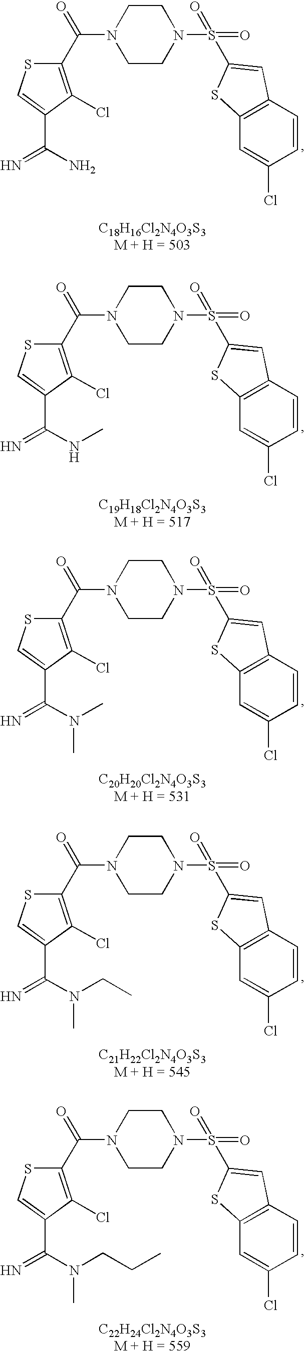

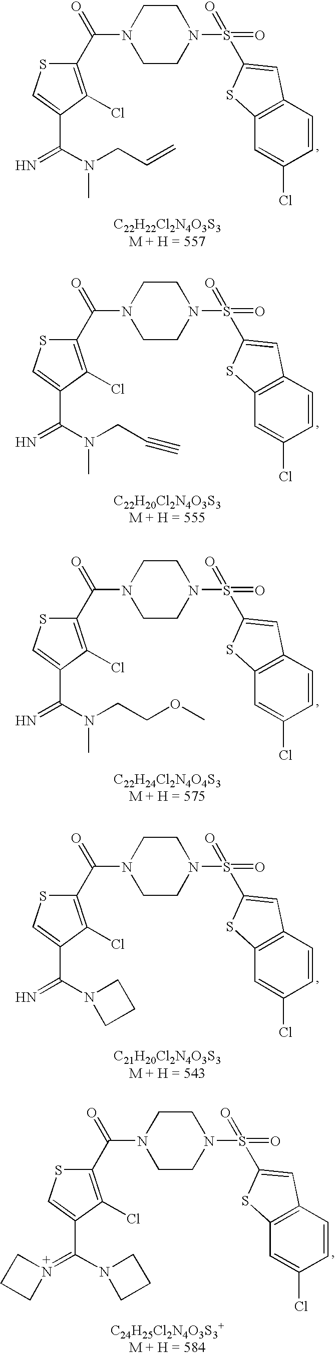

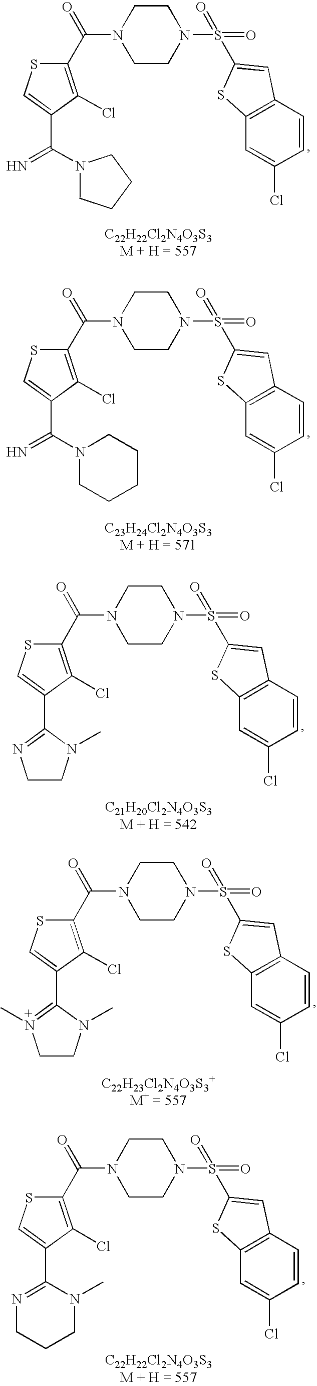

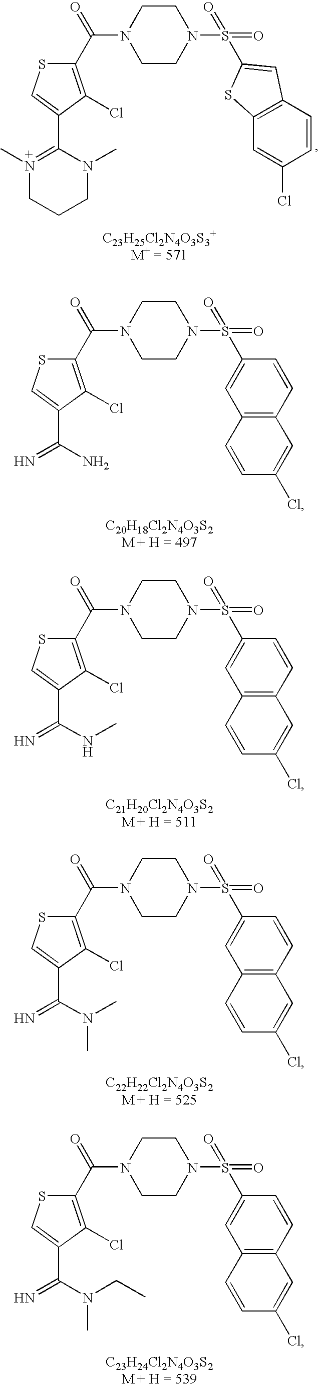

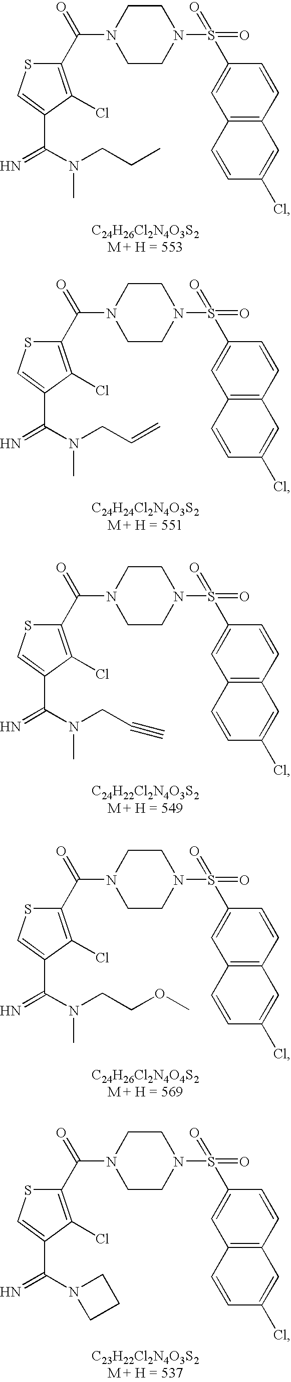

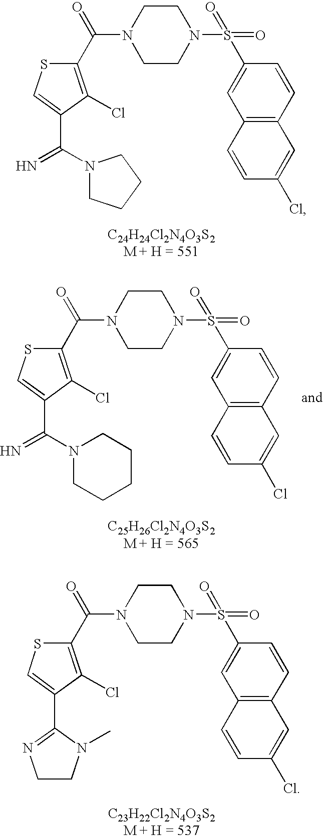

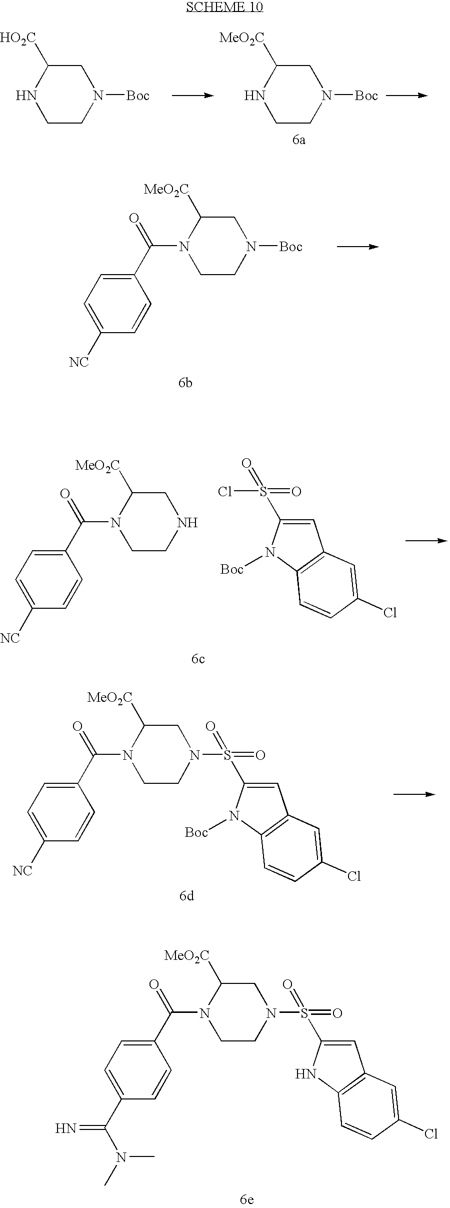

### EXAMPLE 277

### Preparation of methyl 1-({4-[(dimethylamino)iminomethyl]phenyl}carbonyl)-4-[(5-chloroindol-2-yl)sulfonyl]piperazine-2-carboxylate (6e)

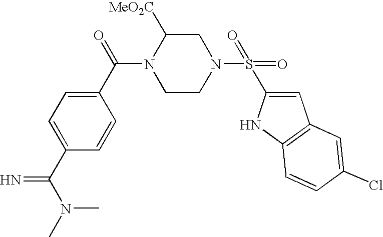

Step 1, Step 2 and Step 3:

4-Boc-piperazine-2-carboxylic acid (500 mg, 2.2 mmol) was dissolved in 25 mL dry THF. To this stirred solution was added trimethylsilyldiazomethane (commercial 2M in THF, 2.8 mL, 5.4 mmol). The reaction was stirred for 6 hrs and quenched with HOAc. It was concentrated in vacuuo, dissolved in 150 mL EtOAc, washed with sat. NaHCO₃ 30 mL×3, dried, evaporated in vacuuo to afford 6a (85%). MS found for C₁₁H₂₀N₂O₄ (M+Na)⁺ 267. It was dissolved in 5 mL dry THF. To it were added 4-cyanobenzoyl chloride (330 mg, 2 mmol) and 1 mL pyridine. The mixture was stirred for 30 min, concentrated in vacuuo and subjected to flash column to isolate compound 6b (80%). MS found for C₁₉H₂₃N₃O₅ (M+Na)⁺ 396. Compound 6b was then stirred in 4N HCl (commercial dioxane solution) for 1 hr to give compound 6c (99%). MS found for C₁₄H₁₅N₃O₃ (M+H)⁺ 274.

Step 4:

Compound 6c (270 mg, 1 mmol) was dissolved in 10 mL dry pyridine. To it was added 1-Boc-5-chloro-2-indolylsulfonylchloride (350 mg, 1 mmol). The mixture was stirred for overnight. It was concentrated in vacuuo and subjected to flash column for separation of compound 6d (340 mg, 58%). MS found for C₂₇H₂₇ClN_{4O}₇S (M+Na)⁺ 609.

Step 5:

Compound 6d (40 mg, 0.07 mmol) was placed in 10 mL dry methanol. In ice bath it was stirred and charged with dry HCl gas directly from lecture bottle till saturation. The mixture was stirred for overnight. It was then evaporated in vacuuo. The dry residue was dissolved in 5 mL dry methanol. To it was added dimethylamine (2M in methanol, 0.4 mL). The mixture was refluxed for 45 min and subjected to RP-HPLC separation to yield the title compound. MS found for 6e C₂₄H₂₆ClN₅O₅S (M+H)⁺ 532.

The following Examples 278-346 were similarly prepared by following the procedure of Example 277.

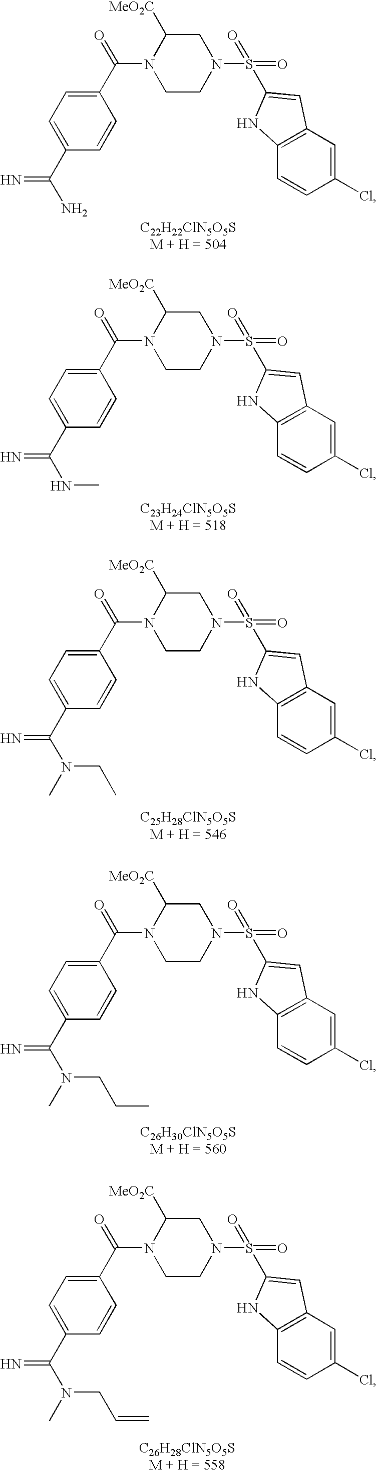

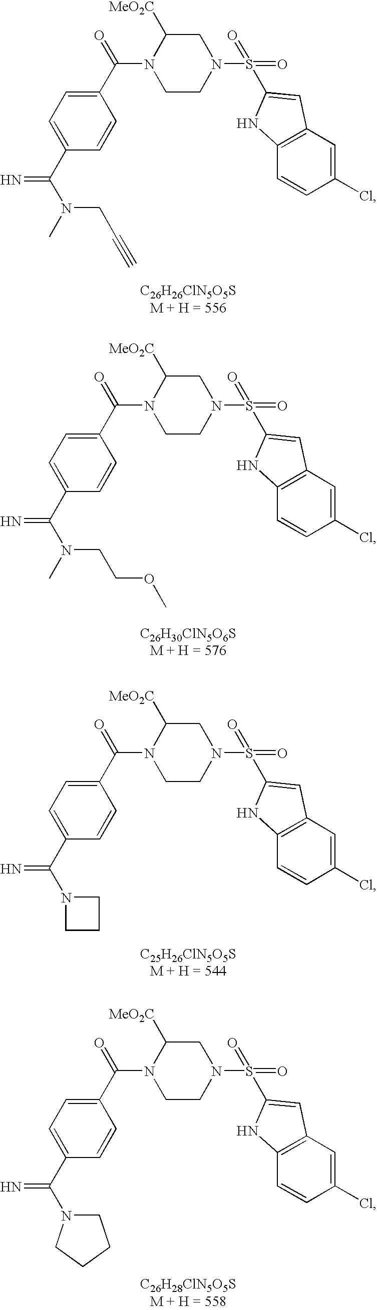

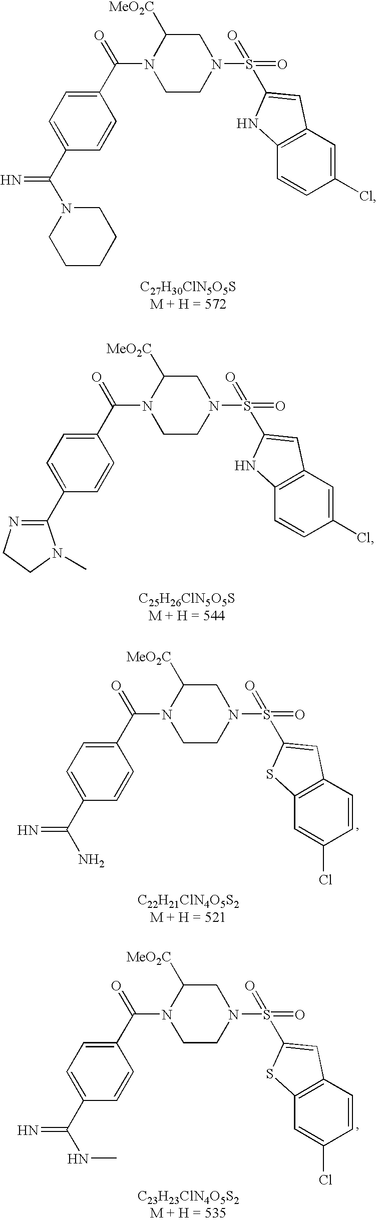

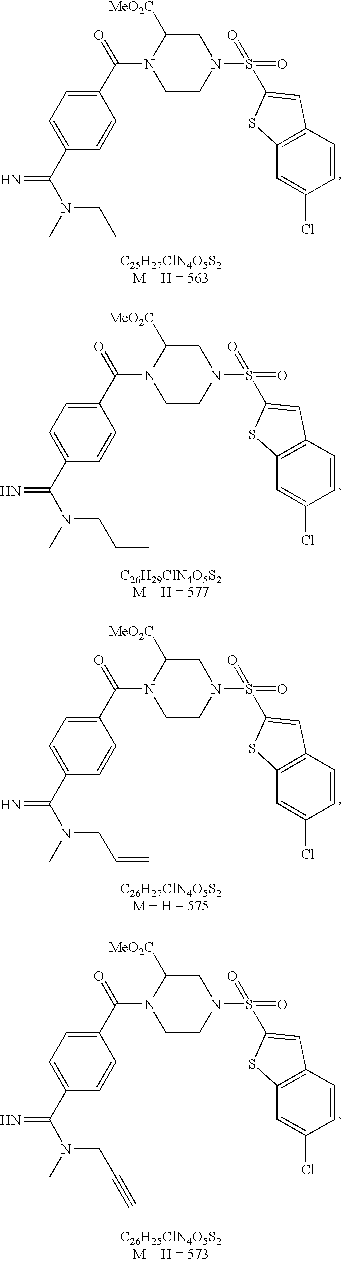

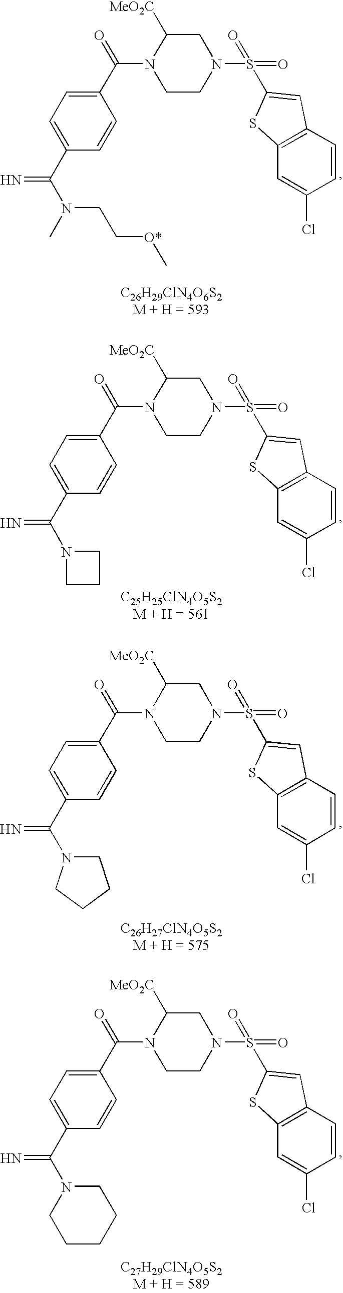

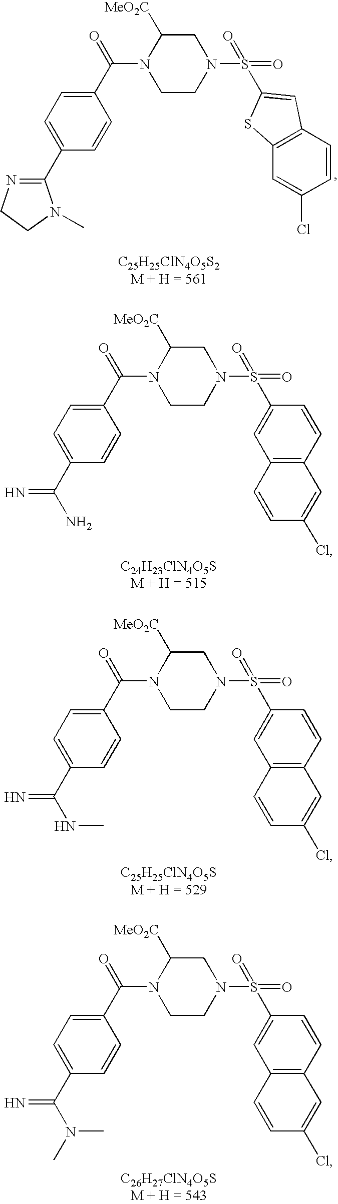

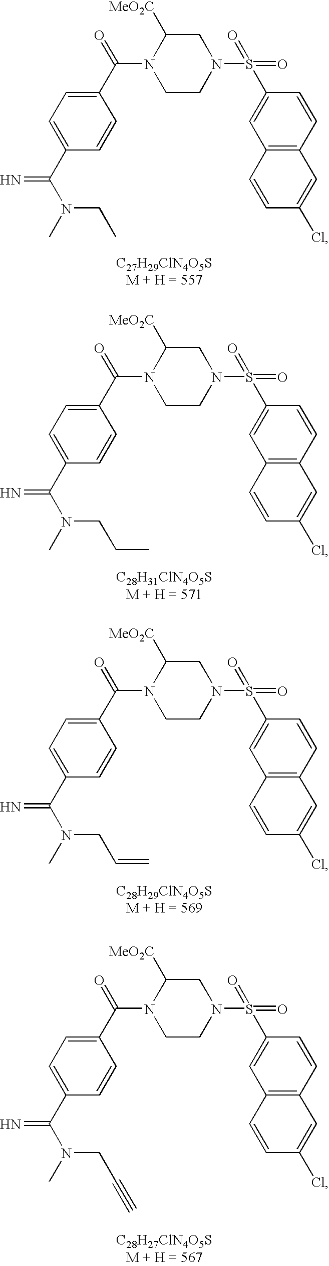

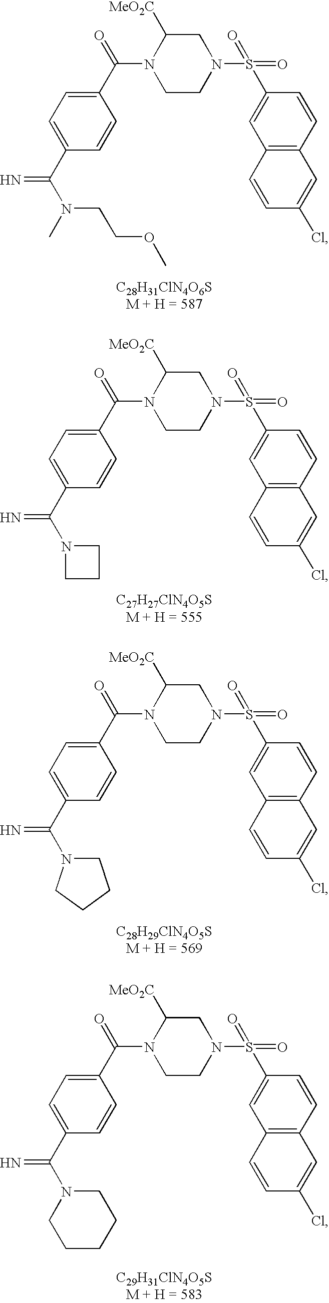

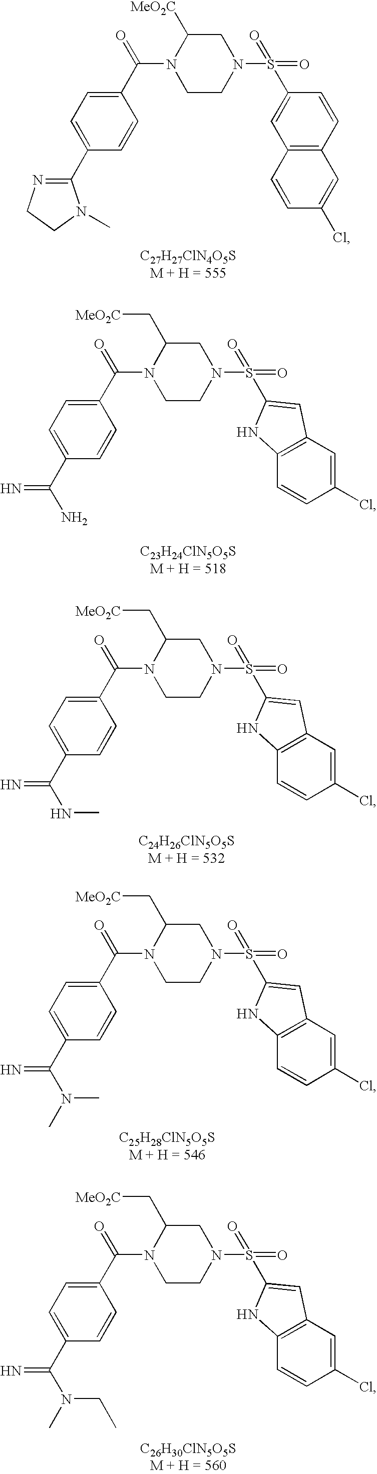

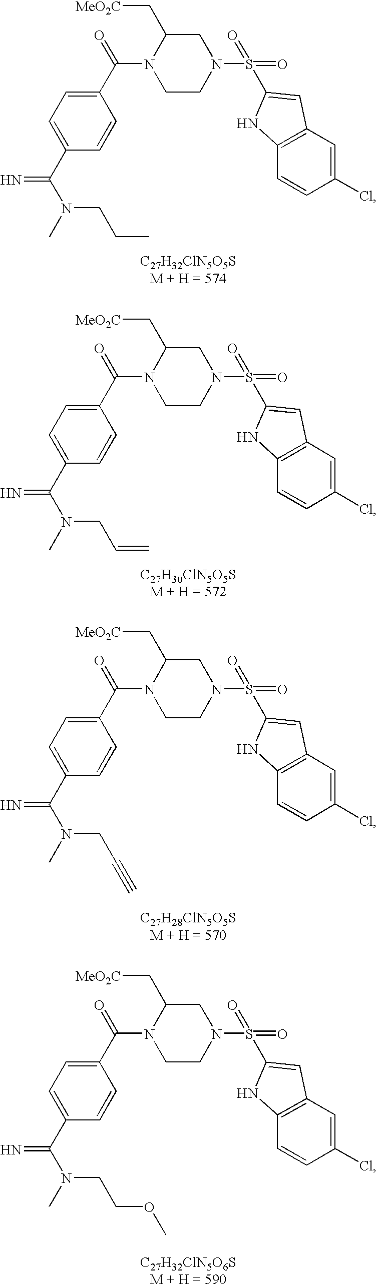

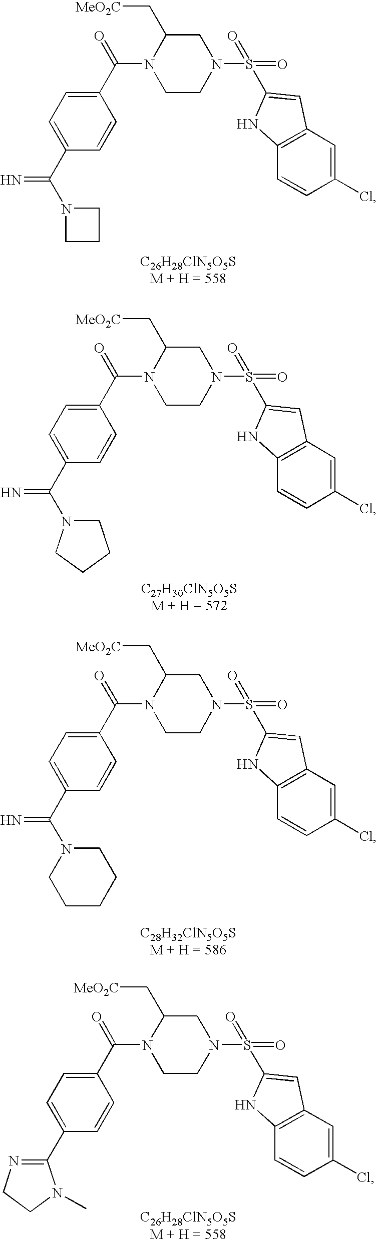

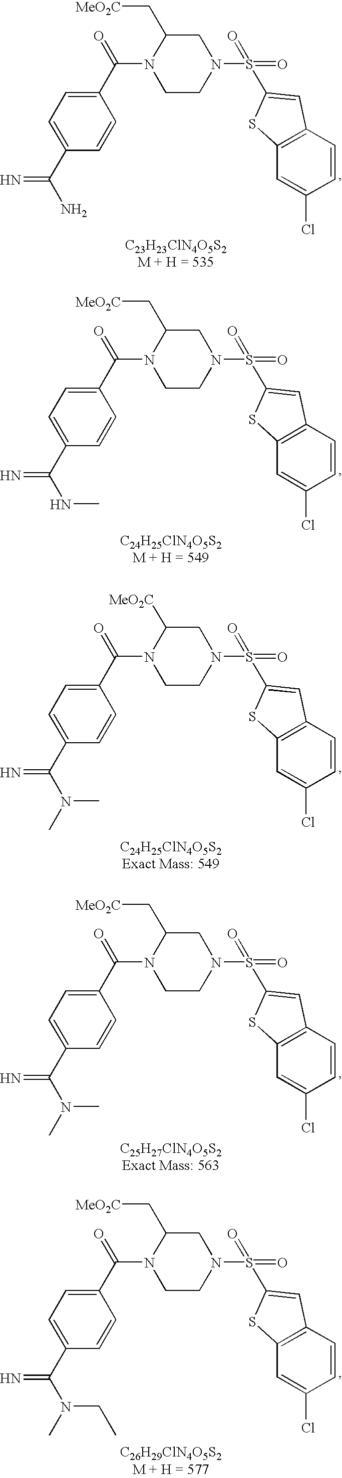

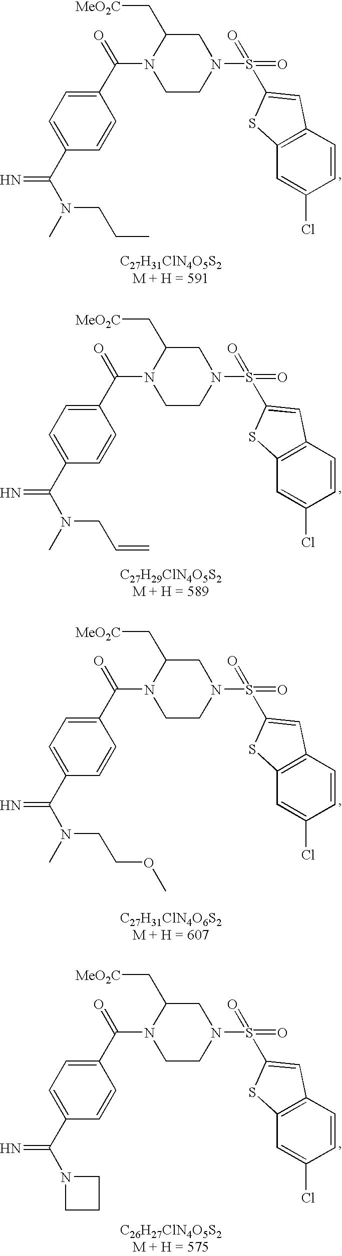

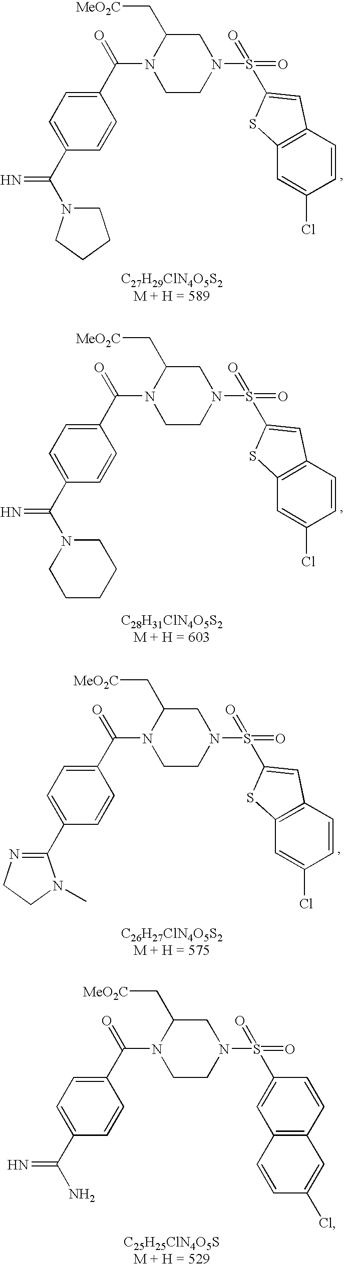

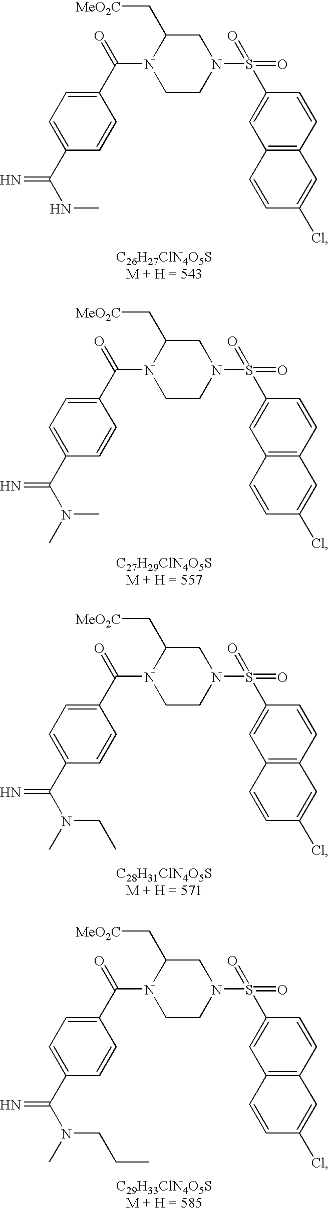

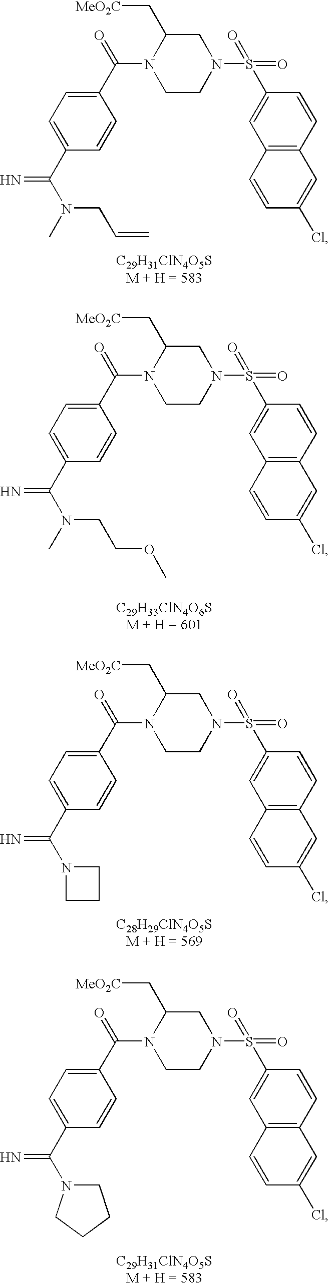

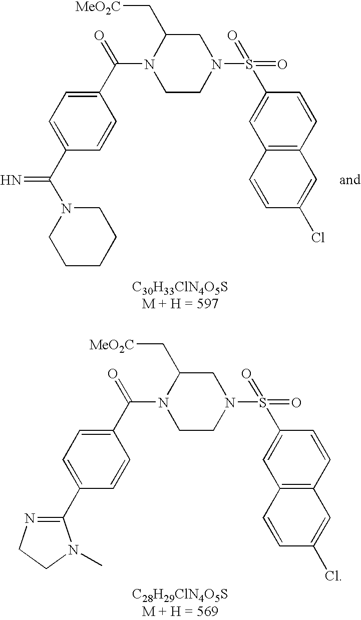

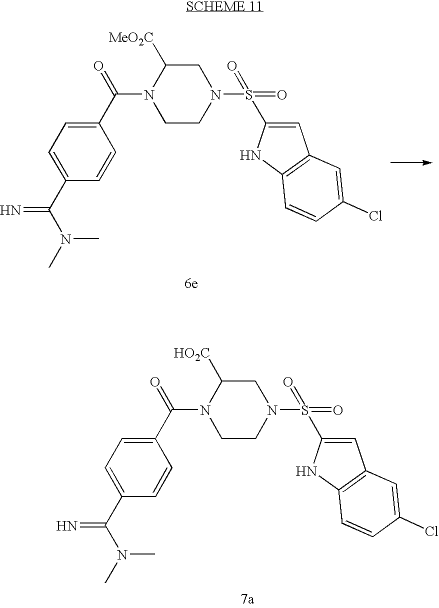

### EXAMPLE 347

### Preparation of 1-({4-[(dimethylamino)iminomethyl]phenyl}carbonyl)-4-[(5-chloroindol-2-yl)sulfonyl]piperazine-2-carboxylic acid (7a)

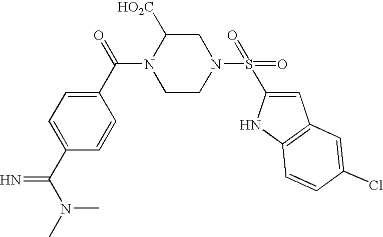

Compound 6e (20 mg) was dissolved in 4 mL methanol. To it were added 2 mL water and LiOH.H₂O (10 mg). The mixture was stirred for 40 min and purified with RP-HPLC to afford the title compound 7a. MS found for C₂₃H₂₄ClN₅O₅S (M+H)⁺ 518.

The following Examples 348-416 were similarly prepared by following the procedure of Example 347.

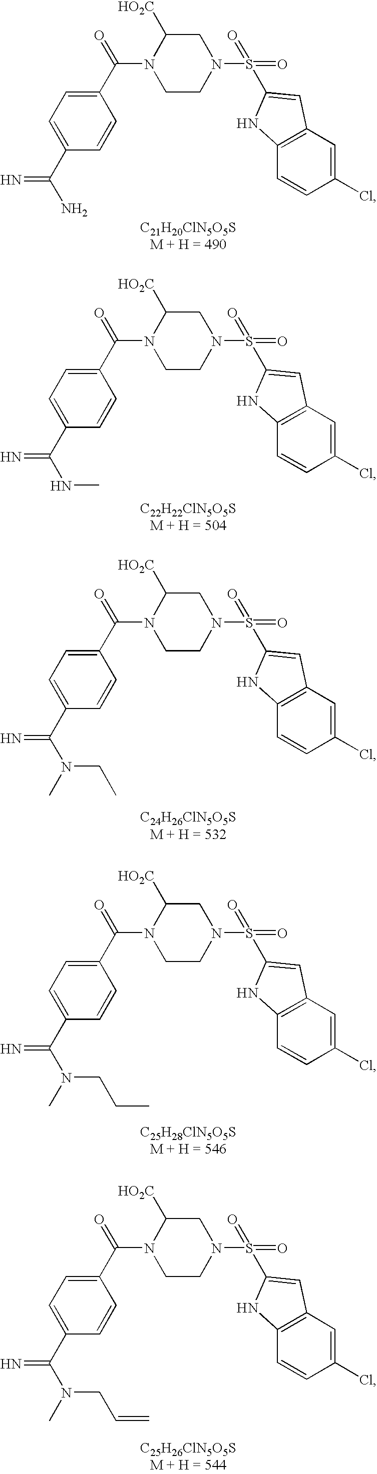

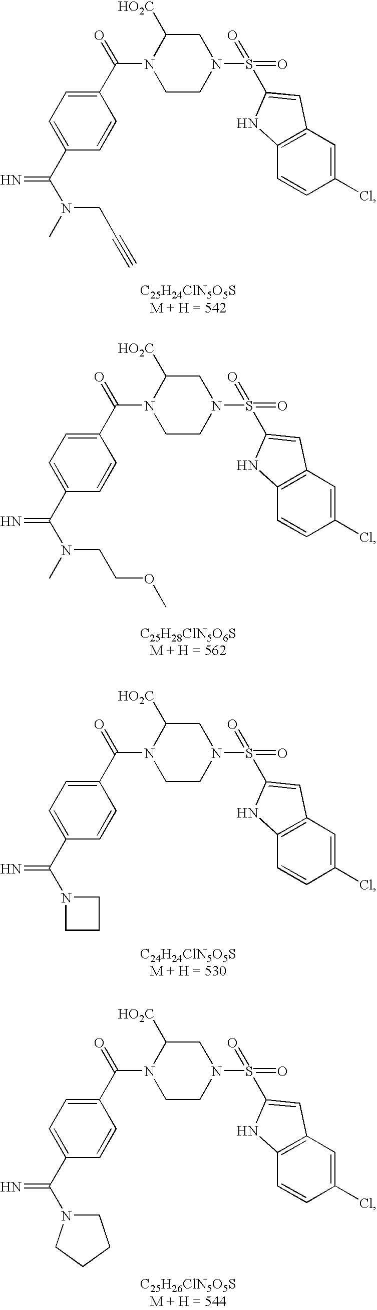

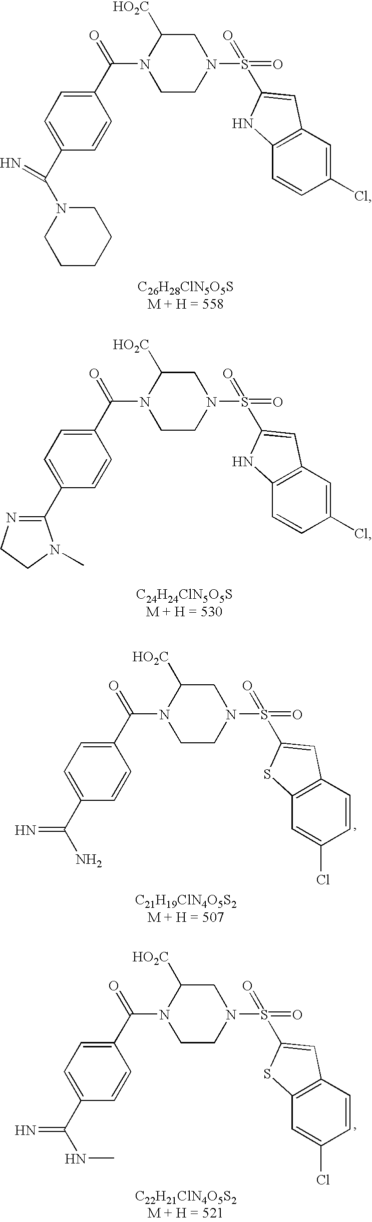

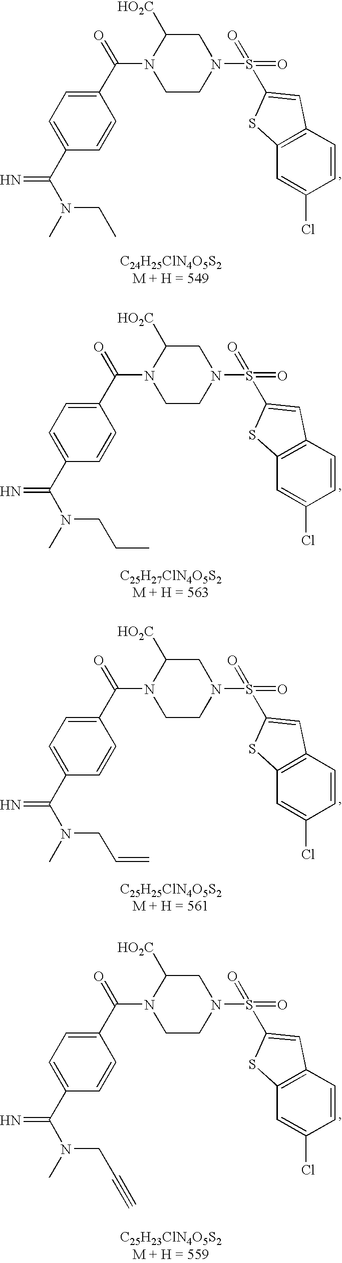

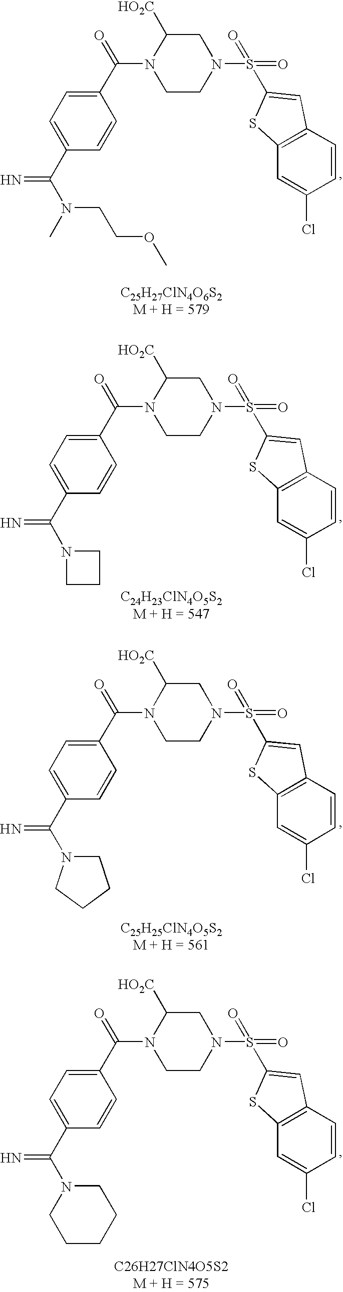

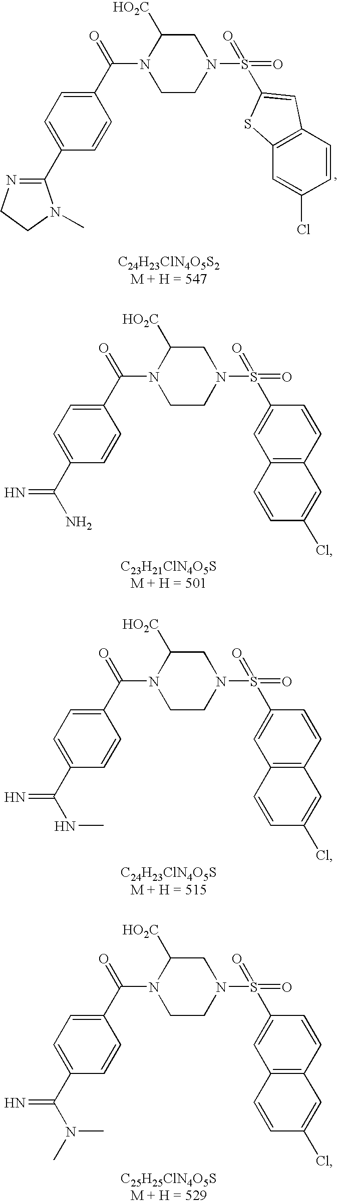

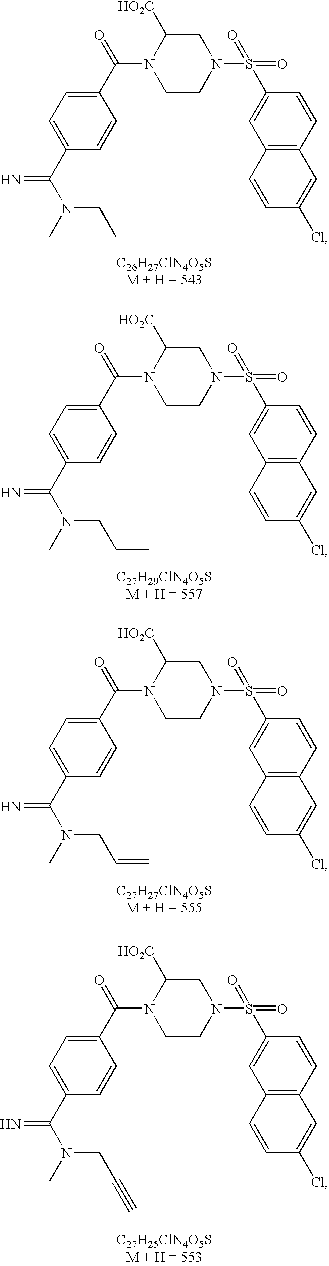

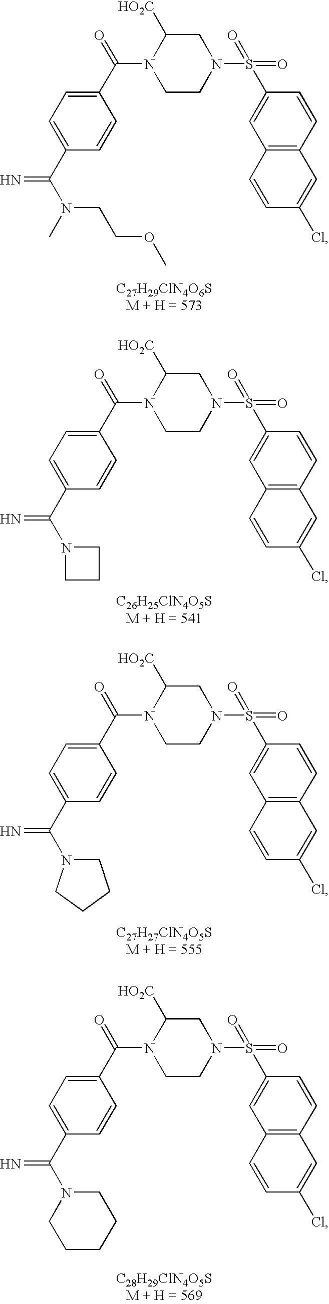

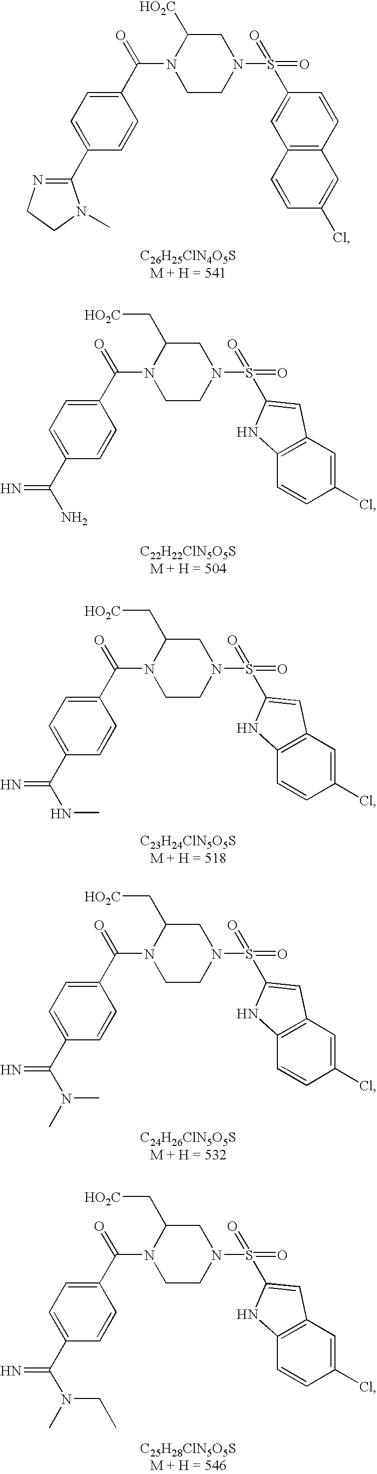

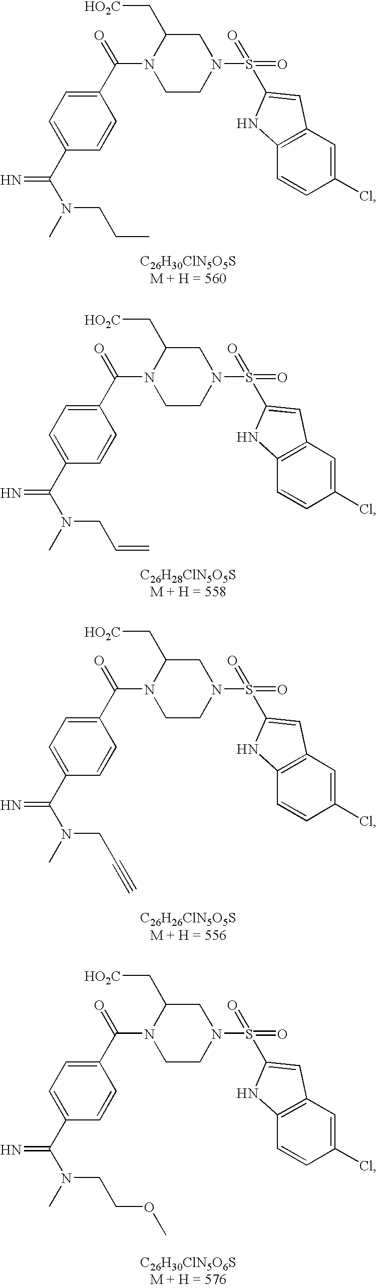

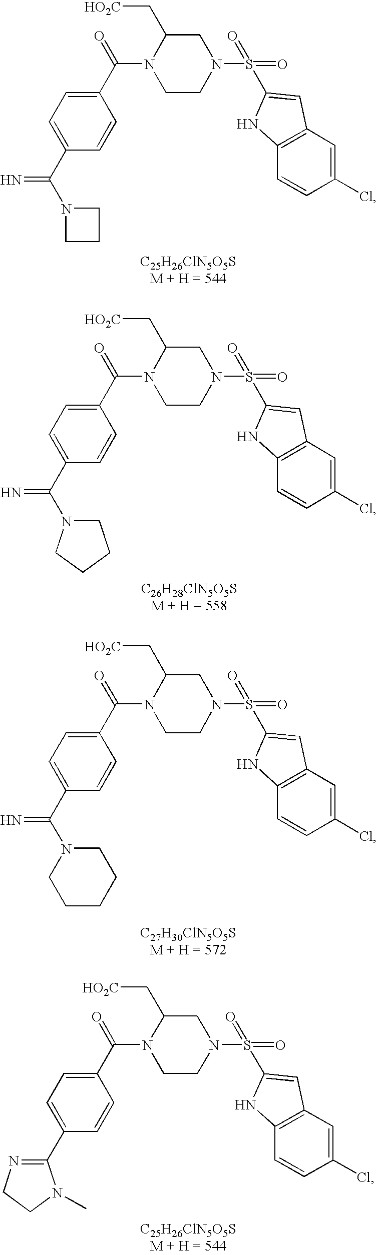

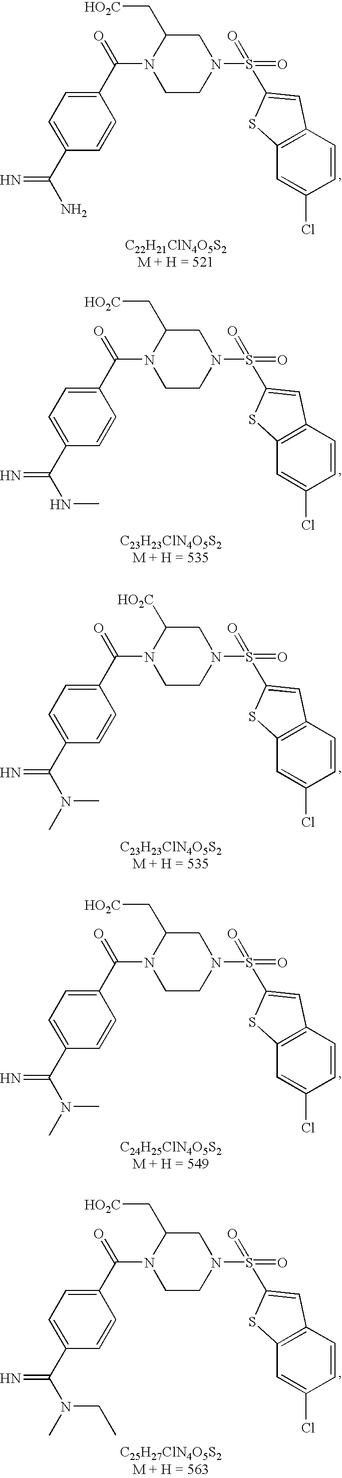

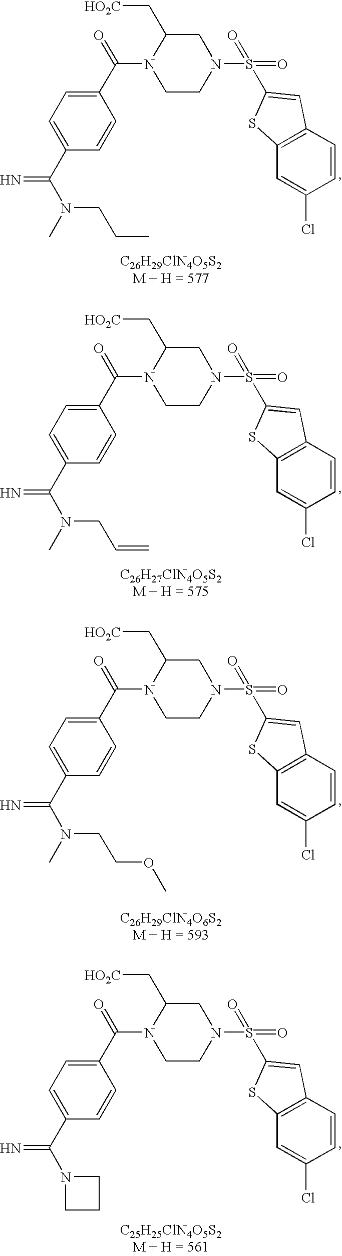

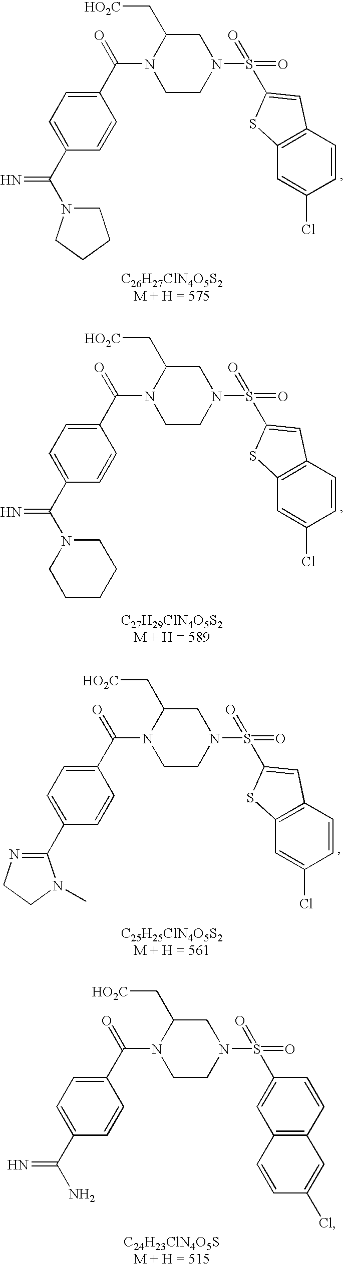

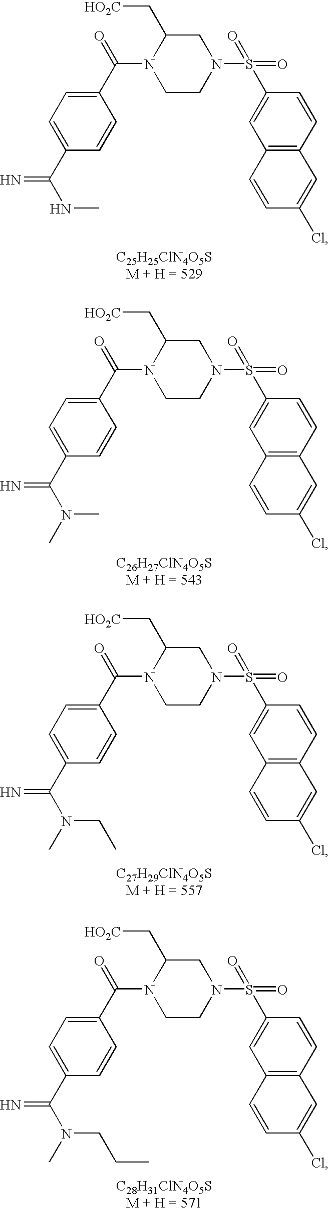

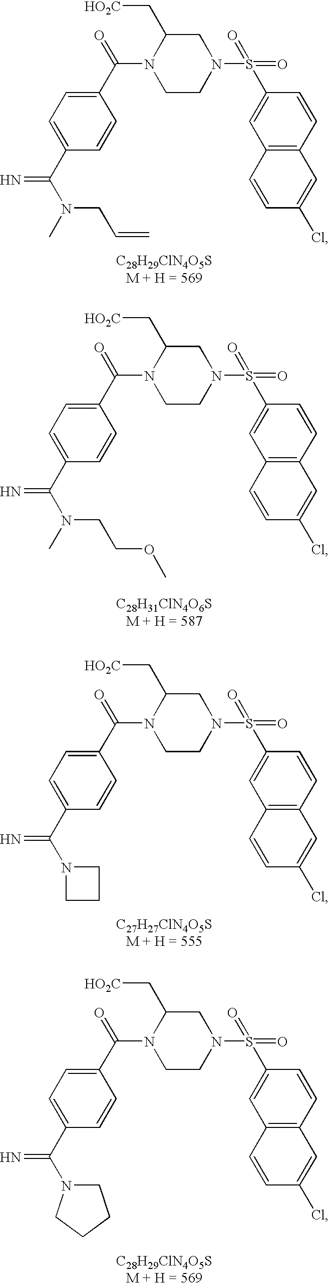

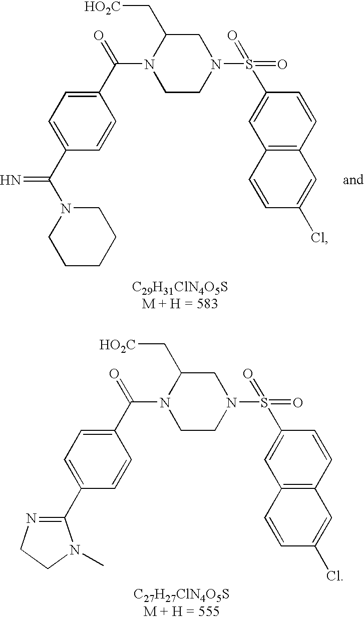

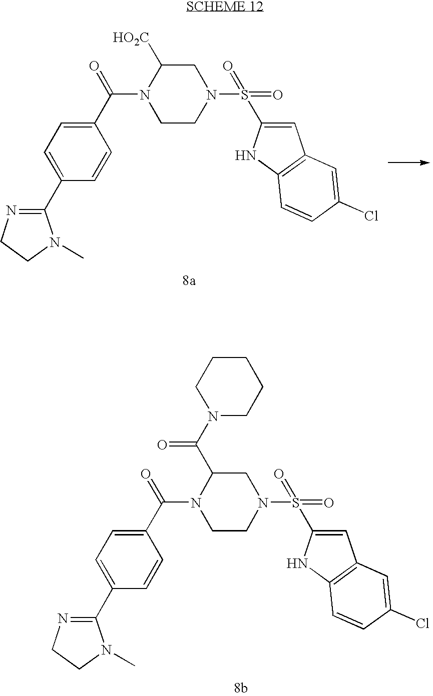

### EXAMPLE 417

### Preparation of 4-[(5-chloroindol-2-yl)sulfonyl]-2-(piperidylcarbonyl)piperazinyl 4-(1-methyl(2-imidazolin-2-yl))phenyl ketone (8b)

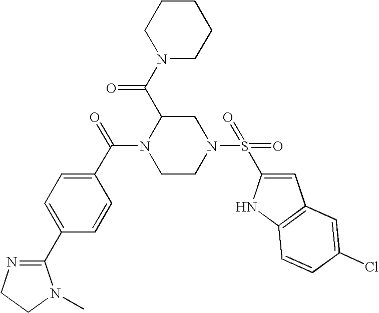

To the solution of compound 8a (Example 117, 50 mg, 0.1 mmol) in 1 mL DMF were added piperidine (17 mg, 0.2 mmol), DIEA (0.17 mL, 1 mmol) and PyBOP (200 mg, 0.4 mmol) in order. The mixture was stirred for overnight and subjected on RP-HPLC to isolate the title compound 8b. MS found for C₂₉H₃₃ClN₆O₄S (M+H)⁺ 597.

The following Examples 418-433 were similarly prepared by following the procedure of Example 417.

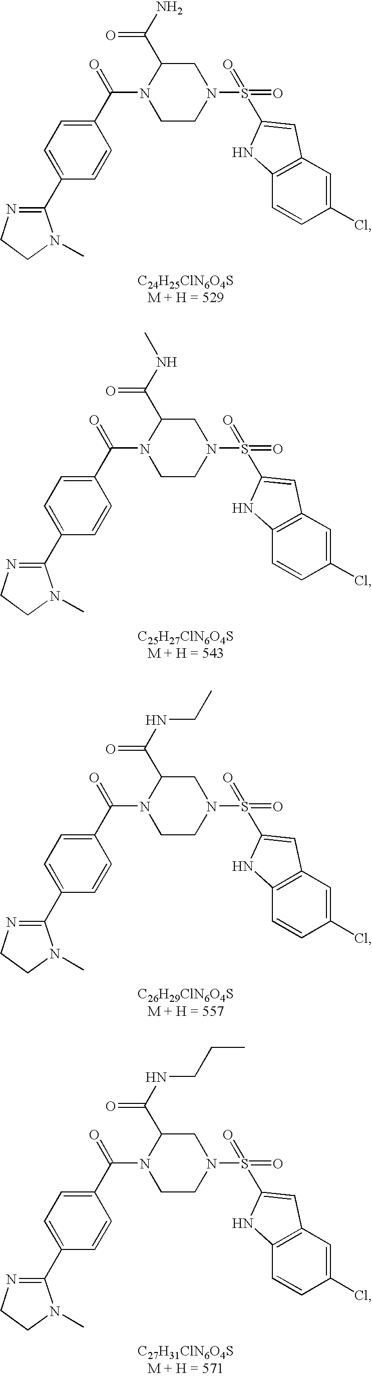

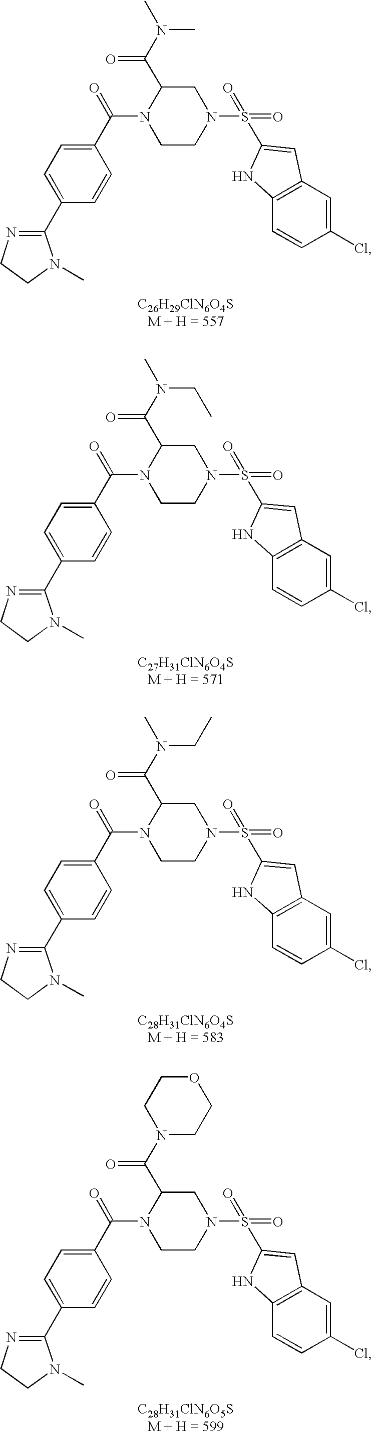

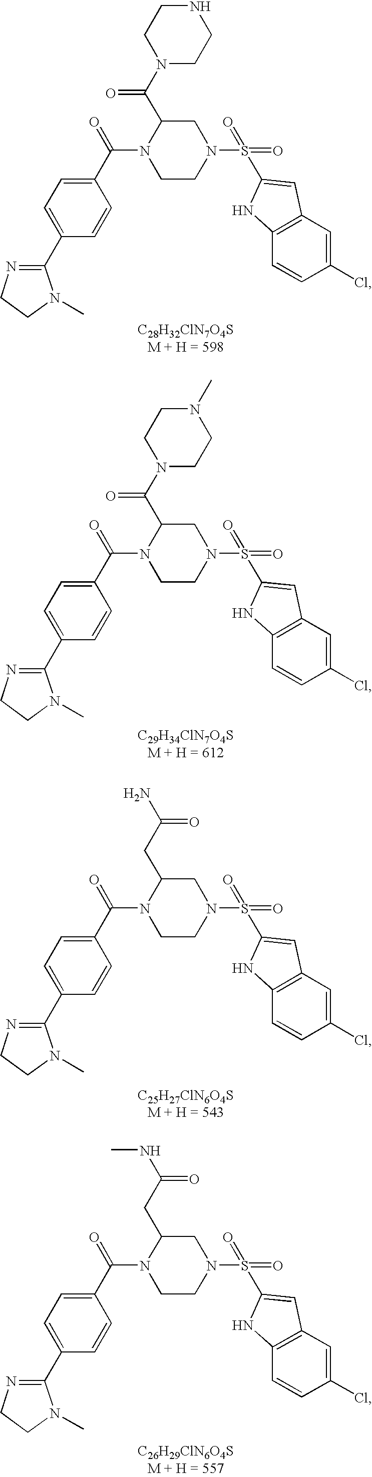

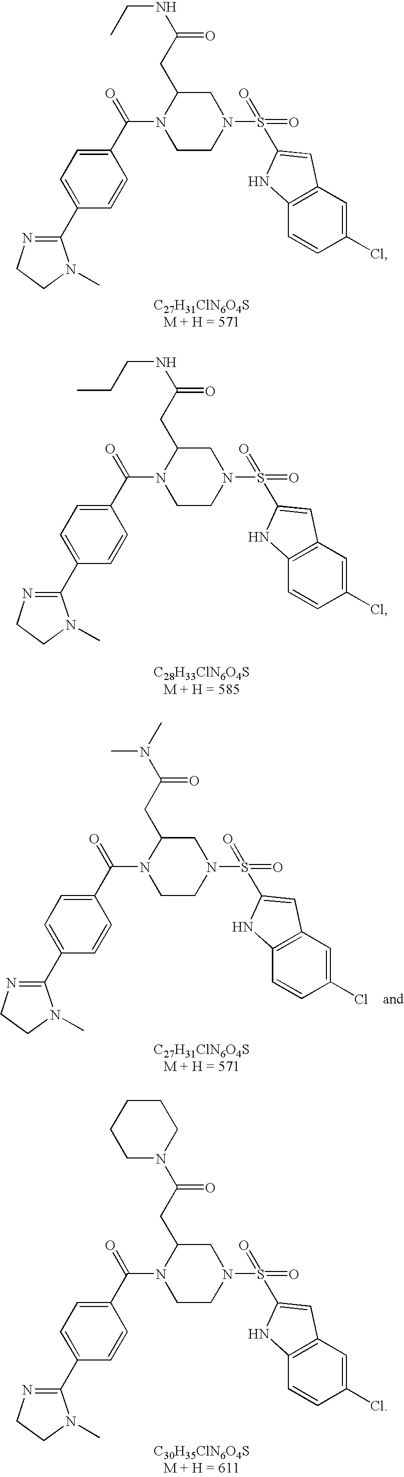

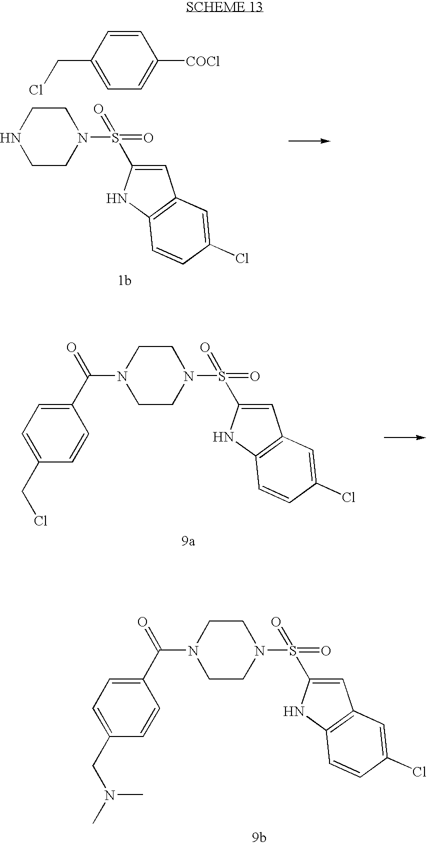

### EXAMPLE 434

### Preparation of 4-[(dimethylamino)methyl]phenyl 4-[(5-chloroindol-2-yl)sulfonyl]piperazinyl ketone (9b)

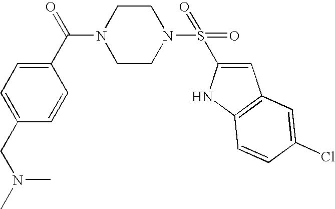

Step 1:

To the solution of compound 1b (300 mg, 1 mmol) in 10 mL dry THF were added 4-chloromethylbenzoyl chloride (220 mg, 1.15 mmol) and pyridine (0.3 mL). The mixture was stirred for 4 hrs, evaporated in vacuuo to remove THF and dissolved in 200 mL chloroform. It was washed with brine and water, dried, evaporated and purified with flash column to yield compound 9a (342 mg, 76%) as a solid. MS found for C₂₀H₁₉Cl₂N₃O₃S (M+H)⁺ 452.

Step 2:

Compound 9a (35 mg, 0.1 mmol) was dissolved in 1 mL dry DMF. To it were added dimethylamine (2M in THF, 0.2 mL, 0.4 mmol) and cesium carbonate (33 mg, 0.1 mmol). The mixture was stirred for overnight. It was filtered via a plastic microfilter and subjected to RP-HPLC purification to isolate the title compound 9b. MS found for C₂₂H₂₅ClN₄O3S (M+H)⁺ 461.

### EXAMPLE 435

### Preparation of 4-[(6-bromo(2-naphthyl))sulfonylpiperazinyl-3-chloro-4[(4-methylpiperazinyl)methyl](2-thienyl)ketone

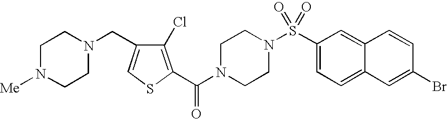

Step 1:

The mixture of tert-butyl piperazinecarboxylate (0.6 g, 3.3 mmoL, 1.0 equiv.), (6-bromo(2-naphthyl))chlorosulfone (1.0 g, 3.3 mmoL, 1.0 equiv.) and triethylamine (1.0 mL) in DCM (15.0 mL) was stirred at rt under argon atmosphere for 4 hrs. The solvent was removed at reduced pressure to give tert-butyl 4-[(6-bromo-2-naphthyl)sulfonyl]piperazinecarboxylate (2.14 g, 100%) as pale brown solid. The solid product was treated with 4N HCl in dioxane solution (20 mL) at rt for 30 min. The solvent was removed at reduced pressure to give 2-bromo-6-(piperazinylsulfonyl)naphthalene in quantitative yield. MS found for C₁₄H₁₅BrN₂O₂S (M+H)⁺ 355

Step 2:

A solution of methyl 3-chloro-4-methylthiophene-2-carboxylate (953 mg, 5 mmol, 1 equiv) in 10 mL of CCl₄ was treated with NBS (895 mg, 1 equiv) and benzoylperoxide (60 mg, 0.05 equiv) at reflux for 4 h. After cooling to rt, the mixture was filtered and the filtrate was evaporated. The residue was then dissolved in 10 mL of DCM, and treated with N-methylpiperazine (0.83 mL, 1.5 equiv) and pyridine (1.25 mL) at rt overnight. The mixture was then washed with water, dried over MgSO₄, filtered and evaporated to give a residue, which was dissolved in 10 mL of MeOH. To the solution was added 10 mL of 1N LiOH (2 equiv) and the mixture was stirred at rt for 4 h, acidified with 1N HCl to pH˜1. The mixture was lyopholized and the residue was subjected to RP-HPLC to give 3-chloro-4-(4-N-methylpiperazin-1-yl)methylthiophene-2-carboxylic acid (280 mg, 29%). MS found for C₁₁H₁₆ClN₂O₂S (M+H)⁺ 275.

Step 3:

To a solution of 3-chloro-4-(4-N-methylpiperazin-1-yl)methylthiophene-2-carboxylic acid (27.5 mg, 0.1 mmol, 1 equiv) in 1 mL of DMF were added 2-bromo-6-(piperazinylsulfonyl)naphthalene (35.5 mg, 1 equiv), BOP (89 mg, 2 equiv), and Et₃N (0.5 mL). The mixture was stirred at rt overnight, diluted with EtOAc, washed with water, dried over MgSO₄, filtered and evaporated to give a residue, which was subjected to RP-HPLC to give the target compound (23 mg, 38%). MS found for C₂₅H₂₉BrClN₄O₃S₂ (M+H)⁺ 611.

### EXAMPLE 436

### Preparation of 4-[(6-bromo(2-naphthyl))sulfonylpiperazinyl-3-chloro-4-(methylaminomethyl)(2-thienyl)ketone

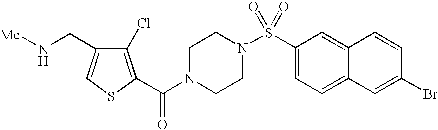

Step 1:

To a solution of 3-chloro-4-methylthiophene-2-carboxylic acid (352 mg, 2 mmol, 1 equiv) in 10 mL of DCM was added 1 mL of (COCl)₂ and 1 drop of DMF. The mixture was stirred at rt until there was no gas coming out. After evaporating the volatile, the residue was redissolved in 10 mL of DCM. To the solution were added 2-bromo-6-(piperazinylsulfonyl)naphthalene (710 mg, 1 equiv), and 2 mL of Et₃N. The mixture was stirred at rt overnight and washed with water and dried over sodium sulfate. Filtration, evaporation followed by flash chromatography to gave 4-[(6-bromo(2-naphthyl))sulfonylpiperazinyl-3 -chloro-4-methyl(2-thienyl)ketone (510 mg, 52%). MS found for C₂₀H₁₉BrClN₂O₃S₂ (M+H)⁺ 513.

Step 2:

A solution of 4-[(6-bromo(2-naphthyl))sulfonylpiperazinyl-3-chloro-4-methyl(2-thienyl)ketone (170 mg, 0.33 mmol, 1 equiv) in 5 mL of CCl₄ was treated with NBS (59 mg, 1 equiv) and benzoylperoxide (5 mg, 0.05 equiv) at reflux for 2 hrs. After cooling to rt, the mixture was filtered and the filtrate was evaporated. The residue was then dissolved in 3 mL of DMF, and treated with 1 mL of 2 N methylamine in THF at rt for 24 hrs. The mixture was then evaporated and subjected to RP-HPLC to give the title compound (280 mg, 29%). MS found for C₂₁H₂₂BrClN₃O₃S₂ (M+H): 542.

### EXAMPLE 437

### Preparation of 4-[(6-bromo(2-naphthyl))sulfonylpiperazinyl-3-chloro-4-(aminomethyl)(2-thienyl)ketone

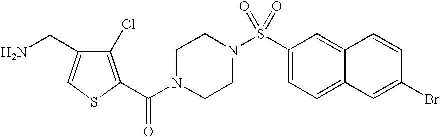

This compound was prepared according to the procedure described above for Example 436. MS found for C₂H₂₀BrClN₃O₃S₂ (M+H)⁺: 528.

The following Examples 438-473 were similarly prepared by following the procedure of Examples 434-437 as described above.

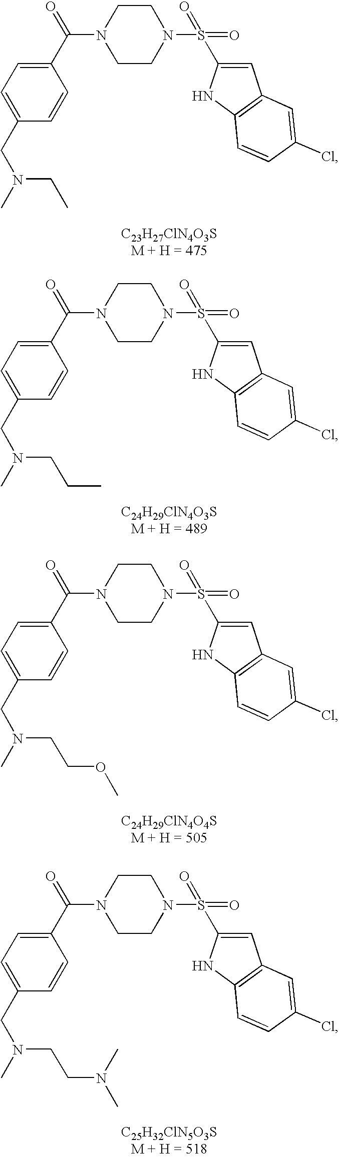

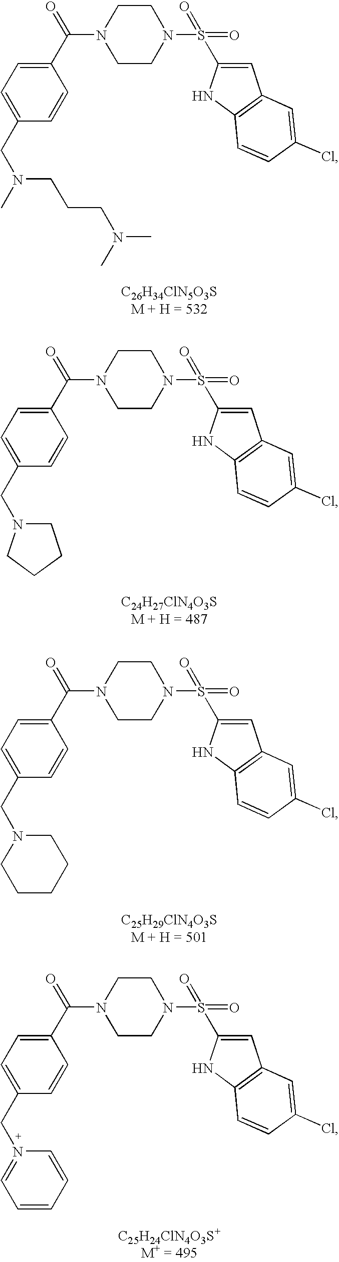

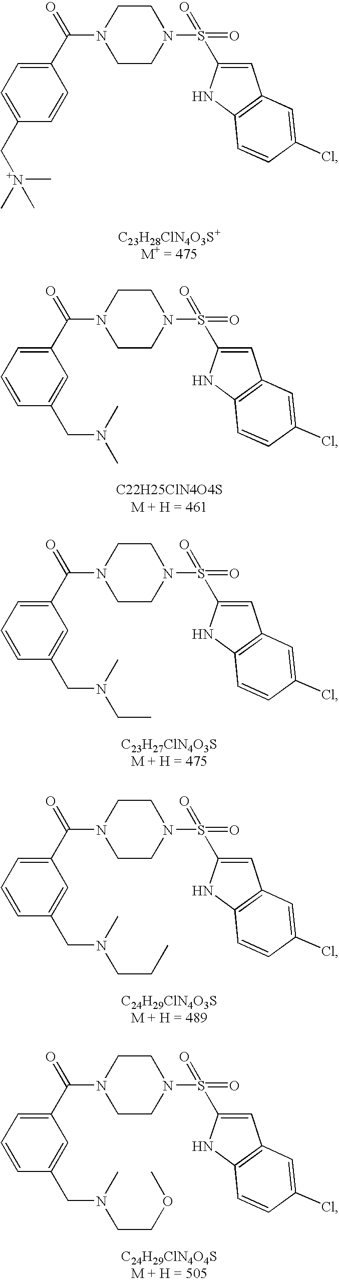

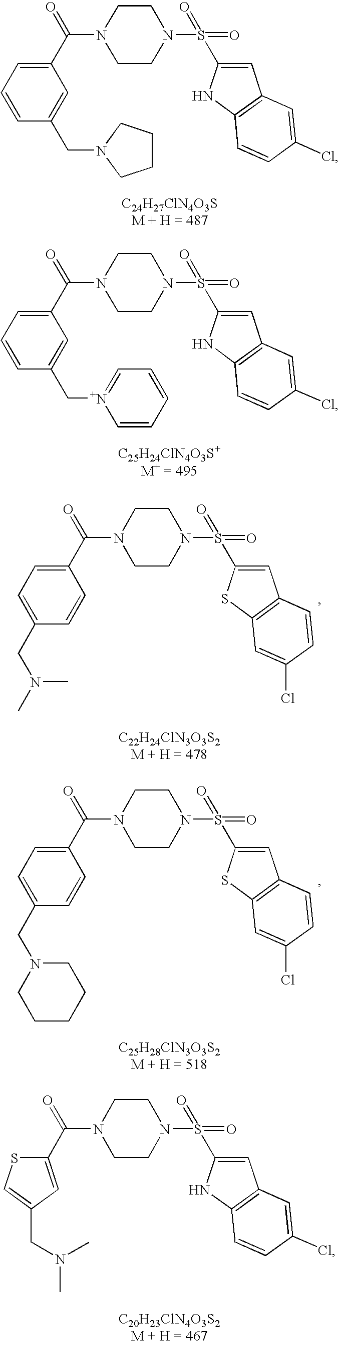

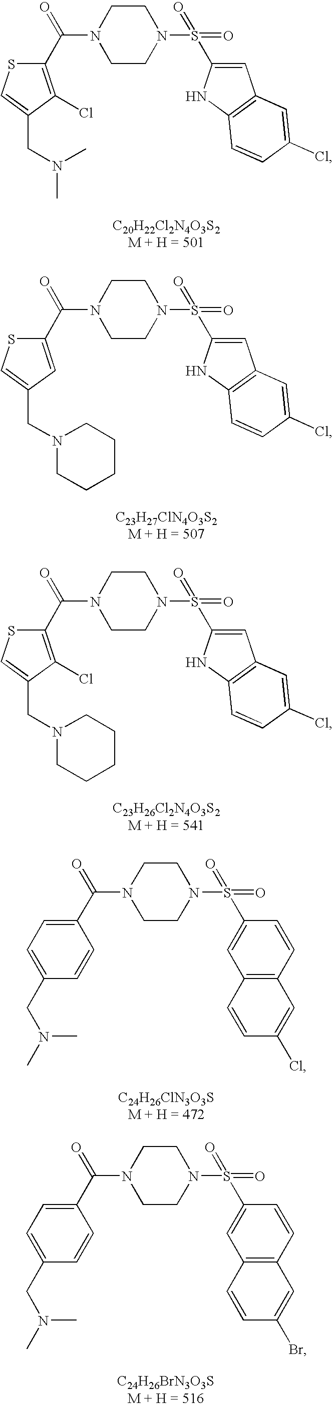

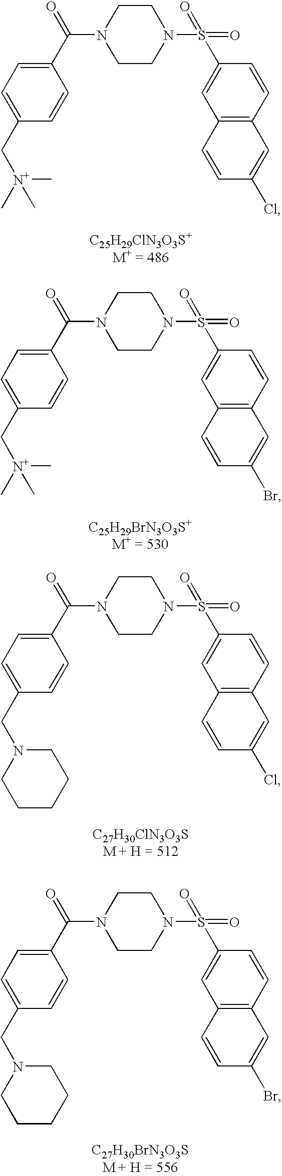

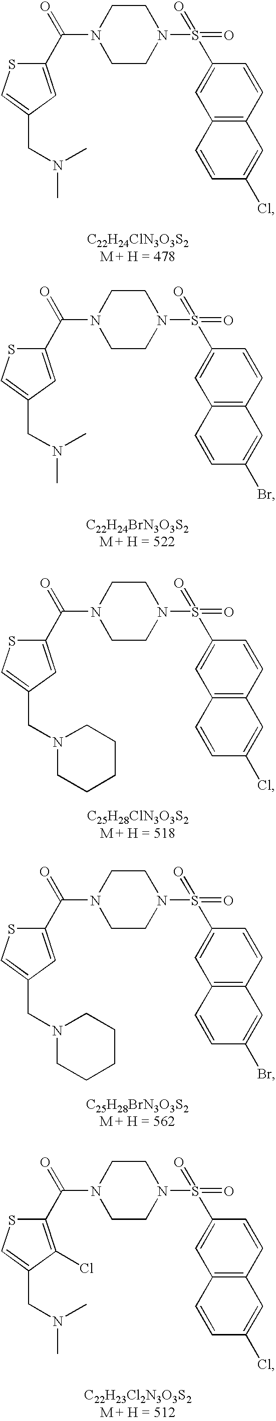

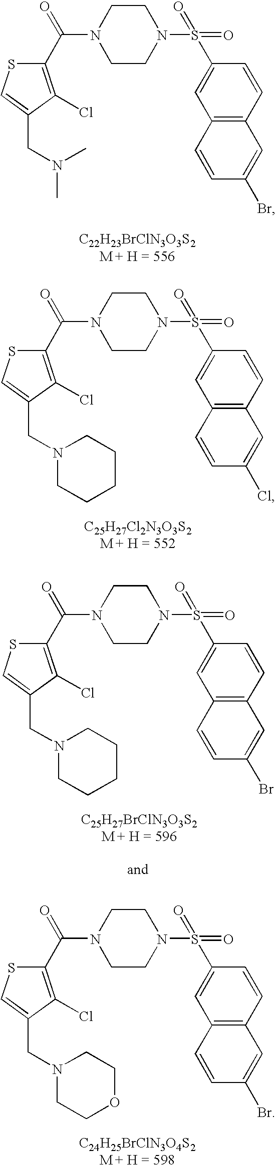

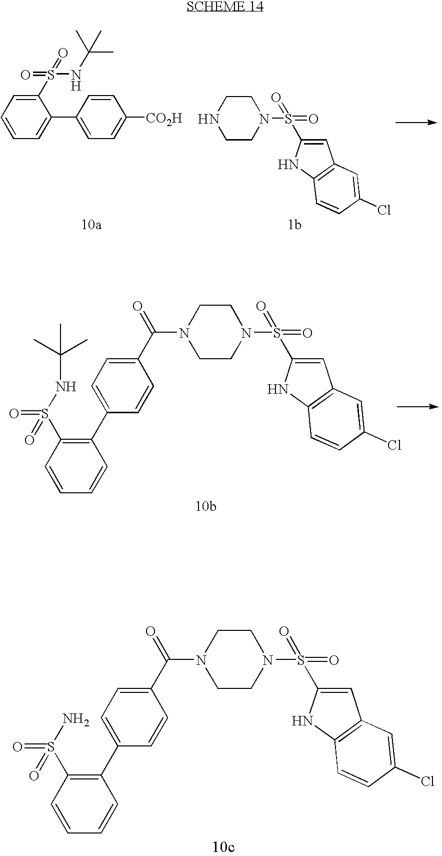

### EXAMPLE 474

### Preparation of 2-[4-({4-[(5-chloroindol-2-yl)sulfonyl]piperazinyl}carbonyl)phenyl]benzenesulfonamide (10c)

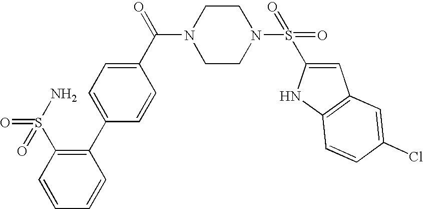

To the solution of compounds 10a (33 mg, 0.1 mmol) and 1b (30 mg, 0.1 mmol) in 1 mL DMF were added DIEA (0.1 mL) and PyBOP (100 mg, 0.2 mmol). The mixture was stirred for overnight and diluted with 100 mL chloroform. It was washed with water 10 mL×2, dried, evaporated in vacuuo to give crude 10b (71%). MS found for C₂₉H₃₁ClN₄O₅S₂ (M+H)⁺ 615. The crude 10b was then treated with 3 mL TFA for 3 hrs. The mixture was evaporated and subjected to RP-HPLC to isolate the title compound 10c. MS found for C₂₅H₂₃ClN₄O₅S₂ (M+H)⁺ 559.

The following Examples 475-507 were similarly prepared by following the procedure of Example 474.

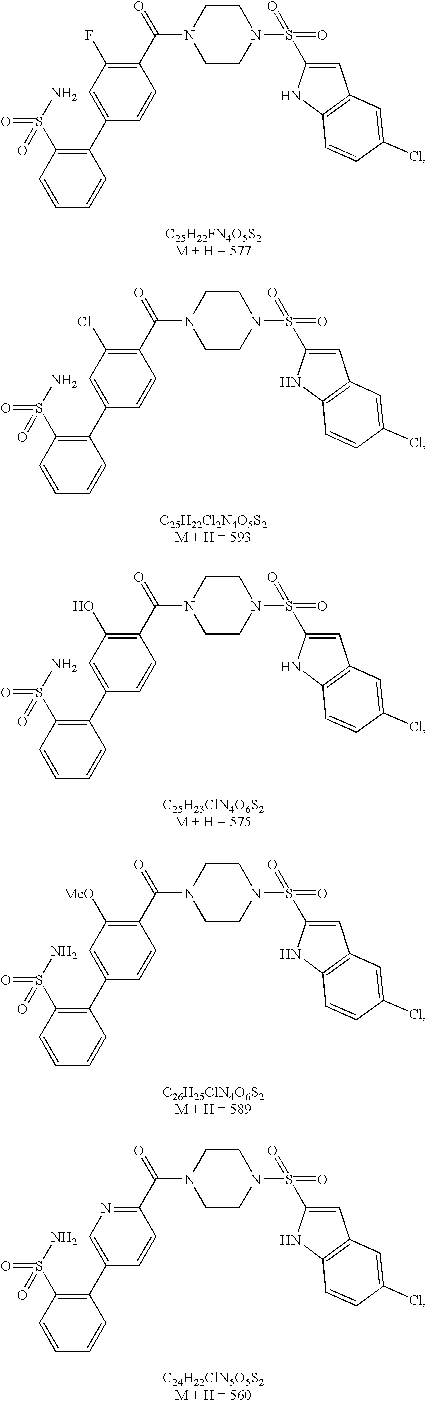

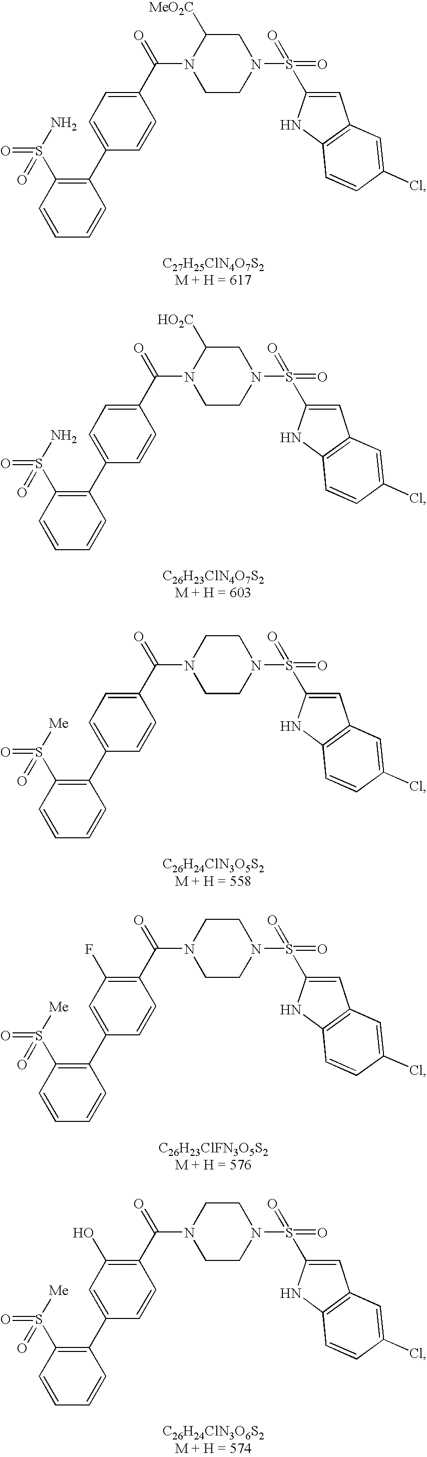

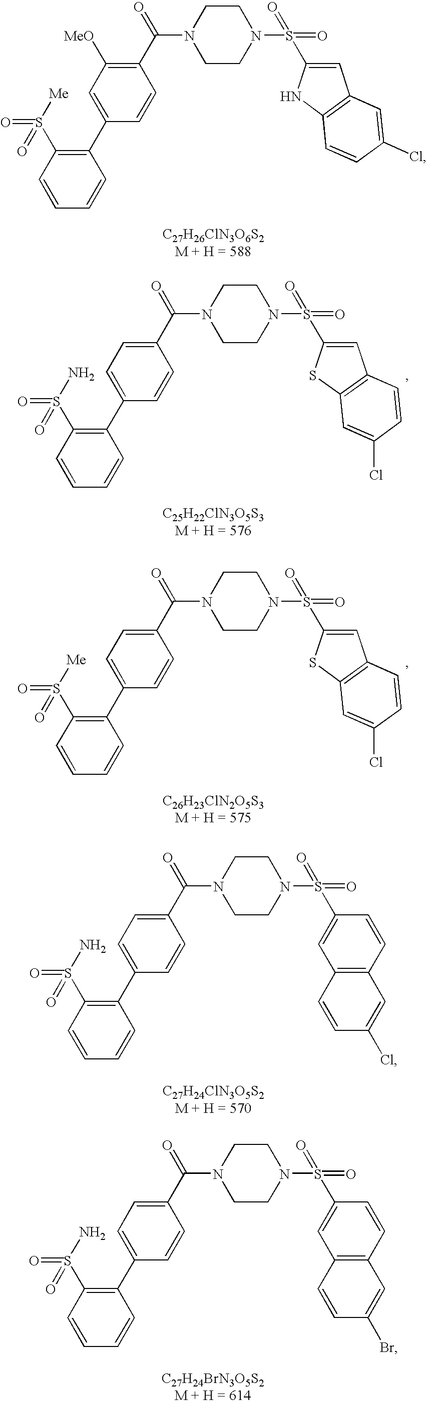

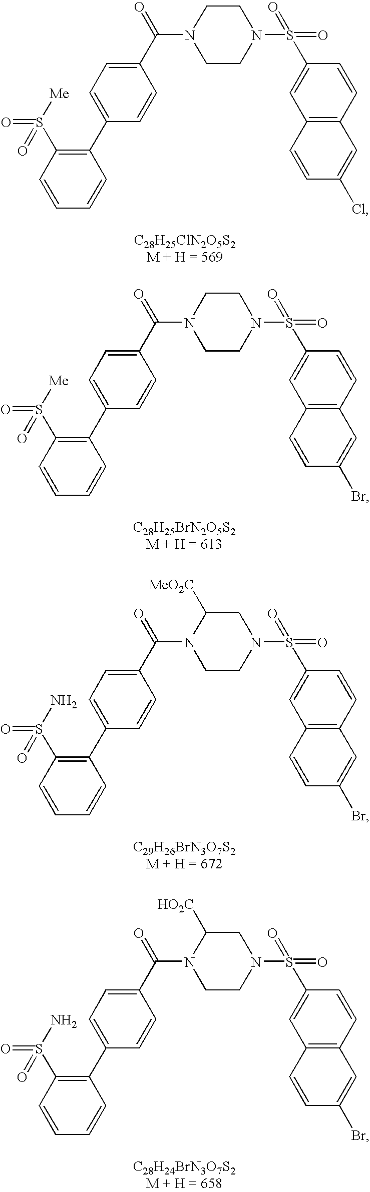

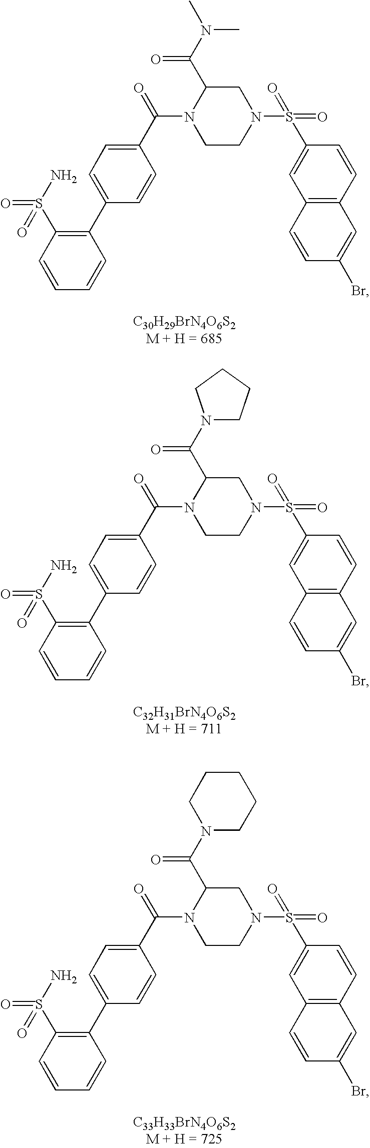

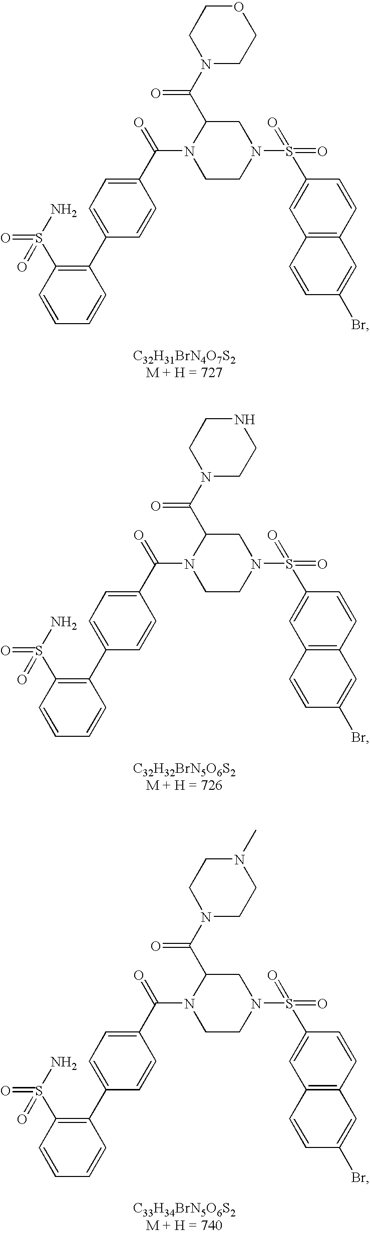

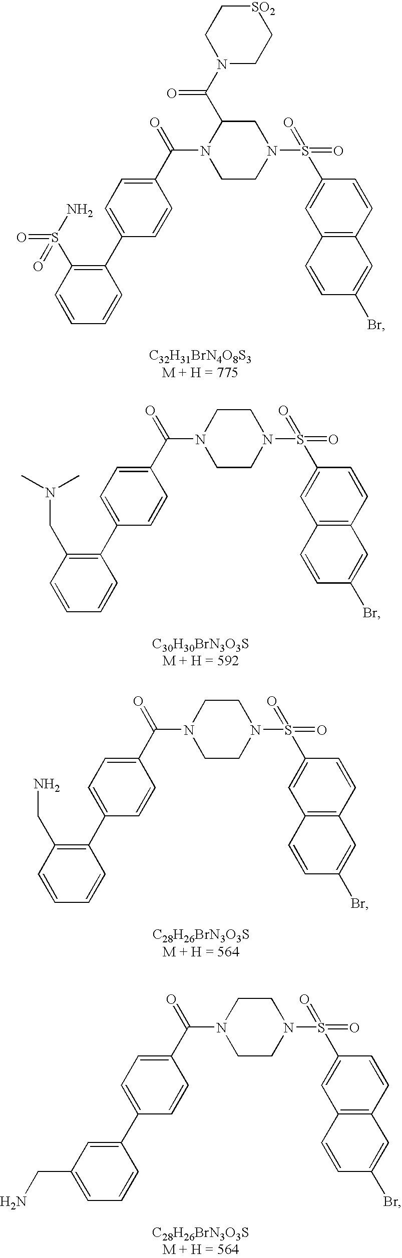

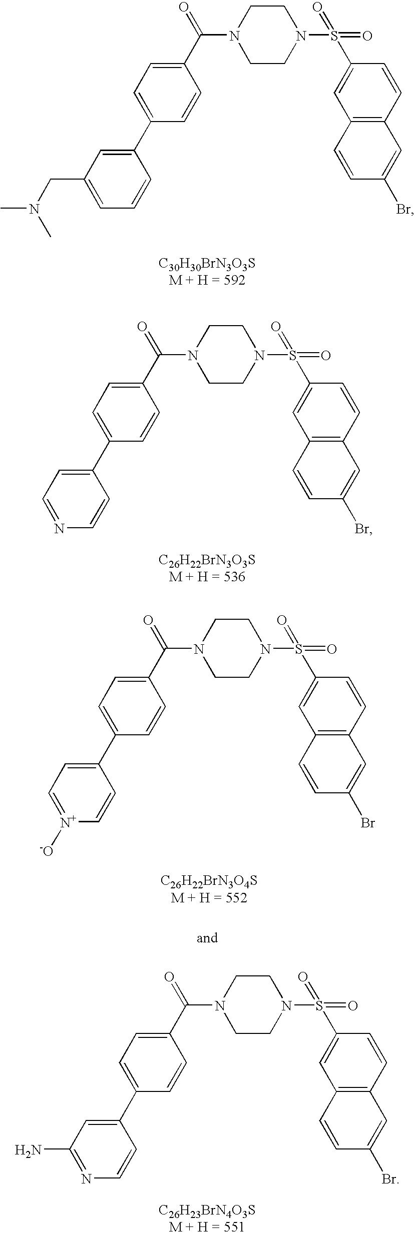

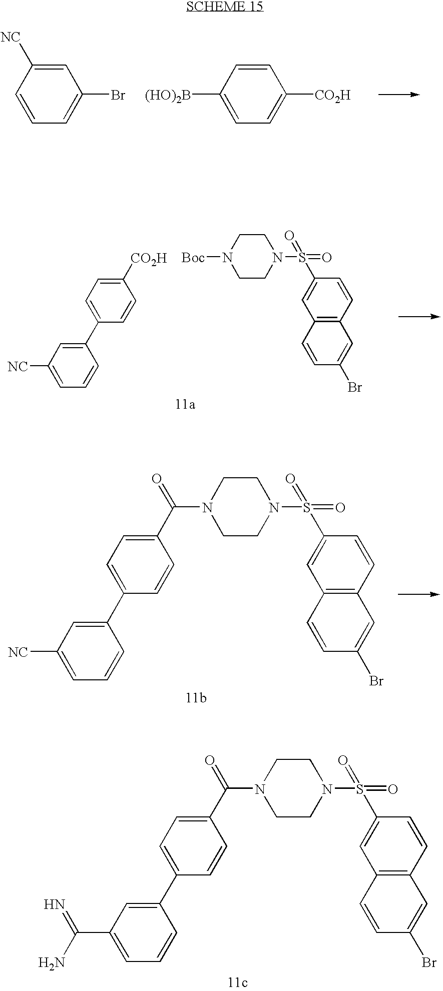

### EXAMPLE 508 AND 509

### Preparation of 3-[4-({4-[(6-bromo-2-naphthyl)sulfonyl]piperazinyl}carbonyl)phenyl]benzenecarbonitrile (11b) and 3-[4-({4-[(6-bromo-2-naphthyl)sulfonyl]piperazinyl}carbonyl)phenyl]benzenecarboxamidine (11c)

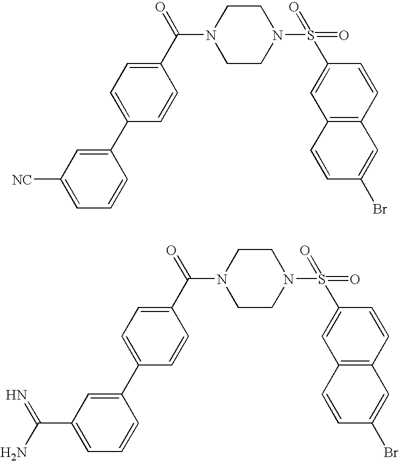

Step 1:

A mixture of 3-bromobenzonitrile (0.91 g, 5.0 mmol), 4-carboxylbenzeneboric acid (0.83 g, 5.0 mmol), Pd(Ph₃P)₄ (0.29 g, 0.25 mmol) and nBu₄NBr (0.08 g, 0.25 mmol) in 2M aq. Na₂CO₃ (8 mL) and toluene (35 mL) was purged with argon, then heated to reflux under argon for 5 hrs. Organic phase was separated, dried over Na₂SO₄, concentrated in vacuuo. The residue was dissolved in EtOAc (10 mL). After standing at room temperature overnight, precipitates came out from the solution, which were collected by filtration to give compound 11a (120 mg, 11%). MS found for C₁₄H₉NO₂ (M+H)⁺ 224.

Step 2:

4-Boc-piperazinyl 6-bromonaphthalenesulfonamide (90 mg, 0.20 mmol) was dissolved in TFA (4 mL). After being stirred at room temperature for 2 hrs, TFA was removed in vacuuo. The residue was dissolved in DCM (3 mL) containing Et₃N (0.11 mL, 0.79 mmol). The solution was then added to a pre-mixed solution of compound 11a (45 mg, 0.20 mmol), BOP (114 mg, 0.26 mmol) and Et₃N (0.06 mL, 0.40 mmol) in DCM (5 mL). The mixture was stirred at room temperature overnight. It was concentrated in vacuuo. The residue was dissolved in EtOAc. The solution was washed with 1N NaOH, 1N HCl, and brine, dried over Na₂SO₄, concentrated in vacuuo. The residue was purified by a silica gel column to give the title compound 11b (50 mg, 45%). MS found for C₂₈H₂₂BrN₃O₃S (M+H)⁺ 560.

Step 3:

To a solution of compound 11b (50 mg, 0.089 mmol) in anhydrous MeOH (5 mL) and CHCl₃ (1 mL) in ice-bath, HCl gas was bubbled until saturation was reached. The solution was then stirred at room temperature for overnight. It was concentrated in vacuuo. The residue was dissolved in MeOH (4 mL), NH₄OAc (54 mg, 0.70 mmol) was added. The mixture was heated to reflux for 2 hrs. It was concentrated in vacuuo. The residue was purified by RP-HPLC to give the title compound 11c. (17 mg, 33%). MS found for C₂₈H₂₅BrN₄O₃S (M+H)⁺ 577.

The following Examples 510-527 were similarly prepared by following the procedure of Examples 508 and 509.

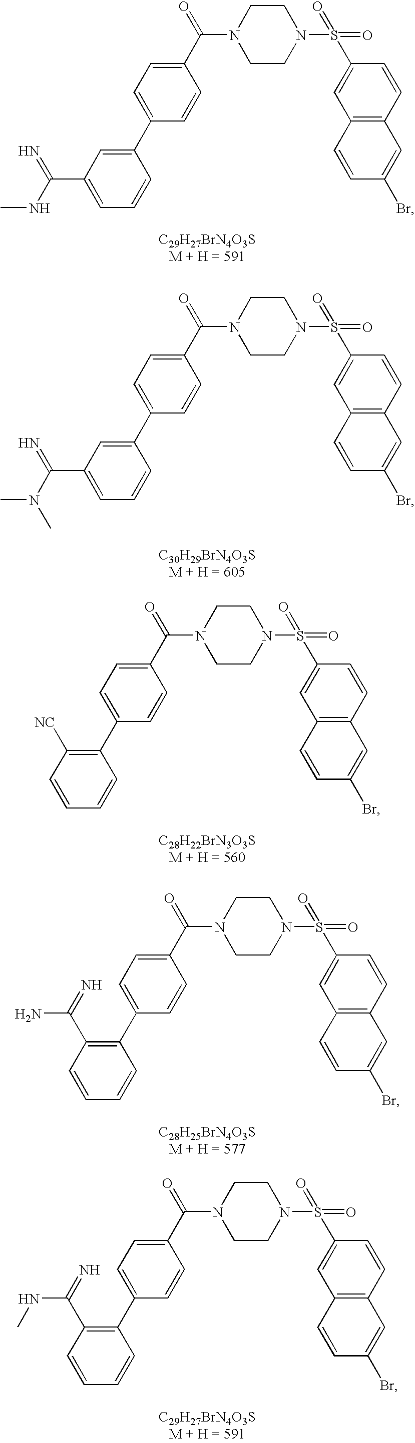

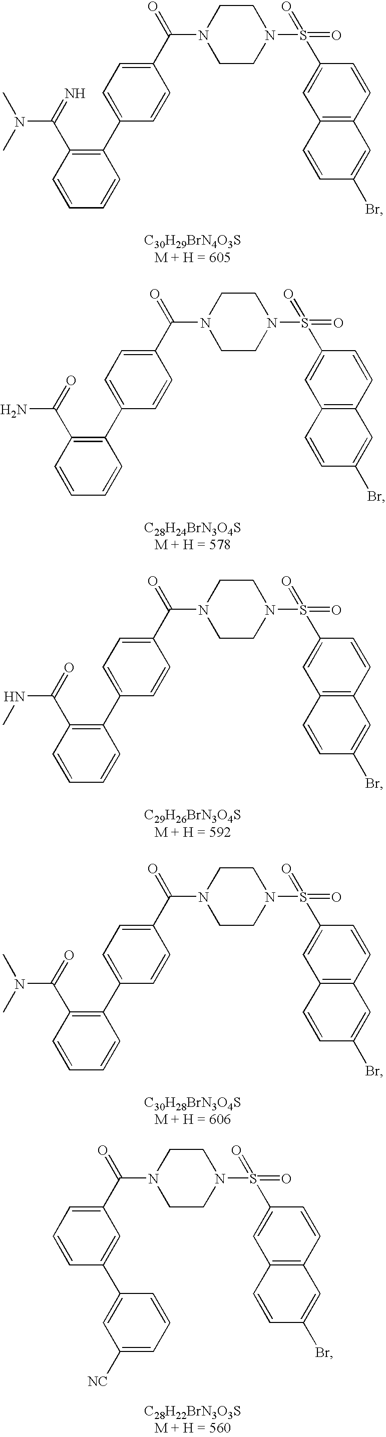

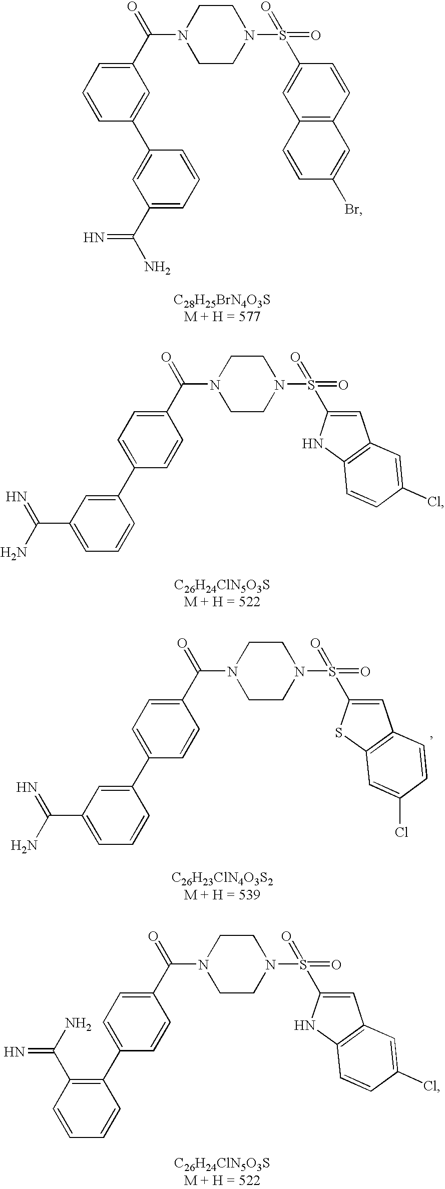

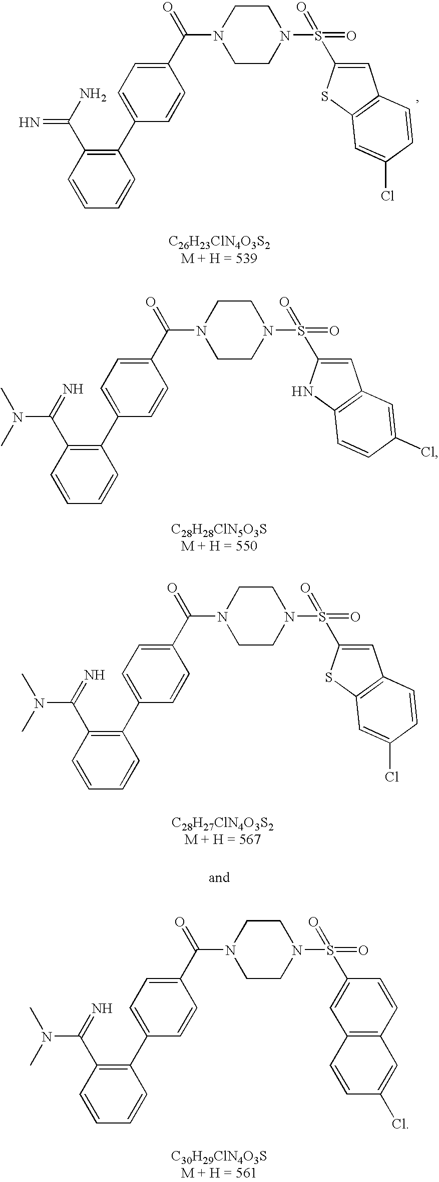

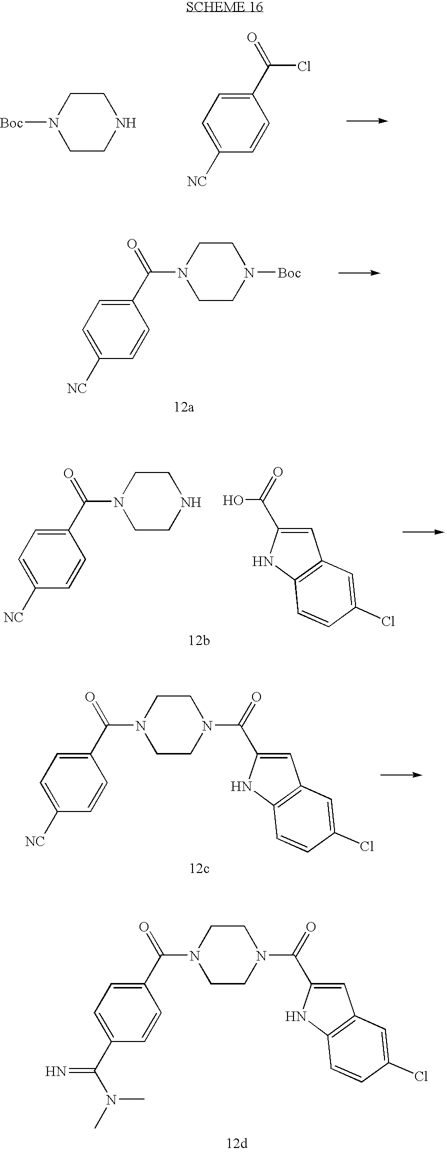

### EXAMPLE 528

### Preparation of 4-({4-[(dimethylamino)iminomethyl]phenyl}carbonyl)piperazinyl 5-chloroindol-2-yl ketone (12d)

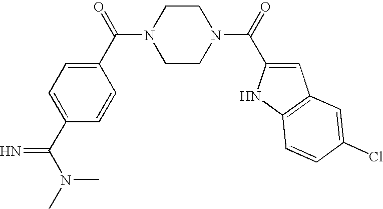

Step 1:

The mixture of 1-Boc-piperazine (3.0 g, 16 mmol) was stirred in 50 mL DCM and 50 mL pyridine. To it was added 4-cyanobenzoyl chloride (3.7 g, 22 mmol). The reaction was allowed for 30 min. It was evaporated in vacuuo, dissolved in DCM, washed with water×2, dried, evaporated in vacuuo to dryness to afford compound 12a (5.0 g, 95%).

Step 2:

Compound 12a was dissolved in 50 mL dioxane. To it was added 20 mL 4N HCl/dioxane. The mixture was stirred for overnight. It was evaporated in vacuuo to afford compound 12b (99%). MS found for C₁₂H₁₃N₃O (M+H)⁺ 216.

Step 3:

Compound 12b (360 mg, 1.67 mmol) was stirred in 15 mL pyridine. In an ice bath, to it were added 5-chloroindole-2-carboxylic acid (327 mg, 1.67 mmol) and 10 min later POCl₃ (0.47 mL, 5 mmol). The reaction was allowed for 1 hr. It was quenched with 1 mL water. The mixture was evaporated in vacuuo to remove pyridine. The residue was taken into 150 mL chloroform, washed with brine. It was dried, evaporated, purified by flash column to afford compound 12c (67%). MS found for C₂₁H₁₇ClN₄O₂ (M+H)⁺ 393.

Step 4:

Compound 12c (25 mg) was stirred in 5 mL dry methanol in ice bath. It was charged with dry HCl gas till saturation. The solution was stirred for overnight. It was evaporated in vacuuo to dryness. It was dissolved in 4 mL dry methanol. To it was added 0.2 mL dimethylamine solution (2M in THF). The new mixture was refluxed for 45 min and subjected to RP-HPLC purification to yield the title compound 12d. MS found for C₂₃H₂₄ClN₅O₂ (M+H)⁺ 438.

The following Examples 529-543 were similarly prepared by following the procedure of Example 528.

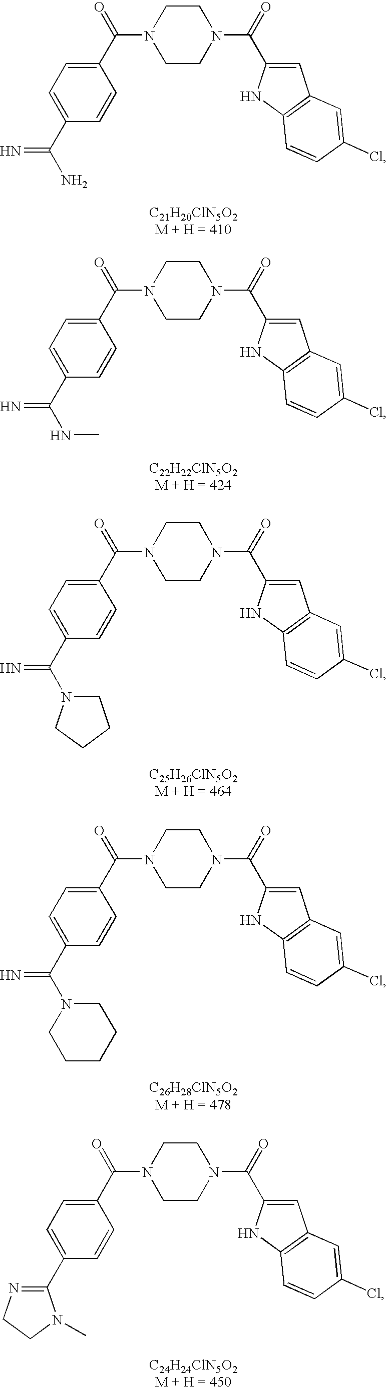

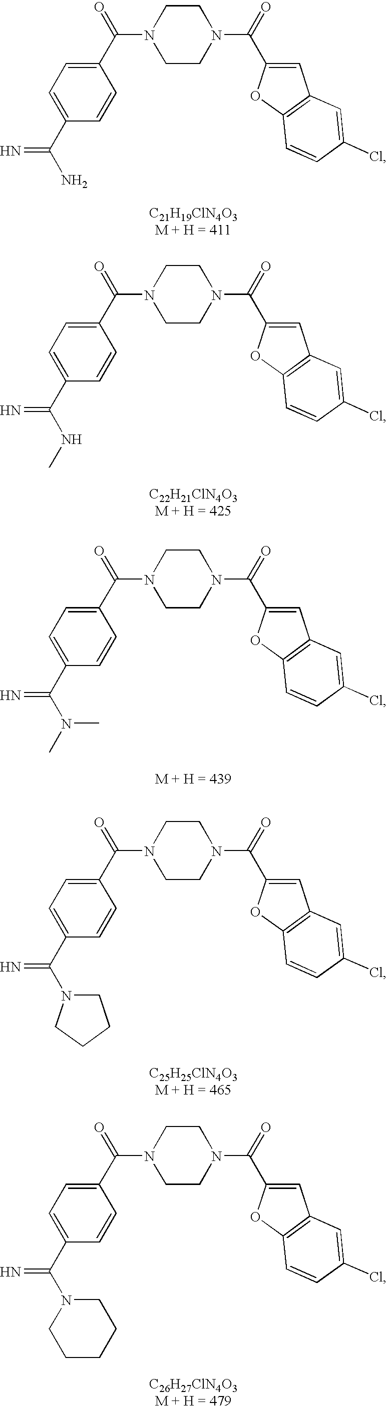

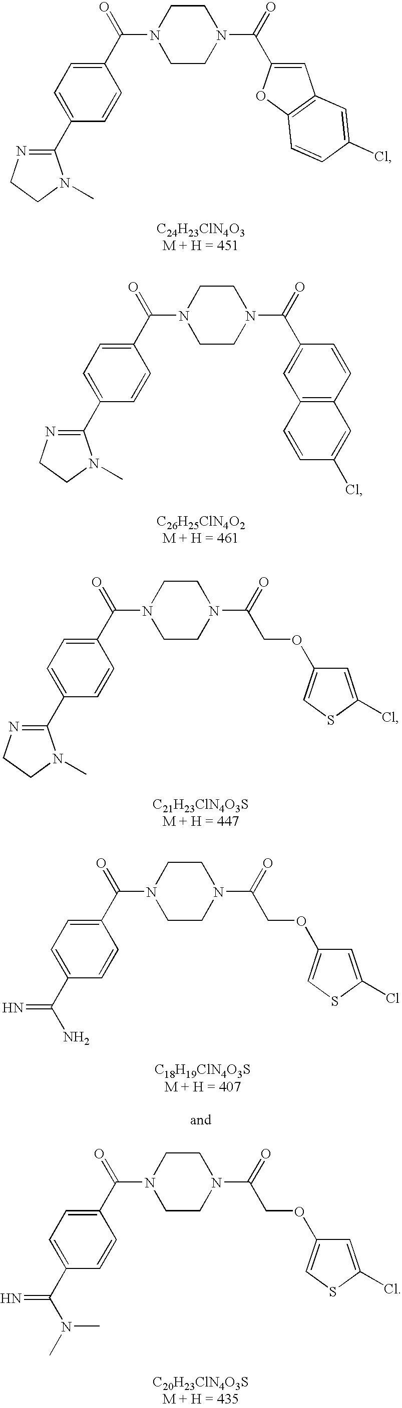

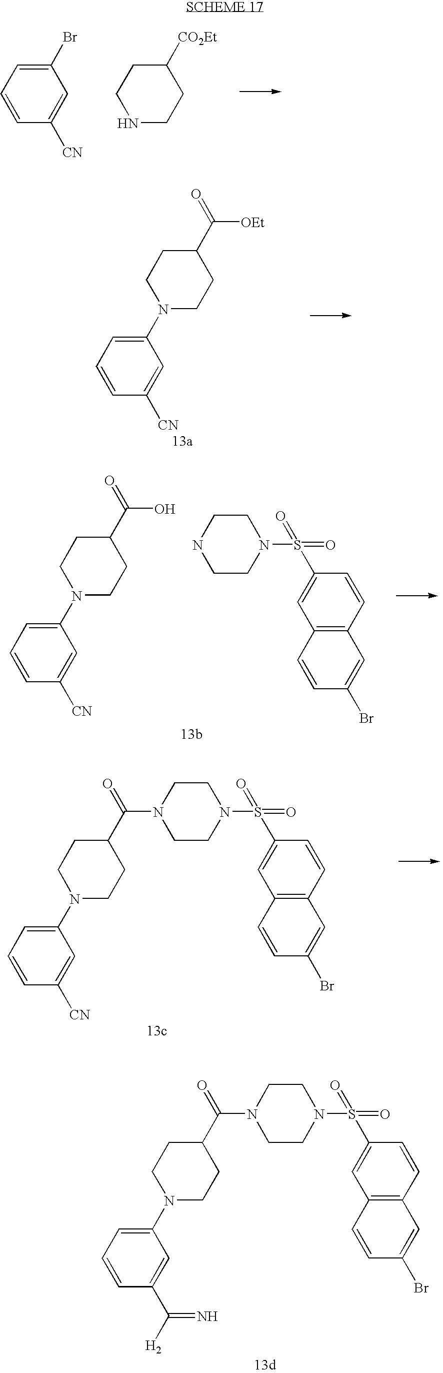

### EXAMPLE 544

### Preparation of 3-[4-({4-[(6-bromo-2-naphthyl)sulfonyl]piperazinyl}carbonyl)piperidyl]benzenecarboxamidine (13d)

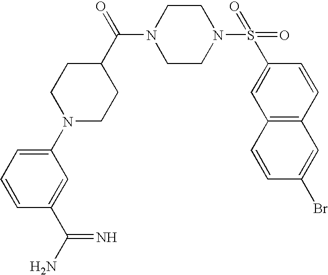

Step 1:

To the mixture of 3-bromobenzonitrile (1.82 g, 10 mmol) and ethyl isonipecotate (3.14 g, 20 mmol) in 20 mL dry toluene were added Pd₂(dba)₃ (92 mg, 0.1 mmol), (S)-BINAP (125 mg, 0.2 mmol) and sodium t-butoxide (1.35 g, 14 mmol). The mixture was degassed with argon stream for 5 min and refluxed under argon for overnight. To the mixture was added 400 mL toluene, washed with water×2, dried, filtered, concentrated in vacuuo, purified with flash column to afford compound 13a in 65% yield. MS found for C₁₅H₁₈N₂O₂ (M+H)⁺ 259.

Step 2:

Compound 13a (3.03 g, 12 mmol) was dissolved in 20 mL methanol. To it was added 1N LiOH aq solution (24 mL). The mixture was stirred for 2 hrs and acidified with 6N HCl till pH=2. A yellow solid precipitated out. It was collected via filtration. The filtrate was extracted with EtOAc×2. The organic extract was dried and evaporated in vacuuo to dryness to yield a yellow solid. It was combined with the yellow solid isolated earlier via filtration. Yield 87%. MS found for 13b C₁₃H₁₄N₂O₂ (M+H)⁺ 231.

Step 3:

6-Bromo-2-(piperazinylsulfonyl)naphthalene (100 mg, 0.28 mmol) was dissolved in 10 mL DCM. To it were added acid 13b (65 mg, 0.28 mmol), Et₃N (57 mg, 0.56 mmol) and then BOP (149 mg, 0.34 mmol). The mixture was stirred for overnight. It was diluted with 100 mL chloroform, washed with water×2, evaporated, purified with flash column to yield compound 13c (62%). MS found for C₂₇H₂₇BrN₄O₃S (M+H)⁺ 567.

Step 4:

Compound 13c (30 mg) was placed in 10 mL dry methanol. In ice bath it was charged with commercial HCl gas till saturation. The solution was stirred for overnight. It was evaporated in vacuuo and pumped to dryness. The residue was dissolved in 5 mL dry methanol. To it was added NH₄OAc (20 mg, stored in a desiccator). The mixture was refluxed for 45 min and directly subjected to RP-HPLC separation of the title compound. MS found for C₂₇H₃₀BrN₅O₃S (M+H)⁺ 584.

The following Examples 545-547 were similarly prepared by following the procedure of Example 544.

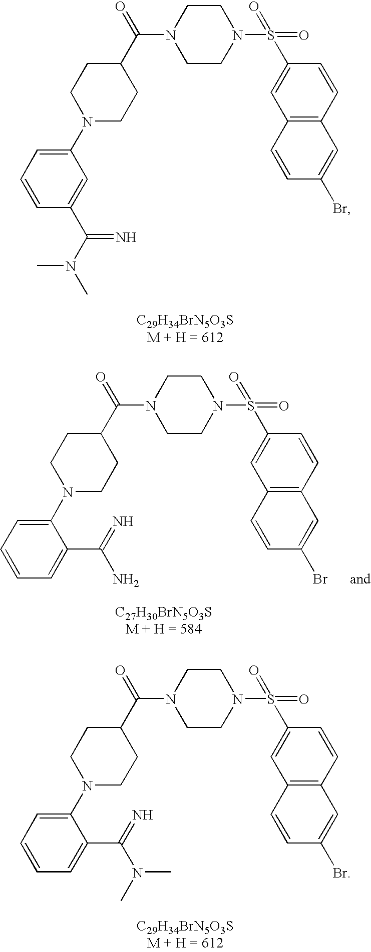

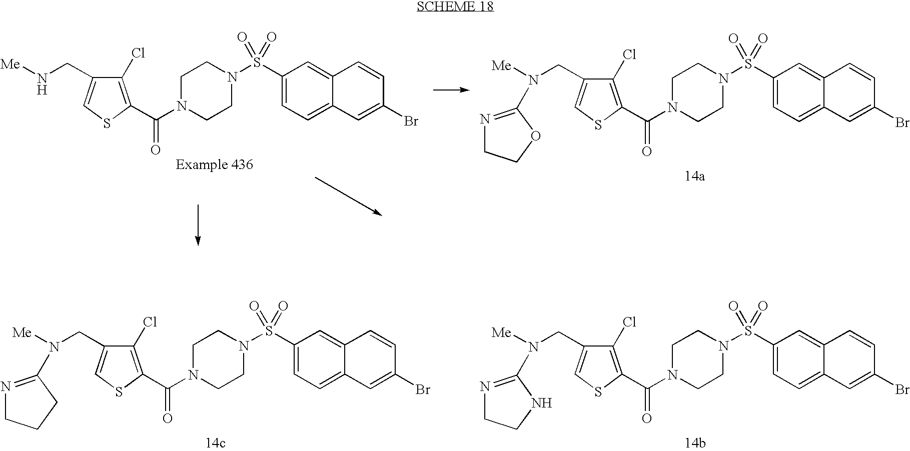

### EXAMPLE 548

### Preparation of 4-[(6-bromo(2-naphthyl))suIfonylpiperazinyl-3-chloro-4[(methyl-1,3-oxazolin-2ylamino)methyl](2-thienyl)ketone (14a)

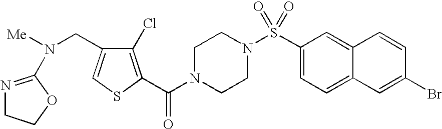

To a solution of 4-[(6-bromo(2-naphthyl))sulfonylpiperazinyl-3-chloro-4-(methylaminomethyl)(2-thienyl)ketone (Example 436, 15 mg, 1 equiv) in 3 mL of THF was added 3 drops of bromoethylisocyanate. The mixture was stirred at rt overnight and to the solution was added 0.5 mL of Et₃N. After stirring at rt for 8 days, the volatile was evaporated and the residue was subjected to RP-HPLC to give the title compound 14a (7.4 mg, 65%). MS found for C₂₄H₂₅BrClN₄O₄S₂ (M+H)⁺ 611.

### EXAMPLE 549

### Preparation of 4-[(6-bromo(2-naphthyl))sulfonylpiperazinyl-3-chloro-4[(2-imidazolin-2ylmethylamino)methyl](2-thienyl)ketone (14b)

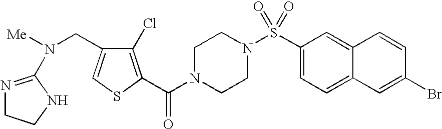

To a solution of 4-[(6-bromo(2-naphthyl))sulfonylpiperazinyl-3-chloro-4-(methylaminomethyl)(2-thienyl)ketone (Example 436, 15 mg, 1 equiv) in 2 mL of DMSO were added 2-thiomethoxyimidazoline.HI (10 mg, 3 equiv) and 5 drops of DIEA. The mixture was stirred at 150° C. for overnight. After removing the volatile, the residue was subjected to RP-HPLC to give the title compound 14b (5 mg, 40%). MS found for C₂₄H₂₆BrClN₅O₃S₂ (M+H)⁺ 610.

### EXAMPLE 550

### Preparation of 4-[(6-bromo(2-naphthyl))suffonylpiperazinyl-3-chloro-4[(methyl-1-pyrrolin-2-ylamino)methyl](2-thienyl)ketone (15c)

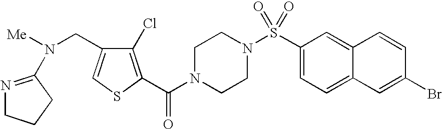

A solution of 2-pyrrolidinone (7.66 mg) in 3 mL of ether was treated with Et₃OBF₄ (0.1 mL) at rt for 3 h. The solvent was evaporated and the residue was dissolved in 1 mL of DMF and to the solution was added 4-[(6-bromo(2-naphthyl))sulfonylpiperazinyl-3-chloro-4-(methylaminomethyl)(2-thienyl)ketone (Example 436, 16 mg). The mixture was stirred at 70° C. for overnight. After removing the volatile, the residue was subjected to RP-HPLC to give the title compound 15c (8 mg, 66%). MS found for C₂₅H₂₇BrClN₄O₃S₂ (M+H)⁺ 609.

The following Examples 551-557 were similarly prepared by following the procedure of Example 550.

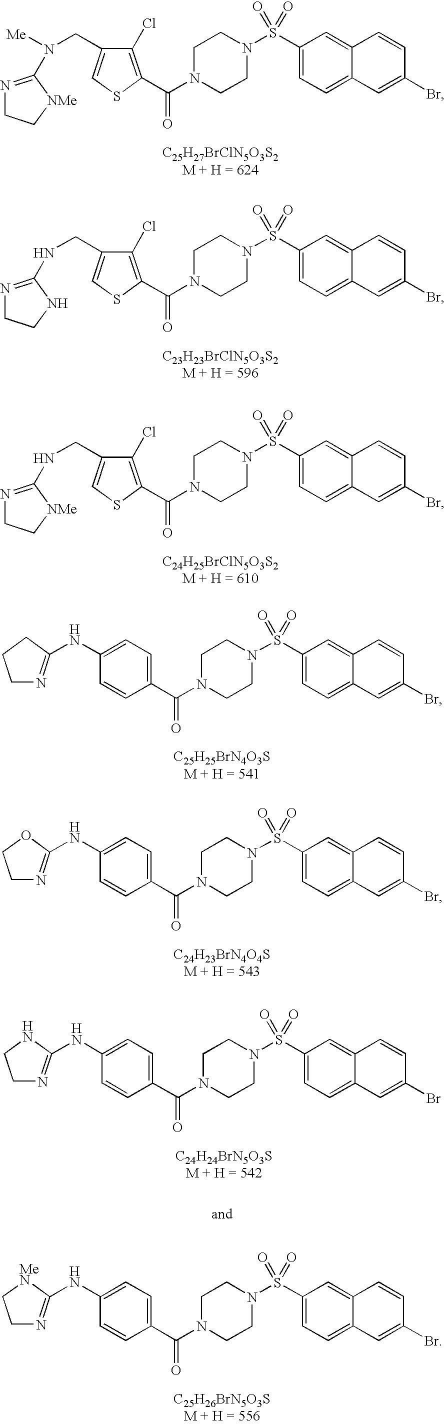

Compositions and Formulations

Compositions or formulations of the compounds of the invention are prepared for storage or administration by mixing the compound having a desired degree of purity with physiologically acceptable carriers, excipients, stabilizers etc., and may be provided in sustained release or timed release formulations. Acceptable carriers or diluents for therapeutic use are well known in the pharmaceutical field, and are described, for example, in Remington's Pharmaceutical Sciences, Mack Publishing Co., (A. R. Gennaro edit. 1985). Such materials are nontoxic to the recipients at the dosages and concentrations employed, and include buffers such as phosphate, citrate, acetate and other organic acid salts, antioxidants such as ascorbic acid, low molecular weight (less than about ten residues) peptides such as polyarginine, proteins, such as serum albumin, gelatin, or immnunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, glucose, mannose or dextrins, chelating agents such as EDTA, sugar alcohols such as mannitol or sorbitol, counterions such as sodium and/or nonionic surfactants such as Tween®, Pluronics® or polyethyleneglycol.

Dosage formulations of the compounds of the invention to be used for therapeutic administration must be sterile. Sterility is readily accomplished by filtration through sterile membranes such as 0.2 micron membranes, or by other conventional methods. Formulations typically will be stored in lyophilized form or as an aqueous solution. The pH of the preparations of the invention typically will be between about 3 and about 11, more preferably from about 5 to about 9 and most preferably from about 7 to about 8. It will be understood that use of certain of the foregoing excipients, carriers, or stabilizers will result in the formation of cyclic polypeptide salts. While the preferred route of administration is by injection, other methods of administration are also anticipated such as intravenously (bolus and/or infusion), subcutaneously, intramuscularly, colonically, rectally, nasally or intraperitoneally, employing a variety of dosage forms such as suppositories, implanted pellets or small cylinders, aerosols, oral dosage formulations and topical formulations such as ointments, drops and dermal patches. The compounds of the invention are desirably incorporated into shaped articles such as implants which may employ inert materials such as biodegradable polymers or synthetic silicones, for example, Silastic, silicone rubber or other polymers commercially available.

The compounds of the invention may also be administered in the form of liposome delivery systems, such as small unilamellar vesicles, large unilamellar vesicles and multilamellar vesicles. Liposomes can be formed from a variety of lipids, such as cholesterol, stearylamine or phosphatidylcholines.

The compounds of the invention may also be delivered by the use of antibodies, antibody fragments, growth factors, hormones, or other targeting moieties, to which the compound molecules are coupled. The compounds of the invention may also be coupled with suitable polymers as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the factor Xa inhibitors of the invention may be coupled to a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like.

Therapeutic compound liquid formulations generally are placed into a container having a sterile access port, for example, an intravenous solution bag or vial having a stopper pierceable by hypodermic injection needle.

Therapeutically effective dosages may be determined by either in vitro or in vivo methods. For each particular compound of the present invention, individual determinations may be made to determine the optimal dosage required. The range of therapeutically effective dosages will naturally be influenced by the route of administration, the therapeutic objectives, and the condition of the patient. For injection by hypodermic needle, it may be assumed the dosage is delivered into the body's fluids. For other routes of administration, the absorption efficiency must be individually determined for each inhibitor by methods well known in pharmacology. Accordingly, it may be necessary for the therapist to titer the dosage and modify the route of administration as required to obtain the optimal therapeutic effect. The determination of effective dosage levels, that is, the dosage levels necessary to achieve the desired result, will be within the ambit of one skilled in the art. Typically, applications of compound are commenced at lower dosage levels, with dosage levels being increased until the desired effect is achieved.

A typical dosage of the compounds and compositions of the invention range from about 0.001 mg/kg to about 1000 mg/kg, preferably from about 0.01 mg/kg to about 100 mg/kg, and more preferably from about 0.10 mg/kg to about 20 mg/kg. Advantageously, the compounds of the invention may be administered several times daily. Other dosage regimens may also be useful (e.g. single daily dose and/or continuous infusion).

Typically, about 0.5 to about 500 mg of a compound or mixture of compounds of the invention, as the free acid or base form or as a pharmaceutically acceptable salt, is compounded with a physiologically acceptable vehicle, carrier, excipient, binder, preservative, stabilizer, dye, flavor, etc., as called for by accepted pharmaceutical practice. The amount of active ingredient in these compositions is such that a suitable dosage in the range indicated is obtained.

Typical adjuvants which may be incorporated into tablets, capsules and the like are a binder such as acacia, corn starch or gelatin, and excipient such as microcrystalline cellulose, a disintegrating agent like corn starch or alginic acid, a lubricant such as magnesium stearate, a sweetening agent such as sucrose or lactose, or a flavoring agent. When a dosage form is a capsule, in addition to the above materials it may also contain a liquid carrier such as water, saline, a fatty oil. Other materials of various types may be used as coatings or as modifiers of the physical form of the dosage unit. Sterile compositions for injection can be formulated according to conventional pharmaceutical practice. For example, dissolution or suspension of the active compound in a vehicle such as an oil or a synthetic fatty vehicle like ethyl oleate, or into a liposome may be desired. Buffers, preservatives, antioxidants and the like can be incorporated according to accepted pharmaceutical practice.

The preferred compounds of the present invention are characterized by their ability to inhibit thrombus formation with acceptable effects on classical measures of coagulation parameters, platelets and platelet function, and acceptable levels of bleeding complications associated with their use. Conditions characterized by undesired thrombosis would include those involving the arterial and venous vasculature.

With respect to the coronary arterial vasculature, abnormal thrombus formation characterizes the rupture of an established atherosclerotic plaque which is the major cause of acute myocardial infarction and unstable angina, as well as also characterizing the occlusive coronary thrombus formation resulting from either thrombolytic therapy or percutaneous transluminal coronary angioplasty (PTCA).

With respect to the venous vasculature, abnormal thrombus formation characterizes the condition observed in patients undergoing major surgery in the lower extremities or the abdominal area who often suffer from thrombus formation in the venous vasculature resulting in reduced blood flow to the affected extremity and a predisposition to pulmonary embolism. Abnormal thrombus formation further characterizes disseminated intravascular coagulopathy commonly occurs within both vascular systems during septic shock, certain viral infections and cancer, a condition wherein there is rapid consumption of coagulation factors and systemic coagulation which results in the formation of life-threatening thrombi occurring throughout the microvasculature leading to widespread organ failure.

The compounds of the invention are useful for the treatment or prophylaxis of those diseases which involve the production and/or action of factor Xa/prothrombinase complex. The compounds of this present invention, selected and used as disclosed herein, find utility as a diagnostic or therapeutic agent for preventing or treating a condition in a mammal characterized by undesired thrombosis or a disorder of coagulation. Disease states treatable or preventable by the administration of compounds of the invention include, without limitation, occlusive coronary thrombus formation resulting from either thrombolytic therapy or percutaneous transluminal coronary angioplasty, thrombus formation in the venous vasculature, disseminated intravascular coagulopathy, the treatment of reocclusion or restenosis of reperfused coronary arteries, thromboembolic complications of surgery and peripheral arterial occlusion, a condition wherein there is rapid consumption of coagulation factors and systemic coagulation which results in the formation of life-threatening thrombi occurring throughout the microvasculature leading to widespread organ failure, hemorrhagic stroke, renal dialysis, blood oxygenation, and cardiac catheterization.

Accordingly, the invention provides a method for preventing or treating a condition in a mammal characterized by undesired thrombosis which administers to a mammal a therapeutically effective amount of a compound of the invention, as described herein. Conditions for prevention or treatment include, for example, (a) the treatment or prevention of any thrombotically mediated acute coronary syndrome including myocardial infarction, unstable angina, refractory angina, occlusive coronary thrombus occurring post-thrombolytic therapy or post-coronary angioplasty, (b) the treatment or prevention of any thrombotically mediated cerebrovascular syndrome including embolic stroke, thrombotic stroke or transient ischemic attacks, (c) the treatment or prevention of any thrombotic syndrome occurring in the venous system including deep venous thrombosis or pulmonary embolus occurring either spontaneously or in the setting of malignancy, surgery or trauma, (d) the treatment or prevention of any coagulopathy including disseminated intravascular coagulation (including the setting of septic shock or other infection, surgery, pregnancy, trauma or malignancy and whether associated with multi-organ failure or not), thrombotic thrombocytopenic purpura, thromboangiitis obliterans, or thrombotic disease associated with heparin induced thrombocytopenia, (e) the treatment or prevention of thrombotic complications associated with extracorporeal circulation (e.g. renal dialysis, cardiopulmonary bypass or other oxygenation procedure, plasmapheresis), (f) the treatment or prevention of thrombotic complications associated with instrumentation (e.g. cardiac or other intravascular catheterization, intra-aortic balloon pump, coronary stent or cardiac valve), and (g) those involved with the fitting of prosthetic devices.

Anticoagulant therapy is also useful to prevent coagulation of stored whole blood and to prevent coagulation in other biological samples for testing or storage. Thus the compounds of the invention can be added to or contacted with any medium containing or suspected to contain factor Xa and in which it is desired that blood coagulation be inhibited, e.g., when contacting the mammal's blood with material such as vascular grafts, stents, orthopedic prostheses, cardiac stents, valves and prostheses, extra corporeal circulation systems and the like.

Thus, the compounds of the invention also find utility in a method for inhibiting the coagulation of biological samples by administration of a compound of the invention.

### Biological Activity Examples

Evaluation of the compounds of the invention is guided by in vitro protease activity assays (see below) and in vivo studies to evaluate antithrombotic efficacy, and effects on hemostasis and hematological parameters.

The compounds of the present invention are dissolved in buffer to give solutions containing concentrations such that assay concentrations range from about 0 to 100 μM. In the assays for thrombin, prothrombinase and factor Xa, a synthetic chromogenic substrate is added to a solution containing test compound and the enzyme of interest and the residual catalytic activity of that enzyme is determined spectrophotometrically. The IC₅₀ of a compound is determined from the substrate turnover. The IC₅₀ is the concentration of test compound giving 50% inhibition of the substrate turnover. The compounds of the present invention desirably have an IC₅₀ of less than about 500 nM in the factor Xa assay, preferably less than about 200 nM, and more preferred compounds have an IC₅₀ of about 100 nM or less in the factor Xa assay. The compounds of the present invention desirably have an IC₅₀ of less than about 4.0 μM in the prothrombinase assay, preferably less than 200 nM, and more preferred compounds have an IC₅₀ of about 10 nM or less in the prothrombinase assay. The compounds of the present invention desirably have an IC₅₀ of greater than about 1.0 μM in the thrombin assay, preferably greater than about 10.0 μM, and more preferred compounds have an IC₅₀ of greater than about 100.0 μM in the thrombin assay.

Amidolytic Assays for Determining Protease Inhibition Activity

The factor Xa and thrombin assays are performed at room temperature, in 0.02 M Tris•HCl buffer, pH 7.5, containing 0.15 M NaCl. The rates of hydrolysis of the para-nitroanilide substrate S-2765 (Chromogenix) for factor Xa, and the substrate Chromozym TH (Boehringer Mannheim) for thrombin following preincubation of the enzyme with inhibitor for 5 minutes at room temperature, and were determined using the Softmax 96-well plate reader (Molecular Devices), monitored at 405 nm to measure the time dependent appearance of p-nitroaniline.

The prothrombinase inhibition assay is performed in a plasma free system with modifications to the method described by Sinha, U. et al., Thromb. Res., 75, 427-436 (1994). Specifically, the activity of the prothrombinase complex is determined by measuring the time course of thrombin generation using the p-nitroanilide substrate Chromozym TH. The assay consists of preincubation (5 minutes) of selected compounds to be tested as inhibitors with the complex formed from factor Xa (0.5 nM), factor Va (2 nM), phosphatidyl serine:phosphatidyl choline (25:75, 20 μM) in 20 mM Tris•HCl buffer, pH 7.5, containing 0.15 M NaCl, 5 mM CaCl₂ and 0.1% bovine serum albumin. Aliquots from the complex-inhibitor mixture are added to prothrombin (1 nM) and Chromozym TH (0.1 mM). The rate of substrate cleavage is monitored at 405 nm for two minutes. Eight different concentrations of inhibitor are assayed in duplicate. A standard curve of thrombin generation by an equivalent amount of untreated complex are used for determination of percent inhibition.

Antithrombotic Efficacy in a Rabbit Model of Venous Thrombosis

A rabbit deep vein thrombosis model as described by Hollenbach, S. et al., Thromb. Haemost. 71, 357-362 (1994); is used to determine the in-vivo antithrombotic activity of the test compounds. Rabbits are anesthetized with I.M. injections of Ketamine, Xylazine, and Acepromazine cocktail. A standardized protocol consists of insertion of a thrombogenic cotton thread and copper wire apparatus into the abdominal vena cava of the anesthetized rabbit. A non-occlusive thrombus is allowed to develop in the central venous circulation and inhibition of thrombus growth is used as a measure of the antithrombotic activity of the studied compounds. Test agents or control saline are administered through a marginal ear vein catheter. A femoral vein catheter is used for blood sampling prior to and during steady state infusion of test compound. Initiation of thrombus formation begins immediately after advancement of the cotton thread apparatus into the central venous circulation. Test compounds are administered from time=30 min to time=150 min at which the experiment is terminated. The rabbits are euthanized and the thrombus excised by surgical dissection and characterized by weight and histology. Blood samples are analyzed for changes in hematological and coagulation parameters.

Effects of Compounds in Rabbit Venous Thrombosis Model

Administration of compounds in the rabbit venous thrombosis model demonstrates antithrombotic efficacy at the higher doses evaluated. There are no significant effects of the compound on the aPTT and PT prolongation with the highest dose (100 μg/kg+2.57 μg/kg/min). Compounds have no significant effects on hematological parameters as compared to saline controls. All measurements are an average of all samples after steady state administration of vehicle or (D)-Arg-Gly-Arg-thiazole. Values are expressed as mean±SD.

Without further description, it is believed that one of ordinary skill in the art can, using the preceding description and the following illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

What is claimed is:

1. A compound of the general formulae (I) or (II): 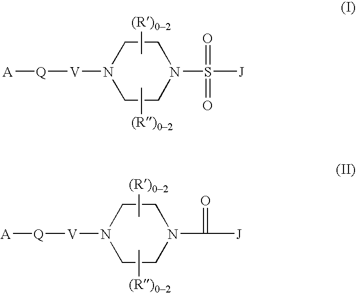
wherein:
wherein:
A is a member selected from the group consisting of: 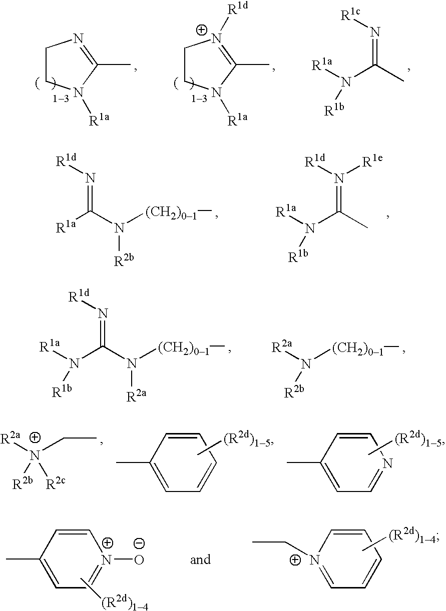
R^{1a}, R^{1b}, R^{1d}, and R^{1e} are each independently a H, C₁₋₆ alkyl, C₂₋₆ alkenyl, C₂₋₆ alkynyl, C₃₋₈cycloalkyl, aryl, —C₁₋₆alkylaryl, —C₁₋₆alkyl-OC₁₋₆alkyl, —C₁₋₆alkyl-NRₐR_{b}, —(CH₂)₁₋₆NRₐC(═O)C₁₋₆ alkyl, —(CH₂)₁₋₆C(═O)OH, —(CH₂)₁₋₆C(═O)OC₁₋₆alkyl, or —(CH₂)₁₋₆C(═O)NRₐR_{b}; or R^{1a} and R^{1b} or R^{1a} and R^{1c} or R^{1a} and R^{1d} or R^{1d} and R^{1e} taken together with the nitrogen atom to which they are each attached can form a substituted or unsubstituted 3 to 8 membered heterocyclic or heteroaromatic amine group which, optionally, contains at least one other heteroatom of N, O or S; wherein R^{1a}, R^{1b}, R^{1d}, or R^{1e} is optionally substituted with at least one of halo, alkyl, alkylideneamine, arylidenamine, cyano, hydroxy, alkoxy, amino, amidino, guanidino, imino, amido, acid, ester, keto, aldehyde, dioxolane, furanyl, piperidinyl, piperazinyl, pyrrolidinyl, aryl, morpholinyl, and thiomorpholinyldioxide;
R^{1c} is H, C₁₋₆alkyl or C₃₋₈cycloalkyl;
R^{2a}, R^{2b} and R^{2c} are each independently a H, C₁₋₆alkyl, —C₂₋₆alkenyl, —C₂₋₆alkynyl, —C₃₋₈cycloalkyl, aryl, —C₁₋₆alkylaryl, —C₁₋₆alkyl-OC₁₋₆alkyl, —C₁₋₆alkyl-NRₐR_{b}, —(CH₂)₁₋₆NRₐC(═O)C₁₋₆ alkyl, —(CH₂)₁₋₆C(═O)OH, —(CH₂)₁₋₆C(═O)OC₁₋₆alkyl, or —(CH₂)₁₋₆C(═O)NRₐR_{b}; or R^{2a} and R^{2b} or R^{1a}, as set forth above, and R^{2a} or R^{1a}, as set forth above, and R^{2b} taken together with the nitrogen atom to which they are each attached can form a substituted or unsubstituted 3 to 8 membered heterocyclic or heteroaromatic amine group which, optionally, contains at least one other heteroatom of N, O or S; wherein R^{2a}, R^{2b} or R^{2c} is optionally substituted with at least one of halo, alkyl, alkylideneamine, arylidenamine, cyano, hydroxy, alkoxy, amino, amidino, guanidino, imino, amido, acid, ester, keto, aldehyde, dioxolane, furanyl, piperidinyl, piperazinyl, pyrrolidinyl, aryl, morpholinyl, and thiomorpholinyldioxide;
R^{2d} is —SO₂NRₐR_{b}, —SO₂C₁₋₆alkyl, —CN, —C₀₋₆alkylNRₐR_{b}, —C(═NH)—NRₐR_{b}, or —C(═O)—NRₐR_{b}, where Rₐ and R_{b} are each as set forth below;
Rₐ and R_{b} are independently H, —C₁₋₆alkyl, —C₂₋₆alkenyl, —C₂₋₆alkynyl, aryl; or Rₐ and R_{b} taken together with the nitrogen to which they are attached form azetidinyl, pyrrolidinyl, piperidinyl, morpholinyl, thiomorpholinyl and its oxidized forms, piperazinyl, 4-methyl-1-piperazinyl, morpholinylcarbaldehyde, piperazinylcarbaldehyde or thiomorpholinylcarbaldehyde and its oxidized forms;
V is —CH₂— or —C(═O)—;
Q is a member selected from the group consisting of: 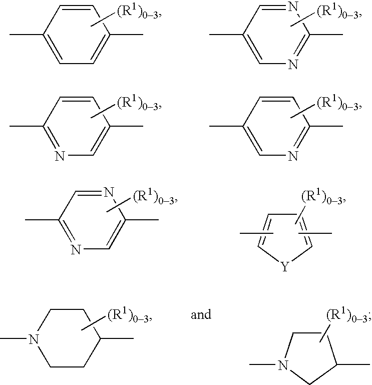
Y is N, NMe, O, or S;
R¹ is H, —Cl, —Br, —I, —F, —C₁₋₆alkyl, —C₂₋₆alkenyl, —C₀₋₆alkylNRₐR_{b}, —C₀₋₆alkylOH, —C₀₋₆alkylCN, —C₀₋₆alkylCO₂H, —C₀₋₆alkylCONRₐR_{b}, —C₀₋₆alkylOC₁₋₆alkyl, —C₀₋₆alkylOCF₃, —SH, —SC₁₋₆alkyl, —SOC₁₋₆alkyl, —SO₂-C₁₋₆alkyl, —CN, —COOH, —COOC₁₋₆alkyl, —CONRₐR_{b}, where Rₐ and R_{b} are each as set forth above;
J is a member selected from the group consisting of: 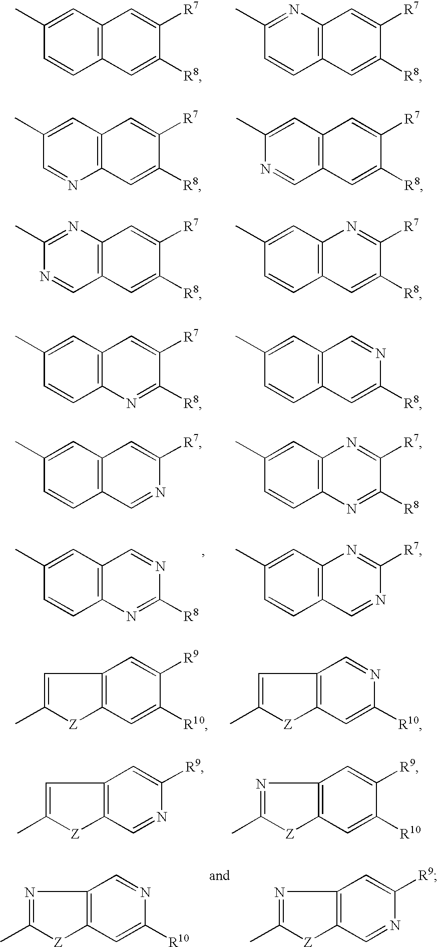
Z is —NR⁶—, —O— or —S—;
R⁶ is H, C₁₋₆alkyl or C₃₋₈cycloalkyl;
R⁷ and R⁸ are independently H, —Cl, —Br, —I or —F, where at least one of R⁷ and R⁸ is not hydrogen; and
R⁹ and R¹⁰ are independently H, —Cl, —Br, —I or —F, where at least one of R⁹ and R¹⁰ is not hydrogen;
R′ and R″ are independently selected from —H, —C₁₋₆alkyl, —C₁₋₆alkyl-OH, —C₁₋₆alkyl-NRₐR_{b}, —C₁₋₆alkylCN, —C₁₋₆alkylCO₂H, —C₁₋₆alkylCO₂C₁₋₆alkyl, and —C₁₋₆alkylCONRₐR_{b}, wherein Rₐ and R_{b} are the same as defined above;
and all pharmaceutically acceptable isomers, salts, hydrates, solvates and prodrug derivatives thereof.

2. A compound of claim 1, wherein:
A is the a member selected from the group consisting of: 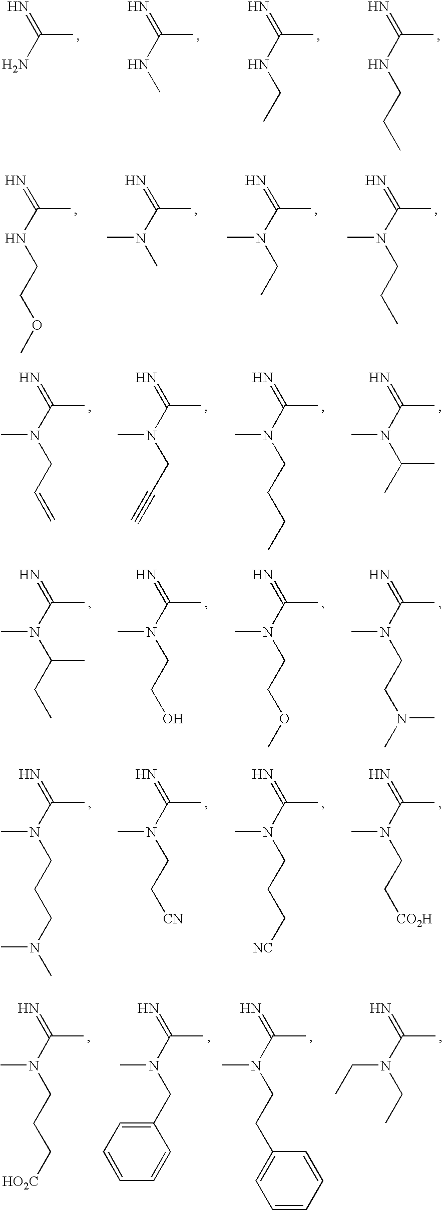 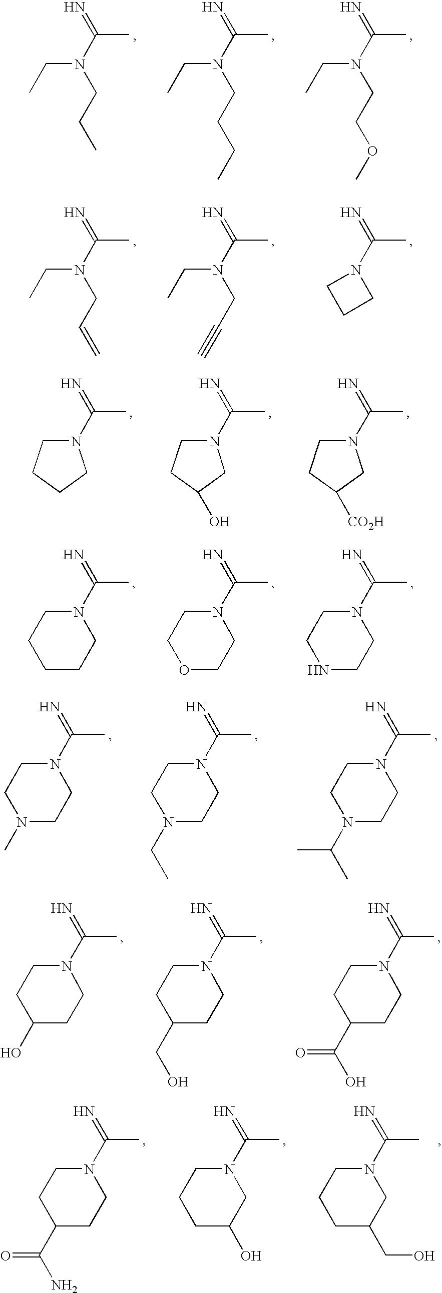 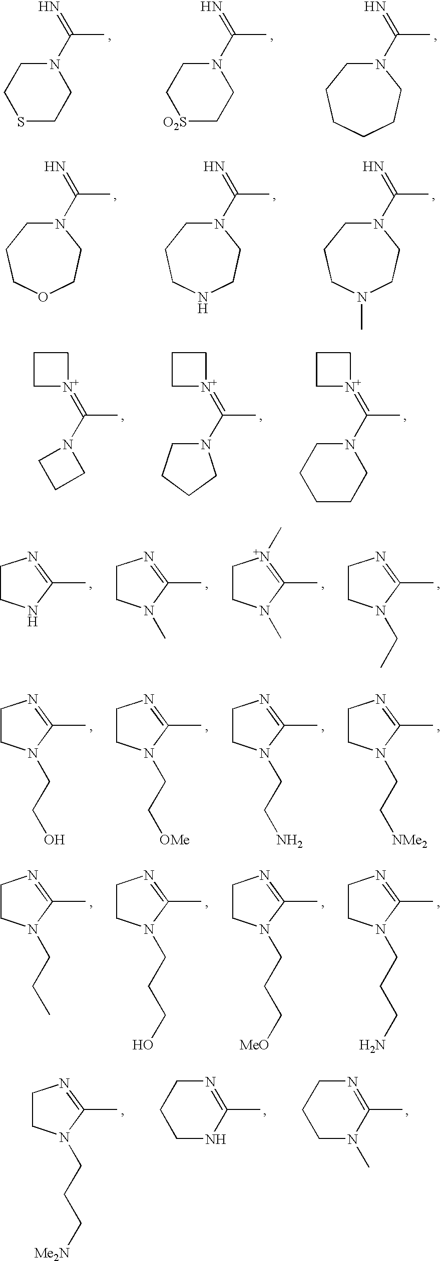 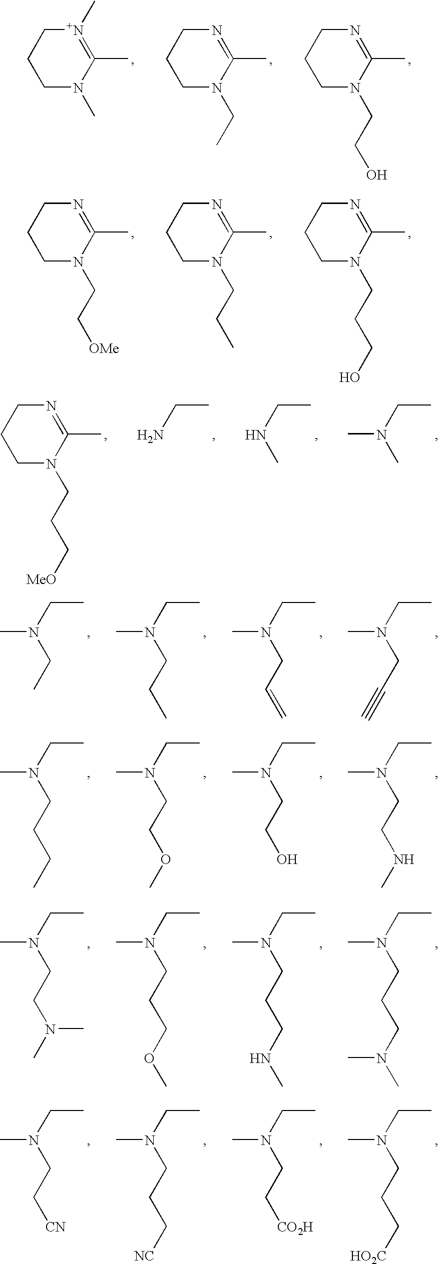 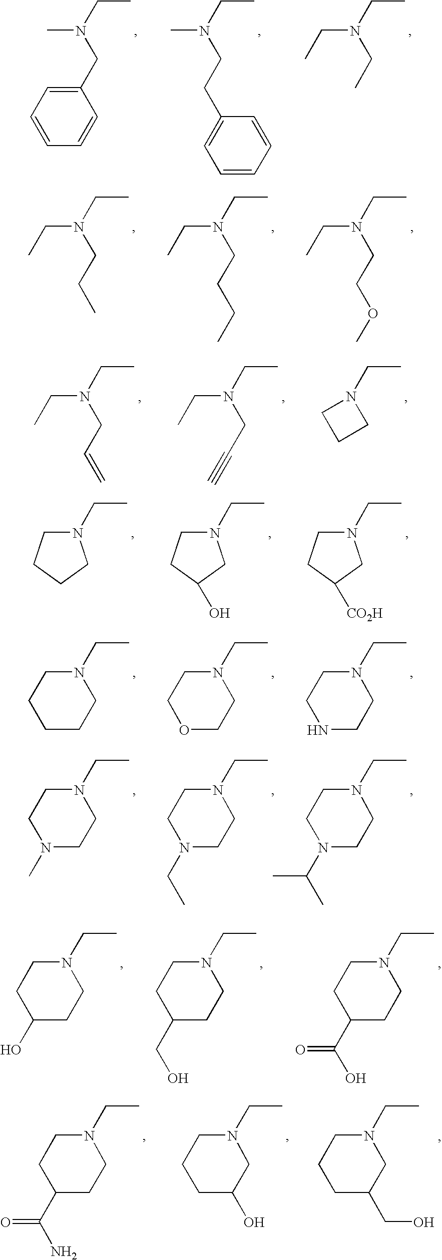 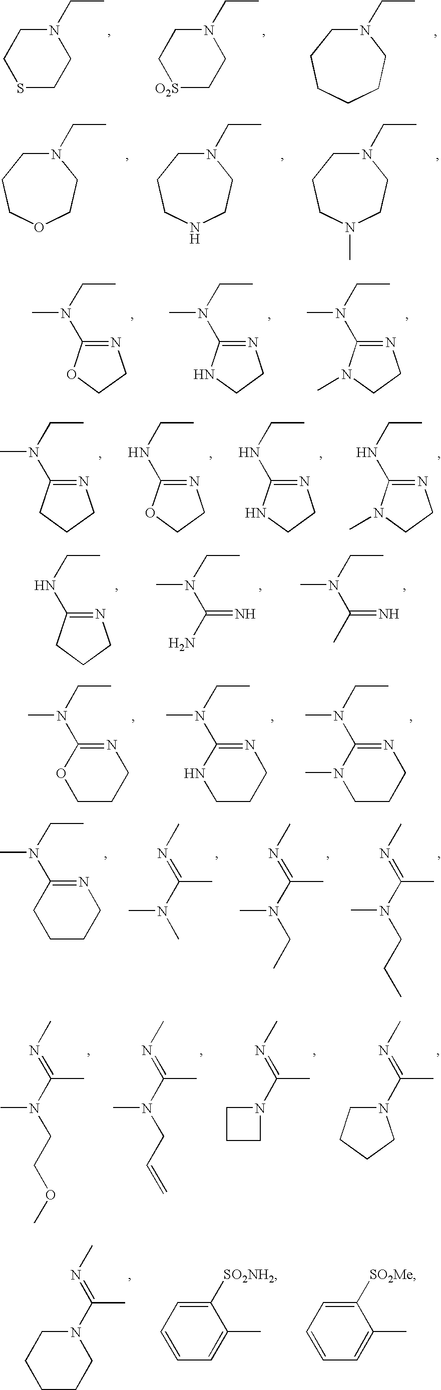 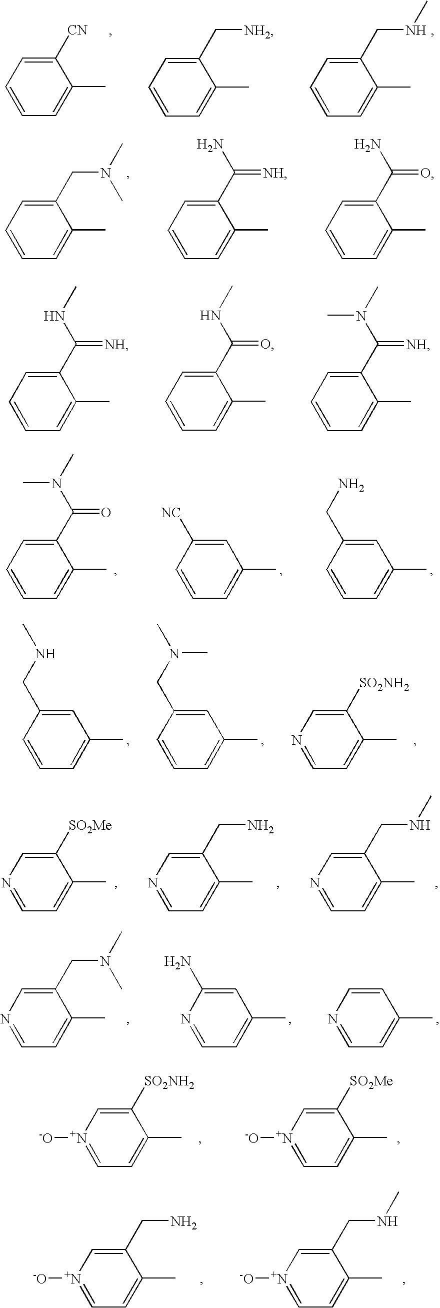 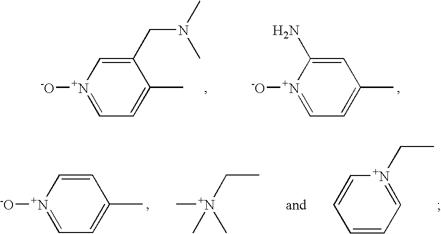
V is —CH₂— or —C(═O)—;
Q is a member selected from the group consisting of: 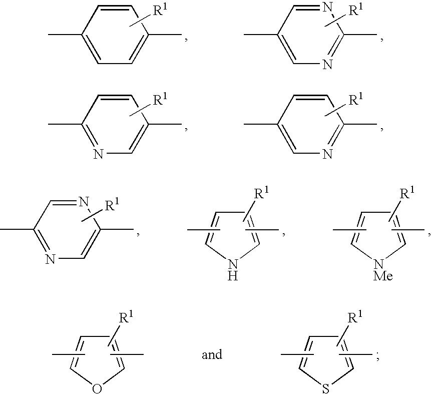
R¹ is H, —Cl, —Br, —I, —F, —C₁₋₆alkyl, —C₂₋₆alkenyl, —C₀₋₆alkylNRₐR_{b}, —C₀₋₆alkylOH, —C₀₋₆alkylOC₁₋₆alkyl, —SH, —SC₁₋₆alkyl, —SOC₁₋₆alkyl, —SO₂-C₁₋₆alkyl, —CN, —COOH, —COOC₁₋₆alkyl, —CONRₐR_{b}, where Rₐ and R_{b} are each as set forth above;
J is a member selected from the group consisting of: 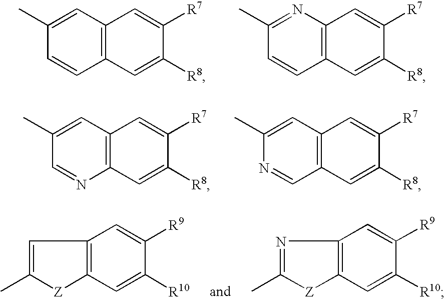
Z is —NH—, —NMe—, —O— or —S—;
R⁷ and R⁸ are independently H, —Cl, —Br, —I or —F, where at least one of R⁷ and R⁸ is not hydrogen;
R⁹ and R¹⁰ are independently H, —Cl, —Br, —I or —F, where at least one of R⁹ and R¹⁰ is not hydrogen; and
R′ and R″ are independently —H, —C₁₋₆alkyl, —C₁₋₆alkyl-OH, —C₀₋₆alkyl-NRₐR_{b}, —C₀₋₆alkylCN, —C₀₋₆alkylCO₂H, —C₀₋₆alkylCO₂C₁₋₆alkyl, and —C₀₋₆alkylCONRₐR_{b}, wherein Rₐ and R_{b} are the same as defined above.

3. A compound of claim 1, wherein:
A is a member selected from the group consisting of: 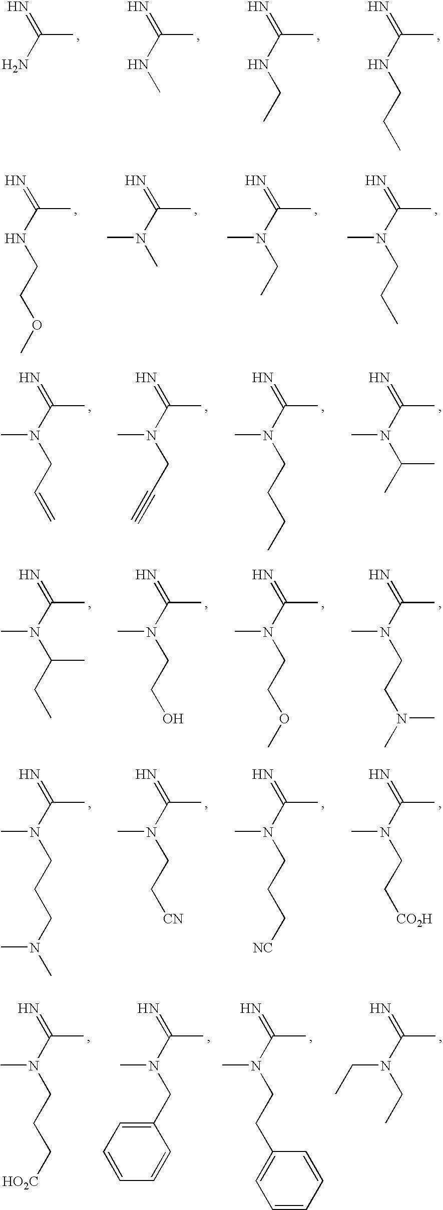 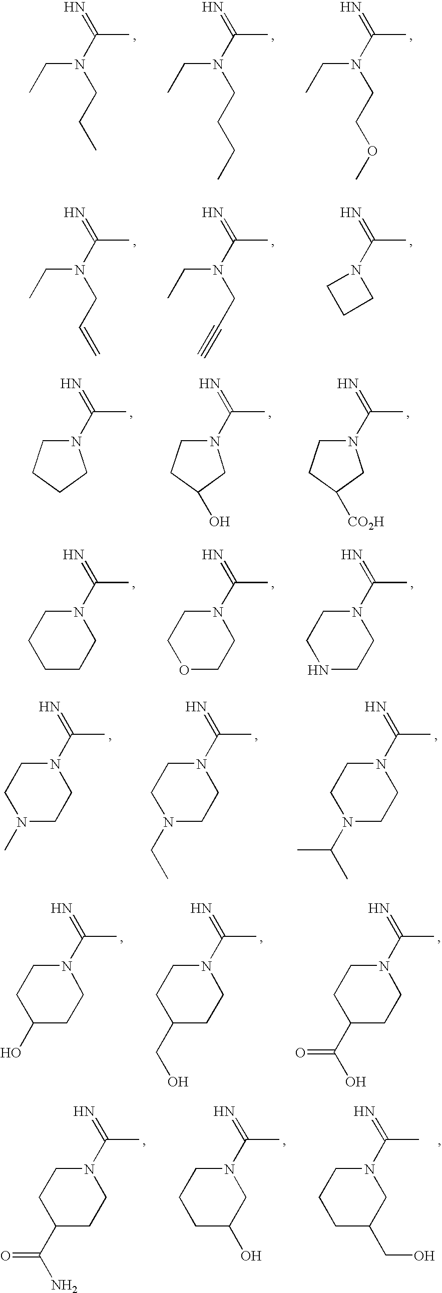 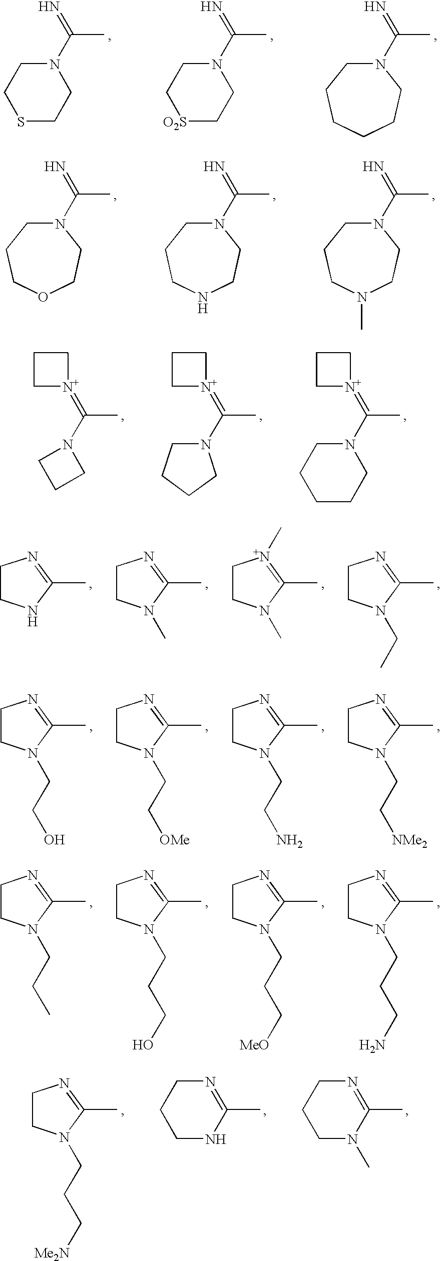 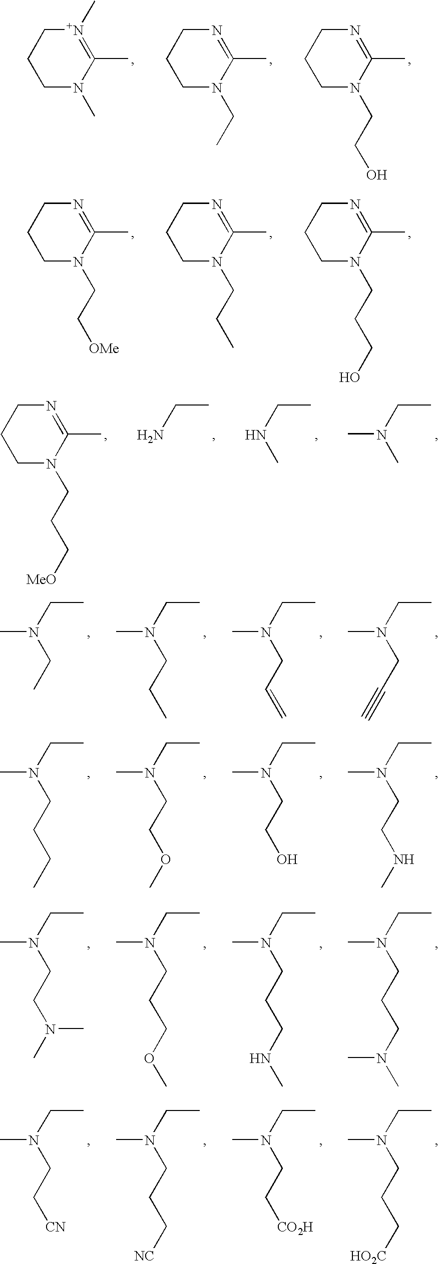 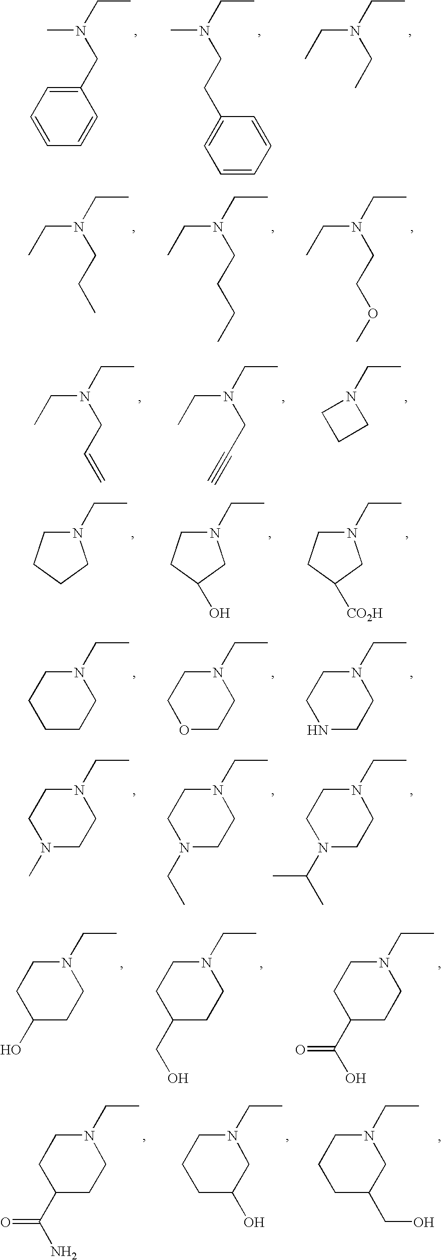 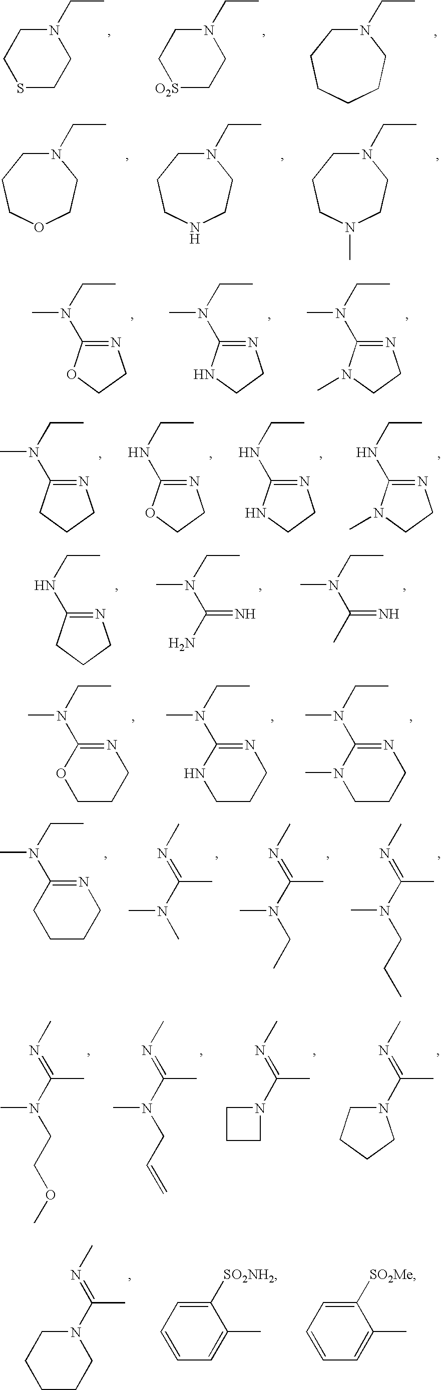 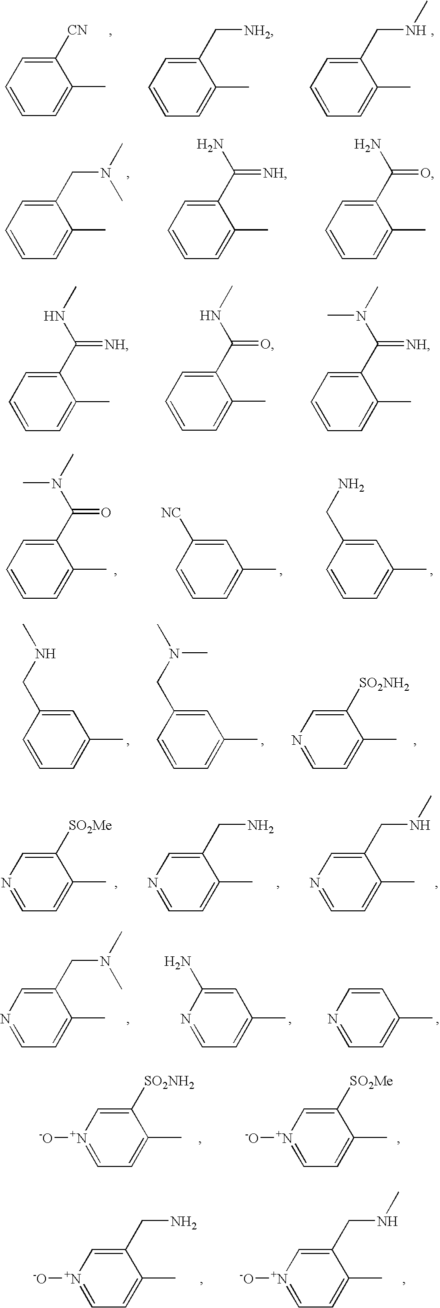 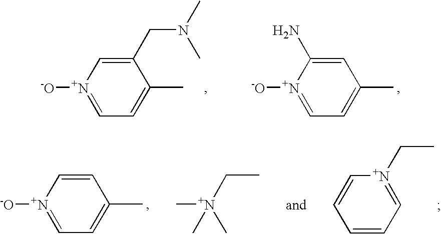
V is —CH₂— or —C(═O)—;
Q is a member selected from the group consisting of: 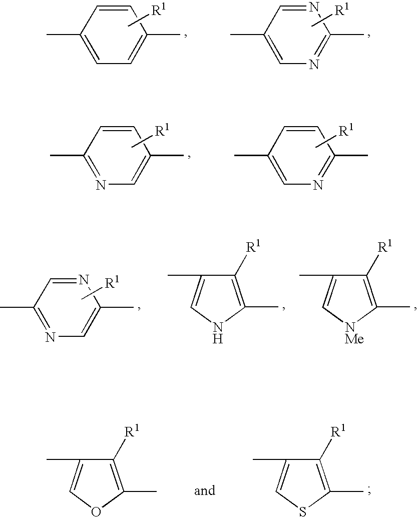
R¹ is a H, —F, —Cl, —OH, —OMe, —OEt, —SMe, —SEt, —NMe₂;
J is a member selected from the group consisting of: 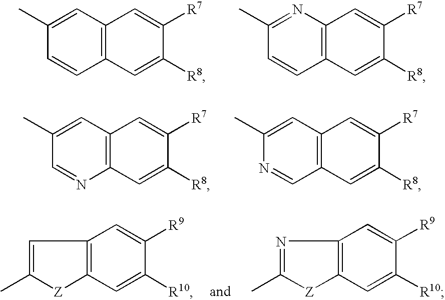
Z is —NH—, —NMe—, —O— or —S—;
R⁷ and R⁸ are independently H, —F, —Cl, or —Br, where at least one of R⁷ and R⁸ is not hydrogen; and
R⁹ and R¹⁰ are independently H, —F, —Cl, or —Br, where at least one of R⁹ and R¹⁰ is not hydrogen; and
R′ and R″ are independently —H, —C₁₋₆alkyl, —C₁₋₆alkyl-OH, —C₀₋₆alkyl-NRₐR_{b}, —C₀₋₆alkylCN, —C₀₋₆alkylCO₂H, —C₀₋₆alkylCO₂C₁₋₆alkyl, and —C₀₋₆alkylCONRₐR_{b}, wherein Rₐ and R_{b} are the same as defined above.

4. A compound of claim 1 of formula (I) having one of the following structures: 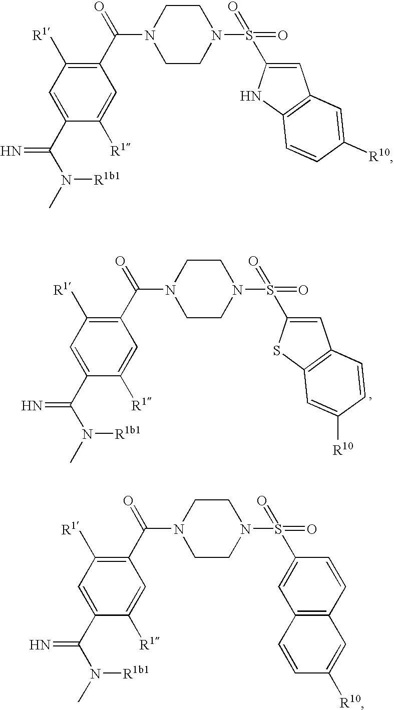 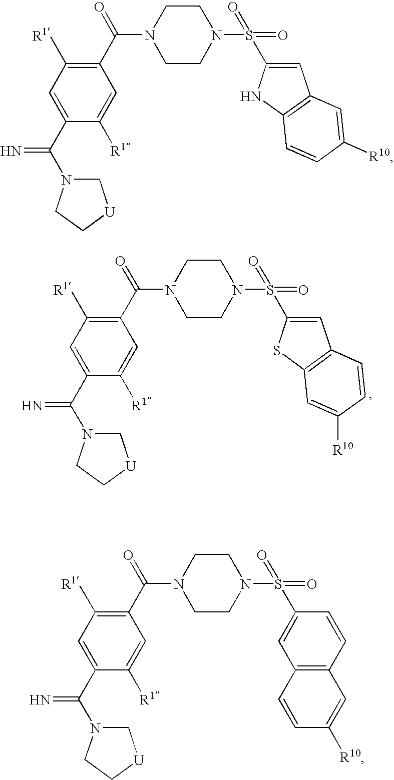 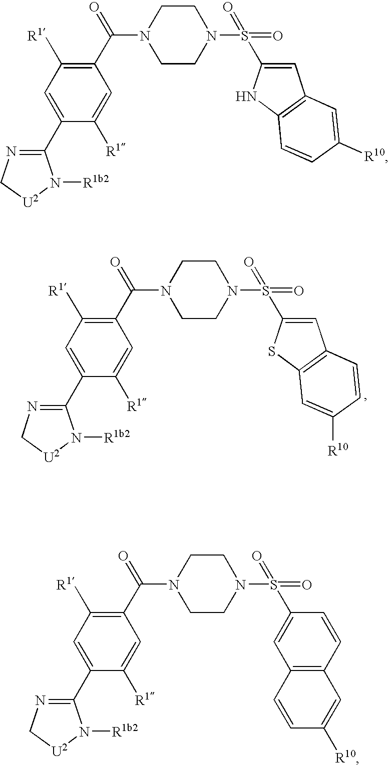 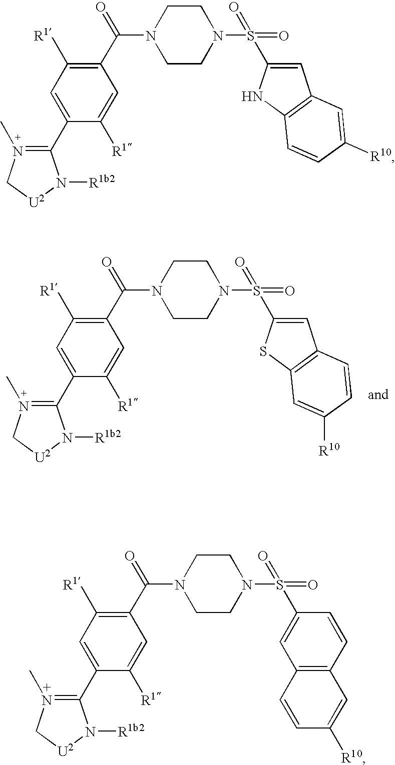
wherein:
R¹⁰ is —Cl or —Br;
R^{1b1} is H, —CH₃, —C₂H₅, —C₃H₇, —C₃H₅, —C₃H₃, —CH₂CH₂OCH₃, —CH₂CH₂N(CH₃)₂, —CH₂CH₂CH₂N(CH₃)₂, —CH₂CH₂CN, —CH₂CH₂CO₂H, —CH₂C₆H₅, or —CH₂CH₂C₆H₅;
R^{1b2} is H, —CH₃, —C₂H₅, —C₃H₇, —CH₂CH₂OH, —CH₂CH₂NH₂, —CH₂CH₂CH₂OH, —CH₂CH₂OCH₃, or —CH₂CH₂CH₂OCH₃;
R^{1′} and R^{1″} are independently H, —CH₃, —C₂H₅, —CH₂OH, —CH₂NH₂, —OH, —OCH₃, —OC₂H₅, —OC₃H₇, —SH, —SCH₃, —SC₂H₅, —SC₃H₇, —SOCH₃, —SO₂CH₃, —SOC₂H₅, —SO₂C₂H₅, —SOC₃H₇, —SO₂C₃H₇, —CN, —CO₂H, —CONH₂, —F or —Cl;
U is a direct link, —CH₂—, —CH₂CH₂—, —CH₂O—, —CH₂NH—, —CH₂N(CH₃)—, —CH(CO₂H)—CH₂—, —CH(CONH₂)—CH₂—, —CH(OH)CH₂ or —CH(CH₂OH)CH₂; and
U² is —CH₂— or —CH₂CH₂—.

5. A compound of claim 1 of formula (I) having one of the following structures: 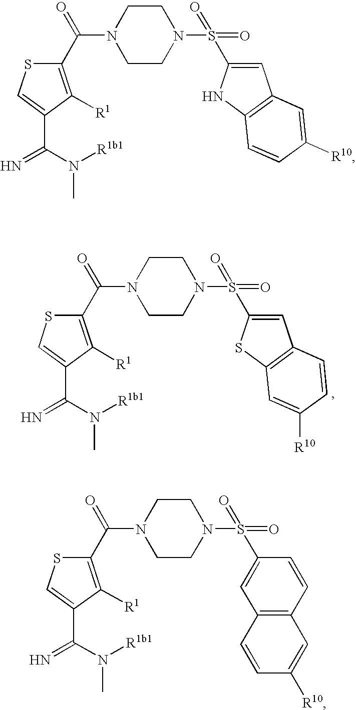 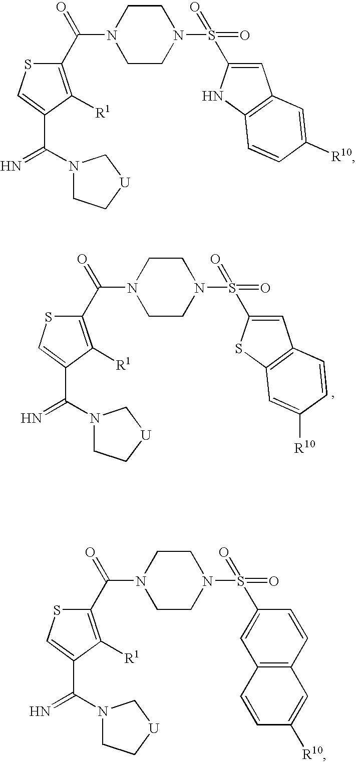 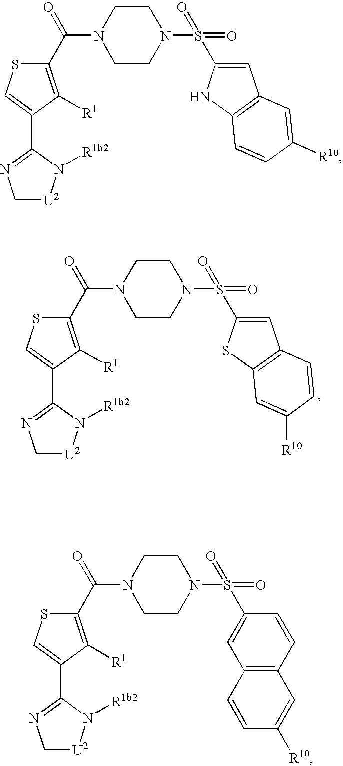 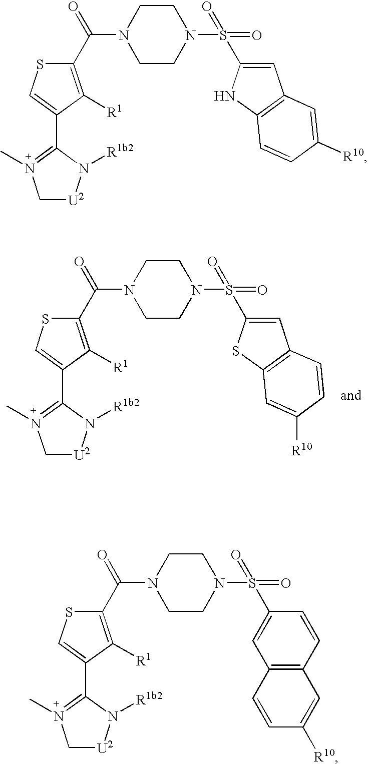
wherein:
R¹⁰ is —Cl or —Br;
R^{1b1} is H, —CH₃, —C₂H₅, —C₃H₇, —C₃H₅, —C₃H₃, —CH₂CH₂OCH₃, —CH₂CH₂N(CH₃)₂, —CH₂CH₂CH₂N(CH₃)₂, —CH₂CH₂CN, —CH₂CH₂CO₂H, —CH₂C₆H₅, or —CH₂CH₂C₆H₅;
R^{1b2} is H, —CH₃, —C₂H₅, —C₃H₇, —CH₂CH₂OH, —CH₂CH₂NH₂, —CH₂CH₂CH₂OH, —CH₂CH₂OCH₃, or —CH₂CH₂CH₂OCH₃;
R¹ is H, —CH₃, —C₂H₅, —CH₂OH, —CH₂NH₂, —OH, —OCH₃, —OC₂H₅, —OC₃H₇, —SH, —SCH₃, —SC₂H₅, —SC₃H₇, —SOCH₃, —SO₂CH₃, —SOC₂H₅, —SO₂C₂H₅, —SOC₃H₇, —SO₂C₃H₇, —CN, —CO₂H, —CONH₂, —F or —Cl;
U is a direct link, —CH₂—, —CH₂CH₂—, —CH₂O—, —CH₂NH—, —CH₂N(CH₃)—, —CH(CO₂H)—CH₂—, —CH(CONH₂)—CH₂—, —CH(OH)CH₂ or —CH(CH₂OH)CH₂; and
U² is —CH₂— or —CH₂CH₂—.

6. A compound of claim 1 of formula (I) having one of the following structures: 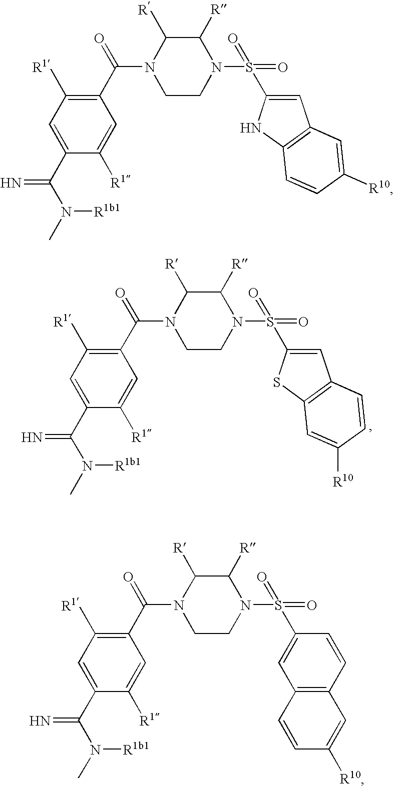 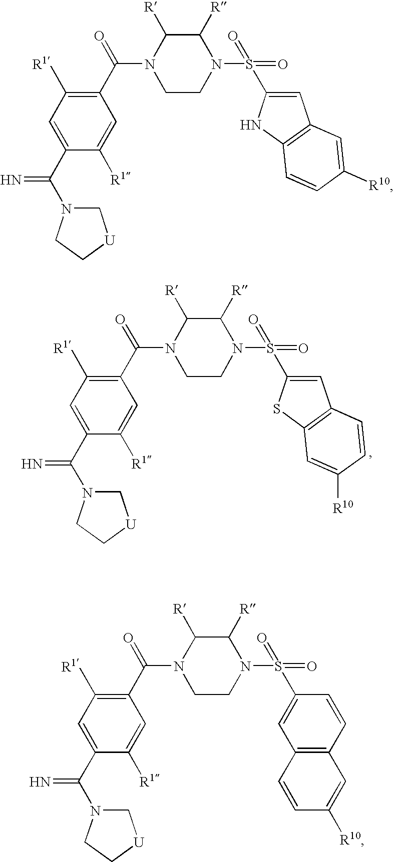 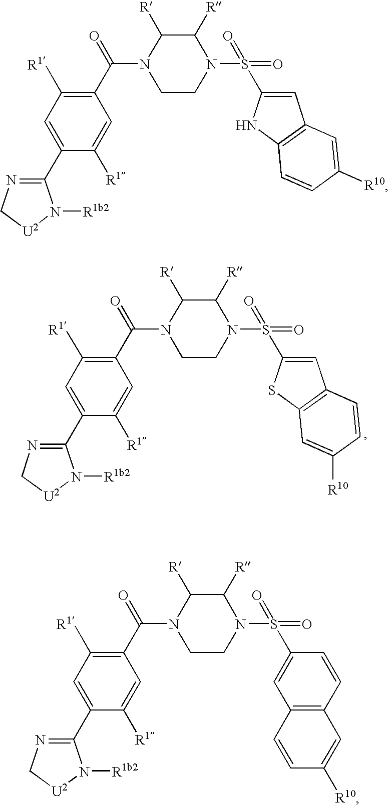 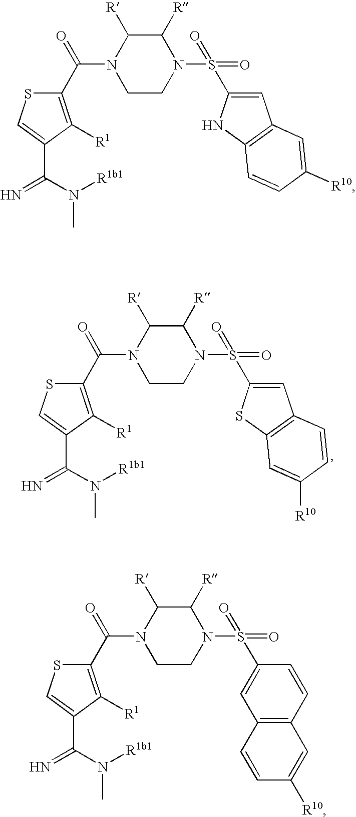 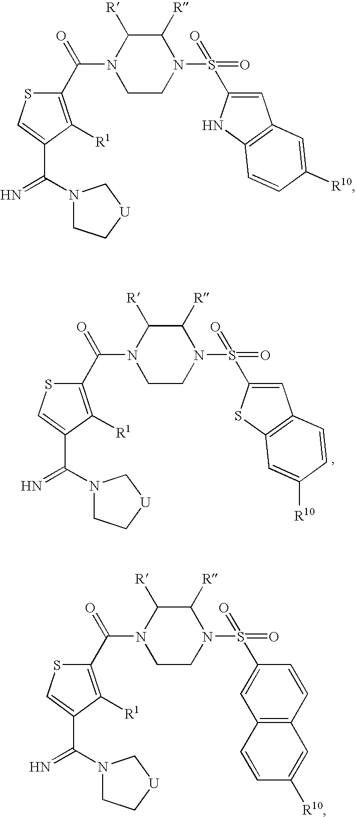 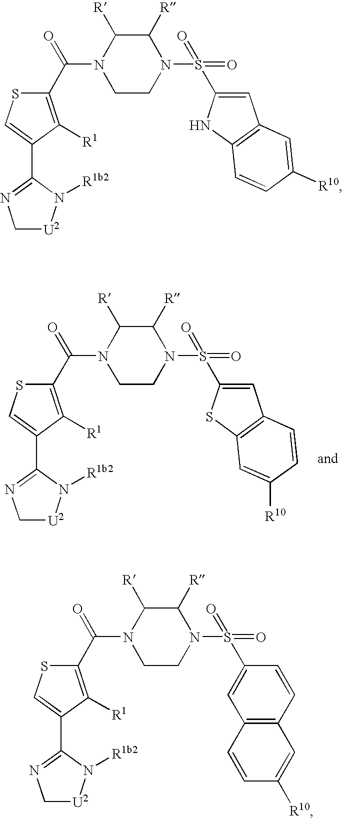
wherein:
R¹⁰ is —Cl or —Br;
R^{1b1} is H, —CH₃, —C₂H₅, —C₃H₇, —C₃H₅, —C₃H₃, —CH₂CH₂OCH₃, —CH₂CH₂N(CH₃)₂, —CH₂CH₂CH₂N(CH₃)₂, —CH₂CH₂CN, —CH₂CH₂CO₂H, —CH₂C₆H₅, or —CH₂CH₂C₆H₅;
R^{1b2} is H, —CH₃, —C₂H₅, —C₃H₇, —CH₂CH₂OH, —CH₂CH₂NH₂, —CH₂CH₂CH₂OH, —CH₂CH₂OCH₃, or —CH₂CH₂CH₂OCH₃;
R′ and R″ are independently H, —CH₃, —C₂H₅, —CO₂CH₃, —CH₂CO₂CH₃, —CO₂H, —CH₂CO₂H, —CONRₐR_{b} or —CH₂CONRₐR_{b}, where Rₐ and R_{b} are independently H, —CH₃, —C₂H₅, —C₃H₇, —C₃H₅ or C₃H₃; or Rₐ and R_{b} taken together with the nitrogen to which they are attached form an azetidinyl, pyrrolidinyl, piperidinyl, morpholinyl, thiomorpholinyl and its oxidized forms, piperazinyl or 4-methyl-1-piperazinyl;
R¹, R^{1′} and R^{1″} are independently H, —CH₃, —C₂H₅, —CH₂OH, —CH₂NH₂, —OH, —OCH₃, —OC₂H₅, —OC₃H₇, —SH, —SCH₃, —SC₂H₅, —SC₃H₇, —SOCH₃, —SO₂CH₃, —SOC₂H₅, —SO₂C₂H₅, —SOC₃H₇, —SO₂C₃H₇, —CN, —CO₂H, —CONH₂, —F or —Cl;
U is a direct link, —CH₂—, —CH₂CH₂—, —CH₂O—, —CH₂NH—, —CH₂N(CH₃)—, —CH(CO₂H)—CH₂—, —CH(CONH₂)—CH₂—, —CH(OH)CH₂ or —CH(CH₂OH)CH₂; and
U² is —CH₂— or —CH₂CH₂—,

7. A compound of claim 1 of formula (I) having one of the following structures: 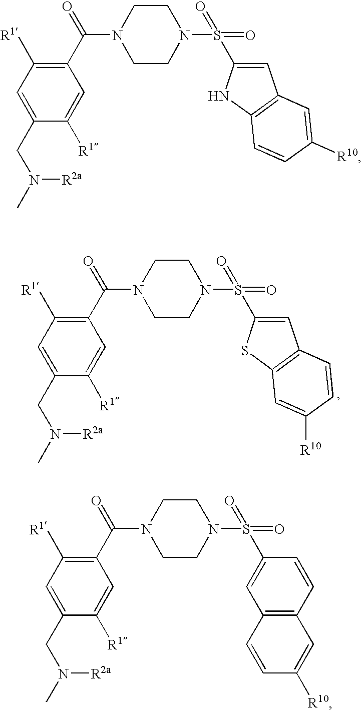 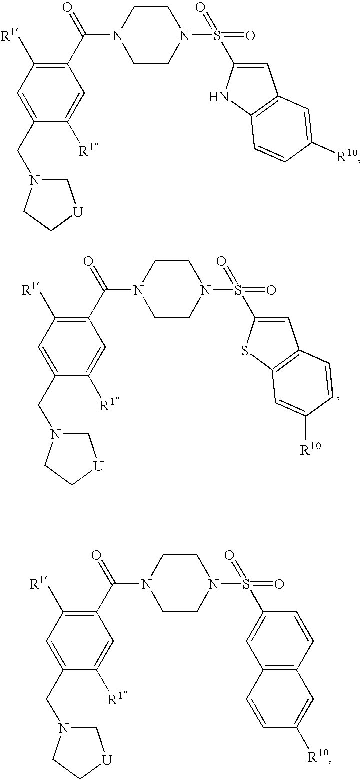 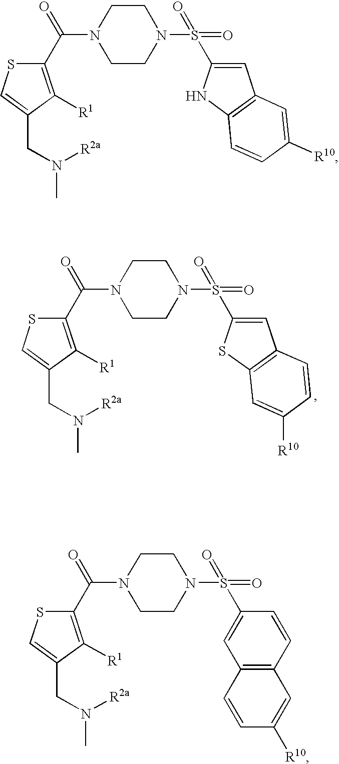 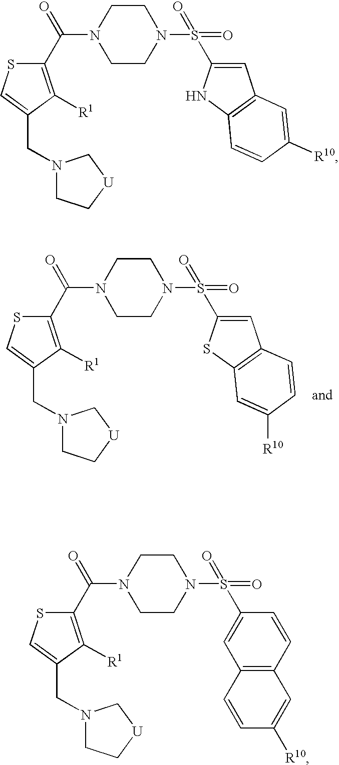
wherein
R¹⁰ is —Cl or —Br;
R^{2a} is H, —CH₃, —C₂H₅, —C₃H₇, —C₃H₅, —C₃H₃, —CH₂CH₂OCH₃, —CH₂CH₂N(CH₃)₂, —CH₂CH₂CH₂N(CH₃)₂, —CH₂CH₂CN, —CH₂CH₂CO₂H, —CH₂C₆H₅, —CH₂CH₂C₆H₅;
R¹, R^{1′}, and R^{1″} are independently H, —CH₃, —C₂H₅, —CH₂OH, —CH₂NH₂, —OH, —OCH₃, —OC₂H₅, —OC₃H₇, —SH, —SCH₃, —SC₂H₅, —SC₃H₇, —SOCH₃, —SO₂CH₃, —SOC₂H₅, —SO₂C₂H₅, —SOC₃H₇, —SO₂C₃H₇, —CN, —CO₂H, —CONH₂, —F or —Cl; and
U is a direct link, —CH₂—, —CH₂CH₂—, —CH₂O—, —CH₂NH—, —CH₂N(CH₃)—, —CH(CO₂H)—CH₂—, —CH(CONH₂)—CH₂—, —CH(OH)CH₂ or —CH(CH₂OH)CH₂.

8. A compound of claim 1 of formula (I) having one of the following structures: 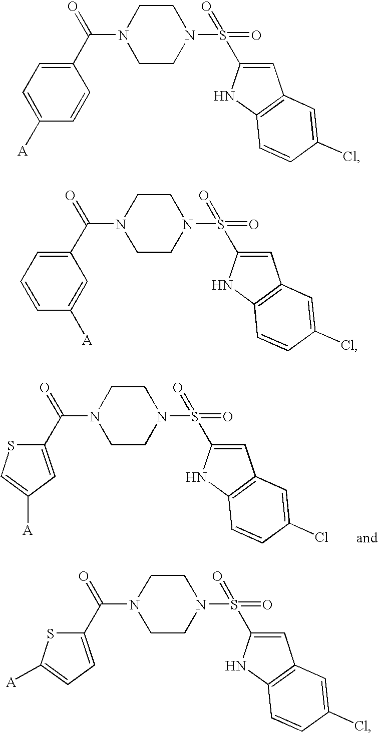
wherein:
A is selected from the group consisting of:

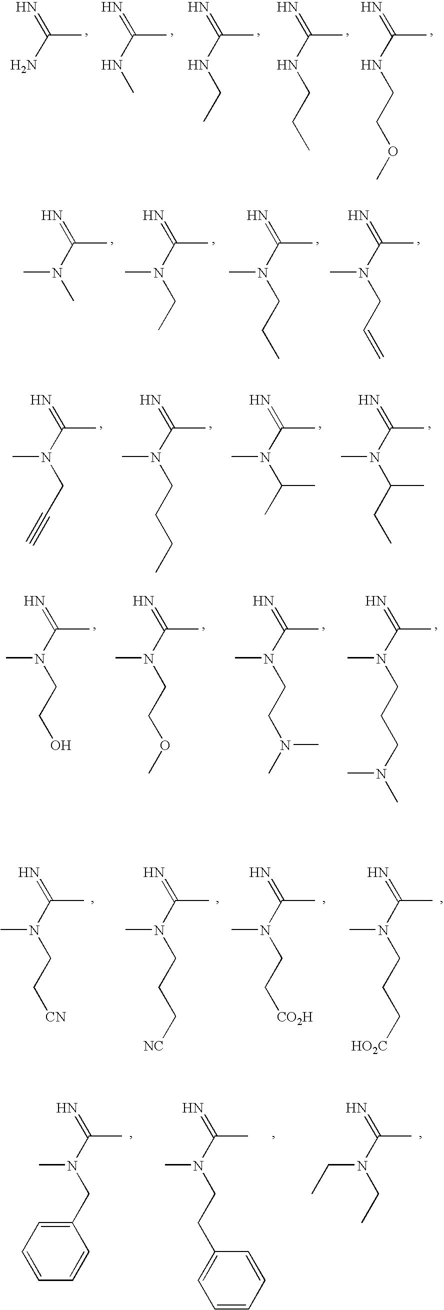

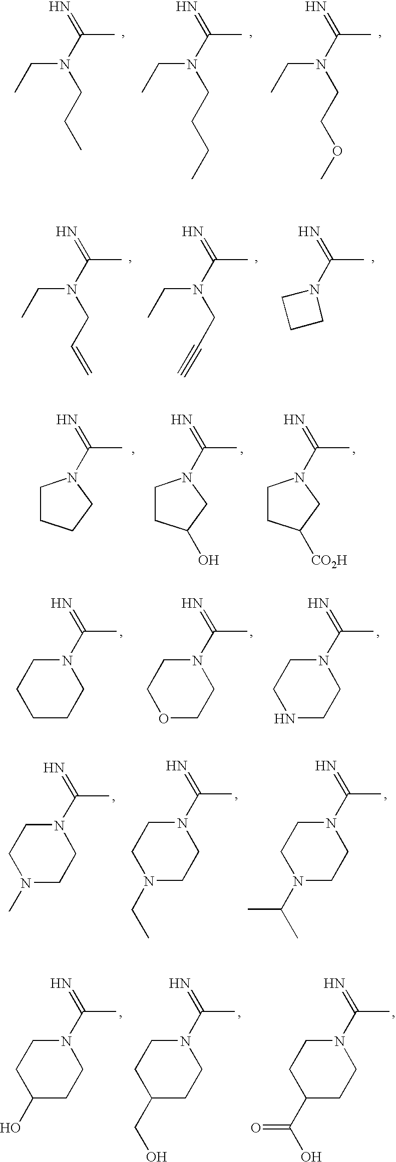

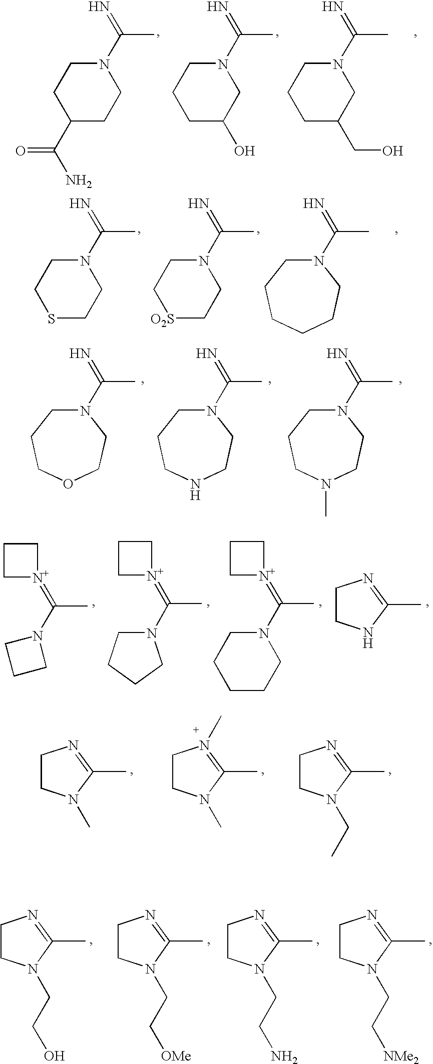

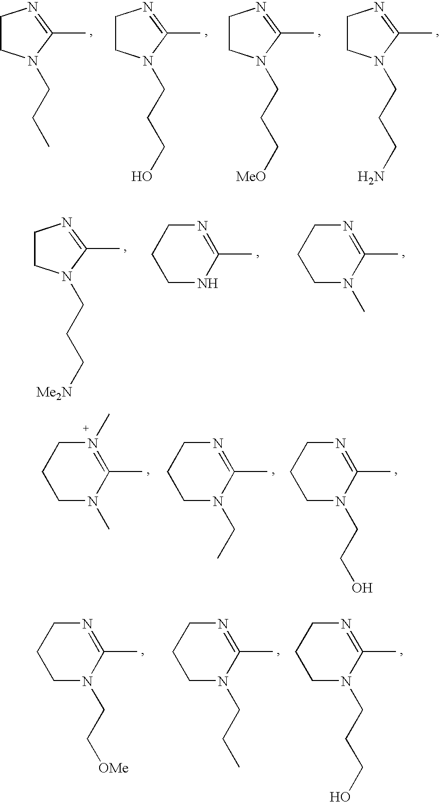

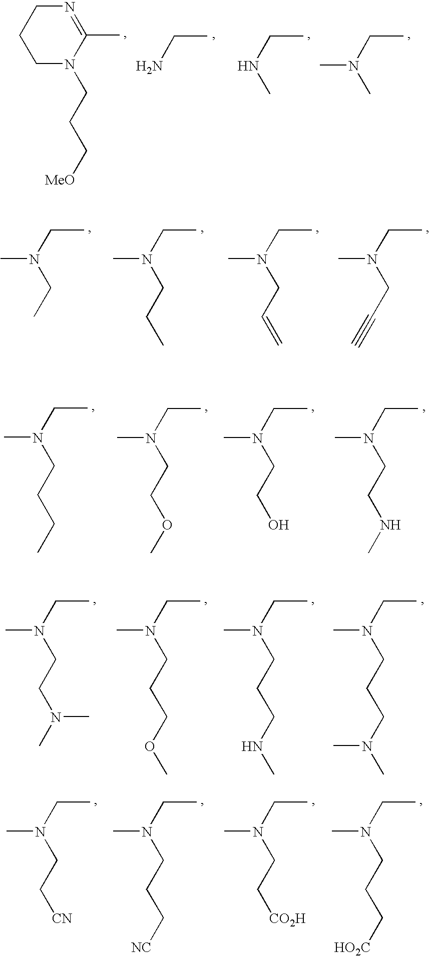

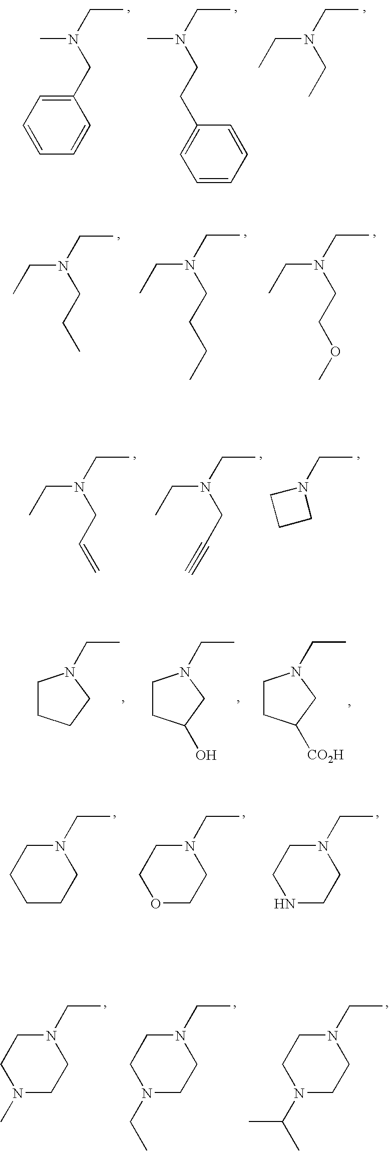

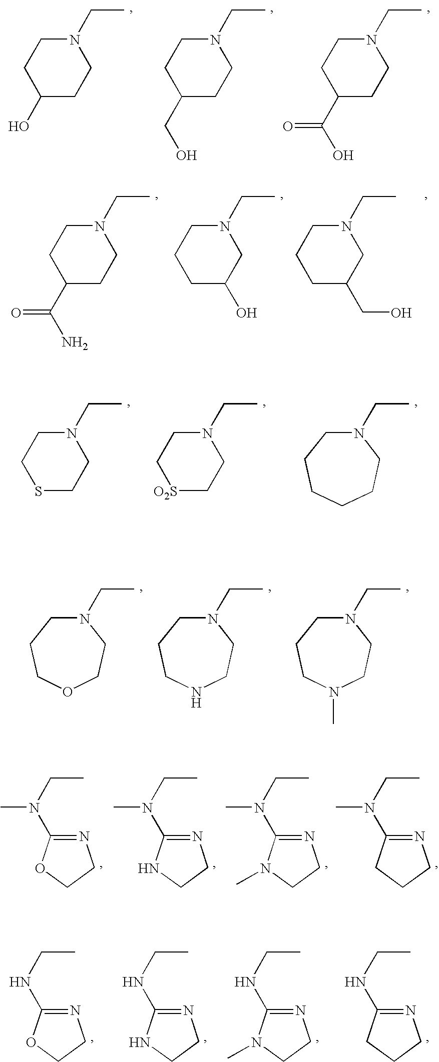

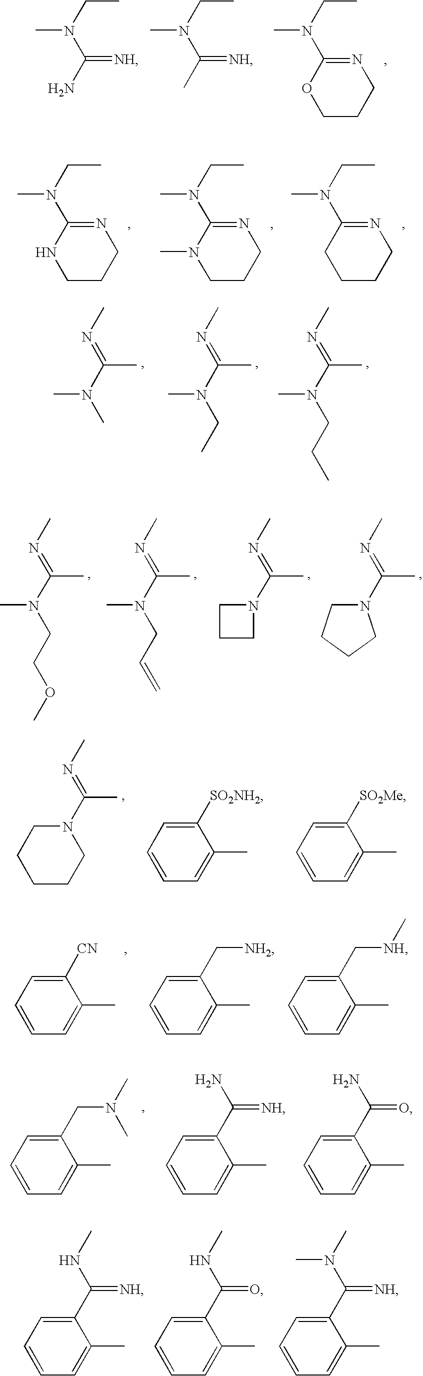

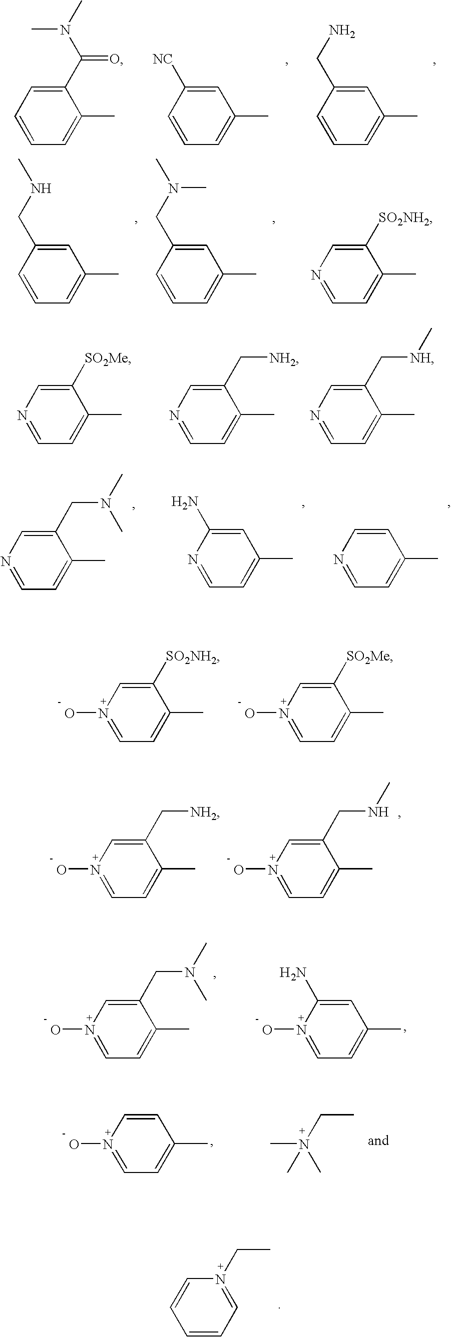

9. A compound of claim 1 of formula (I) having one of the following structures: 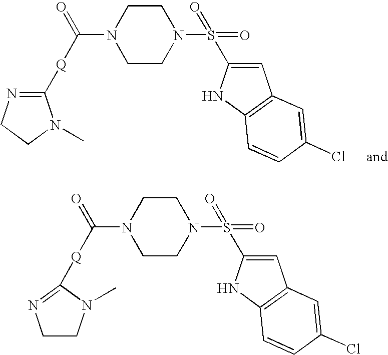
wherein:
Q is selected from the group consisting of:

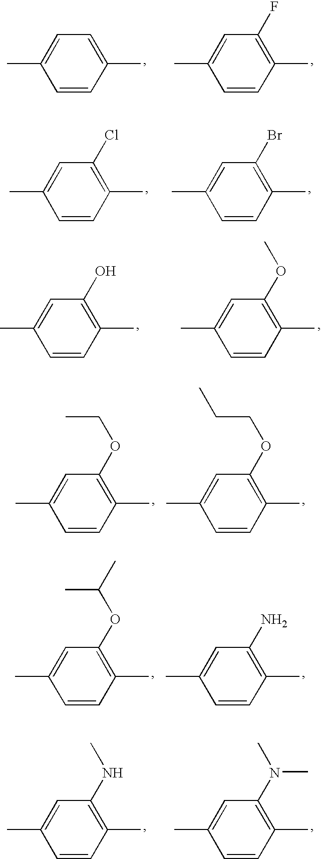

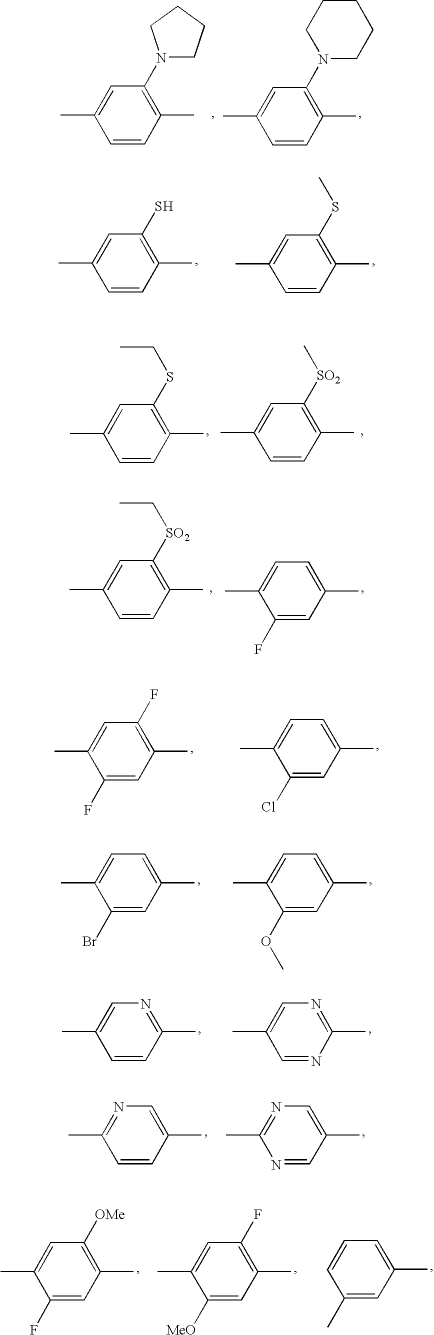

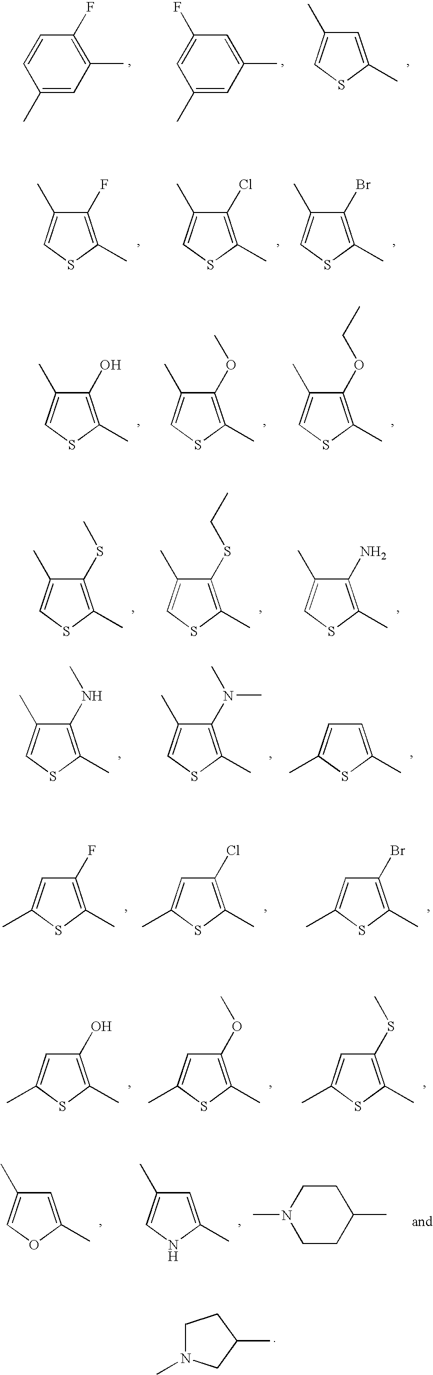

10. A compound of claim 1 of formula (I) having one of the following structures: 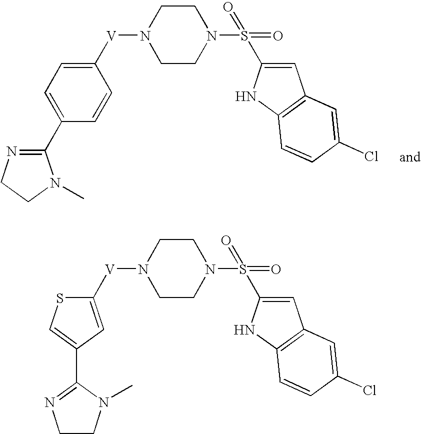
wherein V is —CH₂—, or —C(═O)—.

11. A compound of claim 1 of formula (I) having one of the following structures: 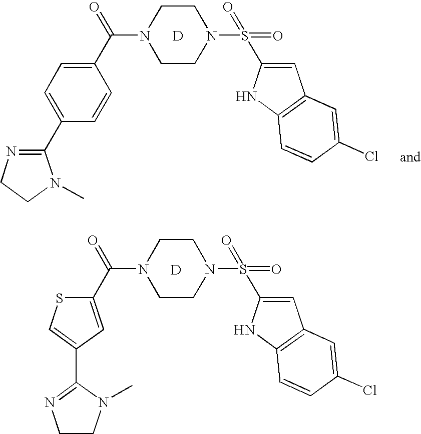
wherein:
D is selected from the group consisting of: 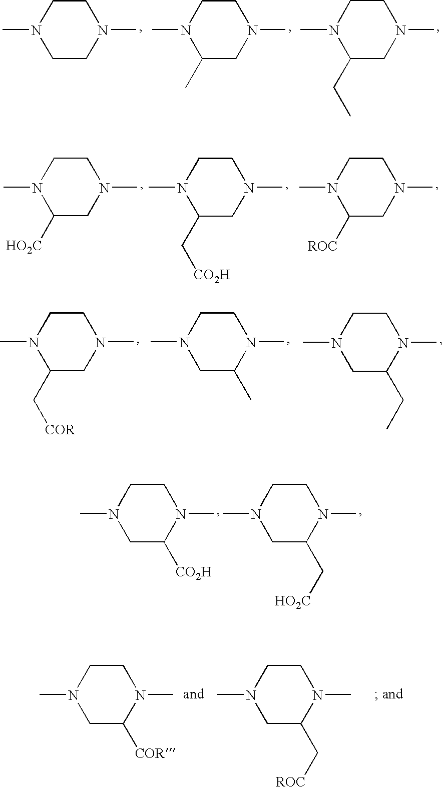
R′″ is selected from the group consisting of:

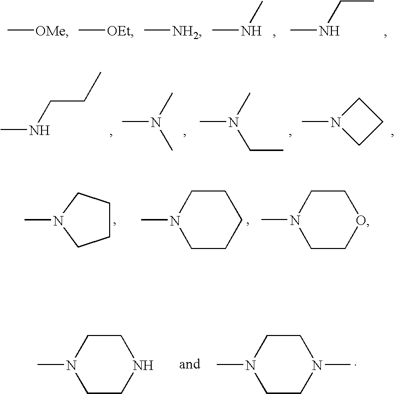

12. A compound of claim 1 of formula (I) having one of the following structures: 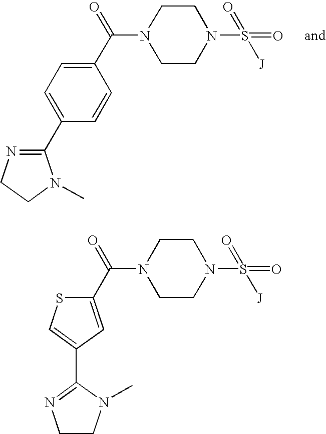
wherein:
J is selected from the group consisting of:

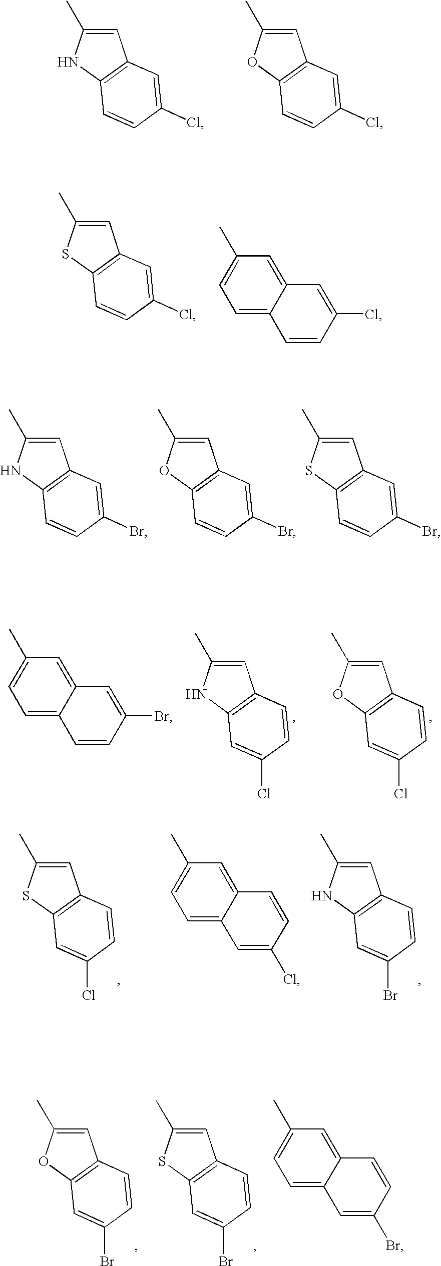

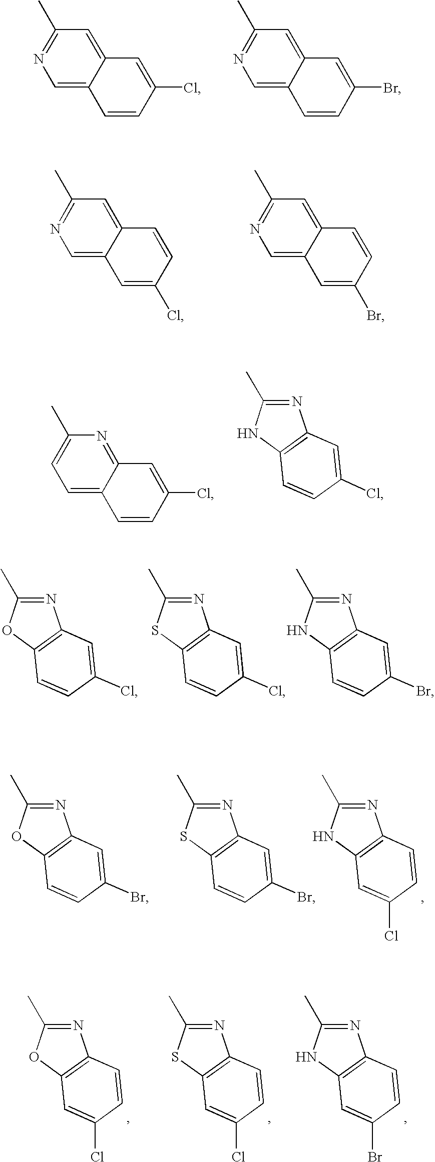

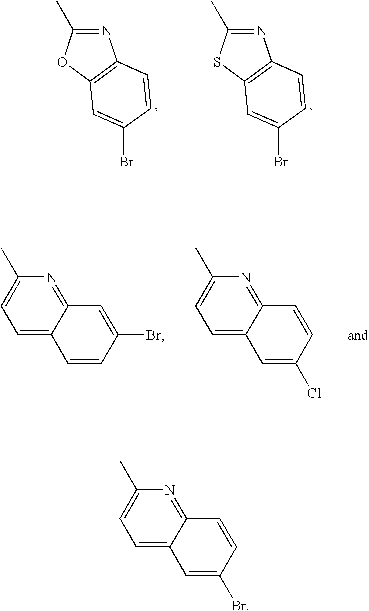

13. A pharmaceutical composition for preventing or treating a condition in a mammal characterized by undesired thrombosis comprising a pharmaceutically acceptable carrier and a pharmaceutically effective amount of a compound of one of claims 1-12.

14. A method for preventing or treating a condition in a mammal characterized by undesired thrombosis comprising administering to said manual a therapeutically effective amount of a compound of one of claims 1-12.

15. The method of claim 14, wherein the condition is selected from the group consisting of:
acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolus, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve, and conditions requiring the fitting of prosthetic devices.

16. A method for inhibiting the coagulation of biological samples comprising the administration of a compound of one of claims 1-12.
